(19) United States
(12) Patent Application Publication
(10) Pub. No.: US 2012/0034253 A1
(43) Pub. Date: Feb. 9, 2012

(54) Influenza Vaccines, Antigens, Compositions, and Methods

(75) Inventors: Vidadi Yusibov, Havertown, PA (US); Vadim Mett, Newark, DE (US); Konstantin Musiychuk, Wilmington, DE (US)

(73) Assignee: FRAUNHOFER USA, INC., Plymouth, MI (US)

(21) Appl. No.: 13/120,770

(22) PCT Filed: Sep. 25, 2009
(86) PCT No.: PCT/US2009/058488

§ 371 Date: Aug. 31, 2011

Related U.S. Application Data
(60) Provisional application No. 61/100,253, filed on Sep. 25, 2008.

(51) Int. Cl. A61K 39/145; A61P 37/04; A61P 31/16; C12P 21/02; C12N 9/24

(52) U.S. Cl. 424/186.1; 435/69.3; 435/200

(57) ABSTRACT

The present invention relates to the intersection of the fields of immunology and protein engineering, and particularly to antigens and compositions useful in inducing or enhancing an immune response agains influenza antigens. Provided are recombinant protein antigens, compositions, and methods for the production of such antigens in plants. In some embodiments, influenza antigens include hemagglutinin polypeptides, neuraminidase polypeptides, and/or combinations thereof.

### CROSS REFERENCE TO RELATED APPLICATIONS

This application claims priority to U.S. Provisional Application Ser. No. 61/100,253, filed on Sep. 25, 2008, which is hereby incorporated by reference in its entirety.

### BACKGROUND OF THE INVENTION

Influenza has a long history characterized by waves of pandemics, epidemics, resurgences and outbreaks. Influenza is a highly contagious disease that could be equally devastating both in developing and developed countries. The influenza virus presents one of the major threats to the human population. In spite of annual vaccination efforts, influenza infections result in substantial morbidity and mortality. Although flu epidemics occur nearly every year, fortunately pandemics do not occur very often. However, recent flu strains have emerged such that we are again faced with the potential of an influenza pandemic. Avian influenza virus of the type H5N1, currently causing an epidemic in poultry in Asia as well as regions of Eastern Europe, has persistently spread throughout the globe. The rapid spread of infection, as well as cross species transmission from birds to human subjects, increases the potential for outbreaks in human populations and the risk of a pandemic. The virus is highly pathogenic, resulting in a mortality rate of over fifty percent in birds as well as the few human cases which have been identified. If the virus were to achieve human to human transmission, it would have the potential to result in rapid, widespread illness and mortality.

### SUMMARY OF THE INVENTION

The present disclosure provides a method of making a composition that induces or enhances an immune response against an influenza polypeptide, wherein the polypeptide is a hemagglutinin polypeptide or an immunogenic portion thereof or a neuraminidase polypeptide or immunogenic portion thereof, the method comprising producing the influenza polypeptide in a plant. The method may further comprise isolating the polypeptide; and/or combining the polypeptide with a pharmaceutically acceptable carrier. In some embodiments, the influenza polypeptide is a hemagglutinin polypeptide. The hemaglutinin polypeptide may be a polypeptide having 80% or greater, for example 90% or greater, 95% or greater, 98% or greater, 99% or greater, sequence identity, to an amino acid sequence selected from the group consisting of SEQ ID NOs.: 1-35, 85, 87, 89, 91, 93, 95, 97, 99, 101, 103, 105, 107, 109, 111 and 112. The hemaglutinin polypeptide may be selected from the group consisting of SEQ ID NOs.: 1-35, 85, 87, 89, 91, 93, 95, 97, 99, 101, 103, 105, 107, 109, 111 and 112. In some embodiments, the influenza polypeptide is a neuraminidase polypeptide. The neuraminidase polypeptide may be a polypeptide having 80% or greater, for example 90% or greater, 95% or greater, 98% or greater, 99% or greater, sequence identity, to an amino acid sequence selected from the group consisting of SEQ ID NOs.: 36-43 and SEQ ID NO: 110. In some embodiments, the influenza polypeptide is a neuraminidase polypeptide selected from the group consisting of SEQ ID NOs.: 36-43 and SEQ ID NO: 110. In some embodiments, the plant may transiently express the polypeptide. In some embodiments, the transient expression may be from an Agrobacterial plasmid. In some embodiments, the transient expression may be from an plant viral vector. In some embodiments, the plant viral vector may be cloned into an Agrobacterial plasmid. In some embodiments, the plant may be transgenic for the polypeptide. In some embodiments, the composition may be combined with at least one vaccine adjuvant such as alum, Quil A, QS21, aluminum hydroxide, aluminum phosphate, mineral oil, MF59, Malp2, incomplete Freund's adjuvant, complete Freund's adjuvant, alhydrogel, 3 De-O-acylated monophosphoryl lipid A (3D-MPL), lipid A, Bortadella pertussis, Mycobacterium tuberculosis, Merck Adjuvant 65, squalene, virosomes, SBAS2, SBAS1, AS03 or unmethylated CpG sequences. Also provided are such influenza peptide compositions produced in plants, for example by the foregoing methods. In some embodiments, the method comprises isolating the influenza polypeptide. The isolated polypeptide may be at least about 70% pure, for example at least about 80%, at least about 90%, at least about 95%, at least about 95%, at least about 95% or at least about 99% pure. In some embodiments, the method comprises isolating the influenza polypeptide. The isolated polypeptide may be at least about 70% pure, for example at least about 80%, at least about 90%, at least about 95%, at least about 95%, at least about 95% or at least about 99% pure.

The plant may be from a genus selected from the group consisting of Brassica, Nicotiana, Petunia, Lycopersicon, Solanum, Capsium, Daucus, Apium, Lactuca, Sinapis or Arabidopsis, for example Nicotiana benthamiana, Brassica carinata, Brassica juncea, Brassica napus, Brassica nigra, Brassica oleraceae, Brassica tournifortii, Sinapis alba and Raphanus sativus. Plants that may be used include alfalfa, radish, mustard, mung bean, broccoli, watercress, soybean, wheat, sunflower, cabbage, clover, petunia, tomato, potato, tobacco, spinach, and lentil. In some embodiments the plant is a sprouted seedling

The present disclosure also provides a method of producing an influenza polypeptide, wherein the polypeptide is a hemagglutinin polypeptide or an immunogenic portion thereof or a neuraminidase polypeptide or immunogenic portion thereof, the method comprising providing a nucleic acid construct comprising a nucleic acid encoding an influenza polypeptide; and introducing the nucleic acid into a plant cell; and maintaining the cell under conditions permitting expression of the influenza polypeptide. The nucleic acid encoding the influenza polypeptide may be operably linked to a regulatory region such as a viral promoter or Agrobacterial vector. The nucleic acid construct may comprise one or more sequences encoding a plant viral protein. In some embodiments, the influenza polypeptide is a hemaglutinin polypeptide. The hemaglutinin polypeptide may be a polypeptide having 80% or greater, for example 90% or greater, 95% or greater, 98% or greater, 99% or greater, sequence identity, to an amino acid sequence selected from the group consisting of SEQ ID NOs.: 1-35, 85, 87, 89, 91, 93, 95, 97, 99, 101, 103, 105, 107, 109, 111 and 112. The hemaglutinin polypeptide may be selected from the group consisting of SEQ ID NOs.: 1-35, 85, 87, 89, 91, 93, 95, 97, 99, 101, 103, 105, 107, 109, 111 and 112. In some embodiments, the influenza polypeptide is a neuraminidase polypeptide. The neuraminidase polypeptide may be a polypeptide having 80% or greater, for example 90% or greater, 95% or greater, 98% or greater, 99% or greater, sequence identity, to an amino acid sequence selected from the group consisting of SEQ ID NOs.: 36-43 and SEQ ID NO: 110. In some embodiments, the influenza polypeptide is a neuraminidase polypeptide selected from the group consisting of SEQ ID NOs.: 36-43 and SEQ ID NO: 110. In some embodiments, the method comprises isolating the influenza polypeptide. The isolated polypeptide may be at least about 70% pure, for example at least about 80%, at least about 90%, at least about 95%, at least about 95%, at least about 95% or at least about 99% pure. The plant may be from a genus selected from the group consisting of Brassica, Nicotiana, Petunia, Lycopersicon, Solanum, Capsium, Daucus, Apium, Lactuca, Sinapis or Arabidopsis, for example Nicotiana benthamiana, Brassica carinata, Brassica juncea, Brassica napus, Brassica nigra, Brassica oleraceae, Brassica tournifortii, Sinapis alba and Raphanus sativus. Plants that may be used include alfalfa, radish, mustard, mung bean, broccoli, watercress, soybean, wheat, sunflower, cabbage, clover, petunia, tomato, potato, tobacco, spinach, and lentil. In some embodiments the plant is a sprouted seedling. Also provided is a plant cell produced by the foregoing methods and a plant containing such a plant cell.

Also provided is a method of inducing or enhancing an immune response against an influenza polypeptide in a subject, the method comprising administering a therapeutically effective amount of an influenza peptide or composition thereof prepared according to the foregoing methods. The peptide or composition thereof may be administered orally, intranasally, subcutaneously, intravenously, intraperitoneally, or intramuscularly. Also provided is a method of inducing or enhancing an immune response against an influenza polypeptide in a subject, by feeding a plant, or an edible portion thereof, or plant cell produced by the above-described to a subject. In these methods, the subject may be an animal, such as a bird, a pig or a horse, or a human A single dose of the composition may comprise up to about 200 μg, for example up to about 0.01, 0.1, 1, 5, 10, 25, 50, 75 or 100 μg of the influenza polypeptide.

The present disclosure also provides improved influenza antigens (e.g., influenza antigen polypeptides), compositions, vaccines, and dosing regimens. The present invention provides influenza antigen polypeptides, such as hemagglutinin polypeptides and/or neuraminidase polypeptides. The present invention provides subunit vaccines comprising at least one plant-produced influenza antigen polypeptide. Subunit vaccines in accordance with the present invention typically comprise at least one plant-produced influenza antigen polypeptide and a pharmaceutically acceptable excipient.

In some embodiments, the vaccine composition is immunogenic and/or protective when administered to a subject at relatively low doses.

In some embodiments, plant-produced influenza polypeptides for use in subunit vaccines are purified from plant materials. In some embodiments, plant-produced influenza polypeptides for use in subunit vaccines are not purified from plant materials.

The present invention provides methods for inducing a protective immune response against influenza infection in a subject comprising administering to a subject an effective amount of a vaccine composition comprising at least one plant-produced influenza antigen polypeptide.

The present invention provides methods and systems for producing influenza antigen polypeptides in plants. Such methods generally involve use of viral expression vectors. In some embodiments, such methods involve binary vectors, such as a “launch vector,” as described herein. In some embodiments, influenza antigen polypeptides are produced in young plants (e.g., sprouted seedlings). The present invention provides nucleic acid constructs useful for expressing influenza antigen polypeptides in plants, as well as host cells containing such nucleic acid constructs therein.

### DEFINITIONS

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Characteristic portion: As used herein, the phrase a “characteristic portion” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, at least 10, at least 15, at least 20 or more amino acids are required to be characteristic of a protein. In general, a characteristic portion is one that, in addition to the sequence identity specified above, shares at least one functional characteristic with the relevant intact protein.

Characteristic sequence: A “characteristic sequence” is a sequence that is found in all members of a family of polypeptides or nucleic acids, and therefore can be used by those of ordinary skill in the art to define members of the family.

Combination therapy: The term “combination therapy,” as used herein, refers to those situations in which two or more different pharmaceutical agents are administered in overlapping regimens so that the subject is simultaneously exposed to both agents.

Dosing regimen: A “dosing regimen,” as used herein, refers to a set of unit doses (typically more than one) that are administered individually separated by periods of time. The recommended set of doses (i.e., amounts, timing, route of administration, etc.) for a particular pharmaceutical agent constitutes its dosing regimen.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, and/or 3′ end formation); (3) translation of an RNA into a polypeptide or protein; (4) post-translational modification of a polypeptide or protein.

Gene: As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs. For the purpose of clarity we note that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a protein-coding nucleic acid.

Gene product: As used herein, the term “gene product” or “expression product” generally refers to an RNA transcribed from the gene (pre- and/or post-processing) or a polypeptide (pre- and/or post-modification) encoded by an RNA transcribed from the gene.

HA polypeptide: As used herein, the term “hemagglutinin polypeptide” or “HA polypeptide” refers to a polypeptide showing at least 50% overall sequence identity with one or more HA polypeptides listed in Table 1. In some embodiments, an HA polypeptide shows at least 60%, at least 70%, at least 80%, at least 85%, at least 95%, at least 96%, at least 97%, at least 98%, at least 99%, or 100% identity with a listed HA polypeptide. In some embodiments, an HA polypeptide further shares at least one characteristic sequence element with the listed HA polypeptides.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% similar.

Identity: As used herein, the term “identity” refers to the overall relatedness between polymeric molecules, e.g. between nucleic acid molecules (e.g. DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. As used herein, the term “overall identity” refers to identity over a long stretch of sequence. In some embodiments, overall identity refers to identity over at least 50, 60, 70, 80, 90, 100, 150, 200, 250, 300, 400, 500, or more amino acids and/or nucleotides. In some embodiments, overall identity refers to identity over the complete length of a given sequence.

Initiation: As used herein, the term “initiation” when applied to a dosing regimen can be used to refer to a first administration of a pharmaceutical agent to a subject who has not previously received the pharmaceutical agent. Alternatively or additionally, the term “initiation” can be used to refer to administration of a particular unit dose of a pharmaceutical agent during therapy of a patient.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Lichenase polypeptide: As used herein, the term “lichenase polypeptide” refers to a polypeptide showing at least 50% overall sequence identity with one or more lichenase polypeptides listed in Table 3. In some embodiments, a lichenase polypeptide shows at least 60%, at least 70%, at least 80%, at least 85%, at least 95%, at least 96%, at least 97%, at least 98%, at least 99%, or 100% identity with a listed lichenase polypeptide. In some embodiments, a lichenase polypeptide further shares at least one characteristic sequence element with the listed lichenase polypeptides.

Low dose: The term “low dose,” as used herein in reference to subunit vaccines, refers to a dosage amount of less than 100 μg of plant-produced antigen (e.g., influenza antigen polypeptide, fusion thereof, and/or immunogenic portion thereof) and/or vaccine composition comprising plant-produced antigen. In some embodiments, a low dose refers to a dosage amount of less than about 90 μg, less than about 80 μg, less than about 70 μg, less than about 60 μg, less than about 50 μg, less than about 40 μg, less than about 30 μg, less than about 25 μg, less than about 20 μg, less than about 15 μg, less than about 5 μg, less than about 4 μg, less than about 3 μg, less than about 2 μg, or less than about 1 μg of plant-produced antigen (e.g., influenza antigen polypeptide, fusion thereof, and/or immunogenic portion thereof) and/or vaccine composition comprising plant-produced antigen.

NA polypeptide: As used herein, the term “neuraminidase polypeptide” or “NA polypeptide” refers to a polypeptide showing at least 50% overall sequence identity with one or more NA polypeptides listed in Table 2. In some embodiments, an NA polypeptide shows at least 60%, at least 70%, at least 80%, at least 85%, at least 95%, at least 96%, at least 97%, at least 98%, at least 99%, or 100% identity with a listed NA polypeptide. In some embodiments, an NA polypeptide further shares at least one characteristic sequence element with the listed NA polypeptides.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more than 10 residues. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention may be specifically directed to “unmodified nucleic acids,” meaning nucleic acids (e.g. polynucleotides and residues, including nucleotides and/or nucleosides) that have not been chemically modified in order to facilitate or achieve delivery.

Operably linked: As used herein, the term “operably linked” refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. A nucleic acid sequence that is operably linked to a second nucleic acid sequence may be covalently linked, either directly or indirectly, to such a sequence, although any effective three-dimensional association is acceptable.

Pharmaceutical agent: As used herein, the phrase “pharmaceutical agent” refers to any agent that, when administered to a subject, has a therapeutic effect and/or elicits a desired biological and/or pharmacological effect.

Pharmaceutically acceptable carrier or excipient: As used herein, the term “pharmaceutically acceptable carrier or excipient” means a non-toxic, inert solid, semi-solid or liquid filler, diluent, encapsulating material or formulation auxiliary of any type.

Portion: As used herein, the phrase a “portion” or “fragment” of a substance, in the broadest sense, is one that shares some degree of sequence and/or structural identity and/or at least one functional characteristic with the relevant intact substance. For example, a “portion” of a protein or polypeptide is one that contains a continuous stretch of amino acids, or a collection of continuous stretches of amino acids, that together are characteristic of a protein or polypeptide. Each such continuous stretch generally will contain at least 5, at least 10, at least 15, at least 20 or more amino acids. In general, a portion is one that, in addition to the sequence identity specified above, shares at least one functional characteristic with the relevant intact protein. In some embodiments, the portion may be biologically active.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain amino acids, D-amino acids, or both and may contain any of a variety of amino acid modifications or analogs known in the art. Useful modifications include, e.g., terminal acetylation, amidation, etc. In some embodiments, proteins may comprise natural amino acids, non-natural amino acids, synthetic amino acids, and combinations thereof. The term “peptide” is generally used to refer to a polypeptide having a length of less than about 100 amino acids.

Similarity: As used herein, the term “similarity” refers to the overall relatedness between polymeric molecules, e.g. between nucleic acid molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Subject: As used herein, the term “subject” or “patient” refers to any organism to which compositions in accordance with the invention may be administered, e.g., for experimental, diagnostic, prophylactic, and/or therapeutic purposes. Typical subjects include animals (e.g., mammals such as mice, rats, rabbits, non-human primates, and humans; insects; worms; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Subunit vaccine: As used herein, a “subunit vaccine” refers to a vaccine composition comprising purified antigens rather than whole organisms. In some embodiments, subunit vaccines comprise an antigen that has been at least partially purified from non-antigenic components. In some embodiments, a subunit vaccine is at least 30%, at least 40%, at least 50%, at least 60%, at least 70%, at least 80%, at least 90%, at least 95%, at least 98%, or at least 99% pure. In some embodiments, subunit vaccines comprise an antigen that has not been at least partially purified from non-antigenic components. In some embodiments, subunit vaccines comprise exactly one antigen. In some embodiments, subunit vaccines comprise two or more (e.g., 2, 3, 4, 5, 6, 7, 8, 9, 10, 15, or more) antigens. In some embodiments, subunit vaccines are administered to a subject at low doses.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition. In some embodiments, an individual who is susceptible to a disease, disorder, and/or condition is an individual having higher risk (typically based on genetic predisposition, environmental factors, personal history, or combinations thereof) of developing a particular disease or disorder, or symptoms thereof, than is observed in the general population.

Therapeutically effective amount: The term “therapeutically effective amount” of a pharmaceutical agent or combination of agents is intended to refer to an amount of agent(s) which confers a therapeutic effect on the treated subject, at a reasonable benefit/risk ratio applicable to any medical treatment. In some embodiments, a therapeutically effective amount is an amount that is sufficient, when administered to a subject suffering from or susceptible to a disease, disorder, and/or condition, to treat, diagnose, prevent, and/or delay the onset of the symptom(s) of the disease, disorder, and/or condition. The therapeutic effect may be objective (i.e., measurable by some test or marker) or subjective (i.e., subject gives an indication of or feels an effect). A therapeutically effective amount is commonly administered in a dosing regimen that may comprise multiple unit doses. For any particular pharmaceutical agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific pharmaceutical agent employed; the duration of the treatment; and like factors as is well known in the medical arts.

Therapeutic agent: As used herein, the phrase “therapeutic agent” refers to any agent that, when administered to a subject, has a therapeutic effect and/or elicits a desired biological and/or pharmacological effect.

Treatment: As used herein, the term “treatment” (also “treat” or “treating”) refers to any administration of a biologically active agent that partially or completely alleviates, ameliorates, relives, inhibits, delays onset of prevents, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

Unit dose: The term “unit dose,” as used herein, refers to a discrete administration of a pharmaceutical agent, typically in the context of a dosing regiment.

Vector: As used herein, “vector” refers to a nucleic acid molecule which can transport another nucleic acid to which it has been linked. In some embodiments, vectors can achieve extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

### BRIEF DESCRIPTION OF THE DRAWING

FIG. 1. Schematic of hemagglutinin (HA) protein and protein domains. Domains 1, 2, and 2, 1 fold together to form a stem domain (SD). Domain 3 is a globular domain (GD). The ranges presented in items 1-6 correspond to amino acid positions of HA.

FIG. 2. Strategy for production of antigens in plants. Antigens were cloned into the “launch vector” system. Launch vectors were then introduced into Agrobacterium and vacuum infiltrated into plants. Antigens were allowed to express and accumulate in the plant biomass. Recombinant HA antigens were purified from the plant biomass.

FIG. 3. Expression data for plant-produced H5HA. (A) Exemplary expression data for four different constructs expressing H5 HA and NA (full-length except lacking the transmembrane anchor) from four different strains (i.e., HA antigens from A/Anhui/1/2005, “H5HA-A” or “HAA”; A/Indonesia/5/05, “H5HA-I” or “HAI”; A/Bar-headed goose/Qinghai/1A/2005, “H5HA-Q” or “HAQ”; and A/Vietnam/04, “H5HA-V” or “HAV”; and also corresponding NA antigens from the same four strains). (B) Exemplary expression data for several different pandemic and seasonal influenza strains.

FIG. 4. Antigenicity for each of HAA, HAI, HAQ, and HAV produced in plants. This demonstrates the antigenicity of the plant-produced antigens shown in FIG. 3A using an ELISA assay. This assay was performed by coating 96 well plates with 1 μg/ml of each H5HA protein. Antigens were then detected using a 1:6000 dilution of either anti-A/Anhui/01105 ferret sera, anti-A/Indonesia/05/2005 ferret sera, anti-A/Vietnam/1194/04 HA sheep anti-sera, or anti-A/Wyoming/03/2003 HA sheep anti-sera. All plant-produced H5HAs showed specific reactivity with anti-serum raised against homologous H5HA, but not against anti-serum generated against A/Wyoming/03/03 an H3 virus.

FIG. 5. Expression of HAA and HAQ. Coomassie gels (left panel) and western blots (right panel) of H5HA-A and H5HA-Q expressed in and purified from plants. Western blots were performed using anti-His antibodies.

FIG. 6. Immunization schedule. Groups of 8 week old female Balb/c mice were immunized subcutaneously with H5HA-Q or H5HA-A in the presence of 10 μg Quil A. Immunizations were administered at days 0, 14, and 28.

FIG. 7. Serum hemagglutination-inhibition and virus neutralization antibody titers. Serum from mice immunized with A/Anhui/01/05 or A/Bar-headedgoose/Qinghai/1A/05 HA produced in plants demonstrated significant hemagglutination inhibition (A) and virus neutralizing (B) antibody titers, even when mice were immunized with doses of antigen as low as 5 μg.

FIG. 8. Serum HI antibody titers resulting from immunization with as low as I yg antigen. Mice were immunized with antigen doses as low as 2.5 μg and 1 μg of HAA. Plant-produced HA elicits high titers of HI with doses as low as 1 μg.

FIG. 9. In vitro characterization of ppH3HAwy. (A) SDS-PAGE followed by western blot analysis of purified ppH3HAwy (lane 3) and iA/Wyo (lane 2). Lane 1 is a molecular weight marker. (B) ELISA analysis of ppH3HAwy with reference sheep anti-H3 HA or anti-N2 NA. Data are shown as mean OD values ±standard deviation at 1:1600 dilutions of sheep anti-H3 (gray bar) and anti-N2NA (open bar) serum. (C) Quantification and analysis of ppH3HAwy by single radial immuno-diffusion (SKID). iA/Wyo was used as a reference antigen.

FIG. 10. ELISA analysis of influenza-specific antibody responses induced by ppH3HAwy. IgG titers are shown for groups of mice that received 30 μg, 10 μg, and 5 μg dose of antigen. Data are shown as mean serum IgG titers ±standard deviations.

FIG. 11. ELISA analysis of IgG subtypes in mice sera and ELISPOT analysis of IFNγ or IL-5 secretion by splenocytes collected from ppH3HAwy-immunized mice. IgG subtype responses were measured in sera collected on day 42 from animals immunized with 5 μg dose of antigen. Data are shown as mean serum IgG subtype titers ±standard deviations (A). The frequency of IFNγ or IL-5 secreting spleen cells of iA/Wyo-immunized mice (B) or ppH3HAwy-immunized mice (C) are shown as the average number of spot-forming cells (SFC)/10⁶ cells ±standard deviations.

FIG. 12. Serum hemagglutination-inhibition and virus neutralization antibody titers. HI titers (A) and VN titers (B) are shown for groups of mice that received 30 μg, 10 μg, and 5 μg dose of antigen. Samples of sera were collected on days 0, 28, and 42. iA/Wyo was used as control. HI titers are expressed as the reciprocal of the highest dilution of serum that inhibited the hemagglutination of 8 hemagglutinin units of virus. VN titers are expressed as the reciprocal of the highest dilution of serum that gave 50% neutralization of 2×10³ TCID50 of virus. Samples without detectable HI or VN titer were assigned a titer of 5 or 10. Data are shown as mean titer ±standard deviations.

FIG. 13. Schematic of timeline of production of proteins in plants.

FIG. 14. In vitro characterization of plant-produced H5HA-I. (A) Coomassie brilliant blue and (B) western blot of expressed H5HA-I using anti-His antibodies. (C) ELISA analysis of H5HA-I with reference ferret sera against A/Indonesia/05/05 or sheep reference sera against A/Wyoming/03/03. Data are shown as mean OD values ±standard deviations.

FIG. 15. Immunogenicity and protective efficacy of plant-produced H5HA-I. (A) Serum from mice immunized with A/Indonesia/05/05 HA produced in plants demonstrated significant hemagglutination inhibition activity, even when mice were immunized with doses of antigen as low as 15 μg. (B) Serum from mice immunized with A/Indonesia/05/05 HA produced in plants demonstrated significant virus neutralization activity, even when mice were immunized with doses of antigen as low as 5 μg.

FIG. 16. Immunogenicity and protective efficacy of plant-produced H5HA-I. (A) Serum from ferrets immunized with A/Indonesia/05/05 HA produced in plants demonstrated significant hemagglutination inhibition activity. (B) Percent survival of ferrets after challenge. (C) Percent weight change of ferrets at 8 days post-challenge. (D) Viral titers in ferret nasal washes at 4 days post challenge.

FIG. 17. Production of HA antigens in plants. (A) Coomassie brilliant blue staining and western blots of produced HAB1-H3 and HAB1-H1 proteins. Total protein expression for each construct was about 800 mg/kg plant biomass. Western blots were performed using an anti-H3N2 polyclonal or an anti-His monoclonal antibody, as indicated. For A/Brisbane/10e/2007, 2 μl, 5 μl, or 10 μl of final product was loaded on each gel. For A/Brisbane/59/07, Coomassie-stained gel was loaded as follows: Lane 1: molecular weight marker; Lane 2: 0.5 μg BSA; Lane 3: 1.0 μg BSA; Lane 4: 2.5 μg BSA; Lane 5: 0.5 μl final product; Lane 6: 1.0 μl final product; Lane 7: 2.0 μl final product; Lane 8: 5.0 μl final product. For A/Brisbane/59/07, gel for western blot was loaded as follows: Lane 1: 200 ng Lic-LF (fusion of lichenase and anthrax lethal factor proteins); Lane 2: 100 ng Lic-LF; Lane 3: 50 ng Lic-LF; Lane 4: molecular weight marker; Lanes 5-7: 1.0 μl soluble extract (3 independent soluble extract samples in 1×PBS, 10 mM Dieca, and 0.1% Triton). (B) Coomassie brilliant blue staining and/or western blots of produced HAB1-B and HAF1-B proteins. Western blots were performed using anti-His antibodies. S: Protein extracted in buffer comprising 1×PBS and 10 mM EDTA. P: Extraction of remaining protein after S fraction, taken by resuspending pellet in 2×SDS-SB and boiling. Total protein expression for HAB1-B was about 800 mg/kg plant biomass. For B/Florida/4/2006, Coomassie-stained gel was loaded as follows: Lane 1: molecular weight marker; Lane 2: material before loading onto Q-column; Lane 3: 2.5 μl of 1:10 dilution of final product; Lane 4: 1 μl of 1:10 dilution of final product; Lane 5: 3 μl of 1:10 dilution of final product; Lane 6: 5 μl of 1:10 dilution of final product; Lane 7: blank; Lane 8: 0.3 μg BSA; Lane 9: 0.5 μg BSA; Lane 10:1.0 μg BSA; and Lane 11: 1.5 μg BSA. For B/Florida/4/2006, gel for western blot was loaded as follows: Lane 1: molecular weight marker; Lane 2: total protein, 15 μl of a 1:10 dilution; Lane 3: total soluble protein, 15 μl of a 1:10 dilution; Lane 4: flow-through from Nickel column, 15 μl of a 1:10 dilution; Lane 5: elution from Nickel column, 0.75 p. 1 of a 1:10 dilution; Lane 6: material before loading onto Q-column, 0.975 μl of a 1:10 dilution; Lane 7: 1 μl of a 1:50 dilution of final product; Lane 8: 2 μl of a 1:50 dilution of final product; and Lane 9: 3 μl of a 1:50 dilution of final product. Total protein expression for HAF1-B was about 325 mg/kg plant biomass.

FIG. 18. Immunization schedule. Mice were immunized with 60 μg, 30 μg, or 15 μg of plant-produced HA from A/Brisbane/59/07 (HAB1-H1) or A/Brisbane/10e/07 (HAB1-H3).

FIG. 19. Immunogenicity of plant-produced HAB1-HI. Serum titers of HA-specific antibodies were determined by ELISA following prime, 1st boost, and 2nd boost of HAB 1-HI antigen. Data are represented as mean antibody titer ±standard deviation.

FIG. 20. Serum hemagglutination-inhibition antibody titers elicited by plant-produced HAB1-HI. HI antibody titers are shown for groups of mice that received 60 μg, 30 μg, or 15 μg dose of antigen. Serum samples were collected on days 0, 28, and 42. HI titers were measured against homologous A/Brisbane/59/07 virus.

FIG. 21. Immunogenicity of plant-produced HAB1-H3. Serum titers of HA-specific antibodies were determined by ELISA following prime, 1st boost, and 2nd boost of HAB1-H3 antigen. Data are represented as mean antibody titer ±standard deviation.

FIG. 22. Serum hemagglutination-inhibition antibody titers elicited by plant-produced HAB1-H3. HI antibody titers are shown for groups of mice that received 60 μg, 30 μg, or 15 μg dose of antigen. Serum samples were collected on days 0, 28, and 42. HI titers were measured against homologous A/Brisbane/10e/07 virus.

FIG. 23. Table of Engineered and Expressed HAs.

FIG. 24. Table of Engineered and Expressed NAs.

FIG. 25. Serum hemagglutination-inhibition and antibody titers elicited by plant-produced HA from A/New Calcdonia/20/99 (H1N1)-HANC3. FIG. 25A depicts HA-specific antibody responses. FIG. 25B depicts hemagglutinin-inhibition activity.

FIG. 26. Serum hemagglutination-inhibition and antibody titers elicited by plant-produced HA from A/Solomon Islands/3/06 HASH. FIG. 26A depicts HA-specific antibody responses. FIG. 26B depicts hemagglutinin-inhibition activity.

FIG. 27. Serum hemagglutination-inhibition and antibody titers elicited by plant-produced HA from A/Brisbane/59/07 (H1N1)-HAB1(H1). FIG. 27A depicts HA-specific antibody responses. FIG. 27B depicts hemagglutinin-inhibition activity. FIG. 27C depicts the results of an SRID assay.

FIG. 28. Serum hemagglutination-inhibition and antibody titers elicited by plant-produced HA from A/Wyoming/03/03 (H3N2)-HAWY1. FIG. 28A depicts the results of an SRID assay. FIG. 28B is a graphical representation of the SRID assay. FIG. 28C depicts HA-specific antibody responses. FIG. 28B depicts hemagglutinin-inhibition activity.

FIG. 29. Serum hemagglutination-inhibition and antibody titers elicited by plant-produced HA from A/Wisconsin/67/05 (H3N2)-HAWI1. FIG. 29A depicts HA-specific antibody responses. FIG. 29B depicts hemagglutinin-inhibition activity.

FIG. 30. Serum hemagglutination-inhibition and antibody titers elicited by plant-produced HA from A/Brisbane/10/07 (H3N2)-HAB1(H3). FIG. 30A depicts HA-specific antibody responses. FIG. 30B depicts hemagglutinin-inhibition activity. FIG. 30C depicts the results of an SRID assay.

FIG. 31. Serum hemagglutination-inhibition and antibody titers elicited by plant-produced HA from A/Brisbane/3/07 HAB1(B). FIG. 31A depicts HA-specific antibody responses. FIG. 31B depicts hemagglutinin-inhibition activity.

FIG. 32. Serum hemagglutination-inhibition and antibody titers elicited by plant-produced HA from B/Florida/4/06 HAF1. FIG. 32A depicts HA-specific antibody responses. FIG. 32B depicts hemagglutinin-inhibition activity.

FIG. 33. Immunogenicity and protective efficacy of plant-produced HA and NA. FIG. 33A depicts HA-specific antibody responses. FIG. 33B depicts hemagglutinin-inhibition activity.

FIG. 34. Serum hemagglutination-inhibition and antibody titers elicited by plant-produced HA from A/Anhui/1/05 (H5N1 Glade 2.3)-HAA1. FIG. 34A depicts HA-specific antibody responses. FIG. 34B depicts hemagglutinin-inhibition activity.

FIG. 35. Serum hemagglutination-inhibition and antibody titers elicited by plant-produced HA from A/Indonesia/5/05 (H5N1 Glade 2.1)-HAI1. FIG. 35A depicts HA-specific antibody responses. FIG. 35B depicts hemagglutinin-inhibition activity. FIG. 35C depicts the results of an SRID assay.

FIG. 36. Serum hemagglutination-inhibition and antibody titers elicited by plant-produced HA from A/B-H G/Qinghai (H5N1 Glade 2.2)-HAQ1. FIG. 36A depicts HA-specific antibody responses. FIG. 36B depicts hemagglutinin-inhibition activity.

FIG. 37. Serum hemagglutination-inhibition and antibody titers elicited by plant-produced HA from A/Viet Nam/1194/04 (H5N1 Glade 2.2)-HAV1R. FIG. 37A depicts HA-specific antibody responses. FIG. 37B depicts hemagglutinin-inhibition activity.

FIG. 38. Serum hemagglutination-inhibition and antibody titers elicited by plant-produced HA from A/Netherlands/219/03 (H7N7)-HANL1. FIG. 38A depicts HA-specific antibody responses. FIG. 38B depicts hemagglutinin-inhibition activity.

FIG. 39. Dose Reduction Study with HAA1. FIG. 39A depicts HA-specific antibody responses. FIG. 39B depicts hemagglutinin-inhibition activity.

FIG. 40. Effect of Quil A adjuvant on immunogenicity of plant-produced HA from A/Anhui/1/05. FIG. 40A depicts HA-specific antibody responses. FIG. 40B depicts hemagglutinin inhibition activity.

FIG. 41. Effect of Quil A adjuvant on immunogenicity of plant-produced HA from A/Indonesia/5/05. FIG. 41A depicts HA-specific antibody responses. FIG. 41B depicts hemagglutinin-inhibition activity.

FIG. 42A shows the effect of Alhydrogel adjuvant on immunogenicity of plant-produced HAI1. FIG. 42B shows the effect of Alhydrogel adjuvant on immunogenicity of plant-produced HAB (H1); FIG. 42C shows the effect of Alhydrogel adjuvant on immunogenicity of plant-produced HAC1 (04).

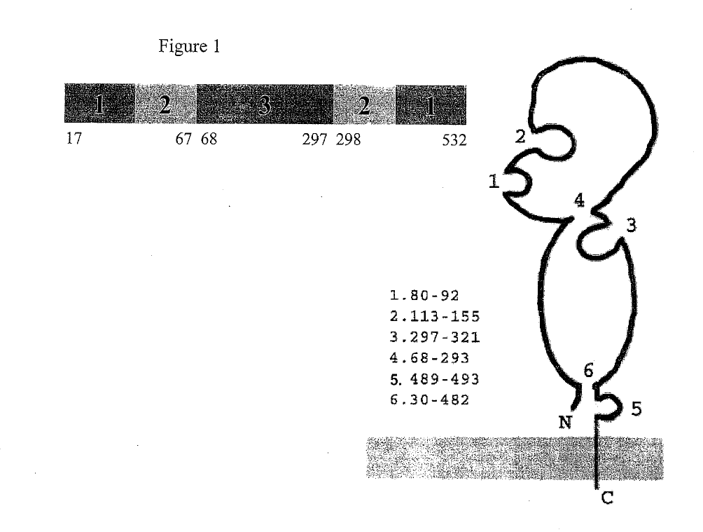

### DETAILED DESCRIPTION OF CERTAIN EMBODIMENTS OF THE INVENTION INFLUENZA AND INFLUENZA THERAPIES

The major defense against influenza is vaccination. Influenza viruses are segmented, negative-strand RNA viruses belonging to the family Orthomyxoviridae. The viral antigens are highly effective immunogens, capable of eliciting both systemic and mucosal antibody responses. Influenza virus hemagglutinin glycoprotein (HA) is generally considered the most important viral antigen with regard to the stimulation of neutralizing antibodies and vaccine design. For some vaccine compositions, the presence of viral neuraminidase (NA) has been shown to be important for generating multi-arm protective immune responses against the virus. Antivirals which inhibit neuraminidase activity have been developed and may be an additional antiviral treatment upon infection. Additional components sometimes considered useful in the development of influenza antivirals and vaccines are the ion channel protein M2 and the matrix protein M1 protein.

Subtypes of the influenza virus are designated by different HA and NA resulting from antigenic shift. Furthermore, new strains of the same subtype result from antigenic drift, or mutations in the HA or NA molecules which generate new and different epitopes. Although 15 antigenic subtypes of HA have been documented, only three of these subtypes H1, H2, and H3, have circulated extensively in humans.

Vaccination has become paramount in the quest for improved quality of life in both industrialized and underdeveloped nations. The majority of available vaccines still follow the basic principles of mimicking aspects of infection in order to induce an immune response that could protect against the relevant infection. However, generation of attenuated viruses of various subtypes and combinations can be time consuming and expensive. Emerging new technologies, in-depth understanding of a pathogen's molecular biology, pathogenesis, and its interactions with an individual's immune system have resulted in new approaches to vaccine development and vaccine delivery. Thus, while technological advances have improved the ability to produce improved influenza antigen vaccine compositions, there remains a need to provide additional sources of vaccines and new antigens for production of vaccines to address emerging subtypes and strains. Improved vaccine design and development for influenza virus subtypes, as well as methods of making and using such compositions of matter are needed.

### Influenza Antigens

In general, influenza antigens can include any immunogenic polypeptide that elicits an immune response against influenza virus. According to the present invention, immunogenic polypeptides of interest can be provided as independent polypeptides, as fusion proteins, as modified polypeptides (e.g., containing additional pendant groups such as carbohydrate groups, methyl groups, alkyl groups [such as methyl groups, ethyl groups, etc.], phosphate groups, lipid groups, amide groups, formyl groups, biotinyl groups, heme groups, hydroxyl groups, iodo groups, isoprenyl groups, myristoyl groups, flavin groups, palmitoyl groups, sulfate group, polyethylene glycol, etc.). In some embodiments, influenza antigen polypeptides for use in accordance with the present invention have an amino acid sequence that is or includes a sequence identical to that of an influenza polypeptide found in nature; in some embodiments influenza antigen polypeptides have an amino acid sequence that is or includes a sequence identical to a characteristic portion (e.g., an immunogenic portion) of an influenza polypeptide found in nature.

In certain embodiments, full length proteins are utilized as influenza antigen polypeptides in vaccine compositions in accordance with the invention. In some embodiments one or more immunogenic portions of influenza polypeptides are used. In certain embodiments, two or three or more immunogenic portions are utilized, as one or more separate polypeptides or linked together in one or more fusion polypeptides.

Influenza antigen polypeptides for use in accordance with the present invention may include full-length influenza polypeptides, fusions thereof, and/or immunogenic portions thereof. Where portions of influenza proteins are utilized, whether alone or in fusion proteins, such portions retain immunological activity (e.g., cross-reactivity with anti-influenza antibodies). Based on their capacity to induce immunoprotective response against viral infection, hemagglutinin and neuraminidase are antigens of interest in generating vaccines. Additional antigens, such as the membrane ion channel M2 or the matrix protein M1, may be useful in production of vaccines (e.g., combination vaccines) in order to improve efficacy of immunoprotection.

Thus, the invention provides plant cells and plants expressing a heterologous protein (e.g., an influenza antigen polypeptide, such as an influenza protein or immunogenic portion thereof; or a fusion protein comprising an influenza protein or immunogenic portion thereof). A heterologous protein in accordance with the invention can comprise any influenza antigen polypeptide of interest, including, but not limited to hemagglutinin (HA), neuraminidase (NA), membrane ion channel M2 (M2), matrix protein M1 (M1), a portion of hemagglutinin (HA), a portion of neuraminidase (NA), a portion of membrane ion channel (M2), a portion of matrix protein M1 (M1), fusion proteins thereof, immunogenic portions thereof, or combinations of hemagglutinin (HA), neuraminidase (NA), membrane ion channel M2 (M2), matrix protein M1 (M1), a portion of hemagglutinin (HA), a portion of neuraminidase (NA), a portion of membrane ion channel (M2) and/or a portion of matrix protein M1 (M1).

Amino acid sequences of a variety of different influenza HA, NA, M2, and M1 proteins (e.g., from different subtypes, or strains or isolates) are known in the art and are available in public databases such as GenBank. Exemplary full length protein sequences for HA and NA of multiple influenza subtypes, strains, and/or clades are provided in Tables 1 and 2 below.

In certain embodiments, full length hemagglutinin (HA) is utilized in vaccine compositions in accordance with the invention. In some embodiments one or more domains of HA is used. In certain embodiments, two or three or more domains are utilized, as one or more separate polypeptides or linked together in one or more fusion polypeptides. Sequences of exemplary HA polypeptides are presented in Table 1.

TABLE 1
Exemplary HA Sequences

| GenBank | | |
|---|---|---|
| Accession | Strain | HA Sequence |
| ABY51347 | A/environment/ | 5′MNIQILAFIACVLTGAKGDKICLGHHAVANGTKVNTLTEK |
| | New | GIEVVNATETVETADVKKICTQGKRATDLGRCGLLGTLIGPP |
| | York/3181- | QCDQFLEFSSDLIIERREGTDVCYPGRFTNEESLRQILRRSGGI |
| | 1/2006 | GKESMGFTYSGIRTNGAASACTRSGSSFYAEMKWLLSNSDN |
| | (H7N2) | SAFPQMTKAYRNPRNKPALIIWGVHHSESASEQTKLYGSGN |
| | | KLITVRSSKYQQSFTPSPGTRRIDFHWLLLDPNDTVTFTFNG |
| | | AFIAPDRASFFRGESLGVQSDAPLDSSCRGDCFHSGGTIVSSL |
| | | PFQNINSRTVGRCPRYVKQKSLLLATGMRNVPEKPKPRGLF |
| | | GAIAGFIENGWEGLINGWYGFRHQNAQGEGTAADYKSTQS |
| | | AIDQITGKLNRLIGKTNQQFELIDNEFNEIEQQIGNVINWTRD |
| | | AMTEIWSYNAELLVAMENQHTIDLADSEMSKLYERVKKQL |
| | | RENAEEDGTGCFEIFHKCDDQCMESIRNNTYDHTQYRTESL |
| | | QNRIQIDPVKLSSGYKDIILWFSFGASCFILLAIAMGLVFICIK |
| | | NGNMQCTICI 3′ (SEQ ID NO: 1) |
| ACC61810 | A/environment/ | 5′MNTQILAFIACVLTGVKGDKICLGHHAVANGTKVNTLTE |
| | New | KGIEVVNATETVETADVKKICTQGKRATDLGRCGLLGTLIG |
| | York/3185- | PPQCDQFLEFSSDLIIERREGTDVCYPGRFTNEESLRQILRRSG |
| | 1/2006 | GIGKESMGFTYSGIRTNGATSACTRSGSSFYAEMKWLLSNS |
| | (H7N2) | DNSAFPQMTKAYRNPRNKPALIIWGVHHSESVSEQTKLYGS |
| | | GNKLITVRSSKYQQSFTPSPGARRIDFHWLLLDPNDTVTFTF |
| | | NGAFIAPDRASFFRGESLGVQSDVPLDSSCRGDCFHSGGTIV |
| | | SSLPFQNINSRTVGKCPRYVKQKSLLLATGMRNVPEKPKPR |
| | | GLFGAIAGFIENGWEGLINGWYGFRHQNAQGEGTAADYKS |
| | | TQSAIDQITGKLNRLIGKTNQQFELIDNEFNEIEQQIGNVINW |
| | | TRDAMTEIWSYNAELLVAMENQHTIDLADSEMSKLYERVK |
| | | KQLRENAEEDGTGCFEIFHKCDDQCMESIRNNTYDHTQYRT |
| | | ESLQNRIQIDPVKLSSGYKDIILWFSFGASCFLLLAIAMGLVFI |
| | | CIKNGNMQCTICI 3′ (SEQ ID NO: 2) |
| ABI26075 | A/guineafowl/ | 5′ |
| | NY/4649- | MNIQILAFIACVLTGAKGDKICLGHHAVANGTKVNTLTEKGI |
| | 18/2006 | EVVNATETVETANIKKICTQGKRPTDLGQCGLLGTLIGPPQC |
| | (H7N2) | DQFLEFSSDLIIERREGTDVCYPGKFTNEESLRQILRRSGGIG |
| | | KESMGFTYSGIRTNGATSACTRSGSSFYAEMKWLLSNSDNA |
| | | AFPQMTKSYRNPRNKPALIIWGVHHSESVSEQTKLYGSGNK |
| | | LIKVRSSKYQQSFTPNPGARRIDFHWLLLDPNDTVTFTFNGA |
| | | FIAPDRASFFRGESIGVQSDAPLDSSCGGNCFHNGGTIVSSLP |
| | | FQNINPRTVGKCPRYVKQKSLLLATGMRNVPEKPKKRGLFG |
| | | AIAGFIENGWEGLINGWYGFRHQNAQGEGTAADYKSTQSAI |
| | | DQITGKLNRLIGKTNQQFELINNEFNEVEQQIGNVINWTQDA |
| | | MTEVWSYNAELLVAMENQHTIDLTDSEMSKLYERVRKQLR |
| | | ENAEEDGTGCFEIFHKCDDHCMESIRNNTYDHTQYRTESLQ |
| | | NRIQIDPVKLSGGYKDIILWFSFGASCFLLLAIAMGLVFICIKN |
| | | GNMQCTICI 3′ (SEQ ID NO: 3) |
| ABR37506 | A/environment/ | 5′MNTQILALIAYMLIGAKGDKICLGHHAVANGTKVNTLTER |
| | Maryland/ | GIEVVNATETVETVNIKKICTQGKRPTDLGQCGLLGTLIGPP |
| | 267/2006 | QCDQFLEFDADLIIERREGTDVCYPGKFTNEESLRQILRGSG |
| | (H7N3) | GIDKESMGFTYSGIRTNGVTSACRRSGSSFYAEMKWLLSNS |
| | | DNAAFPQMTKSYRNPRNKPALIIWGVHHSGSATEQTKLYGS |
| | | GNKLITVGSSKYQQSFTPSPGARPQVNGQSGRIDFHWLLLDP |
| | | NDTVTFTFNGAFIAPDRASFFRGESLGVQSDVPLDSGCEGDC |
| | | FHSRGTIVSSLPFQNINPRTVGKCPRYVKQTSLLLATGMRNV |
| | | PENPKTRGLFGAIAGFIENGWEGLIDGWYGFRHQNAQGEGT |
| | | AADYKSTQSAIDQITGKLNRLIDKTNQQFELIDNEFSEIEQQI |
| | | GNVINWTRDSMTEVWSYNAELLVAMENQHTIDLADSEMN |
| | | KLYERVRKQLRENAEEDGTGCFEIFHKCDDQCMESIRNNTY |
| | | DHTQYRTESLQNRIQIDPVKLSSGYKDIILWFSFGASCFLLLA |
| | | IAMGLVFICIKNGNMRCTICI 3′ (SEQ ID NO: 4) |
| ACF47475 | A/mallard/California/ | 5′MNTQILALIACMLIGAKGDKICLGHHAVANGTKVNTLTER |
| | HKWF1971/ | GIEVVNATETVETANIKKICTQGKRPTDLGQCGLLGTLIGPP |
| | 2007 | QCDQFLEFDADLIIERREGTDVCYPGKFTNEESLRQILRGSG |
| | (H7N7) | GIDKESMGFTYSGIRTNGATSACRRSGSSFYAEMKWLLSNS |
| | | DNAAFPQMTKSYRNPRNKPALIIWGVHHSGSATEQTKLYGS |
| | | GNKLITVGSSKYQQSFTPSPGARPQVNGQSGRIDFHWLLLDP |
| | | NDTVTFTFNGAFIAPDRASFFRGGSLGVQSDVPLDSGCEGDC |
| | | FHSGGTIVSSLPFQNINPRTVGKCPRYVKQTSLLLATGMRNV |
| | | PENPKTRGLFGAIAGFIENGWEGLIDGWYGFRHQNAQGEGT |
| | | AADYKSTQSAIDQITGKLNRLIDKTNQQFELIDNEFNEIEQQI |
| | | GNVINWTRDSMTEVWSYNAELLVAMENQHTIDLADSEMN |
| | | KLYERVRKQLRENAEEDGTGCFEIFHKCDDQCMESIRNNTY |
| | | DHTQYRTESLQNRIQINPVKLSSGYKDIILWFSFGASCFLLLA |
| | | IAMGLVFICIKNGNMRCTICI 3′ (SEQ ID NO: 5) |
| ABP96852 | A/Egypt/2616- | 5′MEKIVLLLAIVSLVKSDQICIGYHANNSTEQVDTIMEKNVT |
| | NAMRU3/2007 | VTHAQDILEKTHNGKLCDLDGVKPLILRDCSVAGWLLGNP |
| | (H5N1) | MCDEFLNVPEWSYIVEKINPANDLCYPGDFNDYEELKHLLS |
| | | RINHFEKIQIIPKSSWSDYEASSGVSSACPYQGRSSFFRNVVW |
| | | LIKKNNAYPTIKRSYNNTNQEDLLVLWGIHHPNDAAEQIRL |
| | | YQNPTTYISIGTSTLNQRLVPKIATRSKVNGQSGRMEFFWTIL |
| | | KSNDAINFESNGNFIAPEYAYKIVKKGDSTIMKSELEYGNCN |
| | | TKCQTPIGAINSSMPFHNIHPLTIGECPKYVKSNRLVLATGLR |
| | | NSPQGERRRRKRGLFGAIAGFIEGGWQGMVDGWYGYHHSN |
| | | EQGSGYAADKESTQKAIDGVTNKVNSIINKMNTQFEAVGRE |
| | | FNNLERRIENLNKKMEDGFLDVWTYNAELLVLMENERTLD |
| | | FHDSNVKNLYDKVRLQLRDNAKELGNGCFEFYHRCDNECM |
| | | ESVRNGTYDYPQYSEEARLKREEISGVKLESMGIYQILSIYST |
| | | VASSLALAIMVAGLFLWMCSNGSLQCRICI 3′ (SEQ ID NO: |
| | | 6) |
| ABV23934 | A/Nigeria/6e/ | 5′DQICIGYHANNSTEQVDTIMEKNVTVTHAQNILEKTHNGK |
| | 07 (H5N1) | LCDLDGVKPLILRDCSVAGWLLGNPMCDEFLNVPEWSYIVE |
| | | KINPANDLCYPGNFNDYEELKHLLSRINHFEKIQIIPKSSWSD |
| | | HEASSGVSSACPYQGRSSFFRNVVWLIKKDNAYPTIKRSYN |
| | | NTNQEDLLVLWGIHHPNDAAEQTRLYQNPTTYISVGTSTLN |
| | | QRLVPKIATRSKVNGQSGRMEFFWTILKPNDAINFESNGNFI |
| | | APENAYKIVKKGDSTIMKSELEYGNCNTKCQTPIGAINSSMP |
| | | FHNIHPLTIGECPKYVKSNKLVLATGLRNSPQGERRRKKRGL |
| | | FGAIAGFIEGGWQGMVDGWYGYHHSNEQGSGYAADKEST |
| | | QKAIDGVTNKVNSIIDKMNTQFEAVGREFNNLERRIENLNK |
| | | KMEDGFLDVWTYNAELLVLMENERTLDFHDSNVKNLYDKI |
| | | RLQLRDNAKELGNGCFEFYHRCDNECMESVRNGTYDYPQY |
| | | SEEARLKREEISGVKLESIGTYQILSIYSTVASSLTLAIMVAGL |
| | | SLWMCSNGSLQCRICI 3′ (SEQ ID NO: 7) |
| ABI16504 | A/China/GD01/ | 5′MEKIVLLLAIVSLVKSDQICIGYHANNSTEQVDTIMEKNVT |
| | 2006 | VTHAQDILEKTHNGKLCDLDGVKPLILRDCSVAGWLLGNP |
| | (H5N1) | MCDEFINVPEWSYIVEKANPANDLCYPGNFNDYEELKHLLS |
| | | RINHFEKIQIISKSSWSDHEASSGVSSACPYQGTPSFFRNVVW |
| | | LIKKNNTYPTIKRSYNNTNQEDLLILWGIHHSNNAAEQTKLY |
| | | QNPTTYISVGTSTLNLRLVPKIATRSKVNGQSGRMDFFWTIL |
| | | KPNDAINFESNGNFIAPEYAYKIVKKGDSAIMKSEVEYGNCN |
| | | TKCQTPIGAINSSMPFHNIHPLTIGECPKYVKSNKLVLATGLR |
| | | NSPLRERRRKRGLFGAIAGFIEGGWQGMVDGWYGYHHSNE |
| | | QGSGYAADKESTQKAIDGVTNKVNSIIDKMNTQFEAVGREF |
| | | NNLERRIENLNKKMEDGFLDVWTYNAELLVLMENERTLDF |
| | | HDSNVKNLYDKVRLQLRDNAKELGNGCFEFYHKCDNECM |
| | | ESVRNGTYDYPQYSEEARLKREEISGVKLESIGTYQILSIYST |
| | | VASSLALAIMVAGLSLWMCSNGSLQCRICI 3′ (SEQ ID NO: |
| | | 8) |
| ABY27653 | A/India/m777/ | 5′MEKIVLLFAIVSLVKSDQICIGYHANNSTEQVDTIMEKNVT |
| | 2007 | VTHAQDILEKKHNGKLCDLDGVKPLILRDCSVAGWLLGNP |
| | (H5N1) | MCDEFINVPEWSYIVEKANPVNDLCYPGDFNDYEELKHLLS |
| | | RINHFEKIQIIPKSSWSSHEASLGVSSACPYQGKTSFFRNVVW |
| | | LIKKNSTYPTIKRSYNNTNQEDLLVLWGIHHPNDAAEQTKL |
| | | YQNPTTYISVGTSTLNQRLVPRIATRSKVNGQSGRMEFFWTI |
| | | LKPNDAINFESNGNFIAPEYAYKIVKKGDSTIMKSELEYGNC |
| | | NTKCQTPMGAINSSMPFHNIHPLTIGECPKYVKSNRLVLATG |
| | | LRNSPQRETRGLFGAIAGFIEGGWQGMVDGWYGYHHSNEQ |
| | | GSGYAADKESTQKAIDGVTNKVNSIIDKMNTQFEAVGREFN |
| | | NLERRIENLNKKMEDGFLDVWTYNAELLVLMENERTLDFH |
| | | DSNVKNLYDKVRLQLRDNAKELGNGCFEFYHKCDNECMES |
| | | VRNGTYDYPQYSEEARLKREEISGVKLESIGIYQILSIYSTVA |
| | | SSLALAIMVAGLSLWMCSNGSLQCRIC 3′ (SEQ ID NO: 9) |
| ABI36046 | A/Indonesia/ | 5′DQICIGYHANNSTEQVDTIMEKNVTVTHAQDILEKTHNGK |
| | CDC326N/ | LCDLDGVKPLILRDCSVAGWLLGNPMCDEFINVPEWSYIVE |
| | 2006 | KANPTNDLCYPGSFNDYEELKHLLSRINHFEKIQIIPKSSWSD |
| | (H5N1) | HEASSGVSSACPYLGSPSFFRNVVWLIKKNSTYPTIKKSYNN |
| | | TNQEDLLVLWGIHHPNDAAEQTRLYQNPTTYISIGTSTLNQR |
| | | LVPKIATRSKVNGQSGRMEFFWTILNPNDAINFESNGNFIAP |
| | | EYAYKIVKKGDSAIMKSELEYGNCNTKCQTPMGAINSSMPF |
| | | HNIHPLTIGECPKYVKSNRLVLATGLRNSPQRESRRKKRGLF |
| | | GAIAGFIEGGWQGMVDGWYGYHHSNEQGSGYAADKESTQ |
| | | KAIDGVTNKVNSIIDKMNTQFEAVGREFNNLERRIENLNKK |
| | | MEDGFLDVWTYNAELLVLMENERTLDFHDSNVKNLYDKV |
| | | RLQLRDNAKELGNGCFEFYHKCDNECMESIRNGTYNYPQY |
| | | SEEARLKREEISGVKLESIGTYQILSIYSTVASSLALAIMMAG |
| | | LSLWMCSNGSLQCRICI 3′ (SEQ ID NO: 10) |
| ACD85624 | A/Mississippi/ | 5′MKTIIALSYILCLVSAQKFPGNDNSTATLCLGHHAVPNGTI |
| | 05/2008 | VKTITNDQIEVTNATELVQSSSTGEICDSPHQILDGENCTLID |
| | (H3N2) | ALLGDPQCDGFQNKKWDLFVERSKAYSNCYPYDVPDYASL |
| | | RSLVASSGTLEFNNESFNWTGVTQNGTSSACIRRSNNSFFSR |
| | | LNWLTHLKFKYPALNVTMPNNEEFDKLYIWGVHHPGTDND |
| | | QIFLYAQASGRITVSTKRSQQTVIPNIRSRPRVRNIPSRISIYW |
| | | TIVKPGDILLINSTGNLIAPRGYFKIRSGKSSIMRSDAPIGKCN |
| | | SECITPNGSIPNDKPFQNVNRITYGACPRYVKQNTLKLATGM |
| | | RNVPEKQTRGIFGAIAGFIENGWEGMVDGWYGFRHQNSEGI |
| | | GQAADLKSTQAAIDQINGKLNRLIGKTNEKFHQIEKEFSEVE |
| | | GRIQDLEKYVEDTKIDLWSYNAELLVALENQHTIDLTDSEM |
| | | NKLFEKTKKQLRENAEDMGNGCFKIYHKCDNACIGSIRNGT |
| | | YDHDVYRDEALNNRFQIKGVELKSGYKDWILWISFAISCFLL |
| | | CVALLGFIMWACQKGNIRCNICI 3′ (SEQ ID NO: 11) |
| ACF10321 | A/New | 5′MKTIIALSYILCLVFAQKLPGNDNSTATLCLGHHAVPNGTI |
| | York/06/2008 | VKTITNDQIEVTNATELVQSSSTGEICDSPHQILDGENCTLID |
| | (H3N2) | ALLGDPQCDGFQNKKWDLFVERSKAYSNCYPYDVPDYASL |
| | | RSLVASSGTLEFKNESFNWTGVTQNGTSSACIRRSNNSFFSR |
| | | LNWLTHLKFKYPALNVTMPNKEKEDKLYIWGVHHPGTDND |
| | | QIFLYAQASGRITVSTKRSQQTVIPNIGSRLRVRDIPSRISIYW |
| | | TIVKPGDILLINSTGNLIAPRGYFKIRSGKSSIMRSDAPIGKCN |
| | | SECITPNGSIPNDKPFQNVNRITYGACPRYVKQNTLKLATGM |
| | | RNVPEKQTRGIFGAIAGFIENGWEGMVDGWYGFRHQNSEG |
| | | TGQAADLKSTQAAIDQINGKLNRLIGKTNEKFHQIEKEFSEV |
| | | EGRIQDLEKYVEDTKIDLWSYNAELLVALENQHTIDLTDSE |
| | | MNKLFEKTKKQLRENAEDMGNGCFKIYHKCDNACIGSIRN |
| | | GTYDHDVYRDEALNNRFQIKGVELKSGYKDWILWISFAISC |
| | | FLLCVALLGFIMWACQKGNIRCNICI 3′ (SEQ ID NO: 12) |
| ACD85628 | A/Idaho/03/ | 5′MKTIIALSYILCLVFAQKLPGNDNSTATLCLGHHAVPNGTI |
| | 2008 | VKTITNDQIEVTNATELVQSSSTGEICDSPHQILDGENCTLID |
| | (H3N2) | ALLGDPQCDGFQNKKWDLFVERSKAYSKCYPYDVPDYASL |
| | | RSLVASSGTLEFNNESFNWTGVTQNGTSSACIRRSNNSFFSR |
| | | LNWLTHLKFKYPALNVTMPNNEKFDKLYIWGVHHPGTDND |
| | | QIFLYAQASGRITVSTKRSQQTVIPNIGSRPRVRDIPSRISIYW |
| | | TIVKPGDILLINSTGNLIAPRGYFKIRSGKSSIMRSDAPIGKCN |
| | | SECITPNGSIPNDKPFQNVNRITYGACPRYVKQNTLKLATGM |
| | | RNVPEKQTRGIFGAIAGFIENGWEGMVDGWYGFRHQNSEGI |
| | | GQAADLKSTQAAIDQINGKLNRLIGKTNEKFHQIEKEFSEVE |
| | | GRIQDLEKYVEDTKIDLWSYNAELLVALENQHTIDLTDSEM |
| | | NKLFEKTKKQLRENAEDMGNGCFKIYHKCDNACIGSIRNGT |
| | | YDHDVYRDEALNNRFQIKGVELKSGYKDWILWISFAISCFLL |
| | | CVALLGFIMWACQKGNIRCNICI 3′ (SEQ ID NO: 13) |
| ACF40065 | A/Louisiana/ | 5′MKTIIALSYILCLVFAQKLPGNDNSTATLCLGHHAVPNGTI |
| | 06/2008 | VKTITNDQIEVTNATELVQSSSTGEICDSPHQILDGENCTLID |
| | (H3N2) | ALLGDPQCDGFQNKKWDLFVERSKAYSNCYPYDVPDYASL |
| | | RSLVASSGTLEFNNESFNWTGVTQNGTSSACIRRSNNSFFSR |
| | | LNWLTHLKFKYPALNVTMPNNEKFDKLYIWGVHHPGTDND |
| | | QIFLYAQASGRITVSTKRSQQTVIPNIGSRPRVRNIPSRISIYW |
| | | TIVKPGDILLINSTGNLIAPRGYFKIRSGKSSIMRSDAPIGKCN |
| | | SECITPNGSIPNDKPFQNVNRITYGACPRYVKQNTLKLATGM |
| | | RNVPEKQTRGIFGAIAGFIENGWEGMVDGWYGFRHQNSEGI |
| | | GQAADLKSTQAAIDQINGKLNRLIGKTNEKFHQIEKEFSEVE |
| | | GRIQDLEKYVEDTKIDLWSYNAELLVALENQHTIDLTDSEM |
| | | NKLFEKTKKQLRENAEDMGNGCFKIYHKCDNACIGSIRNGT |
| | | YDHDVYRDEALNNRFQIKGVELKSGYKDWILWISFAISCFLL |
| | | CVALLGFIMWACQKGNIRCNICI 3′ (SEQ ID NO: 14) |
| ACB11768 | A/Indiana/01/ | 5′MKVKLLVLLCTFTATYADTICIGYHANNSTDTVDTVLEKN |
| | 2008 | VTVTHSVNLLENSHNGKLCLLKGIAPLQLGNCSVAGWILGN |
| | (H1N1) | PECELLISKESWSYIVEKPNPENGTCYPGHFADYEELREQLSS |
| | | VSSFERFEIFPKESSWPNHTVTGVSASCSHNGESSFYRNLLW |
| | | LTGKNGLYPNLSKSYANNKEKEVLVLWGVHHPPNIGDQKA |
| | | LYHTENAYVSVVSSHYSRKFTPEIAKRPKVRDQEGRINYHW |
| | | TLLEPGDTIIFEANGNLIAPRYAFTLSRGFGSGIINSNAPMDK |
| | | CDAKCQTPQGAINSSLPFQNVHPVTIGECPKYVRSAKLRMV |
| | | TGLRNIPSIQSRGLFGAIAGFIEGGWTGMVDGWYGYHHQNE |
| | | QGSGYAADQKSTQNAINGITNKVNSVIEKMNTQFTAVGKEF |
| | | NKLERRMENLNKKVDDGFIDIWTYNAELLVLLENERTLDFH |
| | | DSNVKNLYEKVKSQLKNNAKEIGNGCFEFYHKCNDECMES |
| | | VKNGTYDYPKYSEESKLNREKIDGVKLESMGVYQILAIYST |
| | | VASSLVLLVSLGAISFWMCSNGSLQCRICI 3′ (SEQ ID NO: |
| | | 15) |
| ACB11769 | A/Pennsylvania/ | 5′MKVKLLVLLCTFTATYADTICIGYHANNSTDTVDTVLEKN |
| | 02/2008 | VTVTHSVNLLENSHNGKLCLLKGIAPLQLGNCSVAGWILGN |
| | (H1N1) | PECELLISKESWSYIVEKPNPENGTCYPGHFADYEELREQLSS |
| | | VSSFERFEIFPKESSWPNHTVTGVSASCSHNGESSFYRNLLW |
| | | LTGKNGLYPNLSKSYANNKEKEVLVLWGVHHPPNIGDQKT |
| | | LYHTENAYVSVVSSHYSRKFTPEIAKRPKVRDQEGRINYYW |
| | | TLLEPGDTIIFEANGNLIAPRYAFALSRGFGSGIINSNAPMDK |
| | | CDAKCQTPQGAINSSLPFQNVHPVTIGECPKYVRSAKLRMV |
| | | TGLRNIPSIQSRGLFGAIAGFIEGGWTGMVDGWYGYHHQNE |
| | | QGSGYAADQKSTQNAINGITNKVNSVIEKMNTQFTAVGKEF |
| | | NKLERRMENLNKKVDDGFIDIWTYNAELLVLLENERTLDFH |
| | | DSNVKNLYEKVKSQLKNNAKEIGNGCFEFYHKCNDECMES |
| | | VKNGTYDYPKYSEESKLNREKIDGVKLESMGVYQILAIYST |
| | | VASSLVLLVSLGAISFWMCSNGSLQCRICI 3′ (SEQ ID NO: |
| | | 16) |
| ACD47238 | A/Alaska/02/ | 5′MKVKLLVLLCTFTATYADTICIGYHANNSTDTVDTVLEKN |
| | 2008 | VTVTHSVNLLENSHNGKLCLLKGIAPLQLGNCSVAGWILGN |
| | (H1N1) | PECELLISKESWSYIVEKPNPENGTCYPGHFADYEELREQLSS |
| | | VSSFERFEIFPKESAWPNHTVTGVSASCSHNGEXSFYRNLLW |
| | | LTXKNGLYPNLSKSYANNKEKEVLVLWGVHHPPNIGDQKA |
| | | LYHTENAYVSVVSSHYSRKFTPEIAKRPKVRXQEGRINYYW |
| | | TLLEPGDTIIFEANGNLIAPRYAFALSRGFGSGIINSNAPMDK |
| | | CDAKCQTPQGAINSSLPFQNVHPVTIGECPKYVRSAKLRMV |
| | | TGLRNIPSIQSRGLFGAIAGFIEGGWTGMVDGWYGYHHQNE |
| | | QGSGYAADQKSTQNAINGITNKVNSVIEKMNTQFTAVGKEF |
| | | NKLERRMENLNKKVDDGFIDIWTYNAELLVLLENERTLDFH |
| | | DSNXKNLYEKVKSQLKNNAKEIGNGCFEFYHKCNDECMES |
| | | VKNGTXDYPKYSEESKLNREKIDGVKLESMGVYQILAIYST |
| | | VASSLVLLVSLGAISFWMCSNGSLQCRICI 3′ (SEQ ID NO: |
| | | 17) |
| ACD85766 | A/Indiana/04/ | 5′MKVKLLVLLCTFTATYADTICIGYHANNSTDTVDTVLEKN |
| | 2008 | VTVTHSVNLLENNHNGKLCLLKGIAPLQLGNCSVAGWILGN |
| | (H1N1) | PECELLISKESWSYIVEKPNPENGTCYPGHFADYEELREQLSS |
| | | VSSFERFEMFPKEGSWPNHTVTGVSASCSHNGESSFYRNLL |
| | | WLTGKNGLYPNLXKSYANNKEKEVLVLWGVHHPPNIGDQ |
| | | KALYHTENAYVSVVSSHYSRKFTPEIAKRPKVRDQEGRINY |
| | | YWTLLEPGDTIIFEANGNLIAPRYAFALSRGFGSGIINSNAPM |
| | | DNCDAKCQTPQGAINSSLPFQNVHPVTIGECPKYVRSAKLR |
| | | MVTGLRNIPSIQSRGLFGAIAGFIEGGWTGMVDGWYGYHH |
| | | QNEQGSGYAADQKSTQNAINGITNKVNSVIEKMNTQFTAVX |
| | | KEFNKLERRMENLNKKVDDGFIDIWTYNAELLVLLENERTL |
| | | DFHDSNVKNLYEKVKSQLKNNAKEIGNGCFEFYHKCNDEC |
| | | MESVKNGTYDYPKYSEESKLNREKIDGVKLESMGVYQILAI |
| | | YSTVASSLVLLVSLGAISFWMCSNGSLQCRICI 3′ (SEQ ID |
| | | NO: 18) |
| ACN29380 | B/Brisbane/ | MKAIIVLLMVVTSNADRICTGITSSNSPHVVKTATQGEVNVT |
| | 60/08 | GVIPLTTTPTKSHFANLKGTETRGKLCPKCLNCTDLDVALGR |
| | | PKCTGKIPSARVSILHEVRPVTSGCFPIMHDRTKIRQLPNLLR |
| | | GYEHIRLSTHNVINAENAPGGPYKIGTSGSCPNITNGNGFFAT |
| | | MAWAVPKNDKNKTATNPLTIEVPYICTEGEDQITVWGFHSD |
| | | NETQMAKLYGDSKPQKFTSSANGVTTHYVSQIGGFPNQTED |
| | | GGLPQSGRIVVDYMVQKSGKTGTITYQRGILLPQKVWCASG |
| | | RSKVIKGSLPLIGEADCLHEKYGGLNKSKPYYTGEHAKAIG |
| | | NCPIWVKTPLKLANGTKYRPPAKLLKERGFFGAIAGFLEGG |
| | | WEGMIAGWHGYTSHGAHGVAVAADLKSTQEAINKITKNLN |
| | | SLSELEVKNLQRLSGAMDELHNEILELDEKVDDLRADTISSQ |
| | | IELAVLLSNEGIINSEDEHLLALERKLKKMLGPSAVEIGNGCF |
| | | ETKHKCNQTCLDRIAAGTFDAGEFSLPTFDSLNITAASLNDD |
| | | GLDNHTILLYYSTAASSLAVTLMIAIFVVYMVSRDNVSCSIC |
| | | L (SEQ ID NO: 19) |
| ACQ76318.1 | A/California/ | MKAILVVLLYTFATANADTLCIGYHANNSTDTVDTVLEKNV |
| | 04/2009 | TVTHSVNLLEDKHNGKLCKLRGVAPLHLGKCNIAGWILGNP |
| | H1N1 | ECESLSTASSWSYIVETPSSDNGTCYPGDFIDYEELREQLSSV |
| | | SSFERFEIFPKTSSWPNHDSNKGVTAACPHAGAKSFYKNLIW |
| | | LVKKGNSYPKLSKSYINDKGKEVLVLWGIHHPSTSADQQSL |
| | | YQNADTYVFVGSSRYSKKFKPEIAIRPKVRDQEGRMNYYW |
| | | TLVEPGDKITFEATGNLVVPRYAFAMERNAGSGIIISDTPVH |
| | | DCNTTCQTPKGAINTSLPFQNIHPITIGKCPKYVKSTKLRLAT |
| | | GLRNIPSIQSRGLFGAIAGFIEGGWTGMVDGWYGYHHQNEQ |
| | | GSGYAADLKSTQNAIDEITNKVNSVIEKMNTQFTAVGKEFN |
| | | HLEKRIENLNKKVDDGFLDIWTYNAELLVLLENERTLDYHD |
| | | SNVKNLYEKVRSQLKNNAKEIGNGCFEFYHKCDNTCMESV |
| | | KNGTYDYPKYSEEAKLNREEIDGVKLESTRIYQILAIYSTVA |
| | | SSLVLVVSLGAISFWMCSNGSLQCRICI |
| | | (SEQ ID NO: 20) |
| ACP41935 | A/California/ | MKAILVVMLYTFATANADTLCIGYHANNSTDTVDTVLEKN |
| | 06/09 | VTVTHSVNLLEDKHNGKLCKLRGVAPLHLGKCNIAGWILG |
| | | NPECESLSTASSWSYIVETSSSDNGTCYPGDFIDYEELREQLS |
| | | SVSSFERFEIFPKTSSWPNHDSNKGVTAACPHAGAKSFYKNL |
| | | IWLVKKGNSYPKLSKSYINDKGKEVLVLWGIHHPSTSADQQ |
| | | SLYQNADAYVFVGSSRYSKKFKPEIAIRPKVRDQEGRMNYY |
| | | WTLVEPGDKITFEATGNLVVPRYAFAMERNAGSGIIISDTPV |
| | | HDCNTTCQTPKGAINTSLPFQNIHPITIGKCPKYVKSTKLRLA |
| | | TGLRNVPSIQSRGLFGAIAGFIEGGWTGMVDGWYGYHHQN |
| | | EQGSGYAADLKSTQNAIDEITNKVNSVIEKMNTQFTAVGKE |
| | | FNHLEKRIENLNKKVDDGFLDIWTYNAELLVLLENERTLDY |
| | | HDSNVKNLYEKVRSQLKNNAKEIGNGCFEFYHKCDNTCME |
| | | SVKNGTYDYPKYSEEAKLNREEIDGVKLESTRIYQILAIYST |
| | | VASSLVLVVSLGAISFWMCSNGSLQCRICI (SEQ ID NO: 21) |
| DQ371928 | A/Anhui/1/2005 | 5′MEKIVLLLAIVSLVKSDQICIGYHANNSTEQVDTIMEKNVT |
| | (H5N1) | VTHAQDILEKTHNGKLCDLDGVKPLILRDCSVAGWLLGNP |
| | | MCDEFINVPEWSYIVEKANPANDLCYPGNFNDYEELKHLLS |
| | | RINHFEKIQIIPKSSWSDHEASSGVSSACPYQGTPSFFRNVVW |
| | | LIKKNNTYPTIKRSYNNTNQEDLLILWGIHHSNDAAEQTKLY |
| | | QNPTTYISVGTSTLNQRLVPKIATRSKVNGQSGRMDFFWTIL |
| | | KPNDAINFESNGNFIAPEYAYKIVKKGDSAIVKSEVEYGNCN |
| | | TKCQTPIGAINSSMPFHNIHPLTIGECPKYVKSNKLVLATGLR |
| | | NSPLRERRRKRGLFGAIAGFIEGGWQGMVDGWYGYHHSNE |
| | | QGSGYAADKESTQKAIDGVTNKVNSIIDKMNTQFEAVGREF |
| | | NNLERRIENLNKKMEDGFLDVWTYNAELLVLMENERTLDF |
| | | HDSNVKNLYDKVRLQLRDNAKELGNGCFEFYHKCDNECM |
| | | ESVRNGTYDYPQYSEEARLKREEISGVKLESIGTYQILSIYST |
| | | VASSLALAIMVAGLSLWMCSNGSLQCRICI 3′ (SEQ ID NO: |
| | | 22) |
| ISDN125873 | A/Indonesia/ | 5′MEKIVLLLAIVSLVKSDQICIGYHANNSTEQVDTIMEKNVT |
| | 5/05 | VTHAQDILEKTHNGKLCDLDGVKPLILRDCSVAGWLLGNP |
| | | MCDEFINVPEWSYIVEKANPTNDLCYPGSFNDYEELKHLLS |
| | | RINHFEKIQIIPKSSWSDHEASSGVSSACPYLGSPSFFRNVVW |
| | | LIKKNSTYPTIKKSYNNTNQEDLLVLWGIHHPNDAAEQTRL |
| | | YQNPTTYISIGTSTLNQRLVPKIATRSKVNGQSGRMEFFWTIL |
| | | KPNDAINFESNGNFIAPEYAYKIVKKGDSAIMKSELEYGNCN |
| | | TKCQTPMGAINSSMPFHNIHPLTIGECPKYVKSNRLVLATGL |
| | | RNSPQRESRRKKRGLFGAIAGFIEGGWQGMVDGWYGYHHS |
| | | NEQGSGYAADKESTQKAIDGVTNKVNSIIDKMNTQFEAVGR |
| | | EFNNLERRIENLNKKMEDGFLDVWTYNAELLVLMENERTL |
| | | DFHDSNVKNLYDKVRLQLRDNAKELGNGCFEFYHKCDNEC |
| | | MESIRNGTYNYPQYSEEARLKREEISGVKLESIGTYQILSIYS |
| | | TVASSLALAIMMAGLSLWMCSNGSLQCRICI 3′ (SEQ ID NO: |
| | | 23) |
| DQ137873 | A/bar- | 5′MERIVLLLAIVSLVKSDQICIGYHANNSTEQVDTIMEKNVT |
| | headed | VTHAQDILEKTHNGKLCDLDGVKPLILRDCSVAGWLLGNP |
| | goose/Qinghai/ | MCDEFLNVPEWSYIVEKINPANDLCYPGNFNDYEELKHLLS |
| | 05/05 | RINHFERIQIIPKSSWSDHEASSGVSSACPYQGRSSFFRNVVW |
| | (H5N1) | LIKKNNAYPTIKRSYNNTNQEDLLVLWGIHHPNDAAEQTRL |
| | | YQNPTTYISVGTSTLNQRLVPKIATRSKVNGQSGRMEFFWTI |
| | | LKPNDAINFESNGNFIAPENAYKNCQKGDSTIMKSELEYGNC |
| | | NTKCQTPIGAINSSMPFHNIHPLTIGECPKYVKSNRLVLATGL |
| | | RNSPQGERRRKKRGLFGAIAGFIEGGWQGMVDGWYGYHHS |
| | | NEQGSGYAADKESTQKAIDGVTNKVNSIIDKMNTQFEAVGR |
| | | EFNNLERRIENLNKKMEDGFLDVWTYNAELLVLMENERTL |
| | | DFHDSNVKNLYDKVRLQLRDNAKELGNGCFEFYHRCDNEC |
| | | MESVRNGTYDYPQYSEEARLKREEISGVKLESIGTYQILSIYS |
| | | TVASSLALAIMVAGLSLWMCSNG 3′ (SEQ ID NO: 24) |
| AAT73273 | A/VietNam/ | 5′MEKIVLLFAIVSLVKSDQICIGYHANNSTEQVDTIMEKNVT |
| | 1194/04 | VTHAQDILEKTHNGKLCDLDGVKPLILRDCSVAGWLLGNP |
| | | MCDEFINVPEWSYIVEKANPVNDLCYPGDFNDYEELKHLLS |
| | | RINHFEKIQIIPKSSWSSHEASLGVSSACPYQGKSSFFRNVVW |
| | | LIKKNSTYPTIKRSYNNTNQEDLLVLWGIHHPNDAAEQTKL |
| | | YQNPTTYISVGTSTLNQRLVPRIATRSKVNGQSGRMEFFWTI |
| | | LKPNDAINFESNGNFIAPEYAYKIVKKGDSTIMKSELEYGNC |
| | | NTKCQTPMGAINSSMPFHNIHPLTIGECPKYVKSNRLVLATG |
| | | LRNSPQRERRRKKRGLFGAIAGFIEGGWQGMVDGWYGYHH |
| | | SNEQGSGYAADKESTQKAIDGVTNKVNSIIDKMNTQFEAVG |
| | | REFNNLERRIENLNKKMEDGFLDVWTYNAELLVLMENERT |
| | | LDFHDSNVKNLYDKVRLQLRDNAKELGNGCFEFYHKCDNE |
| | | CMESVRNGTYDYPQYSEEARLKREEISGVKLESIGIYQILSIY |
| | | STVASSLALAIMVAGLSLWMCSNGSLQCRICI 3′ (SEQ ID |
| | | NO: 25) |
| | B/Brisbane/ | 5′MKAIIVLLMVVTSNADRICTGITSSNSPHVVKTATQGEVN |
| | 3/07 | VTGVIPLTTTPTKSYFANLKGTKTRGKLCPDCLNCTDLDVA |
| | | LGRPMCVGTTPSAKASILHEVRPVTSGCFPIMHDRTKIRQLA |
| | | NLLRGYENIRLSTQNVIDAEKAPGGPYRLGTSGSCPNATSKS |
| | | GFFATMAWAVPKDNNKNATNPLTVEVPYICTEGEDQITVW |
| | | GFHSDDKTQMKNLYGDSNPQKFTSSANGVTTHYVSQIGGFP |
| | | DQTEDGGLPQSGRIVVDYMMQKPGKTGTIVYQRGVLLPQK |
| | | VWCASGRSKVIKGSLPLIGEADCLHEKYGGLNKSKPYYTGE |
| | | HAKAIGNCPIWVKTPLKLANGTKYRPPAKLLKERGFFGAIA |
| | | GFLEGGWEGMIAGWHGYTSHGAHGVAVAADLKSTQEAIN |
| | | KITKNLNSLSELEVKNLQRLSGAMDELHNEILELDEKVDDL |
| | | RADTISSQIELAVLLSNEGIINSEDEHLLALERKLKKMLGPSA |
| | | VDIGNGCFETKHKCNQTCLDRIAAGTFNAGEFSLPTFDSLNI |
| | | TAASLNDDGLDNHTILLYYSTAASSLAVTLMLAIFIVYMVSR |
| | | DNVSCSICL 3′ (SEQ ID NO: 26) |
| ACA28844 | A/Brisbane/ | 5′MKVKLLVLLCTFTATYADTICIGYHANNSTDTVDTVLEKN |
| | 59/2007 | VTVTHSVNLLENSHNGKLCLLKGIAPLQLGNCSVAGWILGN |
| | (H1N1) | PECELLISKESWSYIVEKPNPENGTCYPGHFADYEELREQLSS |
| | | VSSFERFEIFPKESSWPNHTVTGVSASCSHNGESSFYRNLLW |
| | | LTGKNGLYPNLSKSYANNKEKEVLVLWGVHHPPNIGDQKA |
| | | LYHTENAYVSVVSSHYSRKFTPEIAKRPKVRDQEGRINYYW |
| | | TLLEPGDTIIFEANGNLIAPRYAFALSRGFGSGIINSNAPMDK |
| | | CDAKCQTPQGAINSSLPFQNVHPVTIGECPKYVRSAKLRMV |
| | | TGLRNIPSIQSRGLFGAIAGFIEGGWTGMVDGWYGYHHQNE |
| | | QGSGYAADQKSTQNAINGITNKVNSVIEKMNTQFTAVGKEF |
| | | NKLERRMENLNKKVDDGFIDIWTYNAELLVLLENERTLDFH |
| | | DSNVKNLYEKVKSQLKNNAKEIGNGCFEFYHKCNDECMES |
| | | VKNGTYDYPKYSEESKLNREKIDGVKLESMGVYQILAIYST |
| | | VASSLVLLVSLGAISFWMCSNGSLQCRICI 3′ (SEQ ID NO: |
| | | 27) |
| ABW23353 | A/Brisbane/ | 5′QKLPGNDNSTATLCLGHHAVPNGTIVKTITNDQIEVTNAT |
| | 10/2007 | ELVQSSSTGEICDSPHQILDGENCTLIDALLGDPQCDGFQNK |
| | (H3N2) | KWDLFVERSKAYSNCYPYDVPDYASLRSLVASSGTLEFNNE |
| | | SFNWTGVTQNGTSSACIRRSNNSFFSRLNWLTHLKFKYPAL |
| | | NVTMPNNEKFDKLYIWGVHHPGTDNDQIFPYAQASGRITVS |
| | | TKRSQQTVIPNIGSRPRVRNIPSRISIYWTIVKPGDILLINSTGN |
| | | LIAPRGYFKIRSGKSSIMRSDAPIGKCNSECITPNGSIPNDKPF |
| | | QNVNRITYGACPRYVKQNTLKLATGMRNVPEKQTRGIFGAI |
| | | AGFIENGWEGMVDGWYGFRHQNSEGIGQAADLKSTQAAID |
| | | QINGKLNRLIGKTNEKFHQIEKEFSEVEGRIQDLEKYVEDTKI |
| | | DLWSYNAELLVALENQHTIDLTDSEMNKLFEKTKKQLREN |
| | | AEDMGNGCFKIYHKCDNACIGSIRNGTYDHDVYRDEALNN |
| | | RFQIKGVELKSGYKDWILWISFAISCFLLCVALLGFIMWACQ |
| | | KGNIRCNI 3′ (SEQ ID NO: 28) |
| ACA33493 | B/Florida/4/ | 5′MKAIIVLLMVVTSNADRICTGITSSNSPHVVKTATQGEVN |
| | 2006 | VTGVIPLTTTPTKSYFANLKGTRTRGKLCPDCLNCTDLDVAL |
| | | GRPMCVGTTPSAKASILHEVKPVTSGCFPIMHDRTKIRQLPN |
| | | LLRGYENIRLSTQNVIDAEKAPGGPYRLGTSGSCPNATSKSG |
| | | FFATMAWAVPKDNNKNATNPLTVEVPYICTEGEDQITVWG |
| | | FHSDDKTQMKNLYGDSNPQKFTSSANGVTTHYVSQIGSFPD |
| | | QTEDGGLPQSGRIVVDYMMQKPGKTGTIVYQRGVLLPQKV |
| | | WCASGRSKVIKGSLPLIGEADCLHEKYGGLNKSKPYYTGEH |
| | | AKAIGNCPIWVKTPLKLANGTKYRPPAKLLKERGFFGAIAGF |
| | | LEGGWEGMIAGWHGYTSHGAHGVAVAADLKSTQEAINKIT |
| | | KNLNSLSELEVKNLQRLSGAMDELHNEILELDEKVDDLRAD |
| | | TISSQIELAVLLSNEGIINSEDEHLLALERKLKKMLGPSAVEIG |
| | | NGCFETKHKCNQTCLDRIAAGTFNAGEFSLPTFDSLNITAAS |
| | | LNDDGLDNHTILLYYSTAASSLAVTLMLAIFIVYMVSRDNV |
| | | SCSICL 3′ (SEQ ID NO: 29) |
| ACR15732 | B/Malaysia/ | 5′MKAIIVLLMVVTSNADRIICTGITSSNSPHVVKTATQGEVN |
| | 2506/2004- | VTGVIPLTTTPTKSHFANLKGTETRGKLCPKCLNCTDLDVAL |
| | like | GRPKCTGNIPSARVSILHEVRPVTSGCFPIMHDRTKIRQLPNL |
| | | LRGYEHIRLSTHNVINAENAPGGPYKIGTSGSCPNVTNGNGF |
| | | FATMAWAVPKNDNNKTATNSLTIEVPYICTEGEDQITVWGF |
| | | HSDNETQMAKLYGDSKPQKFTSSANGVTTHYVSQIGGFPNQ |
| | | TEDGGLPQSGRIVVDYMVQKSGKTGTITYQRGILLPQKVWC |
| | | ASGRSKVIKGSLPLIGEADCLHEKYGGLNKSKPYYTGEHAK |
| | | AIGNCPIWVKTPLKLANGTKYRPPAKLLKERGFFGAIAGFLE |
| | | GGWEGMIAGWHGYTSHGAHGVAVAADLKSTQEAINKITK |
| | | NLNSLSELEVKNLQRLSGAMDELHNEILELDEKVDDLRADT |
| | | ISSQIELAVLLSNEGIINSEDEHLLALERKLKKMLGPSAVEIG |
| | | NGCFETKHKCNQTCLDRIAAGTFDAGEFSLPTFDSLNITAAS |
| | | LNDDGLDNHTILLYYSTAASSLAVTLMIAIFVVYMVSRDNV |
| | | SCSICL 3′ (SEQ ID NO: 30) |
| AAP34324 | A/New | 5′MKAKLLVLLCTFTATYADTICIGYHANNSTDTVDTVLEKN |
| | Caledonia/20/ | VTVTHSVNLLEDSHNGKLCLLKGIAPLQLGNCSVAGWILGN |
| | 99 | PECELLISKESWSYIVETPNPENGTCYPGYFADYEELREQLSS |
| | (H1N1) | VSSFERFEIFPKESSWPNHTVTGVSASCSHNGKSSFYRNLLW |
| | | LTGKNGLYPNLSKSYVNNKEKEVLVLWGVHHPPNIGNQRA |
| | | LYHTENAYVSVVSSHYSRRFTPEIAKRPKVRDQEGRINYYW |
| | | TLLEPGDTIIFEANGNLIAPWYAFALSRGFGSGIITSNAPMDE |
| | | CDAKCQTPQGAINSSLPFQNVHPVTIGECPKYVRSAKLRMV |
| | | TGLRNIPSIQSRGLFGAIAGFIEGGWTGMVDGWYGYHHQNE |
| | | QGSGYAADQKSTQNAINGITNKVNSVIEKMNTQFTAVGKEF |
| | | NKLERRMENLNKKVDDGFLDIWTYNAELLVLLENERTLDF |
| | | HDSNVKNLYEKVKSQLKNNAKEIGNGCFEFYHKCNNECME |
| | | SVKNGTYDYPKYSEESKLNREKIDGVKLESMGVYQILAIYST |
| | | VASSLVLLVSLGAISFWMCSNGSLQCRICI 3′ (SEQ ID NO: |
| | | 31) |
| ABU99109 | A/Solomon | 5′MKVKLLVLLCTFTATYADTICIGYHANNSTDTVDTVLEKN |
| | Islands/3/2006 | VTVTHSVNLLEDSHNGKLCLLKGIAPLQLGNCSVAGWILGN |
| | (H1N1) | PECELLISRESWSYIVEKPNPENGTCYPGHFADYEELREQLSS |
| | | VSSFERFEIFPKESSWPNHTTTGVSASCSHNGESSFYKNLLW |
| | | LTGKNGLYPNLSKSYANNKEKEVLVLWGVHHPPNIGDQRA |
| | | LYHKENAYVSVVSSHYSRKFTPEIAKRPKVRDQEGRINYYW |
| | | TLLEPGDTIIFEANGNLIAPRYAFALSRGFGSGIINSNAPMDE |
| | | CDAKCQTPQGAINSSLPFQNVHPVTIGECPKYVRSAKLRMV |
| | | TGLRNIPSIQSRGLFGAIAGFIEGGWTGMVDGWYGYHHQNE |
| | | QGSGYAADQKSTQNAINGITNKVNSVIEKMNTQFTAVGKEF |
| | | NKLERRMENLNKKVDDGFIDIWTYNAELLVLLENERTLDFH |
| | | DSNVKNLYEKVKSQLKNNAKEIGNGCFEFYHKCNDECMES |
| | | VKNGTYDYPKYSEESKLNREKIDGVKLESMGVYQILAIYST |
| | | VASSLVLLVSLGAISFWMCSNGSLQCRICI 3′ (SEQ ID NO: |
| | | 32) |
| ACF54576 | A/Wisconsin/ | 5′MKTIIALSYILCLVFAQKLPGNDNSTATLCLGHHAVPNGTI |
| | 67/2005 | VKTITNDQIEVTNATELVQSSSTGGICDSPHQILDGENCTLID |
| | (H3N2) | ALLGDPQCDGFQNKKWDLFVERSKAYSNCYPYDVPDYASL |
| | | RSLVASSGTLEFNDESFNWTGVTQNGTSSSCKRRSNNSFFSR |
| | | LNWLTHLKFKYPALNVTMPNNEKFDKLYIWGVHHPVTDND |
| | | QIFLYAQASGRITVSTKRSQQTVIPNIGSRPRIRNIPSRISIYWT |
| | | IVKPGDILLINSTGNLIAPRGYFKIRSGKSSIMRSDAPIGKCNS |
| | | ECITPNGSIPNDKPFQNVNRITYGACPRYVKQNTLKLATGMR |
| | | NVPEKQTRGIFGAIAGFIENGWEGMVDGWYGFRHQNSEGIG |
| | | QAADLKSTQAAINQINGKLNRLIGKTNEKFHQIEKEFSEVEG |
| | | RIQDLEKYVEDTKIDLWSYNAELLVALENQHTIDLTDSEMN |
| | | KLFERTKKQLRENAEDMGNGCFKIYHKCDNACIGSIRNGTY |
| | | DHDVYRDEALNNRFQIKGVELKSGYKDWILWISFAISCFLLC |
| | | VALLGFIMWACQKGNIRCNICI 3′ (SEQ ID NO: 33) |
| AAT08000 | A/Wyoming/ | 5′MKTIIALSYILCLVFSQKLPGNDNSTATLCLGHHAVPNGTI |
| | 3/03 | VKTITNDQIEVTNATELVQSSSTGGICDSPHQILDGENCTLID |
| | (H3N2) | ALLGDPQCDGFQNKKWDLFVERSKAYSNCYPYDVPDYASL |
| | | RSLVASSGTLEFNNESFNWAGVTQNGTSSACKRRSNKSFFS |
| | | RLNWLTHLKYKYPALNVTMPNNEKFDKLYIWGVHHPVTDS |
| | | DQISLYAQASGRITVSTKRSQQTVIPNIGYRPRVRDISSRISIY |
| | | WTIVKPGDILLINSTGNLIAPRGYFKIRSGKSSIMRSDAPIGKC |
| | | NSECITPNGSIPNDKPFQNVNRITYGACPRYVKQNTLKLATG |
| | | MRNVPEKQTRGIFGAIAGFIENGWEGMVDGWYGFRHQNSE |
| | | GTGQAADLKSTQAAINQINGKLNRLIGKTNEKFHQIEKEFSE |
| | | VEGRIQDLEKYVEDTKIDLWSYNAELLVALENQHTIDLTDS |
| | | EMNKLFERTKKQLRENAEDMGNGCFKIYHKCDNACIESIRN |
| | | GTYDHDVYRDEALNNRFQIKGVELKSGYKDWILWISFAISC |
| | | FLLCVALLGFIMWACQKGNIRCNICI 3′ (SEQ ID NO: 34) |
| AAR02640 | A/Netherlands/ | 5′SKSRGYKMNTQILVFALVASIPTNADKICLGHHAVSNGTK |
| | 219/03 | VNTLTERGVEVVNATETVERTNVPRICSKGKRTVDLGQCGL |
| | (H7N7) | LGTITGPPQCDQFLEFSADLIIERREGSDVCYPGKFVNEEALR |
| | | QILRESGGIDKETMGFTYSGIRTNGTTSACRRSGSSFYAEMK |
| | | WLLSNTDNAAFPQMTKSYKNTRKDPALIIWGIHHSGSTTEQ |
| | | TKLYGSGNKLITVGSSNYQQSFVPSPGARPQVNGQSGRIDFH |
| | | WLILNPNDTVTFSFNGAFIAPDRASFLRGKSMGIQSEVQVDA |
| | | NCEGDCYHSGGTIISNLPFQNINSRAVGKCPRYVKQESLLLA |
| | | TGMKNVPEIPKRRRRGLFGAIAGFIENGWEGLIDGWYGFRH |
| | | QNAQGEGTAADYKSTQSAIDQITGKLNRLIEKTNQQFELIDN |
| | | EFTEVERQIGNVINWTRDSMTEVWSYNAELLVAMENQHTID |
| | | LADSEMNKLYERVKRQLRENAEEDGTGCFEIFHKCDDDCM |
| | | ASIRNNTYDHSKYREEAIQNRIQIDPVKLSSGYKDVILWFSFG |
| | | ASCFILLAIAMGLVFICVKNGNMRCTICI 3′ (SEQ ID NO: 35) |
| ACP41953 | A/California/ | MKAILVVLLYTFATANADTLCIGYHANNSTDTVDTVLEKNV |
| | 07/2009 | TVTHSVNLLEDKHNGKLCKLRGVAPLHLGKCNIAGWILGNP |
| | (H1N1) | ECESLSTASSWSYIVETPSSDNGTCYPGDFIDYEELREQLSSV |
| | | SSFERFEIFPKTSSWPNHDSNKGVTAACPHAGAKSFYKNLIW |
| | | LVKKGNSYPKLSKSYINDKGKEVLVLWGIHHPSTSADQQSL |
| | | YQNADAYVFVGSSRYSKKFKPEIAIRPKVRDQEGRMNYYW |
| | | TLVEPGDKITFEATGNLVVPRYAFAMERNAGSGIIISDTPVH |
| | | DCNTTCQTPKGAINTSLPFQNIHPITIGKCPKYVKSTKLRLAT |
| | | GLRNIPSIQSRGLFGAIAGFIEGGWTGMVDGWYGYHHQNEQ |
| | | GSGYAADLKSTQNAIDEITNKVNSVIEKMNTQFTAVGKEFN |
| | | HLEKRIENLNKKVDDGFLDIWTYNAELLVLLENERTLDYHD |
| | | SNVKNLYEKVRSQLKNNAKEIGNGCFEFYHKCDNTCMESV |
| | | KNGTYDYPKYSEEAKLNREEIDGVKLESTRIYQILAIYSTVA |
| | | SSLVLVVSLGAISFWMCSNGSLQCRICI (SEQ ID NO: 111) |
| ACR19260 | A/Texas/05/ | MKAILVVLLYTFATANADTLCIGYHANNSTDTVDTVLEKNV |
| | 2009 | TVTHSVNLLEDKHNGKLCKLRGVAPLHLGKCNIAGWILGNP |
| | (H1N1) | ECESLSTASSWSYIVETSSSDNGTCYPGDFIDYEELREQLSSV |
| | | SSFERFEIFPKTSSWPNHDSNKGVTAACPHAGAKSFYKNLIW |
| | | LVKKGNSYPKLSKSYINDKGKEVLVLWGIHHPSTSADQQSL |
| | | YQNADAYVFVGSSRYSKKFKPEIAIRPKVRDQEGRMNYYW |
| | | TLVEPGDKITFEATGNLVVPRYAFAMERNAGSGIIISDTPVH |
| | | DCNTTCQTPKGAINTSLPFQNIHPITIGKCPKYVKSTKLRLAT |
| | | GLRNVPSIQSRGLFGAIAGFIEGGWTGMVDGWYGYHHQNE |
| | | QGSGYAADLKSTQNAIDEITNKVNSVIEKMNTQFTAVGKEF |
| | | NHLEKRIENLNKKVDDGFLDIWTYNAELLVLLENERTLDYH |
| | | DSNVKNLYEKVRSQLKNNAKEIGNGCFEFYHKCDNTCMES |
| | | VKNGTYDYPKYSEEAKLNREEIDGVKLESTRIYQILAIYSTV |
| | | ASSLVLVVSLGAISFWMCSNGSLQCRICI (SEQ ID NO: 112) |

In certain embodiments, full length neuraminidase (NA) antigen is utilized in vaccine antigens in accordance with the invention. In some embodiments, a domain of NA is used. In certain embodiments two or three or more domains are provided in antigens in accordance with the invention. Certain exemplary embodiments provide influenza antigen polypeptide comprising full length NA, lacking a transmembrane anchor peptide sequence. Sequences of exemplary NA polypeptides are presented in Table 2.

TABLE 2
Exemplary NA Sequences

| GenBank | | |
|---|---|---|
| Accession | Strain | NA Sequence |
| AAT73327 | A/Viet | 5′MNPNQKIITIGSICMVTGIVSLMLQIGNMISIWVSHSIHTGN |
| | Nam/1194/2004 | QHQSEPISNTNLLTEKAVASVKLAGNSSLCPINGWAVYSKD |
| | (H5N1) | NSIRIGSKGDVFVIREPFISCSHLECRTFFLTQGALLNDKHSN |
| | | GTVKDRSPHRTLMSCPVGEAPSPYNSRFESVAWSASACHDG |
| | | TSWLTIGISGPDNGAVAVLKYNGIITDTIKSWRNNILRTQESE |
| | | CACVNGSCFTVMTDGPSNGQASHKIFKMEKGKVVKSVELD |
| | | APNYHYEECSCYPDAGEITCVCRDNWHGSNRPWVSFNQNL |
| | | EYQIGYICSGVFGDNPRPNDGTGSCGPVSSNGAGGVKGFSF |
| | | KYGNGVWIGRTKSTNSRSGFEMIWDPNGWTETDSSFSVKQ |
| | | DIVAITDWSGYSGSFVQHPELTGLDCIRPCFWVELIRGRPKES |
| | | TIWTSGSSISFCGVNSDTVGWSWPDGAELPFTIDK 3′ (SEQ ID |
| | | NO: 36) |
| AAT08001 | A/Wyoming/ | 5′MNPNQKIITIGSVSLTISTICFFMQIAILITTVTLHFKQYEFNS |
| | 3/03. | PPNNQVMLCEPTIIERNITEIVYLTNTTIEKEICPKLAEYRNWS |
| | (H3N2) | KPQCNITGFAPFSKDNSIRLSAGGDIWVTREPYVSCDPDKCY |
| | | QFALGQGTTLNNVHSNDTVHDRTPYRTLLMNELGVPFHLG |
| | | TKQVCIAWSSSSCHDGKAWLHVCVTGDDENATASFIYNGR |
| | | LVDSIVSWSKKILRTQESECVCINGTCTVVMTDGSASGKAD |
| | | TKILFIEEGKIVHTSTLSGSAQHVEECSCYPRYPGVRCVCRD |
| | | NWKGSNRPIVDINIKDYSIVSSYVCSGLVGDTPRKNDSSSSS |
| | | HCLDPNNEEGGHGVKGWAFDDGNDVWMGRTISEKLRSGY |
| | | ETFKVIEGWSNPNSKLQINRQVIVDRGNRSGYSGIFSVEGKS |
| | | CINRCFYVELIRGRKQETEVLWTSNSIVVFCGTSGTYGTGSW |
| | | PDGADINLMPI 3′ (SEQ ID NO: 37) |
| ABU94738 | A/Anhui/5/05 | 5′MNPNQKIITIGSICMVIGIVSLMLQIGNMISIWVSHSIQTGN |
| | | QHQAEPIRNANFLTENAVASVTLAGNSSLCPVRGWAVHSK |
| | | DNSIRIGSKGDVFVIREPFISCSHLECRTFFLTQGALLNDKHS |
| | | NGTVKDRSPHRTLMSCPVGEAPSPYNSRFESVAWSASACHD |
| | | GTSWLTIGISGPDNGAVAVLKYNGIITDTIKSWRNNILRTQES |
| | | ECACVNGSCFTVMTDGPSNGQASYKIFKMEKGKVVKSVEL |
| | | NAPNYHYEECSCYPGAGEITCVCRDNWHGSNRPWVSFNQN |
| | | LEYQIGYICSGVFGDNPRPNDGTGSCGPVSPNGAYGIKGFSF |
| | | KYGNGVWIGRTKSTNSRSGFEMIWDPNGWTETDSNFSVKQ |
| | | DIVAITDWSGYSGSFVQHPELTGLDCIRPCFWVELIRGRPKES |
| | | TIWTSGSSISFCGVNSDTVGWSWPDGAELPFTIDK 3′ (SEQ ID |
| | | NO: 38) |
| ISDN125875 | A/Indonesia/ | 5′MNPNQKIITIGSICMVIGIVSLMLQIGNMISIWVIHSIQTGNQ |
| | 5/05 | HQAESISNTNPLTEKAVASVTLAGNSSLCPIRGWAVHSKDN |
| | | NIRIGSKGDVFVIREPFISCSHLECRTFFLTQGALLNDKHSNG |
| | | TVKDRSPHRTLMSCPVGEAPSPYNSRFESVAWSASACHDGT |
| | | SWLTIGISGPDNEAVAVLKYNGIITDTIKSWRNDILRTQESEC |
| | | ACVNGSCFTVMTDGPSNGQASYKIFKMEKGKVVKSVELDA |
| | | PNYHYEECSCYPDAGEITCVCRDNWHGSNRPWVSFNQNLE |
| | | YQIGYICSGVFGDNPRPNDGTGSCGPMSPNGAYGVKGFSFK |
| | | YGNGVWIGRTKSTNSRSGFEMIWDPNGWTGTDSSFSVKQDI |
| | | VAITDWSGYSGSFVQHPELTGLDCIRPCFWVELIRGRPKESTI |
| | | WTSGSSISFCGVNSDTVSWS 3′ (SEQ ID NO: 39) |
| DQ095657 | A/Bar- | 5′MNPNQKIITIGSICMVIGIVSLMLQIGNMISIWVSHSIQTGN |
| | headed | QRQAEPISNTKFLTEKAVASVTLAGNSSLCPISGWAVYSKDN |
| | Goose/Qing | SIRIGSRGDVFVIREPFISCSHLECRTFFLTQGALLNDKHSNGT |
| | hai/5/ | VKDRSPHRTLMSCPVGEAPSPYNSRFESVAWSASACHDGTS |
| | | WLTIGISGPDNGAVAVLKYNGIITDTIKSWRNNILRTQESEC |
| | | ACVNGSCFTVMTDGPSNGQASYKIFKMEKGKVVKSVELDA |
| | | PNYHYEECSCYPDAGEITCVCRDNWHGSNRPWVSFNQNLE |
| | | YQIGYICSGVFGDNPRPNDGTGSCGPVSPNGAYGVKGFSFK |
| | | YGNGVWIGRTKSTNSRSGFEMIWDPNGWTGTDSSFSVKQDI |
| | | VAITDWSGYSGSFVQHPELTGLDCIRPCFWVELIRGRPKESTI |
| | | WTSGSSISFCGVNSDTVSWSWPDGAELPFTIDK 3′ (SEQ ID |
| | | NO: 40) |
| ISDN126673 | B/Malaysia/ | 5′MLPSTIQTLTLFLTSGGVLLSLYVSASLSYLLYSDILLKFPS |
| | 2506/2004 | TEITAPTMPLDCANASNVQAVNRSATKGVTLLLPEPEWTYP |
| | (egg | RLSCPGSTFQKALLISPHRFGETKGNSAPLIIREPFIACGPKEC |
| | passaged) | KHFALTHYAAQPGGYYNGTRGDRNKLRHLISVKLGKIPTVE |
| | | NSIFHMAAWSGSACHDGKEWTYIGVDGPDNNALLKIKYGE |
| | | AYTDTYHSYANNILRTQESACNCIGGNCYLMITDGSASGVS |
| | | ECRFLKIREGRIIKEIFPTGRIKHTEECTCGFASNKTIECACRD |
| | | NSYTAKRPFVKLNVETDTAEIRLMCTETYLDTPRPDDGSITG |
| | | PCESNGDKGSGGIKGGFVHQRMASKIGRWYSRTMSKTKRM |
| | | GMGLYVKYDGDPWADSDALAFSGVMVSMEEPGWYSFGFE |
| | | IKDKKCDVPCIGIEMVHDGGKETWHSAATAIYCLMGSGQLL |
| | | WDTVTGVNMAL 3′ (SEQ ID NO: 41) |
| CAD57252 | A/New | 5′MNPNQKIITIGSISIAIGIISLMLQIGNIISIWASHSIQTGSQNH |
| | Caledonia/20/ | TGVCNQRIITYENSTWVNHTYVNINNTNVVAGKDKTSVTLA |
| | 99 | GNSSLCSISGWAIYTKDNSIRIGSKGDVFVIREPFISCSHLECR |
| | (H1N1) | TFFLTQGALLNDKHSNGTVKDRSPYRALMSCPLGEAPSPYN |
| | | SKFESVAWSASACHDGMGWLTIGISGPDNGAVAVLKYNGII |
| | | TETIKSWKKRILRTQESECVCVNGSCFTIMTDGPSNGAASYK |
| | | IFKIEKGKVTKSIELNAPNFHYEECSCYPDTGTVMCVCRDN |
| | | WHGSNRPWVSFNQNLDYQIGYICSGVFGDNPRPKDGEGSC |
| | | NPVTVDGADGVKGFSYKYGNGVWIGRTKSNRLRKGFEMIW |
| | | DPNGWTDTDSDFSVKQDVVAITDWSGYSGSFVQHPELTGL |
| | | DCIRPCFWVELVRGLPRENTTIWTSGSSISFCGVNSDTANWS |
| | | WPDGAELPFTIDK 3′ (SEQ ID NO: 42) |
| ISDN136490 | A/Wisconsin/ | 5′MNPNQKIITIGSVSLTISTICFFMQIAILITTVTLHFKQYEFNS |
| | 67/2005 | PPNNQVMLCEPTIIERNITEIVYLTNTTIEKEICPKLAEYRNWS |
| | (H3N2) | KPQCNITGFAPFSKDNSIRLSAGGDIWVTREPYVSCDPDKCY |
| | | QFALGQGTTLNNVHSNDTVHDRTPYRTLLMNELGVPFHLG |
| | | TKQVCIAWSSSSCHDGKAWLHVCVTGDDKNATASFIYNGR |
| | | LVDSIVSWSKEILRTQESECVCINGTCTVVMTDGSASGKADT |
| | | KILFIEEGKIVHTSTLSGSAQHVEECSCYPRYLGVRCVCRDN |
| | | WKGSNRPIVDINIKDYSIVSSYVCSGLVGDTPRKNDSSSSSHC |
| | | LDPNNEEGGHGVKGWAFDDGNDVWMGRTISEKLRSGYETF |
| | | KVIEGWSNPNSKLQINRQVIVDRGNRSGYSGIFSVEGKSCIN |
| | | RCFYVELIRGRKEETEVLWTSNSIVVFCGTSGTYGTGSWPD |
| | | GADINLMPI 3′ (SEQ ID NO: 43) |
| ACP41107.1 | A/California/ | MNPNQKIITIGSVCMTIGMANLILQIGNIISIWISHSIQLGNQN |
| | 04/2009 | QIETCNQSVITYENNTWVNQTYVNISNTNFAAGQSVVSVKL |
| | (H1N1) | AGNSSLCPVSGWAIYSKDNSVRIGSKGDVFVIREPFISCSPLE |
| | | CRTFFLTQGALLNDKHSNGTIKDRSPYRTLMSCPIGEVPSPY |
| | | NSRFESVAWSASACHDGINWLTIGISGPDNGAVAVLKYNGII |
| | | TDTIKSWRNNILRTQESECACVNGSCFTVMTDGPSNGQASY |
| | | KIFRIEKGKIVKSVEMNAPNYHYEECSCYPDSSEITCVCRDN |
| | | WHGSNRPWVSFNQNLEYQIGYICSGIFGDNPRPNDKTGSCG |
| | | PVSSNGANGVKGFSFKYGNGVWIGRTKSISSRNGFEMIWDP |
| | | NGWTGTDNNFSIKQDIVGINEWSGYSGSFVQHPELTGLDCIR |
| | | PCFWVELIRGRPKENTIWTSGSSISFCGVNSDTVGWSWPDG |
| | | AELPFTIDK (SEQ ID NO: 110) |

In addition, the Exemplification presents several additional HA polypeptide sequences that can be used in accordance with the present invention.

While sequences of exemplary influenza antigen polypeptides are provided herein, it will be appreciated that any sequence having immunogenic characteristics of HA and/or NA may be employed. In some embodiments, an influenza antigen polypeptide for use in accordance with the present invention has an amino acid sequence which is about 60% identical, about 70% identical, about 80% identical, about 85% identical, about 90% identical, about 91% identical, about 92% identical, about 93% identical, about 94% identical, about 95% identical, about 96% identical, about 97% identical, about 98% identical, about 99% identical, or 100% identical to a sequence selected from the group consisting of SEQ ID NOs: 1-43, 85, 87, 89, 91, 93, 95, 97, 99, 101, 103, 105, 107, 109, 110, 111, and 112. In some embodiments, such an influenza antigen polypeptide retains immunogenic activity.

In some embodiments, an influenza antigen polypeptide for use in accordance with the present invention has an amino acid sequence which comprises about 100 contiguous amino acids of a sequence selected from the group consisting of SEQ ID NOs: 1-43, 85, 87, 89, 91, 93, 95, 97, 99, 101, 103, 105, 107, 109, 110, 111, and 112. In some embodiments, an influenza antigen polypeptide has an amino acid sequence which is about 60% identical, about 70% identical, about 80% identical, about 85% identical, about 90% identical, about 91% identical, about 92% identical, about 93% identical, about 94% identical, about 95% identical, about 96% identical, about 97% identical, about 98% identical, about 99% identical, or 100% identical to a contiguous stretch of about 100 amino acids of a sequence selected from the group consisting of SEQ ID NOs: 1-43, 85, 87, 89, 91, 93, 95, 97, 99, 101, 103, 105, 107, 109, 110, 111, and 112.

In some embodiments, an influenza antigen polypeptide for use in accordance with the present invention has an amino acid sequence which comprises about 20, about 30, about 40, about 50, about 60, about 70, about 80, about 90, about 100, about 150, about 200, about 250, about 300, about 350, about 400, about 450, about 500, about 550, or more contiguous amino acids of a sequence selected from the group consisting of SEQ ID NOs: 1-43, 85, 87, 89, 91, 93, 95, 97, 99, 101, 103, 105, 107, 109, 110, 111, and 112. In some embodiments, an influenza antigen polypeptide for use in accordance with the present invention has an amino acid sequence which comprises about 60%, about 70%, about 80%, about 90%, about 95% of the contiguous amino acids of a sequence selected from the group consisting of SEQ ID NOs: 1-43, 85, 87, 89, 91, 93, 95, 97, 99, 101, 103, 105, 107, 109, 110, 111, and 112. In some embodiments, an influenza antigen polypeptide has an amino acid sequence which is about 60% identical, about 70% identical, about 80% identical, about 85% identical, about 90% identical, about 91% identical, about 92% identical, about 93% identical, about 94% identical, about 95% identical, about 96% identical, about 97% identical, about 98% identical, about 99% identical, or 100% identical to a contiguous stretch of about 150, 200, 250, 300, 350, or more amino acids of a sequence selected from the group consisting of SEQ ID NOs: 1-43, 85, 87, 89, 91, 93, 95, 97, 99, 101, 103, 105, 107, 109, 110, 111, and 112.

For example, sequences having sufficient identity to influenza antigen polypeptide(s) which retain immunogenic characteristics are capable of binding with antibodies which react with one or more antigens provided herein. Immunogenic characteristics often include three dimensional presentation of relevant amino acids or side groups. One skilled in the art can readily identify sequences with modest differences in sequence (e.g., with difference in boundaries and/or some sequence alternatives, that, nonetheless preserve immunogenic characteristics).

In some embodiments, particular portions and/or domains of any of the exemplary sequences set forth in SEQ ID NOs: 1-43, 85, 87, 89, 91, 93, 95, 97, 99, 101, 103, 105, 107, 109, 110, 111, and 112. may be omitted from an influenza polypeptide. For example, HA and NA polypeptides typically contain a transmembrane anchor sequence. HA and NA polypeptides in which the transmembrane anchor sequence has been omitted are contemplated by the invention.

As exemplary antigens, we have utilized sequences from hemagglutinin and neuraminidase of particular subtypes as described in detail herein. Various subtypes of influenza virus exist and continue to be identified as new subtypes emerge. It will be understood by one skilled in the art that the methods and compositions provided herein may be adapted to utilize sequences of additional subtypes. Such variation is contemplated and encompassed within the methods and compositions provided herein.

Influenza Polypeptide Fusions with Thermostable Proteins

In certain aspects, provided are influenza antigen polypeptide(s) comprising fusion polypeptides which comprise an influenza protein (or a portion or variant thereof) operably linked to a thermostable protein. Inventive fusion polypeptides can be produced in any available expression system known in the art. In certain embodiments, inventive fusion proteins are produced in a plant or portion thereof (e.g., plant, plant cell, root, sprout, etc.).

Enzymes or other proteins which are not found naturally in humans or animal cells are particularly appropriate for use in fusion polypeptides of the present invention. Thermostable proteins that, when fused, confer thermostability to a fusion product are useful. Thermostability allows produced protein to maintain conformation, and maintain produced protein at room temperature. This feature facilitates easy, time efficient and cost effective recovery of a fusion polypeptide. A representative family of thermostable enzymes useful in accordance with the invention is the glucanohydrolase family. These enzymes specifically cleave 1,4-β glucosidic bonds that are adjacent to 1,3-β linkages in mixed linked polysaccharides (Hahn et al., 1994 Proc. Natl. Acad. Sci., USA, 91:10417; incorporated herein by reference). Such enzymes are found in cereals, such as oat and barley, and are also found in a number of fungal and bacterial species, including C. thermocellum (Goldenkova et al., 2002, Mol. Biol. 36:698; incorporated herein by reference). Thus, desirable thermostable proteins for use in fusion polypeptides of the present invention include glycosidase enzymes. Exemplary thermostable glycosidase proteins include those represented by GenBank accession numbers selected from those set forth in Table A, the contents of each of which are incorporated herein by reference by entire incorporation of the GenBank accession information for each referenced number. Exemplary thermostable enzymes of use in fusion proteins in accordance with the invention include Clostridium thermocellum P29716, Brevibacillus brevis P37073, and Rhodthermus marinus P45798, each of which are incorporated herein by reference to their GenBank accession numbers. Representative fusion proteins utilize modified thermostable enzyme isolated from Clostridium thermocellum, however, any thermostable protein may be similarly utilized in accordance with the present invention. Exemplary thermostable glycosidase proteins are listed in Table 3:

TABLE 3
Thermostable Glycosidase Proteins

| GenBank | | |
|---|---|---|
| Accession | Strain | Sequence |
| P29716 | Beta- | 5′MKNRVISLLMASLLLVLSVIVAPFYKAEAATVVNTPFVAV |
| | glucanase | FSNFDSSQWEADWANGSVFNCVWKPSQVTFSNGKMILTLD |
| | Clostridium | REYGGSYPYKSGEYRTKSFFGYGYYEVRMKAAKNVGIVSSF |
| | thermocellum | FTYTGPSDNNPWDEIDIEFLGKDTTKVQFNWYKNGVGGNE |
| | | YLHNLGFDASQDFHTYGFEWRPDYIDFYVDGKKVYRGTRN |
| | | IPVTPGKIMMNLWPGIGVDEWLGRYDGRTPLQAEYEYVKY |
| | | YPNGVPQDNPTPTPTIAPSTPTNPNLPLKGDVNGDGHVNSSD |
| | | YSLFKRYLLRVIDRFPVGDQSVADVNRDGRIDSTDLTMLKR |
| | | YLIRAIPSL 3′ (SEQ ID NO: 44) |
| P37073 | Beta- | 5′MVKSKYLVFISVFSLLFGVFVVGFSHQGVKAEEERPMGTA |
| | glucanase | FYESFDAFDDERWSKAGVWTNGQMFNATWYPEQVTADGL |
| | Brevibacillus | MRLTIAKKTTSARNYKAGELRTNDFYHYGLFEVSMKPAKV |
| | brevis | EGTVSSFFTYTGEWDWDGDPWDEIDIEFLGKDTTRIQFNYFT |
| | | NGVGGNEFYYDLGFDASESFNTYAFEWREDSITWYVNGEA |
| | | VHTATENIPQTPQKIMMNLWPGVGVDGWTGVFDGDNTPVY |
| | | SYYDWVRYTPLQNYQIHQ 3′ (SEQ ID NO: 45) |
| P17989 | Beta- | 5′MNIKKTAVKSALAVAAAAAALTTNVSAKDFSGAELYTLE |
| | glucanase | EVQYGKFEARMKMAAASGTVSSMFLYQNGSEIADGRPWVE |
| | Fibrobacter | VDIEVLGKNPGSFQSNIITGKAGAQKTSEKHHAVSPAADQAF |
| | succinogenes | HTYGLEWTPNYVRWTVDGQEVRKTEGGQVSNLTGTQGLR |
| | | FNLWSSESAAWVGQFDESKLPLFQFINWVKVYKYTPGQGE |
| | | GGSDFTLDWTDNFDTFDGSRWGKGDWTFDGNRVDLTDKNI |
| | | YSRDGMLILALTRKGQESFNGQVPRDDEPAPQSSSSAPASSS |
| | | SVPASSSSVPASSSSAFVPPSSSSATNAIHGMRTTPAVAKEHR |
| | | NLVNAKGAKVNPNGHKRYRVNFEH 3′ (SEQ ID NO: 46) |
| P07883 | Extracellular | 5′MVNRRDLIKWSAVALGAGAGLAGPAPAAHAADLEWEQY |
| | agarase | PVPAAPGGNRSWQLLPSHSDDFNYTGKPQTFRGRWLDQHK |
| | Streptomyces | DGWSGPANSLYSARHSWVADGNLIVEGRRAPDGRVYCGY |
| | coelicolor | VTSRTPVEYPLYTEVLMRVSGLKLSSNFWLLSRDDVNEIDVI |
| | | ECYGNESLHGKHMNTAYHIFQRNPFTELARSQKGYFADGSY |
| | | GYNGETGQVFGDGAGQPLLRNGFHRYGVHWISATEFDFYF |
| | | NGRLVRRLNRSNDLRDPRSRFFDQPMHLILNTESHQWRVDR |
| | | GIEPTDAELADPSINNIYYRWVRTYQAV 3′ (SEQ ID NO: 47) |
| P23903 | Glucan | 5′MKPSHFTEKRFMKKVLGLFLVVVMLASVGVLPTSKVQAA |
| | endo-13- | GTTVTSMEYFSPADGPVISKSGVGKASYGFVMPKFNGGSAT |
| | beta- | WNDVYSDVGVNVKVGNNWVDIDQAGGYIYNQNWGHWSD |
| | glucosidase | GGFNGYWFTLSATTEIQLYSKANGVKLEYQLVFQNINKTTIT |
| | A1 Bacillus | AMNPTQGPQITASFTGGAGFTYPTFNNDSAVTYEAVADDLK |
| | circulans | VYVKPVNSSSWIDIDNNAASGWIYDHNFGQFTDGGGGYWF |
| | | NVTESINVKLESKTSSANLVYTITFNEPTRNSYVITPYEGTTF |
| | | TADANGSIGIPLPKIDGGAPIAKELGNFVYQININGQWVDLS |
| | | NSSQSKFAYSANGYNNMSDANQWGYWADYIYGLWFQPIQ |
| | | ENMQIRIGYPLNGQAGGNIGNNFVNYTFIGNPNAPRPDVSD |
| | | QEDISIGTPTDPAIAGMNLIWQDEFNGTTLDTSKWNYETGY |
| | | YLNNDPATWGWGNAELQHYTNSTQNVYVQDGKLNIKAMN |
| | | DSKSFPQDPNRYAQYSSGKINTKDKLSLKYGRVDFRAKLPT |
| | | GDGVWPALWMLPKDSVYGTWAASGEIDVMEARGRLPGSV |
| | | SGTIHFGGQWPVNQSSGGDYHFPEGQTFANDYHVYSVVWE |
| | | EDNIKWYVDGKFFYKVTNQQWYSTAAPNNPNAPFDEPFYLI |
| | | MNLAVGGNFDGGRTPNASDIPATMQVDYVRVYKEQ 3′ |
| | | (SEQ ID NO: 48) |
| P27051 | Beta- | 5′MSYRVKRMLMLLVTGLFLSLSTFAASASAQTGGSFYEPFN |
| | glucanase | NYNTGLWQKADGYSNGNMFNCTWRANNVSMTSLGEMRL |
| | Bacillus | SLTSPSYNKFDCGENRSVQTYGYGLYEVNMKPAKNVGIVSS |
| | licheniformis | FFTYTGPTDGTPWDEIDIEFLGKDTTKVQFNYYTNGVGNHE |
| | | KIVNLGFDAANSYHTYAFDWQPNSIKWYVDGQLKHTATTQ |
| | | IPQTPGKIMMNLWNGAGVDEWLGSYNGVTPLSRSLHWVRY |
| | | TKR 3′ (SEQ ID NO: 49) |
| P45797 | Beta- | 5′MMKKKSWFTLMITGVISLFFSVSAFAGNVFWEPLSYFNSS |
| | glucanase | TWQKADGYSNGQMFNCTWRANNVNFTNDGKLKLSLTSPA |
| | Paenibacillus | NNKFDCGEYRSTNNYGYGLYEVSMKPAKNTGIVSSFFTYTG |
| | polymyxa | PSHGTQWDEIDIEFLGKDTTKVQFNYYTNGVGGHEKIINLGF |
| | Bacillus | DASTSFHTYAFDWQPGYIKWYVDGVLKHTATTNIPSTPGKI |
| | polymyxa | MMNLWNGTGVDSWLGSYNGANPLYAEYDWVKYTSN 3′ |
| | | (SEQ ID NO: 50) |
| P37073 | Beta- | 5′MVKSKYLVFISVFSLLFGVFVVGFSHQGVKAEEERPMGTA |
| | glucanase | FYESFDAFDDERWSKAGVWTNGQMFNATWYPEQVTADGL |
| | Brevibacillus | MRLTIAKKTTSARNYKAGELRTNDFYHYGLFEVSMKPAKV |
| | brevis | EGTVSSFFTYTGEWDWDGDPWDEIDIEFLGKDTTRIQFNYFT |
| | | NGVGGNEFYYDLGFDASESFNTYAFEWREDSITWYVNGEA |
| | | VHTATENIPQTPQKIMMNLWPGVGVDGWTGVFDGDNTPVY |
| | | SYYDWVRYTPLQNYQIHQ 3′ (SEQ ID NO: 51) |
| P45798 | Beta- | 5′MCTMPLMKLKKMMRRTAFLLSVLIGCSMLGSDRSDKAPH |
| | glucanase | WELVWSDEFDYSGLPDPEKWDYDVGGHGWGNQELQYYTR |
| | Rhodothermus | ARIENARVGGGVLIIEARHEPYEGREYTSARLVTRGKASWT |
| | marinus | YGRFEIRARLPSGRGTWPAIWMLPDRQTYGSAYWPDNGEID |
| | | IMEHVGFNPDVVHGTVHTKAYNHLLGTQRGGSIRVPTARTD |
| | | FHVYAIEWTPEEIRWFVDDSLYYRFPNERLTDPEADWRHWP |
| | | FDQPFHLIMNIAVGGAWGGQQGVDPEAFPAQLVVDYVRVY |
| | | RWVE 3′ (SEQ ID NO: 52) |
| P38645 | Beta- | 5′MTESAMTSRAGRGRGADLVAAVVQGHAAASDAAGDLSF |
| | glucosidase | PDGFIWGAATAAYQIEGAWREDGRGLWDVFSHTPGKVASG |
| | Thermobispora | HTGDIACDHYHRYADDVRLMAGLGDRVYRFSVAWPRIVPD |
| | bispora | GSGPVNPAGLDFYDRLVDELLGHGITPYPTLYHWDLPQTLE |
| | | DRGGWAARDTAYRFAEYALAVHRRLGDRVRCWITLNEPW |
| | | VAAFLATHRGAPGAADVPRFRAVHHLLLGHGLGLRLRSAG |
| | | AGQLGLTLSLSPVIEARPGVRGGGRRVDALANRQFLDPALR |
| | | GRYPEEVLKIMAGHARLGHPGRDLETIHQPVDLLGVNYYSH |
| | | VRLAAEGEPANRLPGSEGIRFERPTAVTAWPGDRPDGLRTL |
| | | LLRLSRDYPGVGLIITENGAAFDDRADGDRVHDPERIRYLTA |
| | | TLRAVHDAIMAGADLRGYFVWSVLDNFEWAYGYHKRGIV |
| | | YVDYTTMRRIPRESALWYRDVVRRNGLRNGE 3′ (SEQ ID |
| | | NO: 53) |
| P40942 | Celloxy- | 5′MNKFLNKKWSLILTMGGIFLMATLSLIFATGKKAFNDQTS |
| | lanase | AEDIPSLAEAFRDYFPIGAAIEPGYTTGQIAELYKKHVNMLV |
| | Clostridium | AENAMKPASLQPTEGNFQWADADRIVQFAKENGMELRFHT |
| | stercorarium | LVWHNQTPTGFSLDKEGKPMVEETDPQKREENRKLLLQRL |
| | | ENYIRAVVLRYKDDIKSWDVVNEVIEPNDPGGMRNSPWYQI |
| | | TGTEYIEVAFRATREAGGSDIKLYINDYNTDDPVKRDILYEL |
| | | VKNLLEKGVPIDGVGHQTHIDIYNPPVERIIESIKKFAGLGLD |
| | | NIITELDMSIYSWNDRSDYGDSIPDYILTLQAKRYQELFDAL |
| | | KENKDIVSAVVFWGISDKYSWLNGFPVKRTNAPLLFDRNFM |
| | | PKPAFWAIVDPSRLRE 3′ (SEQ ID NO: 54) |
| P14002 | Beta- | 5′MAVDIKKIIKQMTLEEKAGLCSGLDFWHTKPVERLGIPSIM |
| | glucosidase | MTDGPHGLRKQREDAEIADINNSVPATCFPSAAGLACSWDR |
| | Clostridium | ELVERVGAALGEECQAENVSILLGPGANIKRSPLCGRNFEYF |
| | thermocellum | SEDPYLSSELAASHIKGVQSQGVGACLKHFAANNQEHRRMT |
| | | VDTIVDERTLREIYFASFENAVKKARPWVVMCAYNKLNGE |
| | | YCSENRYLLTEVLKNEWMHDGFVVSDWGAVNDRVSGLDA |
| | | GLDLEMPTSHGITDKKIVEAVKSGKLSENILNRAVERILKVIF |
| | | MALENKKENAQYDKDAHHRLARQAAAESMVLLKNEDDVL |
| | | PLKKSGTIALIGAFVKKPRYQGSGSSHITPTRLDDIYEEIKKA |
| | | GGDKVNLVYSEGYRLENDGIDEELINEAKKAASSSDVAVVF |
| | | AGLPDEYESEGFDRTHMSIPENQNRLIEAVAEVQSNIVVVLL |
| | | NGSPVEMPWIDKVKSVLEAYLGGQALGGALADVLFGEVNP |
| | | SGKLAETFPVKLSHNPSYLNFPGEDDRVEYKEGLFVGYRYY |
| | | DTKGIEPLFPFGHGLSYTKFEYSDISVDKKDVSDNSIINVSVK |
| | | VKNVGKMAGKEIVQLYVKDVKSSVRRPEKELKGFEKVFLN |
| | | PGEEKTVTFTLDKRAFAYYNTQIKDWHVESGEFLILIGRSSR |
| | | DIVLKESVRVNSTVKIRKRFTVNSAVEDVMSDSSAAAVLGP |
| | | VLKEITDALQIDMDNAHDMMAANIKNMPLRSLVGYSQGRL |
| | | SEEMLEELVDKINNVE 3′ (SEQ ID NO: 55) |
| O33830 | Alpha- | 5′MPSVKIGIIGAGSAVFSLRLVSDLCKTPGLSGSTVTLMDID |
| | glucosidase | EERLDAILTIAKKYVEEVGADLKFEKTMNLDDVIIDADFVIN |
| | Thermotoga | TAMVGGHTYLEKVRQIGEKYGYYRGIDAQEFNMVSDYYTF |
| | maritima | SNYNQLKYFVDIARKIEKLSPKAWYLQAANPIFEGTTLVTRT |
| | | VPIKAVGFCHGHYGVMEIVEKLGLEEEKVDWQVAGVNHGI |
| | | WLNRFRYNGGNAYPLLDKWIEEKSKDWKPENPFNDQLSPA |
| | | AIDMYRFYGVMPIGDTVRNSSWRYHRDLETKKKWYGEPW |
| | | GGADSEIGWKWYQDTLGKVTEITKKVAKFIKENPSVRLSDL |
| | | GSVLGKDLSEKQFVLEVEKILDPERKSGEQHIPFIDALLNDN |
| | | KARFVVNIPNKGIIHGIDDDVVVEVPALVDKNGIHPEKIEPPL |
| | | PDRVVKYYLRPRIMRMEMALEAFLTGDIRIIKELLYRDPRTK |
| | | SDEQVEKVIEEILALPENEEMRKHYLKR 3′ (SEQ ID NO: 56) |
| O43097 | Xylanase | 5′MVGFTPVALAALAATGALAFPAGNATELEKRQTTPNSEG |
| | Thermomyces | WHDGYYYSWWSDGGAQATYTNLEGGTYEISWGDGGNLV |
| | lanuginosus | GGKGWNPGLNARAIHFEGVYQPNGNSYLAVYGWTRNPLV |
| | | EYYIVENFGTYDPSSGATDLGTVECDGSIYRLGKTTRVNAPS |
| | | IDGTQTFDQYWSVRQDKRTSGTVQTGCHFDAWARAGLNV |
| | | NGDHYYQIVATEGYFSSGYARITVADVG 3′ (SEQ ID NO: 57) |
| P54583 | Endo- | 5′MPRALRRVPGSRVMLRVGVVVAVLALVAALANLAVPRP |
| | glucanase | ARAAGGGYWHTSGREILDANNVPVRIAGINWFGFETCNYV |
| | E1 Acidothermus | VHGLWSRDYRSMLDQIKSLGYNTIRLPYSDDILKPGTMPNSI |
| | cellulolyticus | NFYQMNQDLQGLTSLQVMDKIVAYAGQIGLRIILDRHRPDC |
| | | SGQSALWYTSSVSEATWISDLQALAQRYKGNPTVVGFDLH |
| | | NEPHDPACWGCGDPSIDWRLAAERAGNAVLSVNPNLLIFVE |
| | | GVQSYNGDSYWWGGNLQGAGQYPVVLNVPNRLVYSAHD |
| | | YATSVYPQTWFSDPTFPNNMPGIWNKNWGYLFNQNIAPVW |
| | | LGEFGTTLQSTTDQTWLKTLVQYLRPTAQYGADSFQWTFW |
| | | SWNPDSGDTGGILKDDWQTVDTVKDGYLAPIKSSIFDPVGA |
| | | SASPSSQPSPSVSPSPSPSPSASRTPTPTPTPTASPTPTLTPTATP |
| | | TPTASPTPSPTAASGARCTASYQVNSDWGNGFTVTVAVTNS |
| | | GSVATKTWTVSWTFGGNQTITNSWNAAVTQNGQSVTARN |
| | | MSYNNVIQPGQNTTFGFQASYTGSNAAPTVACAAS 3′ (SEQ |
| | | ID NO: 58) |
| P14288 | β-galacto- | 5′MLSFPKGFKFGWSQSGFQSEMGTPGSEDPNSDWHVWVH |
| | sidase | DRENIVSQVVSGDLPENGPGYWGNYKRFHDEAEKIGLNAV |
| | Sulfolobus | RINVEWSRIFPRPLPKPEMQTGTDKENSPVISVDLNESKLRE |
| | acidocaldarius | MDNYANHEALSHYRQILEDLRNRGFHIVLNMYHWTLPIWL |
| | | HDPIRVRRGDFTGPTGWLNSRTVYEFARFSAYVAWKLDDL |
| | | ASEYATMNEPNVVWGAGYAFPRAGFPPNYLSFRLSEIAKW |
| | | NIIQAHARAYDAIKSVSKKSVGIIYANTSYYPLRPQDNEAVEI |
| | | AERLNRWSFFDSIIKGEITSEGQNVREDLRNRLDWIGVNYYT |
| | | RTVVTKAESGYLTLPGYGDRCERNSLSLANLPTSDFGWEFF |
| | | PEGLYDVLLKYWNRYGLPLYVMENGIADDADYQRPYYLVS |
| | | HIYQVHRALNEGVDVRGYLHWSLADNYEWSSGFSMRFGLL |
| | | KVDYLTKRLYWRPSALVYREITRSNGIPEELEHLNRVPPIKP |
| | | LRH 3′ (SEQ ID NO: 59) |
| O52629 | β-galactosidase | 5′MFPEKFLWGVAQSGFQFEMGDKLRRNIDTNTDWWHWVR |
| | Pyrococcus | DKTNIEKGLVSGDLPEEGINNYELYEKDHEIARKLGLNAYRI |
| | woesei | GIEWSRIFPWPTTFIDVDYSYNESYNLIEDVKITKDTLEELDEI |
| | | ANKREVAYYRSVINSLRSKGFKVIVNLNHFTLPYWLHDPIEA |
| | | RERALTNKRNGWVNPRTVIEFAKYAAYIAYKFGDIVDMWS |
| | | TFNEPMVVVELGYLAPYSGFPPGVLNPEAAKLAILHMINAH |
| | | ALAYRQIKKFDTEKADKDSKEPAEVGIIYNNIGVAYPKDPN |
| | | DSKDVKAAENDNFFHSGLFFEAIHKGKLNIEFDGETFIDAPY |
| | | LKGNDWIGVNYYTREVVTYQEPMFPSIPLITFKGVQGYGYA |
| | | CRPGTLSKDDRPVSDIGWELYPEGMYDSIVEAHKYGVPVYV |
| | | TENGIADSKDILRPYYIASHIKMTEKAFEDGYEVKGYFHWA |
| | | LTDNFEWALGFRMRFGLYEVNLITKERIPREKSVSIFREIVAN |
| | | NGVTKKIEEELLRG 3′ (SEQ ID NO: 60) |
| P29094 | Oligo-16- | 5′MERVWWKEAVVYQIYPRSFYDSNGDGIGDIRGIIAKLDYL |
| | glucosidase | KELGVDVVWLSPVYKSPNDDNGYDISDYRDIMDEFGTMAD |
| | Geobacillus | WKTMLEEMHKRGIKLVMDLVVNHTSDEHPWFIESRKSKDN |
| | thermoglucosidasius | PYRDYYIWRPGKNGKEPNNWESVFSGSAWEYDEMTGEYYL |
| | | HLFSKKQPDLNWENPKVRREVYEMMKFWLDKGVDGFRMD |
| | | VINMISKVPELPDGEPQSGKKYASGSRYYMNGPRVHEFLQE |
| | | MNREVLSKYDIMTVGETPGVTPKEGILYTDPSRRELNMVFQ |
| | | FEHMDLDSGPGGKWDIRPWSLADLKKTMTKWQKELEGKG |
| | | WNSLYLNNHDQPRAVSRFGDDGKYRVESAKMLATFLHMM |
| | | QGTPYIYQGEEIGMTNVRFPSIEDYRDIETLNMYKERVEEYG |
| | | EDPQEVMEKIYYKGRDNARTPMQWDDSENAGFTAGTPWIP |
| | | VNPNYKEINVKAALEDPNSVFHYYKKLIQLRKQHDIIVYGT |
| | | YDLILEDDPYIYRYTRTLGNEQLIVITNFSEKTPVFRLPDHIIY |
| | | KTKELLISNYDVDEAEELKEIRLRPWEARVYKIRLP 3′ (SEQ |
| | | ID NO: 61) |
| P49067 | Alpha- | 5′MGDKINFIFGIHNHQPLGNFGWVFEEAYEKCYWPFLETLE |
| | amylase | EYPNMKVAIHTSGPLIEWLQDNRPEYIDLLRSLVKRGQVEIV |
| | Pyrococcus | VAGFYEPVLASIPKEDRIEQIRLMKEWAKSIGFDARGVWLTE |
| | furiosus | RVWQPELVKTLKESGIDYVIVDDYHFMSAGLSKEELYWPY |
| | | YTEDGGEVIAVFPIDEKLRYLIPFRPVDKVLEYLHSLIDGDES |
| | | KVAVFHDDGEKFGIWPGTYEWVYEKGWLREFFDRISSDEKI |
| | | NLMLYTEYLEKYKPRGLVYLPIASYFEMSEWSLPAKQARLF |
| | | VEFVNELKVKGIFEKYRVFVRGGIWKNFFYKYPESNYMHK |
| | | RMLMVSKLVRNNPEARKYLLRAQCNDAYWHGLFGGVYLP |
| | | HLRRAIWNNLIKANSYVSLGKVIRDIDYDGFEEVLIENDNFY |
| | | AVFKPSYGGSLVEFSSKNRLVNYVDVLARRWEHYHGYVES |
| | | QFDGVASIHELEKKIPDEIRKEVAYDKYRRFMLQDHVVPLG |
| | | TTLEDFMFSRQQEIGEFPRVPYSYELLDGGIRLKREHLGIEVE |
| | | KTVKLVNDGFEVEYIVNNKTGNPVLFAVELNVAVQSIMESP |
| | | GVLRGKEIVVDDKYAVGKFALKFEDEMEVWKYPVKTLSQS |
| | | ESGWDLIQQGVSYIVPIRLEDKIRFKLKFEEASG 3′ (SEQ ID |
| | | NO: 62) |
| JC7532 | Cellulase | 5′MMLRKKTKQLISSILILVLLLSLFPAALAAEGNTREDNFKH |
| | Bacillus | LLGNDNVKRPSEAGALQLQEVDGQMTLVDQHGEKIQLRGM |
| | species | STHGLQWFPEILNDNAYKALSNDWDSNMIRLAMYVGENGY |
| | | ATNPELIKQRVIDGIELAIENDMYVIVDWHVHAPGDPRDPV |
| | | YAGAKDFFREIAALYPNNPHIIYELANEPSSNNNGGAGIPNN |
| | | EEGWKAVKEYADPIVEMLRKSGNADDNIIIVGSPNWSQRPD |
| | | LAADNPIDDHHTMYTVHFYTGSHAASTESYPSETPNSERGN |
| | | VMSNTRYALENGVAVFATEWGTSQASGDGGPYFDEADVWI |
| | | EFLNENNISWANWSLTNKNEVSGAFTPFELGKSNATNLDPG |
| | | PDHVWAPEELSLSGEYVRARIKGVNYEPIDRTKYTKVLWDF |
| | | NDGTKQGFGVNSDSPNKELIAVDNENNTLKVSGLDVSNDVS |
| | | DGNFWANARLSANGWGKSVDILGAEKLTMDVIVDEPTTVA |
| | | IAAIPQSSKSGWANPERAVRVNAEDFVQQTDGKYKAGLTIT |
| | | GEDAPNLKNIAFHEEDNNMNNIILFVGTDAADVIYLDNIKVI |
| | | GTEVEIPVVHDPKGEAVLPSVFEDGTRQGWDWAGESGVKT |
| | | ALTIEEANGSNALSWEFGYPEVKPSDNWATAPRLDFWKSDL |
| | | VRGENDYVAFDFYLDPVRATEGAMNINLVFQPPTNGYWVQ |
| | | APKTYTINFDELEEANQVNGLYHYEVKINVRDITNIQDDTLL |
| | | RNMMIIFADVESDFAGRVFVDNVRFEGAATTEPVEPEPVDP |
| | | GEETPPVDEKEAKKEQKEAEKEEKEAVKEEKKEAKEEKKA |
| | | VKNEAKKK 3′ (SEQ ID NO: 63) |
| Q60037 | Xylanase A | 5′MQVRKRRGLLDVSTAVLVGILAGFLGVVLAASGVLSFGK |
| | Thermotoga | EASSKGDSSLETVLALSFEGTTEGVVPFGKDVVLTASQDVA |
| | maritima | ADGEYSLKVENRTSPWDGVEIDLTGKVKSGADYLLSFQVY |
| | | QSSDAPQLFNVVARTEDEKGERYDVILDKVVVSDHWKEILV |
| | | PFSPTFEGTPAKYSLIIVASKNTNFNFYLDKVQVLAPKESGPK |
| | | VIYETSFENGVGDWQPRGDVNIEASSEVAHSGKSSLFISNRQ |
| | | KGWQGAQINLKGILKTGKTYAFEAWVYQNSGQDQTIIMTM |
| | | QRKYSSDASTQYEWIKSATVPSGQWVQLSGTYTIPAGVTVE |
| | | DLTLYFESQNPTLEFYVDDVKIVDTTSAEIKIEMEPEKEIPAL |
| | | KEVLKDYFKVGVALPSKVFLNPKDIELITKHFNSITAENEMK |
| | | PESLLAGIENGKLKFRFETADKYIQFVEENGMVIRGHTLVW |
| | | HNQTPDWFFKDENGNLLSKEAMTERLKEYIHTVVGHFKGK |
| | | VYAWDVVNEAVDPNQPDGLRRSTWYQIMGPDYIELAFKFA |
| | | READPDAKLFYNDYNTFEPRKRDIIYNLVKDLKEKGLIDGIG |
| | | MQCHISLATDIKQIEEAIKKFSTIPGIEIHITELDMSVYRDSSSN |
| | | YPEAPRTALIEQAHKMMQLFEIFKKYSNVITNVTFWGLKDD |
| | | YSWRATRRNDWPLIFDKDHQAKLAYWAIVAPEVLPPLPKES |
| | | RISEGEAVVVGMMDDSYLMSKPIEILDEEGNVKATIRAVWK |
| | | DSTIYIYGEVQDKTKKPAEDGVAIFINPNNERTPYLQPDDTY |
| | | AVLWTNWKTEVNREDVQVKKFVGPGFRRYSFEMSITIPGVE |
| | | FKKDSYIGFDAAVIDDGKWYSWSDTTNSQKTNTMNYGTLK |
| | | LEGIMVATAKYGTPVIDGEIDEIWNTTEEIETKAVAMGSLDK |
| | | NATAKVRVLWDENYLYVLAIVKDPVLNKDNSNPWEQDSV |
| | | EIFIDENNHKTGYYEDDDAQFRVNYMNEQTFGTGGSPARFK |
| | | TAVKLIEGGYIVEAAIKWKTIKPTPNTVIGFNIQVNDANEKG |
| | | QRVGIISWSDPTNNSWRDPSKFGNLRLIK 3′ (SEQ ID NO: 64) |
| P33558 | Xylanase A | 5′MKRKVKKMAAMATSIIMAIMIILHSIPVLAGRIIYDNETGT |
| | Clostridium | HGGYDYELWKDYGNTIMELNDGGTFSCQWSNIGNALFRKG |
| | stercorarium | RKFNSDKTYQELGDIVVEYGCDYNPNGNSYLCVYGWTRNP |
| | | LVEYYIVESWGSWRPPGATPKGTITQWMAGTYEIYETTRVN |
| | | QPSIDGTATFQQYWSVRTSKRTSGTISVTEHFKQWERMGMR |
| | | MGKMYEVALTVEGYQSSGYANVYKNEIRIGANPTPAPSQSP |
| | | IRRDAFSIIEAEEYNSTNSSTLQVIGTPNNGRGIGYIENGNTVT |
| | | YSNIDFGSGATGFSATVATEVNTSIQIRSDSPTGTLLGTLYVS |
| | | STGSWNTYQTVSTNISKITGVHDIVLVFSGPVNVDNFIFSRSS |
| | | PVPAPGDNTRDAYSIIQAEDYDSSYGPNLQIFSLPGGGSAIGY |
| | | IENGYSTTYKNIDFGDGATSVTARVATQNATTIQVRLGSPSG |
| | | TLLGTIYVGSTGSFDTYRDVSATISNTAGVKDIVLVFSGPVN |
| | | VDWFVFSKSGT 3′ (SEQ ID NO: 65) |
| P05117 | Polygalacturonase-2 | 5′MVIQRNSILLLIIIFASSISTCRSNVIDDNLFKQVYDNILEQEF |
| | precursor | AHDFQAYLSYLSKNIESNNNIDKVDKNGIKVINVLSFGAKG |
| | Solanum | DGKTYDNIAFEQAWNEACSSRTPVQFVVPKNKNYLLKQITF |
| | lycopersicum | SGPCRSSISVKIFGSLEASSKISDYKDRRLWIAFDSVQNLVVG |
| | | GGGTINGNGQVWWPSSCKINKSLPCRDAPTALTFWNCKNL |
| | | KVNNLKSKNAQQIHIKFESCTNVVASNLMINASAKSPNTDG |
| | | VHVSNTQYIQISDTIIGTGDDCISIVSGSQNVQATNITCGPGH |
| | | GISIGSLGSGNSEAYVSNVTVNEAKIIGAENGVRIKTWQGGS |
| | | GQASNIKFLNVEMQDVKYPIIIDQNYCDRVEPCIQQFSAVQV |
| | | KNVVYENIKGTSATKVAIKFDCSTNFPCEGIIMENINLVGESG |
| | | KPSEATCKNVHFNNAEHVTPHCTSLEISEDEALLYNY 3′ |
| | | (SEQ ID NO: 66) |
| P04954 | Cellulase D | 5′MSRMTLKSSMKKRVLSLLIAVVFLSLTGVFPSGLIETKVSA |
| | Clostridium | AKITENYQFDSRIRLNSIGFIPNHSKKATIAANCSTFYVVKED |
| | thermocellum | GTIVYTGTATSMFDNDTKETVYIADFSSVNEEGTYYLAVPG |
| | | VGKSVNFKIAMNVYEDAFKTAMLGMYLLRCGTSVSATYNG |
| | | IHYSHGPCHTNDAYLDYINGQHTKKDSTKGWHDAGDYNK |
| | | YVVNAGITVGSMFLAWEHFKDQLEPVALEIPEKNNSIPDFLD |
| | | ELKYEIDWILTMQYPDGSGRVAHKVSTRNFGGFIMPENEHD |
| | | ERFFVPWSSAATADFVAMTAMAARIFRPYDPQYAEKCINAA |
| | | KVSYEFLKNNPANVFANQSGFSTGEYATVSDADDRLWAAA |
| | | EMWETLGDEEYLRDFENRAAQFSKKIEADFDWDNVANLG |
| | | MFTYLLSERPGKNPALVQSIKDSLLSTADSIVRTSQNHGYGR |
| | | TLGTTYYWGCNGTVVRQTMILQVANKISPNNDYVNAALDA |
| | | ISHVFGRNYYNRSYVTGLGINPPMNPHDRRSGADGIWEPWP |
| | | GYLVGGGWPGPKDWVDIQDSYQTNEIAINWNAALIYALAG |
| | | FVNYNSPQNEVLYGDVNDDGKVNSTDLTLLKRYVLKAVST |
| | | LPSSKAEKNADVNRDGRVNSSDVTILSRYLIRVIEKLPI 3′ |
| | | (SEQ ID NO: 67) |
| Q4J929 | N- | 5′MLRSLVLNEKLRARVLERAEEFLLNNKADEEVWFRELVL |
| | glycosylase | CILTSNSSFISAYKSMNYILDKILYMDEKEISILLQESGYRFYN |
| | Sulfolobus | LKAKYLYRAKNLYGKVKKTIKEIADKDQMQAREFIATHIYG |
| | acidocaldarius | IGYKEASHFLRNVGYLDLAIIDRHILRFINNLGIPIKLKSKREY |
| | | LLAESLLRSIANNLNVQVGLLDLFIFFKQTNTIVK 3′ (SEQ ID |
| | | NO: 68) |
| O33833 | Beta- | 5′MFKPNYHFFPITGWMNDPNGLIFWKGKYHMFYQYNPRKP |
| | fructosidase | EWGNICWGHAVSDDLVHWRHLPVALYPDDETHGVFSGSA |
| | Thermotoga | VEKDGKMFLVYTYYRDPTHNKGEKETQCVAMSENGLDFV |
| | maritima | KYDGNPVISKPPEEGTHAFRDPKVNRSNGEWRMVLGSGKD |
| | | EKIGRVLLYTSDDLFHWKYEGVIFEDETTKEIECPDLVRIGE |
| | | KDILIYSITSTNSVLFSMGELKEGKLNVEKRGLLDHGTDFYA |
| | | AQTFFGTDRVVVIGWLQSWLRTGLYPTKREGWNGVMSLPR |
| | | ELYVENNELKVKPVDELLALRKRKVFETAKSGTFLLDVKEN |
| | | SYEIVCEFSGEIELRMGNESEEVVITKSRDELIVDTTRSGVSG |
| | | GEVRKSTVEDEATNRIRAFLDSCSVEFFFNDSIAFSFRIHPEN |
| | | VYNILSVKSNQVKLEVFELENIWL 3′ (SEQ ID NO: 69) |
| P49425 | Endo-14- | 5′MAGPHRSRAAGPPPFAVDEHVALEMVAFRGEVFAGHGLL |
| | beta-mannosidase | ADQRLIAHTGRPALNAQRITQQKQRDQCRGQRHRHHQGGR |
| | Rhodothermus | NLRKAHRTFHEHQSTQDQAHDAPHGQQAKTGHEGLGHEH |
| | marinus | AQAQHQQGQSNVVDRQDGEPVEAQHQKDGAQRAGNAPA |
| | | GRVELEQQPVEAQHQQQEGDVRIGKRRQNAFAPPALDHVH |
| | | GGPGRLQRHGLAVERHVPAVQQHQQRVQRGRQQIDHVLG |
| | | HGLPGRQRLAFRDGPRRPVGVASPVLGQRPCPGHRIVQNLF |
| | | RHGIDPCRVGRCRRSPSELHGMGCADVRARGHGRHMRGQR |
| | | DEHPGRGRPCARRRHVDDDRDRTPQEKLYDVARGLDEPAR |
| | | RVHFDDEADRSVFRGLAQPAPDEPEGRRRDRLVLQRQSVN |
| | | HRRGRLSRHRQQHQPQQQRPHGNQAFLGKYEKRRRKPTAC |
| | | LKSLRRFPDKDAPVLYFVNQLEKTKRRMTLLLVWLIFTGVA |
| | | GEIRLEAEDGELLGVAVDSTLTGYSGRGYVTGFDAPEDSVR |
| | | FSFEAPRGVYRVVFGVSFSSRFASYALRVDDWHQTGSLIKR |
| | | GGGFFEASIGEIWLDEGAHTMAFQLMNGALDYVRLEPVSY |
| | | GPPARPPAQLSDSQATASAQALFAFLLSEYGRHILAGQQQNP |
| | | YRRDFDAINYVRNVTGKEPALVSFDLIDYSPTREAHGVVHY |
| | | QTPEDWIAWAGRDGIVSLMWHWNAPTDLIEDPSQDCYWW |
| | | YGFYTRCTTFDVAAALADTSSERYRLLLRDIDVIAAQLQKF |
| | | QQADIPVLWRPLHEAAGGWFWWGAKGPEPFKQLWRLLYE |
| | | RLVHHHGLHNLIWVYTHEPGAAEWYPGDAYVDIVGRDVY |
| | | ADDPDALMRSDWNELQTLFGGRKLVALTETGTLPDVEVITD |
| | | YGIWWSWFSIWTDPFLRDVDPDRLTRVYHSERVLTRDELPD |
| | | WRSYVLHATTVQPAGDLALAVYPNPGAGRLHVEVGLPVAA |
| | | PVVVEVFNLLGQRVFQYQAGMQPAGLWRRAFELALAPGV |
| | | YLVQVRAGNLVARRRWVSVR 3′ (SEQ ID NO: 70) |
| P06279 | Alpha- | 5′MLTFHRIIRKGWMFLLAFLLTALLFCPTGQPAKAAAPFNG |
| | amylase | TMMQYFEWYLPDDGTLWTKVANEANNLSSLGITALWLPPA |
| | Geobacillus | YKGTSRSDVGYGVYDLYDLGEFNQKGAVRTKYGTKAQYL |
| | stearothermophilus | QAIQAAHAAGMQVYADVVFDHKGGADGTEWVDAVEVNP |
| | | SDRNQEISGTYQIQAWTKFDFPGRGNTYSSFKWRWYHFDG |
| | | VDWDESRKLSRIYKFRGIGKAWDWEVDTENGNYDYLMYA |
| | | DLDMDHPEVVTELKSWGKWYVNTTNIDGFRLDAVKHIKFS |
| | | FFPDWLSDVRSQTGKPLFTVGEYWSYDINKLHNYIMKTNGT |
| | | MSLFDAPLHNKFYTASKSGGTFDMRTLMTNTLMKDQPTLA |
| | | VTFVDNHDTEPGQALQSWVDPWFKPLAYAFILTRQEGYPC |
| | | VFYGDYYGIPQYNIPSLKSKIDPLLIARRDYAYGTQHDYLDH |
| | | SDIIGWTREGVTEKPGSGLAALITDGPGGSKWMYVGKQHA |
| | | GKVFYDLTGNRSDTVTINSDGWGEFKVNGGSVSVWVPRKT |
| | | TVSTIAWSITTRPWTDEFVRWTEPRLVAWP 3′ (SEQ ID NO: |
| | | 71) |
| P45702 | Xylanase | 5′MPTNLFFNAHHSPVGAFASFTLGFPGKSGGLDLELARPPR |
| P45703 | Geobacillus | QNVLIGVESLHESGLYHVLPFLETAEEDESKRYDIENPDPNP |
| P40943 | stearothermophilus | QKPNILIPFAKEEIQREFHVATDTWKAGDLTFTIYSPVKAVP |
| | | NPETADEEELKLALVPAVIVEMTIDNTNGTRARRAFFGFEGT |
| | | DPYTSMRRIDDTCPQLRGVGQGRILSIVSKDEGVRSALHFSM |
| | | EDILTAQLEENWTFGLGKVGALIVDVPAGEKKTYQFAVCFY |
| | | RGGYVTAGMDASYFYTRFFQNIEEVGLYALEQAEVLKEQSF |
| | | RSNKLIEKEWLSDDQTFMMAHAIRSYYGNTQLLEHEGKPIW |
| | | VVNEGEYRMMNTFDLTVDQLFFELKLNPWTVKNVLDLYVE |
| | | RYSYEDRVRFPGEETEYPSGISFTHDMGVANTFSRPHYSSYE |
| | | LYGISGCFSHMTHEQLVNWVLCAAVYIEQTKDWAWRDKR |
| | | LAILEQCLESMVRRDHPDPEQRNGVMGLDSTRTMGGAEITT |
| | | YDSLDVSLGQARNNLYLAGKCWAAYVALEKLFRDVGKEE |
| | | LAALAGEQAEKCAATIVSHVTDDGYIPAIMGEGNDSKIIPAIE |
| | | GLVFPYFTNCHEALDENGRFGAYIQALRNHLQYVLREGICL |
| | | FPDGGWKISSTSNNSWLSKIYLCQFIARHILGWEWDEQGKR |
| | | ADAAHVAWLTHPTLSIWSWSDQIIAGEITGSKYYPRGVTSIL |
| | | WLEEGE 3′ (SEQ ID NO: 72) |
| | | 5′MCSSIPSLREVFANDFRIGAAVNPVTLEAQQSLLIRHVNSL |
| | | TAENHMKFEHLQPEEGRFTFDIAIKSSTSPFSSHGVRGHTLV |
| | | WHNQTPSWVFQDSQGHFVGRDVLLERMKSHISTVVQRYKG |
| | | KVYCWDVINEAVADEGSEWLRSSTWRQIIGDDFIQQAFLYA |
| | | HEADPEALLFYNDYNECFPEKREKIYTLVKSLRDKGIPIHGIG |
| | | MQAHWSLNRPTLDEIRAAIERYASLGVILHITELDISMFEFDD |
| | | HRKDLAAPTNEMVERQAERYEQIFSLFKEYRDVIQNVTFWG |
| | | IADDHTWLDHFPVQGRKNWPLLFDEQHNPKPAFWRVVNI |
| | | 3′ (SEQ ID NO: 73) |
| | | 5′MRNVVRKPLTIGLALTLLLPMGMTATSAKNADSYAKKPH |
| | | ISALNAPQLDQRYKNEFTIGAAVEPYQLQNEKDVQMLKRHF |
| | | NSIVAENVMKPISIQPEEGKFNFEQADRIVKFAKANGMDIRF |
| | | HTLVWHSQVPQWFFLDKEGKPMVNETDPVKREQNKQLLL |
| | | KRLETHIKTIVERYKDDIKYWDVVNEVVGDDGKLRNSPWY |
| | | QIAGIDYIKVAFQAARKYGGDNIKLYMNDYNTEVEPKRTAL |
| | | YNLVKQLKEEGVPIDGIGHQSHIQIGWPSEAEIEKTINMFAAL |
| | | GLDNQITELDVSMYGWPPRAYPTYDAIPKQKFLDQAARYD |
| | | RLFKLYEKLSDKISNVTFWGIADNHTWLDSRADVYYDANG |
| | | NVVVDPNAPYAKVEKGKGKDAPFVFGPDYKVKPAYWAIID |
| | | HK 3′ (SEQ ID NO: 74) |
| P09961 | Alpha- | 5′MTKSIYFSLGIHNHQPVGNFDFVIERAYEMSYKPLINFFFK |
| | amylase 1 | HPDFPINVHFSGFLLLWLEKNHPEYFEKLKIMAERGQIEFVS |
| | Dictyoglomus | GGFYEPILPIIPDKDKVQQIKKLNKYIYDKFGQTPKGMWLAE |
| | thermophilum | RVWEPHLVKYIAEAGIEYVVVDDAHFFSVGLKEEDLFGYYL |
| | | MEEQGYKLAVFPISMKLRYLIPFADPEETITYLDKFASEDKS |
| | | KIALLFDDGEKFGLWPDTYRTVYEEGWLETFVSKIKENFLL |
| | | VTPVNLYTYMQRVKPKGRIYLPTASYREMMEWVLFPEAQK |
| | | ELEELVEKLKTENLWDKFSPYVKGGFWRNFLAKYDESNHM |
| | | QKKMLYVWKKVQDSPNEEVKEKAMEEVFQGQANDAYWH |
| | | GIFGGLYLPHLRTAIYEHLIKAENYLENSEIRFNIFDFDCDGN |
| | | DEIIVESPFFNLYLSPNHGGSVLEWDFKTKAFNLTNVLTRRK |
| | | EAYHSKLSYVTSEAQGKSIHERWTAKEEGLENILFYDNHRR |
| | | VSFTEKIFESEPVLEDLWKDSSRLEVDSFYENYDYEINKDEN |
| | | KIRVLFSGVFRGFELCKSYILYKDKSFVDVVYEIKNVSETPIS |
| | | LNFGWEINLNFLAPNHPDYYFLIGDQKYPLSSFGIEKVNNW |
| | | KIFSGIGIELECVLDVEASLYRYPIETVSLSEEGFERVYQGSAL |
| | | IHFYKVDLPVGSTWRTTIRFWVK 3′ (SEQ ID NO: 75) |
| Q60042 | Xylanase A | 5′MRKKRRGFLNASTAVLVGILAGFLGVVLAATGALGFAVR |
| | Thermotoga | ESLLLKQFLFLSFEGNTDGASPFGKDVVVTASQDVAADGEY |
| | neapolitana | SLKVENRTSVWDGVEIDLTGKVNTGTDYLLSFHVYQTSDSP |
| | | QLFSVLARTEDEKGERYKILADKVVVPNYWKEILVPFSPTFE |
| | | GTPAKFSLIITSPKKTDFVFYVDNVQVLTPKEAGPKVVYETS |
| | | FEKGIGDWQPRGSDVKISISPKVAHSGKKSLFVSNRQKGWH |
| | | GAQISLKGILKTGKTYAFEAWVYQESGQDQTIIMTMQRKYS |
| | | SDSSTKYEWIKAATVPSGQWVQLSGTYTIPAGVTVEDLTLY |
| | | FESQNPTLEFYVDDVKVVDTTSAEIKLEMNPEEEIPALKDVL |
| | | KDYFRVGVALPSKVFINQKDIALISKHSNSSTAENEMKPDSL |
| | | LAGIENGKLKFRFETADKYIEFAQQNGMVVRGHTLVWHNQ |
| | | TPEWFFKDENGNLLSKEEMTERLREYIHTVVGHFKGKVYA |
| | | WDVVNEAVDPNQPDGLRRSTWYQIMGPDYIELAFKFAREA |
| | | DPNAKLFYNDYNTFEPKKRDIIYNLVKSLKEKGLIDGIGMQC |
| | | HISLATDIRQIEEAIKKFSTIPGIEIHITELDISVYRDSTSNYSEA |
| | | PRTALIEQAHKMAQLFKIFKKYSNVITNVTFWGLKDDYSWR |
| | | ATRRNDWPLIFDKDYQAKLAYWAIVAPEVLPPLPKESKISEG |
| | | EAVVVGMMDDSYMMSKPIEIYDEEGNVKATIRAIWKDSTIY |
| | | VYGEVQDATKKPAEDGVAIFINPNNERTPYLQPDDTYVVLW |
| | | TNWKSEVNREDVEVKKFVGPGFRRYSFEMSITIPGVEFKKD |
| | | SYIGFDVAVIDDGKWYSWSDTTNSQKTNTMNYGTLKLEGV |
| | | MVATAKYGTPVIDGEIDDIWNTTEEIETKSVAMGSLEKNAT |
| | | AKVRVLWDEENLYVLAIVKDPVLNKDNSNPWEQDSVEIFID |
| | | ENNHKTGYYEDDDAQFRVNYMNEQSFGTGASAARFKTAV |
| | | KLIEGGYIVEAAIKWKTIKPSPNTVIGFNVQVNDANEKGQRV |
| | | GIISWSDPTNNSWRDPSKFGNLRLIK 3′ (SEQ ID NO: 76) |
| AAN05438 | Beta- | 5′MDDHAEKFLWGVATSAYQIEGATQEDGRGPSIWDAFARR |
| AAN05439 | glycosidase | PGAIRDGSTGEPACDHYRRYEEDIALMQSLGVRAYRFSVAW |
| | Thermus | PRILPEGRGRINPKGLAFYDRLVDRLLASGITPFLTLYHWDLP |
| | thermophilus | LALEERGGWRSRETAFAFAEYAEAVARALADRVPFFATLNE |
| | | PWCSAFLGHWTGEHAPGLRNLEAALRAAHHLLLGHGLAVE |
| | | ALRAAGARRVGIVLNFAPAYGEDPEAVDVADRYHNRYFLD |
| | | PILGKGYPESPFRDPPPVPILSRDLELVARPLDFLGVNYYAPV |
| | | RVAPGTGTLPVRYLPPEGPATAMGWEVYPEGLHHLLKRLG |
| | | REVPWPLYVTENGAAYPDLWTGEAVVEDPERVAYLEAHVE |
| | | AALRAREEGVDLRGYFVWSLMDNFEWAFGYTRRFGLYYV |
| | | DFPSQRRIPKRSALWYRERIARAQT 3′ (SEQ ID NO: 77) |
| | | 5′MTENAEKFLWGVATSAYQIEGATQEDGRGPSIWDAFAQR |
| | | PGAIRDGSTGEPACDHYRRYEEDIALMQSLGVRAYRFSVAW |
| | | PRILPEGRGRINPKGLAFYDRLVDRLLASGITPFLTLYHWDLP |
| | | LALEERGGWRSRETAFAFAEYAEAVARALADRVPFFATLNE |
| | | PWCSAFLGHWTGEHAPGLRNLEAALRAAHHLLLGHGLAVE |
| | | ALRAAGARRVGIVLNFAPAYGEDPEAVDVADRYHNRFFLD |
| | | PILGKGYPESPFRDPPPVPILSRDLELVARPLDFLGVNYYAPV |
| | | RVAPGTGTLPVRYLPPEGPATAMGWEVYPEGLYHLLKRLG |
| | | REVPWPLYVTENGAAYPDLWTGEAVVEDPERVAYLEAHVE |
| | | AALRAREEGVDLRGYFVWSLMDNFEWAFGYTRRFGLYYV |
| | | DFPSQRRIPKRSALWYRERIARAQT 3′ (SEQ ID NO: 78) |
| AAN05437 | Sugar | 5′MAQVGRGASPLSRARVPPLPHPLDGEHLPHDPAGGGHGK |
| | permease | ASSQDAPVGQLPGHLARPAFFHYLKNSFLVCSLTTVFALAV |
| | Thermus | ATFAGYALARFRFPGAELFGGSVLVTQVIPGILFLIPIYIMYIY |
| | thermophilus | VQNWVRSALGLEVRLVGSYGGLVFTYTAFFVPLSIWILRGF |
| | | FASIPKELEEAAMVDGATPFQAFHRVILPLALPGLAATAVYI |
| | | FLTAWDELLFAQVLTTEATATVPVGIRNFVGNYQNRYDLV |
| | | MAAATVATLPVLVLFFFVQRQLIQGLTAGAVKG 3′ (SEQ ID |
| | | NO: 79) |
| AAN05440 | Beta- | 5′MAENAEKFLWGVATSAYQIEGATQEDGRGPSIWDTFARR |
| | glycosidase | PGAIRDGSTGEPACDHYHRYEEDIALMQSLGVGVYRFSVA |
| | Thermus | WPRILPEGRGRINPKGLAFYDRLVDRLLAAGITPFLTLYHWD |
| | filiformis | LPQALEDRGGWRSRETAFAFAEYAEAVARALADRVPFFATL |
| | | NEPWCSAFLGHWTGEHAPGLRNLEAALRAAHHLLLGHGLA |
| | | VEALRAAGAKRVGIVLNFAPVYGEDPEAVDVADRYHNRYF |
| | | LDPILGRGYPESPFQDPPPTPNLSRDLELVARPLDFLGVNYY |
| | | APVRVAPGTGPLPVRYLPPEGPVTAMGWEVYPEGLYHLLK |
| | | RLGREVPWPLYITENGAAYPDLWTGEAVVEDPERVAYLEA |
| | | HVEAALRAREEGVDLRGYFVWSLMDNFEWAFGYTRRFGL |
| | | YYVDFPSQRRIPKRSALWYRERIARAQL 3′ (SEQ ID NO: 80) |
| AAD43138 | Beta- | 5′MKFPKDFMIGYSSSPFQFEAGIPGSEDPNSDWWVWVHDPE |
| | glycosidase | NTAAGLVSGDFPENGPGYWNLNQNDHDLAEKLGVNTIRVG |
| | Thermosphaera | VEWSRIFPKPTFNVKVPVERDENGSIVHVDVDDKAVERLDE |
| | aggregans | LANKEAVNHYVEMYKDWVERGRKLILNLYHWPLPLWLHN |
| | | PIMVRRMGPDRAPSGWLNEESVVEFAKYAAYIAWKMGELP |
| | | VMWSTMNEPNVVYEQGYMFVKGGFPPGYLSLEAADKARR |
| | | NMIQAHARAYDNIKRFSKKPVGLIYAFQWFELLEGPAEVFD |
| | | KFKSSKLYYFTDIVSKGSSIINVEYRRDLANRLDWLGVNYYS |
| | | RLVYKIVDDKPIILHGYGFLCTPGGISPAENPCSDFGWEVYPE |
| | | GLYLLLKELYNRYGVDLIVTENGVSDSRDALRPAYLVSHVY |
| | | SVWKAANEGIPVKGYLHWSLTDNYEWAQGFRQKFGLVMV |
| | | DFKTKKRYLRPSALVFREIATHNGIPDELQHLTLIQ 3′ (SEQ |
| | | ID NO: 81) |

While sequences of exemplary thermostable polypeptides are provided herein, it will be appreciated that any sequence exhibiting thermostability may be employed. In some embodiments, a thermostable polypeptide for use in accordance with the present invention has an amino acid sequence which is about 60% identical, about 70% identical, about 80% identical, about 85% identical, about 90% identical, about 91% identical, about 92% identical, about 93% identical, about 94% identical, about 95% identical, about 96% identical, about 97% identical, about 98% identical, about 99% identical, or 100% identical to a sequence selected from the group consisting of SEQ ID NOs: 44-83. In some embodiments, such a thermostable polypeptide retains thermostability.

In some embodiments, a thermostable polypeptide has an amino acid sequence which comprises about 100 contiguous amino acids of a sequence selected from the group consisting of SEQ ID NOs: 44-83. In some embodiments, a thermostable polypeptide has an amino acid sequence which is about 60% identical, about 70% identical, about 80% identical, about 85% identical, about 90% identical, about 91% identical, about 92% identical, about 93% identical, about 94% identical, about 95% identical, about 96% identical, about 97% identical, about 98% identical, about 99% identical, or 100% identical to a contiguous stretch of about 100 amino acids of a sequence selected from the group consisting of SEQ ID NOs: 44-83.

In some embodiments, a thermostable polypeptide has an amino acid sequence which comprises about 150, about 200, about 250, about 300, about 350, about 400, about 450, about 500, about 550, about 600, about 650, about 700, or more contiguous amino acids of a sequence selected from the group consisting of SEQ ID NOs: 44-83. In some embodiments, a thermostable polypeptide has an amino acid sequence which is about 60% identical, about 70% identical, about 80% identical, about 85% identical, about 90% identical, about 91% identical, about 92% identical, about 93% identical, about 94% identical, about 95% identical, about 96% identical, about 97% identical, about 98% identical, about 99% identical, or 100% identical to a contiguous stretch of about 150, 200, 250, 300, 350, or more amino acids of a sequence selected from the group consisting of SEQ ID NO: 44-83.

When designing fusion proteins and polypeptides in accordance with the invention, it is desirable, of course, to preserve immunogenicity of the antigen. Still further, it is desirable in certain aspects to provide constructs which provide thermostability of a fusion protein. This feature facilitates easy, time efficient and cost effective recovery of a target antigen. In certain aspects, antigen fusion partners may be selected which provide additional advantages, including enhancement of immunogenicity, potential to incorporate multiple vaccine determinants, yet lack prior immunogenic exposure to vaccination subjects. Further beneficial qualities of fusion peptides of interest include proteins which provide ease of manipulation for incorporation of one or more antigens, as well as proteins which have potential to confer ease of production, purification, and/or formulation for vaccine preparations. One of ordinary skill in the art will appreciate that three dimensional presentation can affect each of these beneficial characteristics. Preservation of immunity or preferential qualities therefore may affect, for example, choice of fusion partner and/or choice of fusion location (e.g., N-terminus, C-terminus, internal, combinations thereof). Alternatively or additionally, preferences may affects length of segment selected for fusion, whether it be length of antigen, or length of fusion partner selected.

The present inventors have demonstrated successful fusion of a variety of antigens with a thermostable protein. For example, the present inventors have used the thermostable carrier molecule LicB, also referred to as lichenase, for production of fusion proteins. LicB is 1,3-1,4-β glucanase (LicB) from Clostridium thermocellum (GenBank accession: X63355 [gi:40697]): MKNRVISLLMASLLLVLSVIVAPFYKAEAATVVNTPFVAVFSNFDSSQWEKADWAN GSVFNCVWKPSQVTFSNGKMILTLDREYGGSYPYKSGEYRTKSFFGYGYYEVRMKA AKNVGIVSSFFTYTGP SDNNPWDEIDIEFLGKDTTKVQFNWYKNGVGGNEYLHNLG FDASQDFHTYGFEWRPDYIDFYVDGKKVYRGTRNIPVTPGKIMMNLWPGIGVDEWL GRYDGRTPLQAEYEYVKYYPNGVPQDNPTPTPTIAPSTPTNPNLPLKGDVNGDGHVN SSDYSLFKRYLLRVIDRFPVGDQSVADVNRDGRIDSTDLTMLKRYLIRAIPSL (SEQ ID NO: 82). LicB belongs to a family of globular proteins. Based on the three dimensional structure of LicB, its N- and C-termini are situated close to each other on the surface, in close proximity to the active domain. LicB also has a loop structure exposed on the surface that is located far from the active domain. We have generated constructs such that the loop structure and N- and C-termini of protein can be used as insertion sites for influenza antigen polypeptides. Influenza antigen polypeptides can be expressed as N- or C-terminal fusions or as inserts into the surface loop. Importantly, LicB maintains its enzymatic activity at low pH and at high temperature (up to 75° C.). Thus, use of LicB as a carrier molecule contributes advantages, including likely enhancement of target specific immunogenicity, potential to incorporate multiple vaccine determinants, and straightforward formulation of vaccines that may be delivered nasally, orally or parenterally. Furthermore, production of LicB fusions in plants should reduce the risk of contamination with animal or human pathogens. See examples provided herein.

Fusion proteins in accordance with the invention comprising influenza antigen polypeptides may be produced in any of a variety of expression systems, including both in vitro and in vivo systems. One skilled in the art will readily appreciate that optimization of nucleic acid sequences for a particular expression system is often desirable. For example, an exemplary optimized sequence for expression of influenza antigen-LicB fusions in plants is provided, and is shown in SEQ ID NO: 83:

(SEQ ID NO: 83)

5′MGFVLFSQLPSFLLVSTLLLFLVISHSCRAQNGGSYPYKSGEYRTKS
FFGYGYYEVRMKAAKNVGIVSSFFTYTGPSDNNPWDEIDIEFLGKDTTKV
QFNWYKNGVGGNEYLHNLGFDASQDFHTYGFEWRPDYIDFYVDGKKVY
RGTRNIPVTPGKIMMNLWPGIGVDEWLGRYDGRTPLQAEYEYVKYYPNG
rsklVVNTPFVAVFSNFDSSQWEKADWANGSVFNCVWKPSQVTFSNGK
MILTLDREYvdHHHHHHKDEL 3′.

Note that in SEQ ID NO: 83, the bold/underlined portion corresponds to the signal sequence, the italicized/underlined portion corresponds to the 6×His tag and endoplasmic reticulum retention sequence, and the two portions in lowercase letters correspond to restriction sites.

Thus, any relevant nucleic acid encoding influenza antigen polypeptide(s), fusion protein(s), and immunogenic portions thereof in accordance with the invention is intended to be encompassed within nucleic acid constructs in accordance with the invention.

For production in plant systems, transgenic plants expressing influenza antigen(s) (e.g., influenza polypeptide(s), fusion(s) thereof, and/or immunogenic portion(s) thereof) may be utilized. Alternatively or additionally, transgenic plants may be produced using methods well known in the art to generate stable production crops. Additionally, plants utilizing transient expression systems may be utilized for production of influenza antigen polypeptide(s). When utilizing plant expression systems, whether transgenic or transient expression in plants is utilized, any of nuclear expression, chloroplast expression, mitochondrial expression, or viral expression may be taken advantage of according to the applicability of the system to antigen desired. Furthermore, additional expression systems for production of antigens and fusion proteins in accordance with the present invention may be utilized. For example, mammalian expression systems (e.g., mammalian cell lines [e.g., CHO, etc.]), bacterial expression systems (e.g., E. coli), insect expression systems (e.g., baculovirus), yeast expression systems, and in vitro expression systems (e.g., reticulate lysates) may be used for expression of antigens and fusion proteins in accordance with the invention.

### Production of Influenza Antigens

In accordance with the present invention, influenza antigens (including influenza polypeptide(s), fusions thereof, and/or immunogenic portions thereof) may be produced in any desirable system; production is not limited to plant systems. Vector constructs and expression systems are well known in the art and may be adapted to incorporate use of influenza antigen polypeptides provided herein. For example, influenza antigen polypeptides can be produced in known expression systems, including mammalian cell systems, transgenic animals, microbial expression systems, insect cell systems, and plant systems, including transgenic and transient plant systems. Particularly where influenza antigen polypeptides are produced as fusion proteins, it may be desirable to produce such fusion proteins in non-plant systems.

In some embodiments, influenza antigen polypeptides are desirably produced in plant systems. Plants are relatively easy to manipulate genetically, and have several advantages over alternative sources such as human fluids, animal cell lines, recombinant microorganisms and transgenic animals. Plants have sophisticated post-translational modification machinery for proteins that is similar to that of mammals (although it should be noted that there are some differences in glycosylation patterns between plants and mammals). This enables production of bioactive reagents in plant tissues. Also, plants can economically produce very large amounts of biomass without requiring sophisticated facilities. Moreover, plants are not subject to contamination with animal pathogens. Like liposomes and microcapsules, plant cells are expected to provide protection for passage of antigen to the gastrointestinal tract.

Plants may be utilized for production of heterologous proteins via use of various production systems. One such system includes use of transgenic/genetically-modified plants where a gene encoding target product is permanently incorporated into the genome of the plant. Transgenic systems may generate crop production systems. A variety of foreign proteins, including many of mammalian origin and many vaccine candidate antigens, have been expressed in transgenic plants and shown to have functional activity. (Tacket et al., 2000, J. Infect. Dis., 182:302; and Thanavala et al., 2005, Proc. Natl. Acad. Sci., USA, 102:3378; both of which are incorporated herein by reference). Additionally, administration of unprocessed transgenic plants expressing hepatitis B major surface antigen to non-immunized human volunteers resulted in production of immune response (Kapusta et al., 1999, FASEB J., 13:1796; incorporated herein by reference).

One system for expressing polypeptides in plants utilizes plant viral vectors engineered to express foreign sequences (e.g., transient expression). This approach allows for use of healthy non-transgenic plants as rapid production systems. Thus, genetically engineered plants and plants infected with recombinant plant viruses can serve as “green factories” to rapidly generate and produce specific proteins of interest. Plant viruses have certain advantages that make them attractive as expression vectors for foreign protein production. Several members of plant RNA viruses have been well characterized, and infectious cDNA clones are available to facilitate genetic manipulation. Once infectious viral genetic material enters a susceptible host cell, it replicates to high levels and spreads rapidly throughout the entire plant. There are several approaches to producing target polypeptides using plant viral expression vectors, including incorporation of target polypeptides into viral genomes. One approach involves engineering coat proteins of viruses that infect bacteria, animals or plants to function as carrier molecules for antigenic peptides. Such carrier proteins have the potential to assemble and form recombinant virus-like particles displaying desired antigenic epitopes on their surface. This approach allows for time-efficient production of vaccine candidates, since the particulate nature of a vaccine candidate facilitates easy and cost-effective recovery from plant tissue. Additional advantages include enhanced target-specific immunogenicity, the potential to incorporate multiple vaccine determinants, and ease of formulation into vaccines that can be delivered nasally, orally or parenterally. As an example, spinach leaves containing recombinant plant viral particles carrying epitopes of virus fused to coat protein have generated immune response upon administration (Modelska et al., 1998, Proc. Natl. Acad. Sci., USA, 95:2481; and Yusibov et al., 2002, Vaccine, 19/20:3155; both of which are incorporated herein by reference).

Plant Expression Systems

The teachings of the present invention are applicable to a wide variety of different plants. In general, any plants that are amendable to expression of introduced constructs as described herein are useful in accordance with the present invention. In many embodiments, it will be desirable to use young plants in order to improve the speed of protein/polypeptide production. As indicated here, in many embodiments, sprouted seedlings are utilized. As is known in the art, most sprouts are quick growing, edible plants produced from storage seeds. However, those of ordinary skill in the art will appreciate that the term “sprouted seedling” has been used herein in a more general context, to refer to young plants whether or not of a variety typically classified as “sprouts.” Any plant that is grown long enough to have sufficient green biomass to allow introduction and/or expression of an expression construct as provided for herein (recognizing that the relevant time may vary depending on the mode of delivery and/or expression of the expression construct) can be considered a “sprouted seedling” herein.

In many embodiments, edible plants are utilized (i.e., plants that are edible by—not toxic to—the subject to whom the protein or polypeptide is to be administered).

Any plant susceptible to incorporation and/or maintenance of heterologous nucleic acid and capable of producing heterologous protein may be utilized in accordance with the present invention. In general, it will often be desirable to utilize plants that are amenable to growth under defined conditions, for example in a greenhouse and/or in aqueous systems. It may be desirable to select plants that are not typically consumed by human beings or domesticated animals and/or are not typically part of the human food chain, so that they may be grown outside without concern that expressed polynucleotide may be undesirably ingested. In some embodiments, however, it will be desirable to employ edible plants. In particular embodiments, it will be desirable to utilize plants that accumulate expressed polypeptides in edible portions of a plant.

Often, certain desirable plant characteristics will be determined by the particular polynucleotide to be expressed. To give but a few examples, when a polynucleotide encodes a protein to be produced in high yield (as will often be the case, for example, when antigen proteins are to be expressed), it will often be desirable to select plants with relatively high biomass (e.g., tobacco, which has additional advantages that it is highly susceptible to viral infection, has a short growth period, and is not in the human food chain). Where a polynucleotide encodes antigen protein whose full activity requires (or is inhibited by) a particular post-translational modification, the ability (or inability) of certain plant species to accomplish relevant modification (e.g., a particular glycosylation) may direct selection. For example, plants are capable of accomplishing certain post-translational modifications (e.g., glycosylation), however, plants will not generate sialyation patterns which are found in mammalian post-translational modification. Thus, plant production of antigen may result in production of a different entity than the identical protein sequence produced in alternative systems.

In certain embodiments, crop plants, or crop-related plants are utilized. In certain specific embodiments, edible plants are utilized.

Plants for use in accordance with the present invention include Angiosperms, Bryophytes (e.g., Hepaticae, Musci, etc.), Pteridophytes (e.g., ferns, horsetails, lycopods), Gymnosperms (e.g., conifers, cycase, Ginko, Gnetales), and Algae (e.g., Chlorophyceae, Phaeophyceae, Rhodophyceae, Myxophyceae, Xanthophyceae, and Euglenophyceae). Exemplary plants are members of the family Leguminosae (Fabaceae; e.g., pea, alfalfa, soybean); Gramineae (Poaceae; e.g., corn, wheat, rice); Solanaceae, particularly of the genus Lycopersicon (e.g., tomato), Solanum (e.g., potato, eggplant), Capsium (e.g., pepper), or Nicotiana (e.g., tobacco); Umbelliferae, particularly of the genus Daucus (e.g., carrot), Apium (e.g., celery), or Rutaceae (e.g., oranges); Compositae, particularly of the genus Lactuca (e.g., lettuce); Brassicaceae (Cruciferae), particularly of the genus Brassica or Sinapis. In certain aspects, plants in accordance with the invention may be species of Brassica or Arabidopsis. Some exemplary Brassicaceae family members include Brassica campestris, B. carinata, B. juncea, B. napus, B. nigra, B. oleraceae, B. tournifortii, Sinapis alba, and Raphanus sativus. Some suitable plants that are amendable to transformation and are edible as sprouted seedlings include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as sunflower etc.

A wide variety of plant species may be suitable in the practice of the present invention. A variety of different bean and other species including, for example, adzuki bean, alfalfa, barley, broccoli, bill jump pea, buckwheat, cabbage, cauliflower, clover, collard greens, fenugreek, flax, garbanzo bean, green pea, Japanese spinach, kale, kamut, kohlrabi, marrowfat pea, mung bean, mustard greens, pinto bean, radish, red clover, soy bean, speckled pea, sunflower, turnip, yellow trapper pea, and others may be amenable to the production of heterologous proteins from viral vectors launched from an agrobacterial construct (e.g., introduced by agroinfiltration). In some embodiments, bill jump pea, green pea, marrowfat pea, speckled pea, and/or yellow trapper pea are particularly useful in accordance with this aspect of the invention. In certain embodiments, therefore, the present invention provides production of proteins or polypeptides (e.g., antigens) in one or more of these plants using an agrobacterial vector that launches a viral construct (i.e., an RNA with characteristics of a plant virus) encoding the relevant protein or polypeptide of interest. In some embodiments, the RNA has characteristics of (and/or includes sequences of) AlMV. In some embodiments, the RNA has characteristics of (and/or includes sequences of) TMV.

It will be appreciated that, in one aspect, the present invention provides young plants (e.g., sprouted seedlings) that express a target protein or polypeptide of interest. In some embodiments, the young plants were grown from transgenic seeds; the present invention also provides seeds which can be generated and/or utilized for the methods described herein. Seeds transgenic for any gene of interest can be sprouted and optionally induced for production of a protein or polypeptide of interest. For example, seeds capable of expressing any gene of interest can be sprouted and induced through: i) virus infection, ii) agroinfiltration, or iii) bacteria that contain virus genome. Seeds capable of expressing a transgene for heavy or light chain of any monoclonal antibody can be sprouted and induced for production of full-length molecule through: i) virus infection, ii) agroinfiltration, or iii) inoculation with bacteria that contain virus genome. Seeds capable of expressing a transgene for one or more components of a complex molecule comprising multiple components such as sIgA can be sprouted and used for producing a fully functional molecule through: i) virus infection, ii) agroinfiltration, or iii) inoculation with bacteria that contain virus genome. Seeds from healthy non-transgenic plants can be sprouted and used for producing target sequences through: i) virus infection, ii) agroinfiltration, or iii) inoculation with bacteria that contain a virus genome.

In some embodiments, the young plants were grown from seeds that were not transgenic. Typically, such young plants will harbor viral sequences that direct expression of the protein or polypeptide of interest. In some embodiments, the plants may also harbor agrobacterial sequences, optionally including sequences that “launched” the viral sequences.

Introducing Vectors into Plants

In general, vectors may be delivered to plants according to known techniques. For example, vectors themselves may be directly applied to plants (e.g., via abrasive inoculations, mechanized spray inoculations, vacuum infiltration, particle bombardment, or electroporation). Alternatively or additionally, virions may be prepared (e.g., from already infected plants), and may be applied to other plants according to known techniques.

A wide variety of viruses are known that infect various plant species, and can be employed for polynucleotide expression according to the present invention (see, for example, in The Classification and Nomenclature of Viruses, “Sixth Report of the International Committee on Taxonomy of Viruses” (Ed. Murphy et at), Springer Verlag: New York, 1995; Grierson et al., Plant Molecular Biology, Blackie, London, pp. 126-146, 1984; Gluzman et al., Communications in Molecular Biology: Viral Vectors, Cold Spring Harbor Laboratory, Cold Spring Harbor, N.Y., pp. 172-189, 1988; and Mathew, Plant Viruses Online; all of which are incorporated herein by reference). In certain embodiments, rather than delivering a single viral vector to a plant cell, multiple different vectors are delivered which, together, allow for replication (and, optionally cell-to-cell and/or long distance movement) of viral vector(s). Some or all of the proteins may be encoded by the genome of transgenic plants. In certain aspects, described in further detail herein, these systems include one or more viral vector components.

Vector systems that include components of two heterologous plant viruses in order to achieve a system that readily infects a wide range of plant types and yet poses little or no risk of infectious spread. An exemplary system has been described previously (see, e.g., PCT Publication WO 00/25574 and U.S. Patent Publication 2005/0026291, both of which are incorporated herein by reference). As noted herein, in particular aspects of the present invention, viral vectors are applied to plants (e.g., plant, portion of plant, sprout, etc.), for example, through infiltration or mechanical inoculation, spray, etc. Where infection is to be accomplished by direct application of a viral genome to a plant, any available technique may be used to prepare the genome. For example, many viruses that are usefully employed in accordance with the present invention have ssRNA genomes. ssRNA may be prepared by transcription of a DNA copy of the genome, or by replication of an RNA copy, either in vivo or in vitro. Given the readily availability of easy-to-use in vitro transcription systems (e.g., SP6, T7, reticulocyte lysate, etc.), and also the convenience of maintaining a DNA copy of an RNA vector, it is expected that inventive ssRNA vectors will often be prepared by in vitro transcription, particularly with T7 or SP6 polymerase.

In certain embodiments, rather than introducing a single viral vector type into a plant, multiple different viral vectors are introduced. Such vectors may, for example, trans-complement each other with respect to functions such as replication, cell-to-cell movement, and/or long distance movement. Vectors may contain different polynucleotides encoding influenza antigen polypeptide in accordance with the invention. Selection for plant(s) or portions thereof that express multiple polypeptides encoding one or more influenza antigen polypeptide(s) may be performed as described above for single polynucleotides or polypeptides.

### Plant Tissue Expression Systems

As discussed above, in accordance with the present invention, influenza antigen polypeptides may be produced in any desirable system. Vector constructs and expression systems are well known in the art and may be adapted to incorporate use of influenza antigen polypeptides provided herein. For example, transgenic plant production is known and generation of constructs and plant production may be adapted according to known techniques in the art. In some embodiments, transient expression systems in plants are desirable. Two of these systems include production of clonal roots and clonal plant systems, and derivatives thereof, as well as production of sprouted seedlings systems.

Clonal Plants

Clonal roots maintain RNA viral expression vectors and stably produce target protein uniformly in an entire root over extended periods of time and multiple subcultures. In contrast to plants, where a target gene is eliminated via recombination during cell-to-cell or long distance movement, in root cultures the integrity of a viral vector is maintained and levels of target protein produced over time are similar to those observed during initial screening. Clonal roots allow for ease of production of heterologous protein material for oral formulation of antigen and vaccine compositions. Methods and reagents for generating a variety of clonal entities derived from plants which are useful for production of antigen (e.g., antigen proteins in accordance with the invention) have been described previously and are known in the art (see, for example, PCT Publication WO 05/81905; incorporated herein by reference). Clonal entities include clonal root lines, clonal root cell lines, clonal plant cell lines, and clonal plants capable of production of antigen (e.g., antigen proteins in accordance with the invention). The invention further provides methods and reagents for expression of antigen polynucleotide and polypeptide products in clonal cell lines derived from various plant tissues (e.g., roots, leaves), and in whole plants derived from single cells (clonal plants). Such methods are typically based on use of plant viral vectors of various types.

For example, in one aspect, the invention provides methods of obtaining a clonal root line that expresses a polynucleotide encoding an influenza antigen polypeptide in accordance with the invention comprising steps of: (i) introducing a viral vector that comprises a polynucleotide encoding an influenza antigen polypeptide in accordance with the invention into a plant or portion thereof; and (ii) generating one or more clonal root lines from a plant. Clonal root lines may be generated, for example, by infecting a plant or plant portion (e.g., a harvested piece of leaf) with an Agrobacterium (e.g., A. rhizogenes) that causes formation of hairy roots. Clonal root lines can be screened in various ways to identify lines that maintain virus, lines that express a polynucleotide encoding an influenza antigen polypeptide in accordance with the invention at high levels, etc. The invention further provides clonal root lines, e.g., clonal root lines produced according to inventive methods, and further encompasses methods of expressing polynucleotides and producing polypeptide(s) encoding influenza antigen polypeptide(s) in accordance with the invention using clonal root lines.

The invention further provides methods of generating a clonal root cell line that expresses a polynucleotide encoding an influenza antigen polypeptide in accordance with the invention comprising steps of: (i) generating a clonal root line, cells of which contain a viral vector whose genome comprises a polynucleotide encoding an influenza antigen polypeptide in accordance with the invention; (ii) releasing individual cells from a clonal root line; and (iii) maintaining cells under conditions suitable for root cell proliferation. The invention provides clonal root cell lines and methods of expressing polynucleotides and producing polypeptides using clonal root cell lines.

In one aspect, the invention provides methods of generating a clonal plant cell line that expresses a polynucleotide encoding an influenza antigen polypeptide in accordance with the invention comprising steps of: (i) generating a clonal root line, cells of which contain a viral vector whose genome comprises a polynucleotide encoding an influenza antigen polypeptide in accordance with the invention; (ii) releasing individual cells from a clonal root line; and (iii) maintaining cells in culture under conditions appropriate for plant cell proliferation. The invention further provides methods of generating a clonal plant cell line that expresses a polynucleotide encoding an influenza antigen polypeptide in accordance with the invention comprising steps of: (i) introducing a viral vector that comprises a polynucleotide encoding an influenza antigen polypeptide in accordance with the invention into cells of a plant cell line maintained in culture; and (ii) enriching for cells that contain viral vector. Enrichment may be performed, for example, by (i) removing a portion of cells from the culture; (ii) diluting removed cells so as to reduce cell concentration; (iii) allowing diluted cells to proliferate; and (iv) screening for cells that contain viral vector. Clonal plant cell lines may be used for production of an influenza antigen polypeptide in accordance with the present invention.

The invention includes a number of methods for generating clonal plants, cells of which contain a viral vector that comprises a polynucleotide encoding an influenza antigen polypeptide in accordance with the invention. For example, the invention provides methods of generating a clonal plant that expresses a polynucleotide encoding an influenza antigen polypeptide in accordance with the invention comprising steps of: (i) generating a clonal root line, cells of which contain a viral vector whose genome comprises a polynucleotide encoding an influenza antigen polypeptide in accordance with the invention; (ii) releasing individual cells from a clonal root line; and (iii) maintaining released cells under conditions appropriate for formation of a plant. The invention further provides methods of generating a clonal plant that expresses a polynucleotide encoding an influenza antigen polypeptide in accordance with the invention comprising steps of: (i) generating a clonal plant cell line, cells of which contain a viral vector whose genome comprises a polynucleotide encoding an influenza antigen polypeptide in accordance with the invention; and (ii) maintaining cells under conditions appropriate for formation of a plant. In general, clonal plants according to the invention can express any polynucleotide encoding an influenza antigen polypeptide in accordance with the invention. Such clonal plants can be used for production of an antigen polypeptide.

As noted above, the present invention provides systems for expressing a polynucleotide or polynucleotide(s) encoding influenza antigen polypeptide(s) in accordance with the invention in clonal root lines, clonal root cell lines, clonal plant cell lines (e.g., cell lines derived from leaf, stem, etc.), and in clonal plants. A polynucleotide encoding an influenza antigen polypeptide in accordance with the invention is introduced into an ancestral plant cell using a plant viral vector whose genome includes polynucleotide encoding an influenza antigen polypeptide in accordance with the invention operably linked to (i.e., under control of) a promoter. A clonal root line or clonal plant cell line is established from a cell containing virus according to any of several techniques further described below. The plant virus vector or portions thereof can be introduced into a plant cell by infection, by inoculation with a viral transcript or infectious cDNA clone, by electroporation, by T-DNA mediated gene transfer, etc.

The following sections describe methods for generating clonal root lines, clonal root cell lines, clonal plant cell lines, and clonal plants that express a polynucleotide encoding an influenza antigen polypeptide in accordance with the invention are then described. A “root line” is distinguished from a “root cell line” in that a root line produces actual rootlike structures or roots while a root cell line consists of root cells that do not form rootlike structures. Use of the term “line” is intended to indicate that cells of the line can proliferate and pass genetic information on to progeny cells. Cells of a cell line typically proliferate in culture without being part of an organized structure such as those found in an intact plant. Use of the term “root line” is intended to indicate that cells in the root structure can proliferate without being part of a complete plant. It is noted that the term “plant cell” encompasses root cells. However, to distinguish the inventive methods for generating root lines and root cell lines from those used to directly generate plant cell lines from non-root tissue (as opposed to generating clonal plant cell lines from clonal root lines or clonal plants derived from clonal root lines), the terms “plant cell” and “plant cell line” as used herein generally refer to cells and cell lines that consist of non-root plant tissue. Plant cells can be, for example, leaf, stem, shoot, flower part, etc. It is noted that seeds can be derived from clonal plants generated as derived herein. Such seeds may contain viral vector as will plants obtained from such seeds. Methods for obtaining seed stocks are well known in the art (see, for example, U.S. Patent Publication 2004/093643; incorporated herein by reference).

Clonal Root Lines

The present invention provides systems for generating a clonal root line in which a plant viral vector is used to direct expression of a polynucleotide encoding an influenza antigen polypeptide in accordance with the invention. One or more viral expression vector(s) including a polynucleotide encoding an influenza antigen polypeptide in accordance with the invention operably linked to a promoter is introduced into a plant or a portion thereof according to any of a variety of known methods. For example, plant leaves can be inoculated with viral transcripts. Vectors themselves may be directly applied to plants (e.g., via abrasive inoculations, mechanized spray inoculations, vacuum infiltration, particle bombardment, or electroporation). Alternatively or additionally, virions may be prepared (e.g., from already infected plants), and may be applied to other plants according to known techniques.

Where infection is to be accomplished by direct application of a viral genome to a plant, any available technique may be used to prepare viral genome. For example, many viruses that are usefully employed in accordance with the present invention have ssRNA genomes. ssRNA may be prepared by transcription of a DNA copy of the genome, or by replication of an RNA copy, either in vivo or in vitro. Given the readily available, easy-to-use in vitro transcription systems (e.g., SP6, T7, reticulocyte lysate, etc.), and also the convenience of maintaining a DNA copy of an RNA vector, it is expected that inventive ssRNA vectors will often be prepared by in vitro transcription, particularly with T7 or SP6 polymerase. Infectious cDNA clones can be used. Agrobacterially mediated gene transfer can be used to transfer viral nucleic acids such as viral vectors (either entire viral genomes or portions thereof) to plant cells using, e.g., agroinfiltration, according to methods known in the art.

A plant or plant portion may then be then maintained (e.g., cultured or grown) under conditions suitable for replication of viral transcript. In certain embodiments, virus spreads beyond the initially inoculated cell, e.g., locally from cell to cell and/or systemically from an initially inoculated leaf into additional leaves. However, in some embodiments, virus does not spread. Thus viral vector may contain genes encoding functional MP and/or CP, but may be lacking one or both of such genes. In general, viral vector is introduced into (infects) multiple cells in the plant or portion thereof.

Following introduction of viral vector into a plant, leaves are harvested. In general, leaves may be harvested at any time following introduction of a viral vector. However, it may be desirable to maintain a plant for a period of time following introduction of a viral vector into the plant, e.g., a period of time sufficient for viral replication and, optionally, spread of virus from the cells into which it was initially introduced. A clonal root culture (or multiple cultures) is prepared, e.g., by known methods further described below.

In general, any available method may be used to prepare a clonal root culture from a plant or plant tissue into which a viral vector has been introduced. One such method employs genes that exist in certain bacterial plasmids. These plasmids are found in various species of Agrobacterium that infect and transfer DNA to a wide variety of organisms. As a genus, Agrobacteria can transfer DNA to a large and diverse set of plant types including numerous dicot and monocot angiosperm species and gymnosperms (see, for example, Gelvin, 2003, Microbial. Mol. Biol. Rev., 67:16, and references therein, all of which are incorporated herein by reference). The molecular basis of genetic transformation of plant cells is transfer from bacterium and integration into plant nuclear genome of a region of a large tumor-inducing (Ti) or rhizogenic (Ri) plasmid that resides within various Agrobacterial species. This region is referred to as the T-region when present in the plasmid and as T-DNA when excised from plasmid. Generally, a single-stranded T-DNA molecule is transferred to a plant cell in naturally occurring Agrobacterial infection and is ultimately incorporated (in double-stranded form) into the genome. Systems based on Ti plasmids are widely used for introduction of foreign genetic material into plants and for production of transgenic plants.

Infection of plants with various Agrobacterial species and transfer of T-DNA has a number of effects. For example, A. tumefaciens causes crown gall disease while A. rhizogenes causes development of hairy roots at the site of infection, a condition known as “hairy root disease.” Each root arises from a single genetically transformed cell. Thus root cells in roots are clonal, and each root represents a clonal population of cells. Roots produced by A. rhizogenes infection are characterized by a high growth rate and genetic stability (Giri et al., 2000, Biotech. Adv., 18:1, and references therein, all of which are incorporated herein by reference). In addition, such roots are able to regenerate genetically stable plants (Giri 2000, supra).

In general, the present invention encompasses use of any strain of Agrobacteria, particularly any A. rhizogenes strain, that is capable of inducing formation of roots from plant cells. As mentioned above, a portion of the Ri plasmid (Ri T-DNA) is responsible for causing hairy root disease. While transfer of this portion of the Ri plasmid to plant cells can conveniently be accomplished by infection with Agrobacteria harboring the Ri plasmid, the invention encompasses use of alternative methods of introducing the relevant region into a plant cell. Such methods include any available method of introducing genetic material into plant cells including, but not limited to, biolistics, electroporation, PEG-mediated DNA uptake, Ti-based vectors, etc. The relevant portions of Ri T-DNA can be introduced into plant cells by use of a viral vector. Ri genes can be included in the same vector that contains a polynucleotide encoding an influenza antigen polypeptide in accordance with the invention or in a different viral vector, which can be the same or a different type to that of the vector that contains a polynucleotide encoding an influenza antigen polypeptide in accordance with the invention. It is noted that the entire Ri T-DNA may not be required for production of hairy roots, and the invention encompasses use of portions of Ri T-DNA, provided that such portions contain sufficient genetic material to induce root formation, as known in the art. Additional genetic material, e.g., genes present within the Ri plasmid but not within T-DNA, may be transferred to a plant cell in accordance with the invention, particularly genes whose expression products facilitate integration of T-DNA into the plant cell DNA.

In order to prepare a clonal root line in accordance with certain embodiments, harvested leaf portions are contacted with A. rhizogenes under conditions suitable for infection and transformation. Leaf portions are maintained in culture to allow development of hairy roots. Each root is clonal, i.e., cells in the root are derived from a single ancestral cell into which Ri T-DNA was transferred. In accordance with the invention, a portion of such ancestral cells will contain a viral vector. Thus cells in a root derived from such an ancestral cell may contain viral vector since it will be replicated and will be transmitted during cell division. Thus a high proportion (e.g. at least 50%, at least 75%, at least 80%, at least 90%, at least 95%), all (100%), or substantially all (at least 98%) of cells will contain viral vector. It is noted that since viral vector is inherited by daughter cells within the clonal root, movement of viral vector within the root is not necessary to maintain viral vector throughout the root. Individual clonal hairy roots may be removed from the leaf portion and further cultured. Such roots are also referred to herein as root lines. Isolated clonal roots continue to grow following isolation.

A variety of different clonal root lines have been generated using inventive methods. These root lines were generated using viral vectors containing polynucleotide(s) encoding an influenza antigen polypeptide in accordance with the invention (e.g., encoding influenza polypeptide(s), fusions thereof, and/or immunogenic portions thereof). Root lines were tested by Western blot. Root lines displayed a variety of different expression levels of various polypeptides. Root lines displaying high expression were selected and further cultured. These root lines were subsequently tested again and shown to maintain high levels of expression over extended periods of time, indicating stability. Expression levels were comparable to or greater than expression in intact plants infected with the same viral vector used to generate clonal root lines. In addition, stability of expression of root lines was superior to that obtained in plants infected with the same viral vector. Up to 80% of such virus-infected plants reverted to wild type after 2-3 passages. (Such passages involved inoculating plants with transcripts, allowing infection (local or systemic) to become established, taking a leaf sample, and inoculating fresh plants that are subsequently tested for expression).

Root lines may be cultured on a large scale for production of antigen in accordance with the invention polypeptides as discussed further below. It is noted that clonal root lines (and cell lines derived from clonal root lines) can generally be maintained in medium that does not include various compounds, e.g., plant growth hormones such as auxins, cytokinins, etc., that are typically employed in culture of root and plant cells. This feature greatly reduces expense associated with tissue culture, and the inventors expect that it will contribute significantly to economic feasibility of protein production using plants.

Any of a variety of methods may be used to select clonal roots that express a polynucleotide encoding influenza antigen polypeptide(s) in accordance with the invention. Western blots, ELISA assays, etc., can be used to detect an encoded polypeptide. In the case of detectable markers such as GFP, alternative methods such as visual screens can be performed. If a viral vector that contains a polynucleotide that encodes a selectable marker is used, an appropriate selection can be imposed (e.g., leaf material and/or roots derived therefrom can be cultured in the presence of an appropriate antibiotic or nutritional condition and surviving roots identified and isolated). Certain viral vectors contain two or more polynucleotide(s) encoding influenza antigen polypeptide(s) in accordance with the invention, e.g., two or more polynucleotides encoding different polypeptides. If one of these is a selectable or detectable marker, clonal roots that are selected or detected by selecting for or detecting expression of the marker will have a high probability of also expressing a second polynucleotide. Screening for root lines that contain particular polynucleotides can also be performed using PCR and other nucleic acid detection methods.

Alternatively or additionally, clonal root lines can be screened for presence of virus by inoculating host plants that will form local lesions as a result of virus infection (e.g., hypersensitive host plants). For example, 5 mg of root tissue can be homogenized in 50 μl of phosphate buffer and used to inoculate a single leaf of a tobacco plant. If virus is present in root cultures, within two to three days characteristic lesions will appear on infected leaves. This means that root line contains recombinant virus that carries a polynucleotide encoding an influenza antigen polypeptide in accordance with the invention. If no local lesions are formed, there is no virus, and the root line is rejected as negative. This method is highly time and cost efficient. After initially screening for the presence of virus, roots that contain virus may be subjected to secondary screening, e.g., by Western blot or ELISA to select high expressers. Additional screens, e.g., screens for rapid growth, growth in particular media or under particular environmental conditions, etc., can be applied. These screening methods may, in general, be applied in the development of any of clonal root lines, clonal root cell lines, clonal plant cell lines, and/or clonal plants described herein.

As will be evident to one of ordinary skill in the art, a variety of modifications may be made to the description of the inventive methods for generating clonal root lines that contain a viral vector. Such modifications are within the scope of the invention. For example, while it is generally desirable to introduce viral vector into an intact plant or portion thereof prior to introduction of Ri T-DNA genes, in certain embodiments, the Ri-DNA is introduced prior to introducing viral vector. In addition, it is possible to contact intact plants with A. rhizogenes rather than harvesting leaf portions and then exposing them to bacterium.

Other methods of generating clonal root lines from single cells of a plant or portion thereof that harbor a viral vector can be used (i.e., methods not using A. rhizogenes or genetic material from the Ri plasmid). For example, treatment with certain plant hormones or combinations of plant hormones is known to result in generation of roots from plant tissue.

Clonal Cell Lines Derived from Clonal Root Lines

As described above, the invention provides methods for generating clonal root lines, wherein cells in root lines contain a viral vector. As is well known in the art, a variety of different cell lines can be generated from roots. For example, root cell lines can be generated from individual root cells obtained from a root using a variety of known methods. Such root cell lines may be obtained from various different root cell types within the root. In general, root material is harvested and dissociated (e.g., physically and/or enzymatically digested) to release individual root cells, which are then further cultured. Complete protoplast formation is generally not necessary. If desired, root cells can be plated at very dilute cell concentrations, so as to obtain root cell lines from single root cells. Root cell lines derived in this manner are clonal root cell lines containing viral vector. Such root cell lines therefore exhibit stable expression of a polynucleotide encoding an influenza antigen polypeptide in accordance with the invention. Clonal plant cell lines can be obtained in a similar manner from clonal roots, e.g., by culturing dissociated root cells in the presence of appropriate plant hormones. Screens and successive rounds of enrichment can be used to identify cell lines that express a polynucleotide encoding an influenza antigen polypeptide in accordance with the invention at high levels. However, if the clonal root line from which the cell line is derived already expresses at high levels, such additional screens may be unnecessary.

As in the case of the clonal root lines, cells of a clonal root cell line are derived from a single ancestral cell that contains viral vector and will, therefore, also contain viral vector since it will be replicated and will be transmitted during cell division. Thus a high proportion (e.g. at least 50%, at least 75%, at least 80%, at least 90%, at least 95%), all (100%), or substantially all (at least 98%) of cells will contain viral vector. It is noted that since viral vector is inherited by daughter cells within a clonal root cell line, movement of viral vector among cells is not necessary to maintain viral vector. Clonal root cell lines can be used for production of a polynucleotide encoding an influenza antigen polypeptide in accordance with the invention as described below.

Clonal Plant Cell Lines

The present invention provides methods for generating a clonal plant cell line in which a plant viral vector is used to direct expression of a polynucleotide encoding an influenza antigen polypeptide in accordance with the invention. According to the inventive method, one or more viral expression vector(s) including a polynucleotide encoding an influenza antigen polypeptide in accordance with the invention operably linked to a promoter is introduced into cells of a plant cell line that is maintained in cell culture. A number of plant cell lines from various plant types are known in the art, any of which can be used. Newly derived cell lines can be generated according to known methods for use in practicing the invention. A viral vector is introduced into cells of a plant cell line according to any of a number of methods. For example, protoplasts can be made and viral transcripts then electroporated into cells. Other methods of introducing a plant viral vector into cells of a plant cell line can be used.

A method for generating clonal plant cell lines in accordance with the invention and a viral vector suitable for introduction into plant cells (e.g., protoplasts) can be used as follows: Following introduction of viral vector, a plant cell line may be maintained in tissue culture. During this time viral vector may replicate, and polynucleotide(s) encoding an influenza antigen polypeptide(s) in accordance with the invention may be expressed. Clonal plant cell lines are derived from culture, e.g., by a process of successive enrichment. For example, samples may be removed from culture, optionally with dilution so that the concentration of cells is low, and plated in Petri dishes in individual droplets. Droplets are then maintained to allow cell division.

It will be appreciated that droplets may contain a variable number of cells, depending on the initial density of the culture and the amount of dilution. Cells can be diluted such that most droplets contain either 0 or 1 cell if it is desired to obtain clonal cell lines expressing a polynucleotide encoding an influenza antigen polypeptide in accordance with the invention after only a single round of enrichment. However, it can be more efficient to select a concentration such that multiple cells are present in each droplet and then screen droplets to identify those that contain expressing cells. In general, any appropriate screening procedure can be employed. For example, selection or detection of a detectable marker such as GFP can be used. Western blots or ELISA assays can be used. Individual droplets (100 μl) contain more than enough cells for performance of these assays. Multiple rounds of enrichment are performed to isolate successively higher expressing cell lines. Single clonal plant cell lines (i.e., populations derived from a single ancestral cell) can be generated by further limiting dilution using standard methods for single cell cloning. However, it is not necessary to isolate individual clonal lines. A population containing multiple clonal cell lines can be used for expression of a polynucleotide encoding one or more influenza antigen polypeptide(s) in accordance with the invention.

In general, certain considerations described above for generation of clonal root lines apply to the generation of clonal plant cell lines. For example, a diversity of viral vectors containing one or more polynucleotide(s) encoding an influenza antigen polypeptide(s) in accordance with the invention can be used as can combinations of multiple different vectors. Similar screening methods can be used. As in the case of clonal root lines and clonal root cell lines, cells of a clonal plant cell line are derived from a single ancestral cell that contains viral vector and will, therefore, also contain viral vector since it will be replicated and will be transmitted during cell division. Thus a high proportion (e.g. at least 50%, at least 75%, at least 80%, at least 90%, at least 95%), all (100%), or substantially all (at least 98%) of cells will contain viral vector. It is noted that since viral vector is inherited by daughter cells within a clonal plant cell line, movement of viral vector among cells is not necessary to maintain viral vector. The clonal plant cell line can be used for production of a polypeptide encoding an influenza antigen polypeptide in accordance with the invention as described below.

Clonal Plants

Clonal plants can be generated from clonal roots, clonal root cell lines, and/or clonal plant cell lines produced according to various methods described above. Methods for the generation of plants from roots, root cell lines, and plant cell lines such as clonal root lines, clonal root cell lines, and clonal plant cell lines described herein are well known in the art (see, e.g., Peres et al., 2001, Plant Cell, Tissue, Organ Culture, 65:37; incorporated herein by reference; and standard reference works on plant molecular biology and biotechnology cited elsewhere herein). The invention therefore provides a method of generating a clonal plant comprising steps of (i) generating a clonal root line, clonal root cell line, or clonal plant cell line according to any of the inventive methods described above; and (ii) generating a whole plant from a clonal root line, clonal root cell line, or clonal plant. Clonal plants may be propagated and grown according to standard methods.

As in the case of clonal root lines, clonal root cell lines, and clonal plant cell lines, cells of a clonal plant are derived from a single ancestral cell that contains viral vector and will, therefore, also contain viral vector since it will be replicated and will be transmitted during cell division. Thus a high proportion (e.g. at least 50%, at least 75%, at least 80%, at least 90%, at least 95%), all (100%), or substantially all (at least 98%) of cells will contain viral vector. It is noted that since viral vector is inherited by daughter cells within the clonal plant, movement of viral vector is not necessary to maintain viral vector.

### Sprouts and Sprouted Seedling Plant Expression Systems

According to the present invention, any of a variety of different systems can be used to express proteins or polypeptides in young plants (e.g., sprouted seedlings). In some embodiments, transgenic cell lines or seeds are generated, which are then sprouted and grown for a period of time so that a protein or polypeptide included in the transgenic sequences is produced in young plant tissues (e.g., in sprouted seedlings). Typical technologies for the production of transgenic plant cells and/or seeds include Agrobacterium tumefaciens mediated gene transfer and microprojectile bombardment or electroporation.

Systems and reagents for generating a variety of sprouts and sprouted seedlings which are useful for production of influenza antigen polypeptide(s) according to the present invention have been described previously and are known in the art (see, for example, PCT Publication WO 04/43886; incorporated herein by reference). The present invention further provides sprouted seedlings, which may be edible, as a biomass containing an influenza antigen polypeptide. In certain aspects, biomass is provided directly for consumption of antigen containing compositions. In some aspects, biomass is processed prior to consumption, for example, by homogenizing, crushing, drying, or extracting. In certain aspects, influenza antigen polypeptides are purified from biomass and formulated into a pharmaceutical composition.

Additionally provided are methods for producing influenza antigen polypeptide(s) in sprouted seedlings that can be consumed or harvested live (e.g., sprouts, sprouted seedlings of the Brassica genus). In certain aspects, the present invention involves growing a seed to an edible sprouted seedling in a contained, regulatable environment (e.g., indoors, in a container, etc.). A seed can be a genetically engineered seed that contains an expression cassette encoding an influenza antigen polypeptide, which expression is driven by an exogenously inducible promoter. A variety of exogenously inducible promoters can be used that are inducible, for example, by light, heat, phytohormones, nutrients, etc.

In related embodiments, the present invention provides methods of producing influenza antigen polypeptide(s) in sprouted seedlings by first generating a seed stock for a sprouted seedling by transforming plants with an expression cassette that encodes influenza antigen polypeptide using an Agrobacterium transformation system, wherein expression of an influenza antigen polypeptide is driven by an inducible promoter. Transgenic seeds can be obtained from a transformed plant, grown in a contained, regulatable environment, and induced to express an influenza antigen polypeptide.

In some embodiments methods are provided that involves infecting sprouted seedlings with a viral expression cassette encoding an influenza antigen polypeptide, expression of which may be driven by any of a viral promoter or an inducible promoter. Sprouted seedlings are grown for two to fourteen days in a contained, regulatable environment or at least until sufficient levels of influenza antigen polypeptide have been obtained for consumption or harvesting.

The present invention further provides systems for producing influenza antigen polypeptide(s) in sprouted seedlings that include a housing unit with climate control and a sprouted seedling containing an expression cassette that encodes one or more influenza antigen polypeptides, wherein expression is driven by a constitutive or inducible promoter. Systems can provide unique advantages over the outdoor environment or greenhouse, which cannot be controlled. Thus, the present invention enables a grower to precisely time the induction of expression of influenza antigen polypeptide. It can greatly reduce time and cost of producing influenza antigen polypeptide(s).

In certain aspects, transiently transfected sprouts contain viral vector sequences encoding an inventive influenza antigen polypeptide. Seedlings are grown for a time period so as to allow for production of viral nucleic acid in sprouts, followed by a period of growth wherein multiple copies of virus are produced, thereby resulting in production of influenza antigen polypeptide(s).

In certain aspects, genetically engineered seeds or embryos that contain a nucleic acid encoding influenza antigen polypeptide(s) are grown to sprouted seedling stage in a contained, regulatable environment. The contained, regulatable environment may be a housing unit or room in which seeds can be grown indoors. All environmental factors of a contained, regulatable environment may be controlled. Since sprouts do not require light to grow, and lighting can be expensive, genetically engineered seeds or embryos may be grown to sprouted seedling stage indoors in the absence of light.

Other environmental factors that can be regulated in a contained, regulatable environment of the present invention include temperature, humidity, water, nutrients, gas (e.g., O₂ or CO₂ content or air circulation), chemicals (small molecules such as sugars and sugar derivatives or hormones such as such as phytohormones gibberellic or absisic acid, etc.) and the like.

According to certain methods of the present invention, expression of a nucleic acid encoding an influenza antigen polypeptide may be controlled by an exogenously inducible promoter. Exogenously inducible promoters are caused to increase or decrease expression of a nucleic acid in response to an external, rather than an internal stimulus. A number of environmental factors can act as inducers for expression of nucleic acids carried by expression cassettes of genetically engineered sprouts. A promoter may be a heat-inducible promoter, such as a heat-shock promoter. For example, using as heat-shock promoter, temperature of a contained environment may simply be raised to induce expression of a nucleic acid. Other promoters include light inducible promoters. Light-inducible promoters can be maintained as constitutive promoters if light in a contained regulatable environment is always on. Alternatively or additionally, expression of a nucleic acid can be turned on at a particular time during development by simply turning on the light. A promoter may be a chemically inducible promoter is used to induce expression of a nucleic acid. According to these embodiments, a chemical could simply be misted or sprayed onto seed, embryo, or seedling to induce expression of nucleic acid. Spraying and misting can be precisely controlled and directed onto target seed, embryo, or seedling to which it is intended. The contained environment is devoid of wind or air currents, which could disperse chemical away from intended target, so that the chemical stays on the target for which it was intended.

According to the present invention, time of expression is induced can be selected to maximize expression of an influenza antigen polypeptide in sprouted seedling by the time of harvest. Inducing expression in an embryo at a particular stage of growth, for example, inducing expression in an embryo at a particular number of days after germination, may result in maximum synthesis of an influenza antigen polypeptide at the time of harvest. For example, inducing expression from the promoter 4 days after germination may result in more protein synthesis than inducing expression from the promoter after 3 days or after 5 days. Those skilled in the art will appreciate that maximizing expression can be achieved by routine experimentation. In certain methods, sprouted seedlings are harvested at about 1 day, 2 days, 3 days, 4 days, 5 days, 6 days, 7 days, 8 days, 9 days, 10 days, 11 days, or 12 days after germination.

In cases where the expression vector has a constitutive promoter instead of an inducible promoter, sprouted seedling may be harvested at a certain time after transformation of sprouted seedling. For example, if a sprouted seedling were virally transformed at an early stage of development, for example, at embryo stage, sprouted seedlings may be harvested at a time when expression is at its maximum post-transformation, e.g., at up to about 1 day, up to about 2 days, up to about 3 days, up to about 4 days, up to about 5 days, up to about 6 days, up to about 7 days, up to about 8 days, up to about 9 days, up to about 10 days, up to about 11 days, up to about 12 days, up to about 13 days, up to about 14 days, up to about 15 days, up to about 16 days, up to about 17 days, up to about 18 days, up to about 19 days, up to about 20 days, up to about 21 days, up to about 22 days, up to about 23 days, up to about 24 days, up to about 25 days, up to about 26 days, up to about 27 days, up to about 28 days, up to about 29 days, up to about 30 days post-transformation. It could be that sprouts develop one, two, three or more months post-transformation, depending on germination of seed.

Generally, once expression of influenza antigen polypeptide(s) begins, seeds, embryos, or sprouted seedlings are allowed to grow until sufficient levels of influenza antigen polypeptide(s) are expressed. In certain aspects, sufficient levels are levels that would provide a therapeutic benefit to a subject if harvested biomass were eaten raw. Alternatively or additionally, sufficient levels are levels from which influenza antigen polypeptide can be concentrated or purified from biomass and formulated into a pharmaceutical composition that provides a therapeutic benefit to a subject upon administration. Typically, influenza antigen polypeptide is not a protein expressed in sprouted seedling in nature. At any rate, influenza antigen polypeptide is typically expressed at concentrations above that which would be present in the sprouted seedling in nature.

Once expression of influenza antigen polypeptide is induced, growth is allowed to continue until sprouted seedling stage, at which time sprouted seedlings are harvested. Sprouted seedlings can be harvested live. Harvesting live sprouted seedlings has several advantages including minimal effort and breakage. Sprouted seedlings of the present invention may be grown hydroponically, making harvesting a simple matter of lifting a sprouted seedling from its hydroponic solution. No soil is required for growth of sprouted seedlings in accordance with the invention, but may be provided if deemed necessary or desirable by the skilled artisan. Because sprouts can be grown without soil, no cleansing of sprouted seedling material is required at the time of harvest. Being able to harvest the sprouted seedling directly from its hydroponic environment without washing or scrubbing minimizes breakage of harvested material. Breakage and wilting of plants induces apoptosis. During apoptosis, certain proteolytic enzymes become active, which can degrade pharmaceutical protein expressed in the sprouted seedling, resulting in decreased therapeutic activity of the protein. Apoptosis-induced proteolysis can significantly decrease yield of protein from mature plants. Using methods of the present invention, apoptosis may be avoided when no harvesting takes place until the moment proteins are extracted from the plant.

For example, live sprouts may be ground, crushed, or blended to produce a slurry of sprouted seedling biomass, in a buffer containing protease inhibitors. Buffer may be maintained at about 4° C. In some aspects, sprouted seedling biomass is air-dried, spray dried, frozen, or freeze-dried. As in mature plants, some of these methods, such as air-drying, may result in a loss of activity of pharmaceutical protein. However, because sprouted seedlings are very small and have a large surface area to volume ratio, this is much less likely to occur. Those skilled in the art will appreciate that many techniques for harvesting biomass that minimize proteolysis of expressed protein are available and could be applied to the present invention.

In some embodiments, sprouted seedlings are edible. In certain embodiments, sprouted seedlings expressing sufficient levels of influenza antigen polypeptides are consumed upon harvesting (e.g., immediately after harvest, within minimal period following harvest) so that absolutely no processing occurs before sprouted seedlings are consumed. In this way, any harvest-induced proteolytic breakdown of influenza antigen polypeptide before administration of influenza antigen polypeptide to a subject in need of treatment is minimized. For example, sprouted seedlings that are ready to be consumed can be delivered directly to a subject. Alternatively or additionally, genetically engineered seeds or embryos are delivered to a subject in need of treatment and grown to sprouted seedling stage by a subject. In one aspect, a supply of genetically engineered sprouted seedlings is provided to a subject, or to a doctor who will be treating subject s, so that a continual stock of sprouted seedlings expressing certain desirable influenza antigen polypeptides may be cultivated. This may be particularly valuable for populations in developing countries, where expensive pharmaceuticals are not affordable or deliverable. The ease with which sprouted seedlings in accordance with the invention can be grown makes sprouted seedlings of the present invention particularly desirable for such developing populations.

The regulatable nature of the contained environment imparts advantages to the present invention over growing plants in the outdoor environment. In general, growing genetically engineered sprouted seedlings that express pharmaceutical proteins in plants provides a pharmaceutical product faster (because plants are harvested younger) and with less effort, risk, and regulatory considerations than growing genetically engineered plants. The contained, regulatable environment used in the present invention reduces or eliminates risk of cross-pollinating plants in nature.

For example, a heat inducible promoter likely would not be used outdoors because outdoor temperature cannot be controlled. The promoter would be turned on any time the outdoor temperature rose above a certain level. Similarly, the promoter would be turned off every time the outdoor temperature dropped. Such temperature shifts could occur in a single day, for example, turning expression on in the daytime and off at night. A heat inducible promoter, such as those described herein, would not even be practical for use in a greenhouse, which is susceptible to climatic shifts to almost the same degree as outdoors. Growth of genetically engineered plants in a greenhouse is quite costly. In contrast, in the present system, every variable can be controlled so that the maximum amount of expression can be achieved with every harvest.

In certain embodiments, sprouted seedlings of the present invention are grown in trays that can be watered, sprayed, or misted at any time during development of sprouted seedling. For example, a tray may be fitted with one or more watering, spraying, misting, and draining apparatus that can deliver and/or remove water, nutrients, chemicals etc. at specific time and at precise quantities during development of the sprouted seedling. For example, seeds require sufficient moisture to keep them damp. Excess moisture drains through holes in trays into drains in the floor of the room. Typically, drainage water is treated as appropriate for removal of harmful chemicals before discharge back into the environment.

Another advantage of trays is that they can be contained within a very small space. Since no light is required for sprouted seedlings to grow, trays containing seeds, embryos, or sprouted seedlings may be tightly stacked vertically on top of one another, providing a large quantity of biomass per unit floor space in a housing facility constructed specifically for these purposes. In addition, stacks of trays can be arranged in horizontal rows within the housing unit. Once seedlings have grown to a stage appropriate for harvest (about two to fourteen days) individual seedling trays are moved into a processing facility, either manually or by automatic means, such as a conveyor belt.

The system of the present invention is unique in that it provides a sprouted seedling biomass, which is a source of an influenza antigen polypeptide(s). Whether consumed directly or processed into the form of a pharmaceutical composition, because sprouted seedlings are grown in a contained, regulatable environment, sprouted seedling biomass and/or pharmaceutical composition derived from biomass can be provided to a consumer at low cost. In addition, the fact that the conditions for growth of sprouted seedlings can be controlled makes the quality and purity of product consistent. The contained, regulatable environment in accordance with the invention obviates many safety regulations of the EPA that can prevent scientists from growing genetically engineered agricultural products out of doors.

Transformed Sprouts

A variety of methods can be used to transform plant cells and produce genetically engineered sprouted seedlings. Two available methods for transformation of plants that require that transgenic plant cell lines be generated in vitro, followed by regeneration of cell lines into whole plants include Agrobacterium tumefaciens mediated gene transfer and microprojectile bombardment or electroporation. In some embodiments, transient expression systems are utilized. Typical technologies for producing transient expression of proteins or polypeptides in plant tissues utilize plant viruses. Viral transformation provides more rapid and less costly methods of transforming embryos and sprouted seedlings that can be harvested without an experimental or generational lag prior to obtaining the desired product. For any of these techniques, the skilled artisan would appreciate how to adjust and optimize transformation protocols that have traditionally been used for plants, seeds, embryos, or spouted seedlings.

The present invention provides expression systems having advantages of viral expression systems (e.g., rapid expression, high levels of production) and of Agrobacterium transformation (e.g., controlled administration). In particular, as discussed in detail below, the present invention provides systems in which an agrobacterial construct (i.e., a construct that replicates in Agrobacterium and therefore can be delivered to plant cells by delivery of Agrobacterium) includes a plant promoter that, after being introduced into a plant, directs expression of viral sequences (e.g., including viral replication sequences) carrying a gene for a protein or polypeptide of interest. This system allows controlled, high level transient expression of proteins or polypeptides in plants.

A variety of different embodiments of expression systems, some of which produce transgenic plants and others of which provide for transient expression, are discussed in further detail individually below. For any of these techniques, the skilled artisan reading the present specification would appreciate how to adjust and optimize protocols for expression of proteins or polypeptides in young plant tissues (e.g., sprouted seedlings).

Agrobacterium Transformation

Agrobacterium is a representative genus of the gram-negative family Rhizobiaceae. This species is responsible for plant tumors such as crown gall and hairy root disease. In dedifferentiated plant tissue, which is characteristic of tumors, amino acid derivatives known as opines are produced by the Agrobacterium and catabolized by the plant. The bacterial genes responsible for expression of opines are a convenient source of control elements for chimeric expression cassettes. According to the present invention, an Agrobacterium transformation system may be used to generate young plants (e.g., sprouted seedlings, including edible sprouted seedlings), which are merely harvested earlier than mature plants. Agrobacterium transformation methods can easily be applied to regenerate sprouted seedlings expressing influenza antigen polypeptides.

In general, transforming plants with Agrobacterium involves transformation of plant cells grown in tissue culture by co-cultivation with an Agrobacterium tumefaciens carrying a plant/bacterial vector. The vector contains a gene encoding an influenza antigen polypeptide. The Agrobacterium transfers vector to plant host cell and is then eliminated using antibiotic treatment. Transformed plant cells expressing influenza antigen polypeptide are selected, differentiated, and finally regenerated into complete plantlets (HeHens et al., 2000, Plant Mol. Biol., 42:819; Pilon-Smits et al., 1999, Plant Physiolog., 119:123; Barfield et al., 1991, Plant Cell Reports, 10:308; and Riva et al., 1998, J. Biotech., 1(3); all of which are incorporated by reference herein).

Agrobacterial expression vectors for use in the present invention include a gene (or expression cassette) encoding an influenza antigen polypeptide designed for operation in plants, with companion sequences upstream and downstream of the expression cassette. Companion sequences are generally of plasmid or viral origin and provide necessary characteristics to the vector to transfer DNA from bacteria to the desired plant host.

The basic bacterial/plant vector construct may desirably provide a broad host range prokaryote replication origin, a prokaryote selectable marker. Suitable prokaryotic selectable markers include resistance toward antibiotics such as ampicillin or tetracycline. Other DNA sequences encoding additional functions that are well known in the art may be present in the vector.

Agrobacterium T-DNA sequences are required for Agrobacterium mediated transfer of DNA to the plant chromosome. The tumor-inducing genes of T-DNA are typically removed during construction of an agrobacterial expression construct and are replaced with sequences encoding an influenza antigen polypeptide. T-DNA border sequences are retained because they initiate integration of the T-DNA region into the plant genome. If expression of influenza antigen polypeptide is not readily amenable to detection, the bacterial/plant vector construct may include a selectable marker gene suitable for determining if a plant cell has been transformed, e.g., nptII kanamycin resistance gene. On the same or different bacterial/plant vector (Ti plasmid) are Ti sequences. Ti sequences include virulence genes, which encode a set of proteins responsible for excision, transfer and integration of T-DNA into the plant genome (Schell, 1987, Science, 237:1176-86; incorporated herein by reference). Other sequences suitable for permitting integration of heterologous sequence into the plant genome may include transposon sequences, and the like, for homologous recombination.

On the same or different bacterial/plant vector (Ti plasmid) are Ti sequences. Ti sequences include the virulence genes, which encode a set of proteins responsible for the excision, transfer and integration of the T-DNA into the plant genome (Schell, 1987, Science, 237:1176-83; incorporated herein by reference). Other sequences suitable for permitting integration of the heterologous sequence into the plant genome may also include transposon sequences, and the like, for homologous recombination.

Certain constructs will include an expression cassette encoding an antigen protein. One, two, or more expression cassettes may be used in a given transformation. The recombinant expression cassette contains, in addition to an influenza antigen polypeptide encoding sequence, at least the following elements: a promoter region, plant 5′ untranslated sequences, initiation codon (depending upon whether or not an expressed gene has its own), and transcription and translation termination sequences. In addition, transcription and translation terminators may be included in expression cassettes or chimeric genes of the present invention. Signal secretion sequences that allow processing and translocation of a protein, as appropriate, may be included in the expression cassette.

A variety of promoters, signal sequences, and transcription and translation terminators are described, for example, in Lawton et al. (1987, Plant Mol. Biol., 9:315-24; incorporated herein by reference) or in U.S. Pat. No. 5,888,789 (incorporated herein by reference). In addition, structural genes for antibiotic resistance are commonly utilized as a selection factor (Fraley et al., 1983, Proc. Natl. Acad. Sci., USA, 80:4803-7; incorporated herein by reference). Unique restriction enzyme sites at the 5′ and 3′ ends of the cassette allow for easy insertion into a pre-existing vector.

Other binary vector systems for Agrobacterium-mediated transformation, carrying at least one T-DNA border sequence are described in PCT Publication WO 2000/020612 (incorporated herein by reference). Further discussion of Agrobacterium-mediated transformation is found in Gelvin (2003, Microbiol. Mol. Biol. Rev., 67:16-37; and references therein; all of which are incorporated herein by reference) and Lorence and Verpoorte (2004, Methods Mol. Biol., 267:329-50; incorporated herein by reference).

In certain embodiments, bacteria other than Agrobacteria are used to introduce a nucleic acid sequence into a plant. See, e.g., Broothaerts et al. (2005, Nature, 433:629-33; incorporated herein by reference).

Seeds are prepared from plants that have been infected with Agrobacteria (or other bacteria) such that the desired heterologous gene encoding a protein or polypeptide of interest is introduced. Such seeds are harvested, dried, cleaned, and tested for viability and for the presence and expression of a desired gene product. Once this has been determined, seed stock is typically stored under appropriate conditions of temperature, humidity, sanitation, and security to be used when necessary. Whole plants may then be regenerated from cultured protoplasts, e.g., as described in Evans et al. (Handbook of Plant Cell Cultures, Vol. 1, MacMillan Publishing Co., New York, N.Y., 1983; incorporated herein by reference); and in Vasil (ed., Cell Culture and Somatic Cell Genetics of Plants, Acad. Press, Orlando, Fla., Vol. I, 1984, and Vol. III, 1986; incorporated herein by reference). In certain aspects, plants are regenerated only to sprouted seedling stage. In some aspects, whole plants are regenerated to produce seed stocks and sprouted seedlings are generated from seeds of the seed stock.

In certain embodiments, the plants are not regenerated into adult plants. For example, in some embodiments, plants are regenerated only to the sprouted seedling stage. In other embodiments, whole plants are regenerated to produce seed stocks and young plants (e.g., sprouted seedlings) for use in accordance with the present invention are generated from the seeds of the seed stock.

All plants from which protoplasts can be isolated and cultured to give whole, regenerated plants can be transformed by Agrobacteria according to the present invention so that whole plants are recovered that contain a transferred gene. It is known that practically all plants can be regenerated from cultured cells or tissues, including, but not limited to, all major species of plants that produce edible sprouts. Some suitable plants include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as sunflower etc.

Means for regeneration of plants from transformed cells vary from one species of plants to the next. However, those skilled in the art will appreciate that generally a suspension of transformed protoplants containing copies of a heterologous gene is first provided. Callus tissue is formed and shoots may be induced from callus and subsequently rooted. Alternatively or additionally, embryo formation can be induced from a protoplast suspension. These embryos germinate as natural embryos to form plants. Steeping seed in water or spraying seed with water to increase the moisture content of the seed to between 35%-45% initiates germination. For germination to proceed, seeds are typically maintained in air saturated with water under controlled temperature and airflow conditions. The culture media will generally contain various amino acids and hormones, such as auxin and cytokinins. It is advantageous to add glutamic acid and proline to the medium, especially for such species as alfalfa. Shoots and roots normally develop simultaneously. Efficient regeneration will depend on the medium, the genotype, and the history of the culture. If these three variables are controlled, then regeneration is fully reproducible and repeatable.

Mature plants, grown from the transformed plant cells, are selfed and non-segregating, homozygous transgenic plants are identified. The inbred plant produces seeds containing inventive antigen-encoding sequences. Such seeds can be germinated and grown to sprouted seedling stage to produce influenza antigen polypeptide(s) according to the present invention.

In related embodiments, transgenic seeds (e.g., carrying the transferred gene encoding an influenza antigen polypeptide, typically integrated into the genome) may be formed into seed products and sold with instructions on how to grow young plants to the appropriate stage (e.g., sprouted seedling stage) for harvesting and/or administration or harvesting into a formulation as described herein. In some related embodiments, hybrids or novel varieties embodying desired traits may be developed from inbred plants in accordance with the invention.

Direct Integration

Direct integration of DNA fragments into the genome of plant cells by microprojectile bombardment or electroporation may also be used to introduce expression constructs encoding influenza antigen polypeptides into plant tissues in accordance with the present invention (see, e.g., Kikkert, et al., 1999, Plant: J. Tiss. Cult. Assoc., 35:43; and Bates, 1994, Mol. Biotech., 2:135; both of which are incorporated herein by reference). More particularly, vectors that express influenza antigen polypeptide(s) of the present invention can be introduced into plant cells by a variety of techniques. As described above, vectors may include selectable markers for use in plant cells. Vectors may include sequences that allow their selection and propagation in a secondary host, such as sequences containing an origin of replication and selectable marker. Typically, secondary hosts include bacteria and yeast. In some embodiments, a secondary host is bacteria (e.g., Escherichia coli, the origin of replication is a colE1-type origin of replication) and a selectable marker is a gene encoding ampicillin resistance. Such sequences are well known in the art and are commercially available (e.g., Clontech, Palo Alto, Calif. or Stratagene, La Jolla, Calif.).

Vectors of the present invention may be modified to intermediate plant transformation plasmids that contain a region of homology to an Agrobacterium tumefaciens vector, a T-DNA border region from Agrobacterium tumefaciens, and chimeric genes or expression cassettes described above. Further vectors may include a disarmed plant tumor inducing plasmid of Agrobacterium tumefaciens.

According to some embodiments, direct transformation of vectors invention may involve microinjecting vectors directly into plant cells by use of micropipettes to mechanically transfer recombinant DNA (see, e.g., Crossway, 1985, Mol. Gen. Genet., 202:179, incorporated herein by reference). Genetic material may be transferred into a plant cell using polyethylene glycols (see, e.g., Krens et al., 1982, Nature 296:72; incorporated herein by reference). Another method of introducing nucleic acids into plants via high velocity ballistic penetration by small particles with a nucleic acid either within the matrix of small beads or particles, or on the surface (see, e.g., Klein et al., 1987, Nature 327:70; and Knudsen et al., Planta, 185:330; both of which are incorporated herein by reference). Yet another method of introduction is fusion of protoplasts with other entities, either minicells, cells, lysosomes, or other fusible lipid-surfaced bodies (see, e.g., Fraley et al., 1982, Proc. Natl. Acad. Sci., USA, 79:1859; incorporated herein by reference). Vectors in accordance with the invention may be introduced into plant cells by electroporation (see, e.g., Fromm et al. 1985, Proc. Natl. Acad. Sci., USA, 82:5824; incorporated herein by reference). According to this technique, plant protoplasts are electroporated in the presence of plasmids containing a gene construct. Electrical impulses of high field strength reversibly permeabilize biomembranes allowing introduction of plasmids. Electroporated plant protoplasts reform the cell wall divide and form plant callus, which can be regenerated to form sprouted seedlings in accordance with the invention. Those skilled in the art will appreciate how to utilize these methods to transform plants cells that can be used to generate edible sprouted seedlings.

Viral Transformation

Similar to conventional expression systems, plant viral vectors can be used to produce full-length proteins, including full length antigen. According to the present invention, plant virus vectors may be used to infect and produce antigen(s) in seeds, embryos, sprouted seedlings, etc. In this regard infection includes any method of introducing a viral genome, or portion thereof, into a cell, including, but not limited to, the natural infectious process of a virus, abrasion, inoculation, etc. The term includes introducing a genomic RNA transcript, or a cDNA copy thereof, into a cell. The viral genome need not be a complete genome but will typically contain sufficient sequences to allow replication. The genome may encode a viral replicase and may contain any cis-acting nucleic acid elements necessary for replication. Expression of high levels of foreign genes encoding short peptides as well as large complex proteins (e.g., by tobamoviral vectors) is described (see, e.g., McCormick et al., 1999, Proc. Natl. Acad. Sci., USA, 96:703; Kumagai et al. 2000, Gene, 245:169; and Verch et al., 1998, J. Immunol. Methods, 220:69; all of which are incorporated herein by reference). Thus, plant viral vectors have a demonstrated ability to express short peptides as well as large complex proteins.

In certain embodiments, young plants (e.g., sprouts), which express influenza antigen polypeptide, are generated utilizing a host/virus system. Young plants produced by viral infection provide a source of transgenic protein that has already been demonstrated to be safe. For example, sprouts are free of contamination with animal pathogens. Unlike, for example, tobacco, proteins from an edible sprout could at least in theory be used in oral applications without purification, thus significantly reducing costs.

In addition, a virus/young plant (e.g., sprout) system offers a much simpler, less expensive route for scale-up and manufacturing, since the relevant genes (encoding the protein or polypeptide of interest) are introduced into the virus, which can be grown up to a commercial scale within a few days. In contrast, transgenic plants can require up to 5-7 years before sufficient seeds or plant material is available for large-scale trials or commercialization.

According to the present invention, plant RNA viruses have certain advantages, which make them attractive as vectors for foreign protein expression. The molecular biology and pathology of a number of plant RNA viruses are well characterized and there is considerable knowledge of virus biology, genetics, and regulatory sequences. Most plant RNA viruses have small genomes and infectious cDNA clones are available to facilitate genetic manipulation. Once infectious virus material enters a susceptible host cell, it replicates to high levels and spreads rapidly throughout the entire sprouted seedling (one to ten days post inoculation, e.g., 1 day, 2 days, 3 days, 4 days, 5 days, 6 days, 7 days, 8 days, 9 days, 10 days, or more than 10 days post-inoculation). Virus particles are easily and economically recovered from infected sprouted seedling tissue. Viruses have a wide host range, enabling use of a single construct for infection of several susceptible species. These characteristics are readily transferable to sprouts.

Foreign sequences can be expressed from plant RNA viruses, typically by replacing one of the viral genes with desired sequence, by inserting foreign sequences into the virus genome at an appropriate position, or by fusing foreign peptides to structural proteins of a virus. Moreover, any of these approaches can be combined to express foreign sequences by trans-complementation of vital functions of a virus. A number of different strategies exist as tools to express foreign sequences in virus-infected plants using tobacco mosaic virus (TMV), alfalfa mosaic virus (AlMV), and chimeras thereof.

The genome of AlMV is a representative of the Bromoviridae family of viruses and consists of three genomic RNAs (RNAs1-3) and subgenomic RNA (RNA4). Genomic RNAs1 and 2 encode virus replicase proteins P1 and 2, respectively. Genomic RNA3 encodes cell-to-cell movement protein P3 and coat protein (CP). CP is translated from subgenomic RNA4, which is synthesized from genomic RNA3, and is required to start infection. Studies have demonstrated the involvement of CP in multiple functions, including genome activation, replication, RNA stability, symptom formation, and RNA encapsidation (see e.g., Bol et al., 1971, Virology, 46:73; Van Der Vossen et al., 1994, Virology 202:891; Yusibov et al., Virology, 208:405; Yusibov at al., 1998, Virology, 242:1; Bol et al., (Review, 100 refs.), 1999, J. Gen. Virol., 80:1089; De Graaff, 1995, Virology, 208:583; Jaspars et al., 1974, Adv. Virus Res., 19:37; Loesch-Fries, 1985, Virology, 146:177; Neeleman et al., 1991, Virology, 181:687; Neeleman at al., 1993, Virology, 196: 883; Van Der Kuyl at al., 1991, Virology, 183:731; and Van Der Kuyl at al., 1991, Virology, 185:496; all of which are incorporated herein by reference).

Encapsidation of viral particles is typically required for long distance movement of virus from inoculated to un-inoculated parts of seed, embryo, or sprouted seedling and for systemic infection. According to the present invention, inoculation can occur at any stage of plant development. In embryos and sprouts, spread of inoculated virus should be very rapid. Virions of AlMV are encapsidated by a unique CP (241d)), forming more than one type of particle. The size (30- to 60-nm in length and 18 nm in diameter) and shape (spherical, ellipsoidal, or bacilliform) of the particle depends on the size of the encapsidated RNA. Upon assembly, the N-terminus of AlMV CP is thought to be located on the surface of the virus particles and does not appear to interfere with virus assembly (Bol at al., 1971, Virology, 6:73; incorporated herein by reference). Additionally, ALMV CP with an additional 38-amino acid peptide at its N-terminus forms particles in vitro and retains biological activity (Yusibov et al., 1995, J. Gen. Virol., 77:567; incorporated herein by reference).

AlMV has a wide host range, which includes a number of agriculturally valuable crop plants, including plant seeds, embryos, and sprouts. Together, these characteristics make ALMV CP an excellent candidate as a carrier molecule for polypeptides and AlMV an attractive candidate vector for expression of foreign sequences in a plant at the sprout stage of development. Moreover, upon expression from a heterologous vector such as TMV, AlMV CP encapsidates TMV genome without interfering with virus infectivity (Yusibov et at, 1997, Proc. Natl. Acad. Sci., USA, 94:5784; incorporated herein by reference). This allows use of TMV as a carrier virus for AlMV CP fused to foreign sequences.

TMV, the prototype of tobamoviruses, has a genome consisting of a single plus-sense RNA encapsidated with a 17.0 kD CP, which results in rod-shaped particles (300 nm in length). CP is the only structural protein of TMV and is required for encapsidation and long distance movement of virus in an infected host (Saito et al., 1990, Virology 176:329; incorporated herein by reference). 183 and 126 kD proteins are translated from genomic RNA and are required for virus replication (Ishikawa et al., 1986, Nucleic Acids Res., 14:8291; incorporated herein by reference). 30 kD protein is the cell-to-cell movement protein of virus (Meshi et al., 1987, EMBO J., 6:2557). Movement and coat proteins are translated from subgenomic mRNAs (Hunter et al., 1976, Nature, 260:759; Bruening et al., 1976, Virology, 71:498; and Beachy et al., 1976, Virology, 73:498; all of which are incorporated herein by reference).

Other methods that may be utilized to introduce a gene encoding an influenza polypeptide into plant cells include transforming the flower of a plant. Transformation of Arabidopsis thaliana can be achieved by dipping plant flowers into a solution of Agrobacterium tumefaciens (Curtis et al., 2001, Transgenic Res., 10:363; and Qing et at, 2000, Molecular Breeding: New Strategies in Plant Improvement 1:67; both of which are incorporated herein by reference). Transformed plants are formed in the population of seeds generated by “dipped” plants. At a specific point during flower development, a pore exists in the ovary wall through which Agrobacterium tumefaciens gains access to the interior of the ovary. Once inside the ovary, the Agrobacterium tumefaciens proliferates and transforms individual ovules (Desfeux et al., 2000, Plant Physiology, 123:895; incorporated herein by reference). Transformed ovules follow the typical pathway of seed formation within the ovary.

Agrobacterium-Mediated Transient Expression

As indicated herein, in many embodiments of the present invention, systems for rapid (e.g., transient) expression of proteins or polypeptides in plants are desirable. Among other things, the present invention provides a powerful system for achieving such rapid expression in plants (particularly in young plants, e.g., sprouted seedlings) that utilizes an agrobacterial construct to deliver a viral expression system encoding an influenza polypeptide.

Specifically, according to the present invention, a “launch vector” is prepared that contains agrobacterial sequences including replication sequences and also contains plant viral sequences (including self-replication sequences) that carry a gene encoding the protein or polypeptide of interest. A launch vector is introduced into plant tissue, preferably by agroinfiltration, which allows substantially systemic delivery. For transient transformation, non-integrated T-DNA copies of the launch vector remain transiently present in the nucleolus and are transcribed leading to the expression of the carrying genes (Kapila et al., 1997, Plant Science, 122:101-108; incorporated herein by reference). Agrobacterium-mediated transient expression, differently from viral vectors, cannot lead to the systemic spreading of the expression of the gene of interest. One advantage of this system is the possibility to clone genes larger than 2 kb to generate constructs that would be impossible to obtain with viral vectors (Voinnet et al., 2003, Plant J., 33:949-56; incorporated herein by reference). Furthermore, using such technique, it is possible to transform the plant with more than one transgene, such that multimeric proteins (e.g., antibodies subunits of complexed proteins) can be expressed and assembled. Furthermore, the possibility of co-expression of multiple transgenes by means of co-infiltration with different Agrobacterium can be taken advantage of, either by separate infiltration or using mixed cultures.

In certain embodiments, a launch vector includes sequences that allow for selection (or at least detection) in Agrobacteria and also for selection/detection in infiltrated tissues. Furthermore, a launch vector typically includes sequences that are transcribed in the plant to yield viral RNA production, followed by generation of viral proteins. Furthermore, production of viral proteins and viral RNA yields rapid production of multiple copies of RNA encoding the pharmaceutically active protein of interest. Such production results in rapid protein production of the target of interest in a relatively short period of time. Thus, a highly efficient system for protein production can be generated.

The agroinfiltration technique utilizing viral expression vectors can be used to produce limited quantity of protein of interest in order to verify the expression levels before deciding if it is worth generating transgenic plants. Alternatively or additionally, the agroinfiltration technique utilizing viral expression vectors is useful for rapid generation of plants capable of producing huge amounts of protein as a primary production platform. Thus, this transient expression system can be used on industrial scale.

Further provided are any of a variety of different Agrobacterial plasmids, binary plasmids, or derivatives thereof such as pBIV, pBI1221, pGreen, etc., which can be used in these and other aspects of the invention. Numerous suitable vectors are known in the art and can be directed and/or modified according to methods known in the art, or those described herein so as to utilize in the methods described provided herein.

An exemplary launch vector, pBID4, contains the 35 S promoter of cauliflower mosaic virus (a DNA plant virus) that drives initial transcription of the recombinant viral genome following introduction into plants, and the nos terminator, the transcriptional terminator of Agrobacterium nopaline synthase. The vector further contains sequences of the tobacco mosaic virus genome including genes for virus replication (126/183K) and cell-t-cell movement (MP). The vector further contains a gene encoding a polypeptide of interest, inserted into a unique cloning site within the tobacco mosaic virus genome sequences and under the transcriptional control of the coat protein subgenomic mRNA promoter. Because this “target gene” (i.e., gene encoding a protein or polypeptide of interest) replaces coding sequences for the TMV coat protein, the resultant viral vector is naked self-replicating RNA that is less subject to recombination than CP-containing vectors, and that cannot effectively spread and survive in the environment. Left and right border sequences (LB and RB) delimit the region of the launch vector that is transferred into plant cells following infiltration of plants with recombinant Agrobacterium carrying the vector. Upon introduction of agrobacteria carrying this vector into plant tissue (typically by agroinfiltration but alternatively by injection or other means), multiple single-stranded DNA (ssDNA) copies of sequence between LB and RB are generated and released in a matter of minutes. These introduced sequences are then amplified by viral replication. Translation of the target gene results in accumulation of large amounts of target protein or polypeptide in a short period of time. A launch vector can include coat proteins and movement protein sequences. Exemplary sequences from AlMV and TMV are SEQ ID NOs 113-114 and SEQ ID NOs 115-116 respectively.

In some embodiments, Agrobacterium-mediated transient expression produces up to about 5 g or more of target protein per kg of plant tissue. For example, in some embodiments, up to about 4 g, about 3 g, about 2 g, about 1 g, or about 0.5 g of target protein is produced per kg of plant tissue. In some embodiments, at least about 20 mg to about 500 mg, or about 50 mg to about 500 mg of target protein, or about 50 mg to about 200 mg, or about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 1750 mg, about 2000 mg, about 2500 mg, about 3000 mg or more of protein per kg of plant tissue is produced.

In some embodiments, these expression levels are achieved within about 6, about 5, about 4, about 3, or about 2 weeks from infiltration. In some embodiments, these expression levels are achieved within about 10, about 9, about 8, about 7, about 6, about 5, about 4, about 3, about 2 days, or even about 1 day, from introduction of the expression construct. Thus, the time from introduction (e.g., infiltration) to harvest is typically less than about 2 weeks, about 10 days, about 1 week or less. This allows production of protein within about 8 weeks or less from the selection of amino acid sequence (even including time for “preliminary” expression studies). Also, each batch of protein can typically be produced within about 8 weeks, about 6 weeks, about 5 weeks, or less. Those of ordinary skill in the art will appreciate that these numbers may vary somewhat depending on the type of plant used. Most sprouts, including peas, will fall within the numbers given. Nicotiana benthamiana, however, may be grown longer, particularly prior to infiltration, as they are slower growing (from a much smaller seed). Other expected adjustments will be clear to those of ordinary skill in the art based on biology of the particular plants utilized.

The present inventors have used a launch vector system to produce a variety of target proteins and polypeptides in a variety of different young plants. In some embodiments, certain pea varieties including for example, marrowfat pea, bill jump pea, yellow trapper pea, speckled pea, and green pea are particularly useful in the practice of this aspect of the invention.

The inventors have also found that various Nicotiana plants are particularly useful in the practice of this aspect of the invention, including in particular Nicotiana benthamiana. The present invention teaches that young Nicotiana plants (particularly young Nicotiana benthamiana plants) are useful in the practice of the invention. In general, in some embodiments, Nicotiana benthamiana plants are grown for a time sufficient to allow development of an appropriate amount of biomass prior to infiltration (i.e., to delivery of agrobacteria containing the launch vector). Typically, the plants are grown for a period of more than about 3 weeks, more typically more than about 4 weeks, or between about 5 to about 6 weeks to accumulate biomass prior to infiltration.

The present inventors have further surprisingly found that, although both TMV and AlMV sequences can prove effective in such launch vector constructs, in some embodiments, AlMV sequences are particularly efficient at ensuring high level production of delivered protein or polypeptides.

Thus, in certain particular embodiments of the present invention, proteins or polypeptides of interest are produced in young pea plants or young Nicotania plants (e.g., Nicotiana benthamiana) from a launch vector that directs production of AlMV sequences carrying the gene of interest.

Expression Constructs

Many features of expression constructs useful in accordance with the present invention will be specific to the particular expression system used, as discussed above. However, certain aspects that may be applicable across different expression systems are discussed in further detail here.

To give but one example, in many embodiments of the present invention, it will be desirable that expression of the protein or polypeptide (or nucleic acid) of interest be inducible. In many such embodiments, production of an RNA encoding the protein or polypeptide of interest (and/or production of an antisense RNA) is under the control of an inducible (e.g. exogenously inducible) promoter. Exogenously inducible promoters are caused to increase or decrease expression of a transcript in response to an external, rather than an internal stimulus. A number of environmental factors can act as such an external stimulus. In certain embodiments, transcription is controlled by a heat-inducible promoter, such as a heat-shock promoter.

Externally inducible promoters may be particularly useful in the context of controlled, regulatable growth settings. For example, using a heat-shock promoter the temperature of a contained environment may simply be raised to induce expression of the relevant transcript. In will be appreciated, of course, that a heat inducible promoter could never be used in the outdoors because the outdoor temperature cannot be controlled. The promoter would be turned on any time the outdoor temperature rose above a certain level. Similarly, the promoter would be turned off every time the outdoor temperature dropped. Such temperature shifts could occur in a single day, for example, turning expression on in the daytime and off at night. A heat inducible promoter, such as those described herein, would likely not even be practical for use in a greenhouse, which is susceptible to climatic shifts to almost the same degree as the outdoors. Growth of genetically engineered plants in a greenhouse is quite costly. In contrast, in the present system, every variable can be controlled so that the maximum amount of expression can be achieved with every harvest.

Other externally-inducible promoters than can be utilized in accordance with the present invention include light inducible promoters. Light-inducible promoters can be maintained as constitutive promoters if the light in the contained regulatable environment is always on. Alternatively, expression of the relevant transcript can be turned on at a particular time during development by simply turning on the light.

In yet other embodiments, a chemically inducible promoter is used to induce expression of the relevant transcript. According to these embodiments, the chemical could simply be misted or sprayed onto a seed, embryo, or young plant (e.g., seedling) to induce expression of the relevant transcript. Spraying and misting can be precisely controlled and directed onto a particular seed, embryo, or young plant (e.g., seedling) as desired. A contained environment is devoid of wind or air currents, which could disperse the chemical away from the intended recipient, so that the chemical stays on the recipient for which it was intended.

### Production and Isolation of Antigen

In general, standard methods known in the art may be used for culturing or growing plants, plant cells, and/or plant tissues in accordance with the invention (e.g., clonal plants, clonal plant cells, clonal roots, clonal root lines, sprouts, sprouted seedlings, plants, etc.) for production of antigen(s). A wide variety of culture media and bioreactors have been employed to culture hairy root cells, root cell lines, and plant cells (see, for example, Gin et al., 2000, Biotechnol. Adv., 18:1; Rao et al., 2002, Biotechnol. Adv., 20:101; and references in both of the foregoing, all of which are incorporated herein by reference). Clonal plants may be grown in any suitable manner.

In a certain embodiments, influenza antigen polypeptides in accordance with the invention may be produced by any known method. In some embodiments, an influenza antigen polypeptide is expressed in a plant or portion thereof. Proteins are isolated and purified in accordance with conventional conditions and techniques known in the art. These include methods such as extraction, precipitation, chromatography, affinity chromatography, electrophoresis, and the like. The present invention involves purification and affordable scaling up of production of influenza antigen polypeptide(s) using any of a variety of plant expression systems known in the art and provided herein, including viral plant expression systems described herein.

In many embodiments of the present invention, it will be desirable to isolate influenza antigen polypeptide(s) for vaccine products. Where a protein in accordance with the invention is produced from plant tissue(s) or a portion thereof, e.g., roots, root cells, plants, plant cells, that express them, methods described in further detail herein, or any applicable methods known in the art may be used for any of partial or complete isolation from plant material. Where it is desirable to isolate the expression product from some or all of plant cells or tissues that express it, any available purification techniques may be employed. Those of ordinary skill in the art are familiar with a wide range of fractionation and separation procedures (see, for example, Scopes et al., Protein Purification: Principles and Practice, 3^{rd} Ed., Janson et al., 1993; Protein Purification. Principles, High Resolution Methods, and Applications, Wiley-VCH, 1998; Springer-Verlag, NY, 1993; and Roe, Protein Purification Techniques, Oxford University Press, 2001; each of which is incorporated herein by reference). Often, it will be desirable to render the product more than about 50%, about 60%, about 70%, about 80%, about 85%, about 90%, about 91%, about 92%, about 93%, about 94%, about 95%, about 96%, about 97%, about 98%, or about 99% pure. See, e.g., U.S. Pat. Nos. 6,740,740 and 6,841,659 (both of which are incorporated herein by reference) for discussion of certain methods useful for purifying substances from plant tissues or fluids.

Those skilled in the art will appreciate that a method of obtaining desired influenza antigen polypeptide(s) product(s) is by extraction. Plant material (e.g., roots, leaves, etc.) may be extracted to remove desired products from residual biomass, thereby increasing the concentration and purity of product. Plants may be extracted in a buffered solution. For example, plant material may be transferred into an amount of ice-cold water at a ratio of one to one by weight that has been buffered with, e.g., phosphate buffer. Protease inhibitors can be added as required. The plant material can be disrupted by vigorous blending or grinding while suspended in buffer solution and extracted biomass removed by filtration or centrifugation. The product carried in solution can be further purified by additional steps or converted to a thy powder by freeze-drying or precipitation. Extraction can be carried out by pressing. Plants or roots can be extracted by pressing in a press or by being crushed as they are passed through closely spaced rollers. Fluids expressed from crushed plants or roots are collected and processed according to methods well known in the art. Extraction by pressing allows release of products in a more concentrated form. However, overall yield of product may be lower than if product were extracted in solution.

In some embodiments, produced proteins or polypeptides are not isolated from plant tissue but rather are provided in the context of live plants (e.g., sprouted seedlings). In some embodiments, where the plant is edible, plant tissue containing expressed protein or polypeptide is provided directly for consumption. Thus, the present invention provides edible young plant biomass (e.g., edible sprouted seedlings) containing expressed protein or polypeptide.

Where edible plants (e.g., sprouted seedlings) express sufficient levels of pharmaceutical proteins or polypeptides and are consumed live, in some embodiments absolutely no harvesting occurs before the sprouted seedlings are consumed. In this way, it is guaranteed that there is no harvest-induced proteolytic breakdown of the pharmaceutical protein before administration of the pharmaceutical protein to a subject in need of treatment. For example, young plants (e.g., sprouted seedlings) that are ready to be consumed can be delivered directly to a subject. Alternatively, genetically engineered seeds or embryos are delivered to a subject in need of treatment and grown to the sprouted seedling stage by the subject. In some embodiments, a supply of genetically engineered sprouted seedlings is provided to a subject, or to a doctor who will be treating subjects, so that a continual stock of sprouted seedlings expressing certain desirable pharmaceutical proteins may be cultivated. This may be particularly valuable for populations in developing countries, where expensive pharmaceuticals are not affordable or deliverable. The ease with which the sprouted seedlings in accordance with the invention can be grown makes the sprouted seedlings of the present invention particularly desirable for such developing populations.

In some embodiments, plant biomass is processed prior to consumption or formulation, for example, by homogenizing, crushing, drying, or extracting. In some embodiments, the expressed protein or polypeptide is isolated or purified from the biomass and formulated into a pharmaceutical composition.

For example, live plants (e.g., sprouts) may be ground, crushed, or blended to produce a slurry of biomass, in a buffer containing protease inhibitors. Preferably the buffer is at about 4° C. In certain embodiments, the biomass is air-dried, spray dried, frozen, or freeze-dried. As in mature plants, some of these methods, such as air-drying, may result in a loss of activity of the pharmaceutical protein or polypeptide. However, because plants (e.g., sprouted seedlings) may be very small and typically have a large surface area to volume ratio, this is much less likely to occur. Those skilled in the art will appreciate that many techniques for harvesting the biomass that minimize proteolysis of the pharmaceutical protein or polypeptide are available and could be applied to the present invention.

### Vaccines

The present invention provides vaccine compositions comprising a least one influenza antigen polypeptide, fusion thereof, and/or immunogenic portion(s) thereof, which are intended to elicit a physiological effect upon administration to a subject. A vaccine protein may have healing curative or palliative properties against a disorder or disease and can be administered to ameliorate relieve, alleviate, delay onset of, reverse or lessen symptoms or severity of a disease or disorder. A vaccine comprising an influenza antigen polypeptide may have prophylactic properties and can be used to prevent or delay the onset of a disease or to lessen the severity of such disease, disorder, or pathological condition when it does emerge. A physiological effect elicited by treatment of a subject with antigen according to the present invention can include an effective immune response such that infection by an organism is thwarted. Considerations for administration of influenza antigen polypeptides to a subject in need thereof are discussed in further detail in the section below entitled “Administration.”

In general, active vaccination involves the exposure of a subject's immune system to one or more agents that are recognized as unwanted, undesired, and/or foreign and elicit an endogenous immune response. Typically, such an immune response results in the activation of antigen-specific naive lymphocytes that then give rise to antibody-secreting B cells or antigen-specific effector and memory T cells or both. This approach can result in long-lived protective immunity that may be boosted from time to time by renewed exposure to the same antigenic material.

In some embodiments, a vaccine composition comprising at least one influenza antigen polypeptide is a subunit vaccine. In general, a subunit vaccine comprises purified antigens rather than whole organisms. Subunit vaccines are not infectious, so they can safely be given to immunosuppressed people, and they are less likely to induce unfavorable immune reactions and/or other adverse side effects. One potential disadvantage of subunit vaccines are that the antigens may not retain their native conformation, so that antibodies produced against the subunit may not recognize the same protein on the pathogen surface; and isolated protein does not stimulate the immune system as well as a whole organism vaccine. Therefore, in some situations, it may be necessary to administer subunit vaccines in higher doses than a whole-agent vaccine (e.g., live attenuated vaccines, inactivated pathogen vaccines, etc.) in order to achieve the same therapeutic effect. In contrast, whole-agent vaccines, such as vaccines that utilize live attenuated or inactivated pathogens, typically yield a vigorous immune response, but their use has limitations. For example, live vaccine strains can sometimes cause infectious pathologies, especially when administered to immune-compromised recipients. Moreover, many pathogens, particularly viruses (such as influenza), undergo continuous rapid mutations in their genome, which allow them to escape immune responses to antigenically distinct vaccine strains.

In some embodiments, subunit vaccines in accordance with the present invention comprising plant-produced influenza antigen polypeptides (e.g., HA and/or NA polypeptides, as described herein) can be administered at very low doses and stimulate immune responses. In some embodiments, less than about 100 μg, less than about 90 μg, less than about 80 μg, less than about 70 μg, less than about 60 μg, less than about 50 μg, less than about 40 μg, less than about 35 μg, less than about 30 μg, less than about 25 μg, less than about 20 μg, less than about 15 μg, less than about 5 μg, less than about 4 μg, less than about 3 μg, less than about 2 μg, or less than about 1 μg of plant-produced influenza antigen polypeptide and/or immunogenic portion thereof can be used to stimulate an immune response and/or to prevent, delay the onset of, and/or provide protection against influenza infection.

In some embodiments, the present invention provides subunit vaccines against influenza. In some embodiments, subunit vaccines comprise an antigen that has been at least partially purified from non-antigenic components. For example, a subunit vaccine may be an influenza antigen polypeptide, fusion thereof, and/or immunogenic portion thereof that is expressed in a live organism (such as a plant, virus, bacterium, yeast, mammalian cell, egg, etc.), but is at least partially purified from the non-antigen components of the live organism. In some embodiments, a subunit vaccine is at least 30%, at least 40%, at least 50%, at least 60%, at least 70%, at least 80%, at least 90%, at least 95%, at least 98%, or at least 99% purified from the non-antigen components of the organism in which the antigen was expressed. In some embodiments, a subunit vaccine may be an influenza antigen polypeptide, fusion thereof, and/or immunogenic portion thereof that is chemically-synthesized.

In some embodiments, a subunit vaccine may be an influenza antigen polypeptide, fusion thereof, and/or immunogenic portion thereof that is expressed in a live organism (such as a plant, virus, bacterium, yeast, mammalian cell, egg, etc.), but is not at least partially purified from the non-antigen components of the live organism. For example, a subunit vaccine may be an influenza antigen polypeptide, fusion thereof, and/or immunogenic portion thereof that is expressed in a live organism that is administered directly to a subject in order to elicit an immune response. In some embodiments, a subunit vaccine may be an influenza antigen polypeptide, fusion thereof, and/or immunogenic portion thereof that is expressed in a plant, as described herein, wherein the plant material is administered directly to a subject in order to elicit an immune response.

The present invention provides pharmaceutical influenza antigen polypeptides, fusions thereof, and/or immunogenic portions thereof, active as subunit vaccines for therapeutic and/or prophylactic treatment of influenza infection. In certain embodiments, influenza antigen polypeptides may be produced by plant(s) or portion thereof (e.g., root, cell, sprout, cell line, plant, etc.) in accordance with the invention. In certain embodiments, provided influenza antigen polypeptides are expressed in plants, plant cells, and/or plant tissues (e.g., sprouts, sprouted seedlings, roots, root culture, clonal cells, clonal cell lines, clonal plants, etc.), and can be used directly from plant or partially purified or purified in preparation for pharmaceutical administration to a subject.

The present invention provides plants, plant cells, and plant tissues expressing influenza antigen polypeptides that maintain pharmaceutical activity when administered to a subject in need thereof. Exemplary subjects include vertebrates (e.g., mammals such as humans). According to the present invention, subjects include veterinary subjects such as bovines, ovines, canines, felines, birds, pigs etc. In certain aspects, an edible plant or portion thereof (e.g., sprout, root) is administered orally to a subject in a therapeutically effective amount. In some aspects one or more influenza antigen polypeptides are provided in a pharmaceutical preparation, as described herein.

Where it is desirable to formulate an influenza vaccine comprising plant material, it will often be desirable to have utilized a plant that is not toxic to the relevant recipient (e.g., a human or other animal). Relevant plant tissue (e.g., cells, roots, leaves) may simply be harvested and processed according to techniques known in the art, with due consideration to maintaining activity of the expressed product. In certain embodiments, it is desirable to have expressed influenza antigen polypeptides in an edible plant (and, specifically in edible portions of the plant) so that the material can subsequently be eaten. For instance, where vaccine antigen is active after oral delivery (when properly formulated), it may be desirable to produce antigen protein in an edible plant portion, and to formulate expressed influenza antigen polypeptide for oral delivery together with some or all of the plant material with which the protein was expressed.

Vaccine compositions in accordance with the invention comprise one or more influenza antigen polypeptides. In certain embodiments, exactly one influenza antigen polypeptide is included in an administered vaccine composition. In certain embodiments, at least two influenza antigen polypeptides are included in an administered vaccine composition. In some aspects, combination vaccines may include one thermostable fusion protein comprising an influenza antigen polypeptide; in some aspects, two or more thermostable fusion proteins comprising influenza antigen polypeptides are provided.

In some embodiments, vaccine compositions comprise exactly one HA polypeptide. In some embodiments, vaccine compositions comprise exactly one NA polypeptide. In some embodiments, vaccine compositions comprise exactly two HA polypeptides. In some embodiments, vaccine compositions comprise exactly two NA polypeptides. In some embodiments, vaccine compositions comprise exactly three HA polypeptides. In some embodiments, vaccine compositions comprise exactly three NA polypeptides. In some embodiments, vaccine compositions comprise four or more (e.g., 4, 5, 6, 7, 8, 9, 10, 15, or more) HA polypeptides. In some embodiments, vaccine compositions comprise four or more (e.g., 4, 5, 6, 7, 8, 9, 10, 15, or more) NA polypeptides.

In some embodiments, vaccine compositions comprise exactly one HA polypeptide and exactly one NA polypeptide. In some embodiments, vaccine compositions comprise exactly two HA polypeptides and exactly two NA polypeptides. In some embodiments, vaccine compositions comprise exactly three HA polypeptides and exactly three NA polypeptides. In some embodiments, vaccine compositions comprise four or more (e.g., 4, 5, 6, 7, 8, 9, 10, 15, or more) HA polypeptides and four or more (e.g., 4, 5, 6, 7, 8, 9, 10, 15, or more) NA polypeptides. In some embodiments, vaccine compositions comprise exactly one HA polypeptide and two or more (e.g., 2, 3, 4, 5, 6, 7, 8, 9, 10, 15, or more) NA polypeptides. In some embodiments, vaccine compositions comprise two or more (e.g., 2, 3, 4, 5, 6, 7, 8, 9, 10, 15, or more) HA polypeptides and exactly one NA polypeptide.

In some embodiments, vaccine compositions comprise polytopes (i.e., tandem fusions of two or more amino acid sequences) of two or more influenza antigen polypeptides and/or immunogenic portions thereof. For example, in some embodiments, a polytope comprises exactly one HA polypeptide. In some embodiments, a polytope comprises comprise exactly one NA polypeptide. In some embodiments, a polytope comprises exactly two HA polypeptides. In some embodiments, a polytope comprises exactly two NA polypeptides. In some embodiments, a polytope comprises exactly three HA polypeptides. In some embodiments, a polytope comprises exactly three NA polypeptides. In some embodiments, a polytope comprises four or more (e.g., 4, 5, 6, 7, 8, 9, 10, 15, or more) HA polypeptides. In some embodiments, a polytope comprises four or more (e.g., 4, 5, 6, 7, 8, 9, 10, 15, or more) NA polypeptides.

In some embodiments, a polytope comprises exactly one HA polypeptide and exactly one NA polypeptide. In some embodiments, a polytope comprises exactly two HA polypeptides and exactly two NA polypeptides. In some embodiments, a polytope comprises exactly three HA polypeptides and exactly three NA polypeptides. In some embodiments, a polytope comprises four or more (e.g., 4, 5, 6, 7, 8, 9, 10, 15, or more) HA polypeptides and four or more (e.g., 4, 5, 6, 7, 8, 9, 10, 15, or more) NA polypeptides. In some embodiments, a polytope comprises exactly one HA polypeptide and two or more (e.g., 2, 3, 4, 5, 6, 7, 8, 9, 10, 15, or more) NA polypeptides. In some embodiments, a polytope comprises two or more (e.g., 2, 3, 4, 5, 6, 7, 8, 9, 10, 15, or more) HA polypeptides and exactly one NA polypeptide.

Where combination vaccines are utilized, it will be understood that any combination of influenza antigen polypeptides may be used for such combinations. Compositions may include multiple influenza antigen polypeptides, including multiple antigens provided herein. Furthermore, compositions may include one or more antigens provided herein with one or more additional antigens. Combinations of influenza antigen polypeptides include influenza antigen polypeptides derived from one or more various subtypes or strains such that immunization confers immune response against more than one infection type. Combinations of influenza antigen polypeptides may include at least one, at least two, at least three, at least four or more antigens derived from different subtypes or strains. In some combinations, at least two or at least three antigens from different subtypes are combined in one vaccine composition. Furthermore, combination vaccines may utilize influenza antigen polypeptides and antigen from one or more unique infectious agents.

Additional Vaccine Components

Vaccine compositions in accordance with the invention may include additionally any suitable adjuvant to enhance the immunogenicity of the vaccine when administered to a subject. For example, such adjuvant(s) may include, without limitation, saponins, such as extracts of Quillaja saponaria (QS), including purified subfractions of food grade QS such as Quil A and QS21; alum; metallic salt particles (e.g., aluminum hydroxide, aluminum phosphate, etc.); mineral oil; MF59; Malp2; incomplete Freund's adjuvant; complete Freund's adjuvant; alhydrogel; 3 De-O-acylated monophosphoryl lipid A (3 D-MPL); lipid A; Bortadella pertussis; Mycobacterium tuberculosis; Merck Adjuvant 65 (Merck and Company, Inc., Rahway, N.J.); squalene; virosomes; oil-in-water emulsions (e.g., SBAS2); liposome formulations (e.g., SBAS1); AS03 etc. Further adjuvants include immunomodulatory oligonucleotides, for example unmethylated CpG sequences as disclosed in WO 96/02555. Combinations of different adjuvants, such as those mentioned hereinabove, are contemplated as providing an adjuvant which is a preferential stimulator of TH1 cell response. For example, QS21 can be formulated together with 3 D-MPL. The ratio of QS21:3 D-MPL will typically be in the order of 1:10 to 10:1; 1:5 to 5:1; and often substantially 1:1. The desired range for optimal synergy may be 2.5:1 to 1:1 3 D-MPL: QS21. Doses of purified QS extracts suitable for use in a human vaccine formulation are from 0.01 mg to 10 mg per kilogram of bodyweight.

It should be noted that certain thermostable proteins (e.g., lichenase) may themselves demonstrate immunoresponse potentiating activity, such that use of such protein whether in a fusion with an influenza antigen polypeptide or separately may be considered use of an adjuvant. Thus, inventive vaccine compositions may further comprise one or more adjuvants. Certain vaccine compositions may comprise two or more adjuvants. Furthermore, depending on formulation and routes of administration, certain adjuvants may be desired in particular formulations and/or combinations.

In certain situations, it may be desirable to prolong the effect of an inventive vaccine by slowing the absorption of one or more components of the vaccine product (e.g., protein) that is subcutaneously or intramuscularly injected. This may be accomplished by use of a liquid suspension of crystalline or amorphous material with poor water solubility. The rate of absorption of product then depends upon its rate of dissolution, which in turn, may depend upon size and form. Alternatively or additionally, delayed absorption of a parenterally administered product is accomplished by dissolving or suspending the product in an oil vehicle. Injectable depot forms are made by forming microcapsule matrices of protein in biodegradable polymers such as polylactide-polyglycolide. Depending upon the ratio of product to polymer and the nature of the particular polymer employed, rate of release can be controlled. Examples of biodegradable polymers include poly(orthoesters) and poly(anhydrides). Depot injectable formulations may be prepared by entrapping product in liposomes or microemulsions, which are compatible with body tissues. Alternative polymeric delivery vehicles can be used for oral formulations. For example, biodegradable, biocompatible polymers such as ethylene vinyl acetate, polyanhydrides, polyglycolic acid, collagen, polyorthoesters, and polylactic acid, etc., can be used. Antigen(s) or an immunogenic portions thereof may be formulated as microparticles, e.g., in combination with a polymeric delivery vehicle.

Enterally administered preparations of vaccine antigens may be introduced in solid, semi-solid, suspension or emulsion form and may be compounded with any pharmaceutically acceptable carriers, such as water, suspending agents, and emulsifying agents. Antigens may be administered by means of pumps or sustained-release forms, especially when administered as a preventive measure, so as to prevent the development of disease in a subject or to ameliorate or delay an already established disease. Supplementary active compounds, e.g., compounds independently active against the disease or clinical condition to be treated, or compounds that enhance activity of an inventive compound, can be incorporated into or administered with compositions. Flavorants and coloring agents can be used.

Inventive vaccine products, optionally together with plant tissue, are particularly well suited for oral administration as pharmaceutical compositions. Oral liquid formulations can be used and may be of particular utility for pediatric populations. Harvested plant material may be processed in any of a variety of ways (e.g., air drying, freeze drying, extraction etc.), depending on the properties of the desired therapeutic product and its desired form. Such compositions as described above may be ingested orally alone or ingested together with food or feed or a beverage. Compositions for oral administration include plants; extractions of plants, and proteins purified from infected plants provided as dry powders, foodstuffs, aqueous or non-aqueous solvents, suspensions, or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medial parenteral vehicles including sodium chloride solution, Ringer's dextrose solution, dextrose plus sodium chloride solution, Ringer's solution containing lactose or fixed oils. Examples of dry powders include any plant biomass that has been dried, for example, freeze dried, air dried, or spray dried. For example, plants may be air dried by placing them in a commercial air dryer at about 120° F. until biomass contains less than 5% moisture by weight. The dried plants may be stored for further processing as bulk solids or further processed by grinding to a desired mesh sized powder. Alternatively or additionally, freeze-drying may be used for products that are sensitive to air-drying. Products may be freeze dried by placing them into a vacuum drier and dried frozen under a vacuum until the biomass contains less than about 5% moisture by weight. Dried material can be further processed as described herein.

Plant-derived material may be administered as or together with one or more herbal preparations. Useful herbal preparations include liquid and solid herbal preparations. Some examples of herbal preparations include tinctures, extracts (e.g., aqueous extracts, alcohol extracts), decoctions, dried preparations (e.g., air-dried, spray dried, frozen, or freeze-dried), powders (e.g., lyophilized powder), and liquid. Herbal preparations can be provided in any standard delivery vehicle, such as a capsule, tablet, suppository, liquid dosage, etc. Those skilled in the art will appreciate the various formulations and modalities of delivery of herbal preparations that may be applied to the present invention.

Pharmaceutical formulations of the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, at least 96%, at least 97%, at least 98%, at least 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g., cornstarch, starch paste, etc.); gelatin; sugars (e.g., sucrose, glucose, dextrose, dextrin, molasses, lactose, lactitol, mannitol, etc.); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate [VEEGUM®], larch arabogalactan, etc.); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such a CREMOPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato starch, tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Vaccine products, optionally together with plant tissue, are particularly well suited for oral administration as pharmaceutical compositions. Oral liquid formulations can be used and may be of particular utility for pediatric populations. Harvested plant material may be processed in any of a variety of ways (e.g., air drying, freeze drying, extraction etc.), depending on the properties of the desired therapeutic product and its desired form. Such compositions as described above may be ingested orally alone or ingested together with food or feed or a beverage. Compositions for oral administration include plants; extractions of plants, and proteins purified from infected plants provided as dry powders, foodstuffs, aqueous or non-aqueous solvents, suspensions, or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medial parenteral vehicles including sodium chloride solution, Ringer's dextrose solution, dextrose plus sodium chloride solution, Ringer's solution containing lactose or fixed oils. Examples of dry powders include any plant biomass that has been dried, for example, freeze dried, air dried, or spray dried. For example, plants may be air dried by placing them in a commercial air dryer at about 120° F. until biomass contains less than 5% moisture by weight. Dried plants may be stored for further processing as bulk solids or further processed by grinding to a desired mesh sized powder. Alternatively or additionally, freeze-drying may be used for products that are sensitive to air-drying. Products may be freeze dried by placing them into a vacuum drier and dried frozen under a vacuum until the biomass contains less than about 5% moisture by weight. Dried material can be further processed as described herein.

Plant-derived material may be administered as or together with one or more herbal preparations. Useful herbal preparations include liquid and solid herbal preparations. Some examples of herbal preparations include tinctures, extracts (e.g., aqueous extracts, alcohol extracts), decoctions, dried preparations (e.g., air-dried, spray dried, frozen, or freeze-dried), powders (e.g., lyophilized powder), and liquid. Herbal preparations can be provided in any standard delivery vehicle, such as a capsule, tablet, suppository, liquid dosage, etc. Those skilled in the art will appreciate the various formulations and modalities of delivery of herbal preparations that may be applied to the present invention.

In some methods, a plant or portion thereof expressing an influenza antigen polypeptide according to the present invention, or biomass thereof, is administered orally as medicinal food. Such edible compositions are typically consumed by eating raw, if in a solid form, or by drinking, if in liquid form. The plant material can be directly ingested without a prior processing step or after minimal culinary preparation. For example, a vaccine antigen may be expressed in a sprout which can be eaten directly. For instance, vaccine antigens expressed in an alfalfa sprout, mung bean sprout, or spinach or lettuce leaf sprout, etc. In some embodiments, plant biomass may be processed and the material recovered after the processing step is ingested.

Processing methods useful in accordance with the present invention are methods commonly used in the food or feed industry. Final products of such methods typically include a substantial amount of an expressed antigen and can be conveniently eaten or drunk. The final product may be mixed with other food or feed forms, such as salts, carriers, favor enhancers, antibiotics, and the like, and consumed in solid, semi-solid, suspension, emulsion, or liquid form. Such methods can include a conservation step, such as, e.g., pasteurization, cooking, or addition of conservation and preservation agents. Any plant may be used and processed in the present invention to produce edible or drinkable plant matter. The amount of influenza antigen polypeptide in a plant-derived preparation may be tested by methods standard in the art, e.g., gel electrophoresis, ELISA, or western blot analysis, using a probe or antibody specific for product. This determination may be used to standardize the amount of vaccine antigen protein ingested. For example, the amount of vaccine antigen may be determined and regulated, for example, by mixing batches of product having different levels of product so that the quantity of material to be drunk or eaten to ingest a single dose can be standardized. A contained, regulatable environment in accordance with the invention, however, should minimize the need to carry out such standardization procedures.

A vaccine protein produced in a plant cell or tissue and eaten by a subject may be preferably absorbed by the digestive system. One advantage of the ingestion of plant tissue that has been only minimally processed is to provide encapsulation or sequestration of the protein in cells of the plant. Thus, product may receive at least some protection from digestion in the upper digestive tract before reaching the gut or intestine and a higher proportion of active product would be available for uptake.

Dosage forms for topical and/or transdermal administration of a compound in accordance with this invention may include ointments, pastes, creams, lotions, gels, powders, solutions, sprays, inhalants and/or patches. Generally, the active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, the rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition in accordance with the invention may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 mn. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and the active ingredient may constitute 0.1% to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions in accordance with the invention formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 ran to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e., by rapid inhalation through the nasal passage from a container of the powder held close to the nose.

Formulations suitable for nasal administration may, for example, comprise from about as little as 0.1% (w/w) and as much as 100% (w/w) of the active ingredient, and may comprise one or more of the additional ingredients described herein. A pharmaceutical composition in accordance with the invention may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 nm to about 200 nm, and may further comprise one or more of the additional ingredients described herein.

A pharmaceutical composition in accordance with the invention may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

In certain situations, it may be desirable to prolong the effect of a vaccine by slowing the absorption of one or more components of the vaccine product (e.g., protein) that is subcutaneously or intramuscularly injected. This may be accomplished by use of a liquid suspension of crystalline or amorphous material with poor water solubility. The rate of absorption of product then depends upon its rate of dissolution, which in turn, may depend upon size and form. Alternatively or additionally, delayed absorption of a parenterally administered product is accomplished by dissolving or suspending the product in an oil vehicle. Injectable depot forms are made by forming microcapsule matrices of protein in biodegradable polymers such as polylactide-polyglycolide. Depending upon the ratio of product to polymer and the nature of the particular polymer employed, rate of release can be controlled. Examples of biodegradable polymers include poly(orthoesters) and poly(anhydrides). Depot injectable formulations may be prepared by entrapping product in liposomes or microemulsions, which are compatible with body tissues. Alternative polymeric delivery vehicles can be used for oral formulations. For example, biodegradable, biocompatible polymers such as ethylene vinyl acetate, polyanhydrides, polyglycolic acid, collagen, polyorthoesters, and polylactic acid, etc., can be used. Antigen(s) or an immunogenic portions thereof may be formulated as microparticles, e.g., in combination with a polymeric delivery vehicle.

General considerations in the formulation and/or manufacture of pharmaceutical agents may be found, for example, in Remington: The Science and Practice of Pharmacy 21^{st} ed., Lippincott Williams & Wilkins, 2005.

### Administration

Among other things, present invention provides subunit vaccines. In some embodiments, subunit vaccines in accordance with the present invention may be administered to a subject at low doses in order to stimulate an immune response and/or confer protectivity. As used herein, the term “low-dose vaccine” generally refers to a vaccine that is immunogenic and/or protective when administered to a subject at low-doses. According to the present invention, administration of a low-dose vaccine comprises administration of a subunit vaccine composition comprising less than 100 μg of an influenza antigen polypeptide, fusion thereof, and/or immunogenic portion thereof.

In some embodiments, administration of a low-dose subunit vaccine comprises administering a subunit vaccine comprising less than about 100 μg, less than about 90 μg, less than about 80 μg, less than about 70 μg, less than about 60 μg, less than about 50 μg, less than about 40 μg, less than about 35 μg, less than about 30 μg, less than about 25 μg, less than about 20 μg, less than about 15 μg, less than about 5 μg, less than about 4 μg, less than about 3 μg, less than about 2 μg, or less than about 1 μg of plant-produced influenza antigen polypeptide, fusion thereof, and/or immunogenic portion thereof to a subject in need thereof. In some embodiments, the plant-produced influenza antigen polypeptide, fusion thereof, and/or immunogenic portion thereof has been at least partially purified from non-antigenic components, as described herein. In some embodiments, the plant-produced influenza antigen polypeptide, fusion thereof, and/or immunogenic portion thereof has not been at least partially purified from non-antigenic components, as described herein. Suitable vaccine compositions for administration to a subject are described in further detail in the section above, entitled “Vaccines.”

Influenza antigen polypeptides, fusions thereof, and/or immunogenic portions thereof in accordance with the invention and/or pharmaceutical compositions thereof (e.g., vaccines) may be administered using any amount and any route of administration effective for treatment.

The exact amount required will vary from subject to subject, depending on the species, age, and general condition of the subject, the severity of the infection, the particular composition, its mode of administration, its mode of activity, and the like. Influenza antigen polypeptides are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disorder being treated and the severity of the disorder; the activity of the specific influenza antigen polypeptide employed; the specific pharmaceutical composition administered; the half-life of the composition after administration; the age, body weight, general health, sex, and diet of the subject; the time of administration, route of administration, and rate of excretion of the specific compound employed; the duration of the treatment; drugs used in combination or coincidental with the specific compound employed; and like factors, well known in the medical arts.

Pharmaceutical compositions of the present invention (e.g., vaccines) may be administered by any route. In some embodiments, pharmaceutical compositions of the present invention are administered by a variety of routes, including oral (PO), intravenous (IV), intramuscular (IM), intra-arterial, intramedullary, intrathecal, subcutaneous (SQ), intraventricular, transdermal, interdermal, intradermal, rectal (PR), vaginal, intraperitoneal (IP), intragastric (IG), topical (e.g., by powders, ointments, creams, gels, lotions, and/or drops), mucosal, intranasal, buccal, enteral, vitreal, sublingual; by intratracheal instillation, bronchial instillation, and/or inhalation; as an oral spray, nasal spray, and/or aerosol; and/or through a portal vein catheter. In general, the most appropriate route of administration will depend upon a variety of factors including the nature of the agent being administered (e.g., its stability in the environment of the gastrointestinal tract), the condition of the subject (e.g., whether the subject is able to tolerate a particular mode of administration), etc.

In some embodiments, vaccines in accordance with the invention are delivered by multiple routes of administration (e.g., by subcutaneous injection and by intranasal inhalation). For vaccines involving two or more doses, different doses may be administered via different routes.

In some embodiments, vaccines in accordance with the invention are delivered by subcutaneous injection. In some embodiments, vaccines in accordance with the invention are administered by intramuscular and/or intravenous injection. In some embodiments, vaccines in accordance with the invention are delivered by intranasal inhalation.

In some embodiments, vaccines in accordance with the invention are delivered by oral and/or mucosal routes. Oral and/or mucosal delivery has the potential to prevent infection of mucosal tissues, the primary gateway of infection for many pathogens. Oral and/or mucosal delivery can prime systemic immune response. There has been considerable progress in the development of heterologous expression systems for oral administration of antigens that stimulate the mucosal-immune system and can prime systemic immunity. Previous efforts at delivery of oral vaccine however, have demonstrated a requirement for considerable quantities of antigen in achieving efficacy. Thus, economical production of large quantities of target antigens is a prerequisite for creation of effective oral vaccines. Development of plants expressing antigens, including thermostable antigens, represents a more realistic approach to such difficulties.

In certain embodiments, an influenza antigen polypeptide expressed in a plant or portion thereof is administered to a subject orally by direct administration of a plant to a subject. In some aspects a vaccine protein expressed in a plant or portion thereof is extracted and/or purified, and used for the preparation of a pharmaceutical composition. It may be desirable to formulate such isolated products for their intended use (e.g., as a pharmaceutical agent, vaccine composition, etc.). In some embodiments, it will be desirable to formulate products together with some or all of plant tissues that express them.

In certain embodiments, an influenza antigen polypeptide expressed in a plant or portion thereof is administered to a subject orally by direct administration of a plant to a subject. In some aspects a vaccine protein expressed in a plant or portion thereof is extracted and/or purified, and used for preparation of a pharmaceutical composition. It may be desirable to formulate such isolated products for their intended use (e.g., as a pharmaceutical agent, vaccine composition, etc.). In some embodiments, it will be desirable to formulate products together with some or all of plant tissues that express them.

A vaccine protein produced in a plant cell or tissue and eaten by a subject may be preferably absorbed by the digestive system. One advantage of the ingestion of plant tissue that has been only minimally processed is to provide encapsulation or sequestration of the protein in cells of the plant. Thus, product may receive at least some protection from digestion in the upper digestive tract before reaching the gut or intestine and a higher proportion of active product would be available for uptake.

Where it is desirable to formulate product together with plant material, it will often be desirable to have utilized a plant that is not toxic to the relevant recipient (e.g., a human or other animal). Relevant plant tissue (e.g., cells, roots, leaves) may simply be harvested and processed according to techniques known in the art, with due consideration to maintaining activity of the expressed product. In certain embodiments, it is desirable to have expressed influenza antigen polypeptide in an edible plant (and, specifically in edible portions of the plant) so that the material can subsequently be eaten. For instance, where vaccine antigen is active after oral delivery (when properly formulated), it may be desirable to produce antigen protein in an edible plant portion, and to formulate expressed influenza antigen polypeptide for oral delivery together with some or all of the plant material with which a protein was expressed.

In certain embodiments, influenza antigen polypeptides in accordance with the present invention and/or pharmaceutical compositions thereof (e.g., vaccines) in accordance with the invention may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg of subject body weight per day to obtain the desired therapeutic effect. The desired dosage may be delivered more than three times per day, three times per day, two times per day, once per day, every other day, every third day, every week, every two weeks, every three weeks, every four weeks, every two months, every six months, or every twelve months. In certain embodiments, the desired dosage may be delivered using multiple administrations (e.g., two, three, four, five, six, seven, eight, nine, ten, eleven, twelve, thirteen, fourteen, or more administrations).

Compositions are administered in such amounts and for such time as is necessary to achieve the desired result. In certain embodiments, a “therapeutically effective amount” of a pharmaceutical composition is that amount effective for treating, attenuating, or preventing a disease in a subject. Thus, the “amount effective to treat, attenuate, or prevent disease,” as used herein, refers to a nontoxic but sufficient amount of the pharmaceutical composition to treat, attenuate, or prevent disease in any subject. For example, the “therapeutically effective amount” can be an amount to treat, attenuate, or prevent infection (e.g., influenza infection), etc.

It will be appreciated that influenza antigen polypeptides in accordance with the present invention and/or pharmaceutical compositions thereof can be employed in combination therapies. The particular combination of therapies (e.g., therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, influenza antigen polypeptides useful for treating, preventing, and/or delaying the onset of influenza infection may be administered concurrently with another agent useful for treating, preventing, and/or delaying the onset of influenza infection), or they may achieve different effects (e.g., control of any adverse effects). The invention encompasses the delivery of pharmaceutical compositions in combination with agents that may improve their bioavailability, reduce and/or modify their metabolism, inhibit their excretion, and/or modify their distribution within the body.

Pharmaceutical compositions in accordance with the present invention may be administered either alone or in combination with one or more other therapeutic agents. By “in combination with,” it is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In will be appreciated that therapeutically active agents utilized in combination may be administered together in a single composition or administered separately in different compositions. In general, each agent will be administered at a dose and/or on a time schedule determined for that agent.

In general, it is expected that agents utilized in combination with be utilized at levels that do not exceed the levels at which they are utilized individually. In some embodiments, the levels utilized in combination will be lower than those utilized individually.

In certain embodiments, vaccine compositions comprising at least one influenza antigen polypeptide are administered in combination with other influenza vaccines. In certain embodiments, vaccine compositions comprising at least one influenza antigen polypeptide are administered in combination with other influenza therapeutics. In certain embodiments, vaccine compositions comprising at least one influenza antigen polypeptide are administered in combination with antiviral drugs, such as neuraminidase inhibitors (e.g., oseltamivir [TAMIFLU®], zanamivir [RELENZAAND®] and/or M2 inhibitors (e.g., adamantane, adamantane derivatives, rimantadine, etc.).

### Kits

In one aspect, the present invention provides a pharmaceutical pack or kit including influenza antigen polypeptides according to the present invention. In certain embodiments, pharmaceutical packs or kits include plants, plant cells, and/or plant tissues producing an influenza antigen polypeptide according to the present invention, or preparations, extracts, or pharmaceutical compositions containing vaccine in one or more containers filled with optionally one or more additional ingredients of pharmaceutical compositions in accordance with the invention. In some embodiments, pharmaceutical packs or kits include pharmaceutical compositions comprising purified influenza antigen polypeptides according to the present invention, in one or more containers optionally filled with one or more additional ingredients of pharmaceutical compositions in accordance with the invention. In certain embodiments, the pharmaceutical pack or kit includes an additional approved therapeutic agent (e.g., influenza antigen polypeptide, influenza vaccine, influenza therapeutic) for use as a combination therapy. Optionally associated with such container(s) can be a notice in the form prescribed by a governmental agency regulating the manufacture, use or sale of pharmaceutical products, which notice reflects approval by the agency of manufacture, use, or sale for human administration.

Kits are provided that include therapeutic and/or prophylactic reagents. As but one non-limiting example, influenza vaccine can be provided (e.g., as an oral, injectable, and/or intranasal formulation) and administered as therapy. Pharmaceutical doses or instructions therefor may be provided in the kit for administration to an individual suffering from or at risk for influenza infection.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain information, exemplification and guidance, which can be adapted to the practice of this invention in its various embodiments and the equivalents thereof.

Provided herein are a subunit vaccine compositions comprising: a plant-produced influenza polypeptide antigen; and a pharmaceutically acceptable excipient; wherein the subunit vaccine composition elicits an immune response upon administration to a subject. In some embodiments, the plant-produced influenza polypeptide antigen is a hemagglutinin polypeptide and can be a hemagglutinin polypeptide selected from the group consisting of SEQ ID NOs.: 1-35, 85, 87, 89, 91, 93, 95, 97, 99, 101, 103, 105, 107, 109, 111 and 112. In some embodiments, the plant-produced influenza polypeptide antigen is a neuraminidase polypeptide and can be a neuraminidase polypeptide selected from the group consisting of SEQ ID NOs.: 36-43 and 110. In some embodiments, a single dose of the subunit vaccine composition comprises no more than 200 μg of the plant-produced influenza polypeptide antigen, no more than 100 μg of the plant-produced influenza polypeptide antigen, no more than 75 μg of the plant-produced influenza polypeptide antigen, no more than 50 μg of the plant-produced influenza polypeptide antigen, no more than 25 μg of the plant-produced influenza polypeptide antigen, no more than 10 μg of the plant-produced influenza polypeptide antigen, no more than 5 μg of the plant-produced influenza polypeptide antigen, no more than 1 μg of the plant-produced influenza polypeptide antigen.

In some embodiments, the plant-produced influenza polypeptide antigen is purified from plant materials. The plant-produced influenza polypeptide antigen can be about 70% pure, about 80% pure, about 90% pure, about 95% pure, about 99% pure. In some embodiments, the plant-produced influenza polypeptide antigen is not purified from plant materials. The plant-produced influenza polypeptide antigen can be administered to a subject as a whole plant or plant extract.

In some embodiments, the subunit vaccine composition further comprises at least one vaccine adjuvant. The adjuvant can be selected from the group consisting of alum, Quil A, QS21, aluminum hydroxide, aluminum phosphate, mineral oil, MF59, Malp2, incomplete Freund's adjuvant, complete Freund's adjuvant, alhydrogel, 3 De-O-acylated monophosphoryl lipid A (3D-MPL), lipid A, Bortadella pertussis, Mycobacterium tuberculosis, Merck Adjuvant 65, squalene, virosomes, SBAS2, SBAS1, and unmethylated CpG sequences.

In some embodiments, the antigen is produced in a plant selected from a transgenic plant or a plant transiently expressing the antigen. The antigen can be expressed in the plant from a launch vector.

Also provided are methods for inducing a protective immune response against influenza infection in a subject comprising administering to a subject an effective amount of a subunit vaccine composition. The composition can be administered orally, intranasally, subcutaneously, intravenously, intraperitoneally, or intramuscularly. The composition can be administered orally via feeding plant cells to the subject. The subject can be human; in some embodiments, The subject is selected from the group consisting of a bird, a pig, and a horse.

Also provided are methods for producing an influenza antigen polypeptide comprising: preparing a nucleic acid construct encoding an influenza antigen polypeptide; introducing the nucleic acid into a plant cell; and incubating the plant cell under conditions favorable for expression of the influenza antigen polypeptide; thereby producing the influenza antigen polypeptide. The expression of the antigen protein can be under control of a viral promoter. In some embodiments, the nucleic acid construct further comprises vector nucleic acid sequence. The vector can be a binary vector. The nucleic acid construct can further comprise sequences encoding viral proteins. The plant cell can be selected from the group consisting of alfalfa, radish, mustard, mung bean, broccoli, watercress, soybean, wheat sunflower, cabbage, clover, petunia, tomato, potato, nicotine, spinach, and lentil cell. The plant cell can be of a genus selected from the Brassica genus, the Nicotiana genus, and the Petunia genus. The influenza antigen polypeptide can be produced in sprouted seedlings. Some embodiments further comprise recovering partially purified or purified influenza antigen polypeptide which is produced.

Also provided are isolated nucleic acid constructs comprising nucleic acid sequence encoding an influenza antigen polypeptide, wherein the influenza antigen polypeptide comprises a sequence as set forth in any one of SEQ ID NOs: 1-43, 85, 87, 89, 91, 93, 95, 97, 99, 101, 103, 105, 107, 109, 110, 111, and 112. The isolated nucleic acid constructs can further comprise vector nucleic acid sequences. The isolated nucleic acid constructs can further comprise viral promoter nucleic acid sequence. The vector can be a binary vector.

The constructs can further comprise nucleic acid sequences encoding viral proteins.

Also provided are host cells comprising the nucleic acid constructs. The host cell can be a plant cell. The plant cell can be selected from the group consisting of alfalfa, radish, mustard, mung bean, broccoli, watercress, soybean, wheat sunflower, cabbage, clover, petunia, tomato, potato, tobacco, spinach, and lentil. The plant cell can be a genus selected from the Brassica genus, the Nicotiana genus, and the Petunia genus.

### EXEMPLIFICATION

### Example 1

### Recombinant hemagglutinin (HA) Antigens from Two H5N1 Influenza Strains

In this Example, the immunogenicity of two recombinant hemagglutinin (HA) antigens from H5N1 influenza strains A/Anhui/1/2005 and A/Bar-headed goose/Qinghai/1A/2005 as vaccine candidates was assessed. These plant-produced HA antigens were immunogenic, generating high titers of serum hemagglutination inhibition (HI) and virus neutralizing (VN) antibodies in mice.

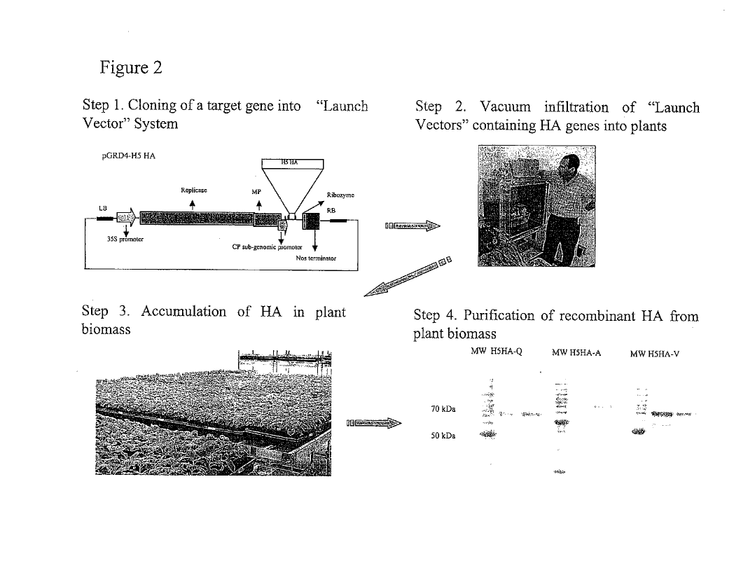

HA antigens were produced in plants according to the scheme presented in FIG. 2. HA antigens were cloned into the “launch vector” system (see, e.g., Musiychuk et al., 2007, Influenza and Other Respiratory Viruses, 1:19-25; and PCT Publication WO 07/095,304; both of which are incorporated herein by reference), specifically into vector pGR-D4 (except for Vietnam and Wyoming strains, pB1-D4). The nucleotide sequence of HA from A/Anhui/1/2005 (DQ371928) that was cloned into launch vectors is:

(SEQ ID NO: 84)

5′ATGGGATTCGTGCTTTTCTCTCAGCTTCCTTCTTTCCTTCTTGTGTCT
ACTCTTCTTCTTTTCCTTGTGATTTCTCACTCTTGCAGGGCTGATCAGAT
ATGCATTGGATACCACGCTAACAACTCTACTGAGCAAGTGGATACAATTA
TGGAAAAGAACGTGACTGTTACTCACGCTCAGGATATTCTTGAAAAGAC
TCACAACGGAAAGTTGTGCGATCTTGATGGTGTTAAGCCACTTATTCTTA
GGGACTGCAGTGTTGCTGGATGGCTTCTTGGAAACCCAATGTGCGATGA
GTTCATTAACGTGCCAGAGTGGTCTTATATTGTGGAGAAGGCTAACCCAG
CTAACGATCTTTGCTACCCAGGAAACTTCAACGATTACGAAGAGCTTAA
GCACCTTCTTTCTAGGATTAACCACTTCGAGAAGATTCAGATTATTCCAA
AGTCATCTTGGAGTGATCACGAGGCTTCATCTGGTGTTTCTTCAGCTTGC
CCATACCAAGGTACTCCATCTTTCTTCAGGAACGTTGTTTGGCTTATTAA
GAAGAACAACACTTACCCAACTATTAAGAGGTCTTACAACAACACTAAC
CAGGAAGATTTGCTTATTCTTTGGGGAATTCACCACTCTAATGATGCTGC
TGAACAGACTAAGTTGTACCAGAACCCAACTACTTACATTTCTGTGGGA
ACTTCTACTCTTAACCAGAGGCTTGTGCCAAAGATTGCTACTAGGTCTA
AGGTGAACGGACAATCTGGAAGGATGGATTTCTTCTGGACTATTCTTAA
GCCAAACGATGCTATTAACTTCGAGTCTAACGGAAACTTCATTGCTCCA
GAGTACGCTTACAAGATTGTGAAGAAAGGTGATAGTGCTATTGTGAAGT
CTGAGGTGGAGTACGGAAACTGTAACACTAAGTGCCAGACTCCAATTG
GAGCTATTAACTCTTCTATGCCATTCCACAACATTCACCCACTTACTATT
GGAGAGTGCCCAAAGTACGTGAAGTCTAACAAGTTGGTGCTTGCTACTG
GACTTAGGAACTCTCCACTTAGAGAGAGAAGAAGAAAGAGGGGACTTT
TCGGAGCTATTGCTGGATTCATTGAGGGAGGATGGCAGGGAATGGTTG
ATGGATGGTACGGATACCATCACTCTAATGAGCAGGGATCTGGATATG
CTGCTGATAAGGAATCTACTCAGAAAGCTATTGATGGTGTTACTAACAA
GGTGAACTCTATTATTGATAAGATGAACACTCAGTTCGAAGCTGTTGGA
AGAGAGTTCAACAACCTTGAGAGAAGGATTGAGAACCTTAACAAGAAA
ATGGAAGATGGATTCCTTGATGTGTGGACTTACAACGCTGAGTTGCTTG
TGCTTATGGAAAACGAGAGGACTCTTGATTTCCACGATTCTAACGTGA
AGAACCTTTACGATAAAGTGAGGCTTCAGCTTAGGGATAACGCTAAAG
AGCTTGGAAACGGTTGCTTCGAGTTCTACCACAAGTGCGATAACGAGT
GCATGGAATCTGTTAGGAACGGAACTTACGATTACCCACAGTACTCTG
AAGAAGCTAGGCTTAAGAGGGAAGAGATTTCTGGTGTTAAGTTGGAGT
CTATTGGAACTTACCAGATTCATCACCATCACCACCACAAGGATGAG
CTTTGA 3′.

Note that for SEQ ID NO: 84 above, as for all sequences in the specification, bold/underlined portions correspond to the signal peptide sequence, and italicized/underlined portions correspond to the 6×His tag and endoplasmic reticulum (ER) retention signal. For all sequences that have one or more of the signal peptide sequence, the 6×His tag, or the ER retention signal, the present invention contemplates any of these sequences that lack the signal peptide sequence, the 6×His tag, the ER retention signal, both the signal peptide sequence and the 6×His tag, both the signal peptide sequence and the ER retention signal, both the 6×His tag and the ER retention signal, and/or all three of the signal peptide sequence, the 6×His tag, and the ER retention signal.

The protein sequence encoded for by SEQ ID NO: 84 is:

(SEQ ID NO: 85)

5′MGFVLFSQLPSFLLVSTLLLFLVISHSCRADQICIGYHANNSTEQVDT
IMEKNVTVTHAQDILEKTHNGKLCDLDGVKPLILRDCSVAGWLLGNPMCD
EFINVPEWSYIVEKANPANDLCYPGNFNDYEELKHLLSRINHFEKIQIIP
KSSWSDHEASSGVSSACPYQGTPSFFRNVVWLIKKNNTYPTIKRSYNNT
NQEDLLILWGIHHSNDAAEQTKLYQNPTTYISVGTSTLNQRLVPKIATRS
KVNGQSGRMDFFWTILKPNDAINFESNGNFIAPEYAYKIVKKGDSAIVK
SEVEYGNCNTKCQTPIGAINSSMPFHNIHPLTIGECPKYVKSNKLVLAT
GLRNSPLRERRRKRGLFGAIAGFIEGGWQGMVDGWYGYHHSNEQGS
GYAADKESTQKAIDGVTNKVNSIIDKMNTQFEAVGREFNNLERRIENLN
KKMEDGFLDVWTYNAELLVLMENERTLDFHDSNVKNLYDKVRLQLRD
NAKELGNGCFEFYHKCDNECMESVRNGTYDYPQYSEEARLKREEISG
VKLESIGTYQIHHHHHHKDEL 3′.

The nucleotide sequence of HA from A/Bar-headed goose/Qinghai/1A/2005 (DQ137873) that was cloned into launch vectors is:

(SEQ ID NO: 86)

5′ATGGGTTTCGTGCTTTTCTCTCAGCTTCCTTCTTTCCTTCTTGTGTCT
ACTCTTCTTCTTTTCCTTGTGATTTCTCATTCTTGCAGGGCTGATCAA
ATCTGCATTGGTTACCATGCTAACAATTCTACTGAGCAAGTGGATACAA
TTATGGAAAAGAATGTGACTGTGACTCATGCTCAGGATATTCTTGAAAA
GACTCATAATGGAAAGTTGTGCGATCTTGATGGTGTTAAGCCTCTTATT
CTTAGGGACTGCAGTGTTGCTGGTTGGTTGCTTGGAAATCCTATGTGC
GATGAGTTCCTTAATGTGCCTGAGTGGTCTTACATTGTGGAGAAGATTA
ATCCTGCTAATGATCTTTGCTACCCTGGAAATTTCAATGATTACGAAGA
GCTTAAACATCTTCTTTCTAGGATTAATCATTTCGAGAAGATTCAGATTA
TTCCTAAGTCATCTTGGAGTGATCATGAGGCTTCATCTGGTGTTTCTTCA
GCTTGCCCTTATCAGGGAAGGTCATCTTTCTTCAGGAATGTTGTTTGGC
TTATTAAGAAGAATAACGCTTACCCTACTATTAAGAGGTCTTACAACAA
TACTAATCAGGAGGATCTTCTTGTTCTTTGGGGTATTCATCATCCTAATG
ATGCTGCTGAACAGACTAGGCTTTACCAGAATCCTACTACTTACATTTC
TGTGGGAACTTCTACTCTTAATCAGAGGCTTGTGCCTAAGATTGCTACT
AGGTCTAAAGTGAATGGTCAGTCTGGAAGGATGGAATTCTTCTGGACT
ATTCTTAAGCCAAATGATGCTATTAATTTCGAGTCTAATGGAAATTTCA
TTGCTCCTGAGAATGCTTACAAGATTGTGAAGAAGGGTGATAGTACTAT
TATGAAGTCTGAGCTTGAGTACGGTAATTGCAATACTAAGTGCCAGAC
TCCTATTGGTGCTATTAATTCTTCTATGCCTTTCCATAATATTCATCCTC
TTACTATTGGTGAGTGCCCTAAGTACGTGAAGTCTAATAGGCTTGTGC
TTGCTACTGGTCTTAGGAATTCTCCTCAGGGTGAAAGAAGAAGAAAG
AAGAGGGGACTTTTCGGAGCTATTGCTGGTTTTATTGAGGGAGGATGG
CAGGGAATGGTTGATGGTTGGTACGGTTACCATCATTCTAATGAGCAG
GGTTCTGGTTATGCTGCTGATAAGGAATCTACTCAGAAAGCTATTGAT
GGTGTTACTAATAAGGTGAACTCTATTATTGATAAGATGAATACTCAG
TTCGAGGCTGTTGGTAGAGAGTTCAACAATCTTGAGAGAAGGATTGA
GAATCTTAATAAGAAAATGGAAGATGGTTTCCTTGATGTGTGGACTTA
CAATGCTGAGTTGCTTGTGCTTATGGAAAATGAGAGGACTCTTGATTT
CCATGATTCTAATGTGAAGAATCTTTACGATAAAGTGAGGCTTCAGC
TTAGGGATAATGCTAAAGAACTTGGAAATGGTTGCTTCGAGTTCTAC
CATAGATGCGATAATGAGTGCATGGAATCTGTGAGGAATGGTACTTA
CGATTACCCTCAGTACTCTGAAGAAGCTAGGCTTAAGAGGGAAGAG
ATTTCTGGTGTTAAGTTGGAGTCTATTGGTACTTACCAGATTCATCA
TCATCATCATCATAAGGATGAGCTTTGATGA 3′.

The protein sequence encoded for by SEQ ID NO: 86 is:

(SEQ ID NO: 87)

MGFVLFSQLPSFLLVSTLLLFLVISHSCRADQICIGYHANNSTEQVDTIM
EKNVTVTHAQDILEKTHNGKLCDLDGVKPLILRDCSVAGWLLGNPMCDEF
LNVPEWSYIVEKINPANDLCYPGNFNDYEELKHLLSRINHFEKIQIIPKS
SWSDHEASSGVSSACPYQGRSSFFRNVVWLIKKNNAYPTIKRSYNNTNQ
EDLLVLWGIHHPNDAAEQTRLYQNPTTYISVGTSTLNQRLVPKIATRSKV
NGQSGRMEFFWTILKPNDAINFESNGNFIAPENAYKIVKKGDSTIMKSE
LEYGNCNTKCQTPIGAINSSMPFHNIHPLTIGECPKYVKSNRLVLATGLR
NSPQGERRRKKRGLFGAIAGFIEGGWQGMVDGWYGYHHSNEQGSGY
AADKESTQKAIDGVTNKVNSIIDKMNTQFEAVGREFNNLERRIENLNKK
MEDGFLDVWTYNAELLVLMENERTLDFHDSNVKNLYDKVRLQLRDN
AKELGNGCFEFYHRCDNECMESVRNGTYDYPQYSEEARLKREEISG
VKLESIGTYQIHHHHHHKDEL 3′.

Launch vectors were then introduced into Agrobacterium and vacuum infiltrated into Nicotiana benthamiana. HA antigens were allowed to express and accumulate in the plant biomass for 5-7 days prior to harvesting.

Recombinant HA antigens were purified from the plant biomass. Briefly, plant cells were lysed in 50 mM NaPi, pH 8.0, 0.5 M NaCl, and 20 mM imidazole. Triton was added to a final concentration of 0.5% and incubated for 20 minutes at 4° C. Extracts were spun for 30 minutes at 78,000×g at 4° C. or for 40 minutes at 4° C. at 48,000×g. Supernatant was filtered through Miracloth prior to loading on Ni-NTA columns. In some instances, an optional additional clarification was performed, utilizing TFF (tangential flow filtration) microfiltration step (0.1 μm-0.2 μm pore size). Cleared extracts were loaded onto a Ni-NTA column (pre-equilibrated with lysis buffer), and the columns were washed thoroughly with Buffer A (50 mM NaPi, pH 7.5, 0.5 M NaCl, 20 mM imidazole, and 0.5% Triton) followed by a wash with Buffer Al (same as Buffer A without the Triton). Proteins were eluted with imidazole. Eluted proteins were optionally further purified using anion exchange chromatography (Q Column) or ultrafiltration.

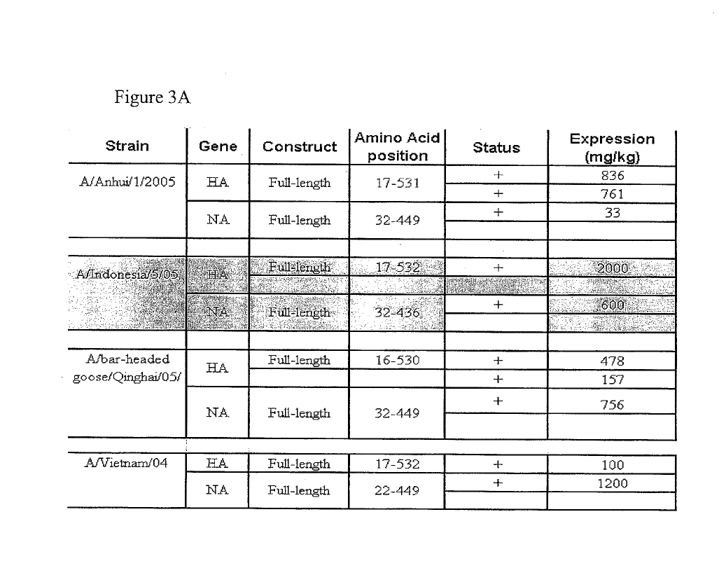

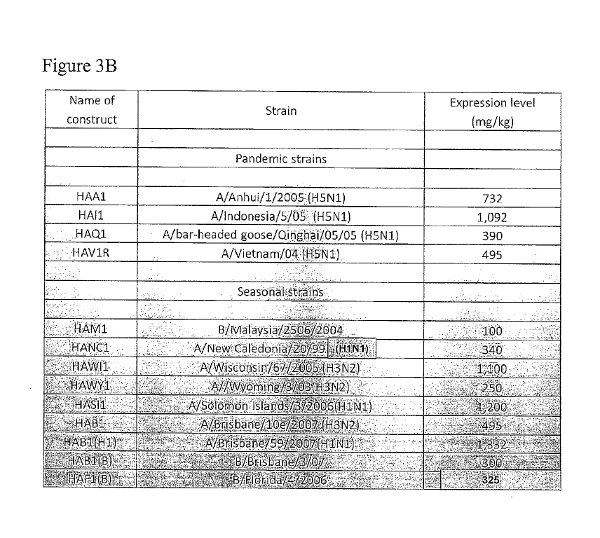

FIG. 3A presents exemplary expression data for four different constructs expressing full-length H5HA from four different strains (i.e., H5 antigens from A/Anhui/1/2005, “H5HA-A” or “HAA”; A/Indonesia/5/05, “H5HA-I” or “HAI”; A/Bar-headed goose/Qinghai/1A/2005, “H5HA-Q” or “HAQ”; and A/Vietnam/04, “H5HA-V” or “HAV”).

The nucleotide sequence of HA from A/Indonesia/5/05 (ISDN125873) that was cloned into launch vectors is:

(SEQ ID NO: 88)

5′ATGGGTTTCGTGCTTTTCTCTCAGCTTCCTTCTTTCCTTCTTGTGT
CTACTCTTCTTCTTTTCCTTGTGATTTCTCATTCTTGCAGGGCTGAT
CAAATCTGCATTGGTTACCATGCTAACAATTCTACTGAGCAAGTGGA
TACAATTATGGAAAAGAATGTGACTGTGACTCATGCTCAGGATATTCT
TGAAAAGACTCATAATGGAAAGTTGTGCGATCTTGATGGTGTTAAGCC
TCTTATTCTTAGGGACTGCAGTGTTGCTGGTTGGTTGCTTGGAAATCC
TATGTGCGATGAGTTCATTAATGTGCCTGAGTGGTCTTACATTGTGGAG
AAGGCTAATCCTACTAATGATCTTTGCTACCCTGGTTCTTTCAATGATTA
CGAAGAGCTTAAACATCTTCTTTCTAGGATTAATCATTTCGAGAAGATT
CAGATTATTCCTAAGTCATCTTGGAGTGATCATGAGGCTTCATCTGGTG
TTTCTTCAGCTTGCCCTTACCTTGGATCTCCTTCTTTCTTCAGGAATGTT
GTTTGGCTTATTAAGAAGAATTCTACTTACCCTACTATTAAGAAGTCTTA
CAACAATACTAATCAGGAGGATCTTCTTGTTCTTTGGGGTATTCATCATC
CTAATGATGCTGCTGAACAGACTAGGCTTTACCAGAATCCTACTACTTA
CATTTCTATTGGTACTTCTACTCTTAATCAGAGGCTTGTGCCTAAGATTG
CTACTAGGTCTAAAGTGAATGGTCAGTCTGGAAGGATGGAATTCTTCTG
GACTATTCTTAAGCCAAATGATGCTATTAATTTCGAGTCTAATGGAAATT
TCATTGCTCCTGAGTACGCTTACAAGATTGTGAAGAAAGGTGATAGTGC
TATTATGAAGTCTGAGCTTGAGTACGGTAATTGCAATACTAAGTGCCAG
ACTCCTATGGGTGCTATTAATTCTTCTATGCCTTTCCATAATATTCATCC
TCTTACTATTGGTGAGTGCCCTAAGTACGTGAAGTCTAATAGGCTTGTGC
TTGCTACTGGTCTTAGGAATTCTCCTCAGAGAGAGTCTAGAAGAAAGAA
GAGGGGACTTTTCGGAGCTATTGCTGGTTTTATTGAGGGAGGATGGCAG
GGAATGGTTGATGGTTGGTATGGTTACCATCATTCTAATGAGCAGGGTTC
TGGTTATGCTGCTGATAAGGAATCTACTCAGAAAGCTATTGATGGTGTTA
CTAATAAGGTGAACTCTATTATTGATAAGATGAATACTCAGTTCGAGGCT
GTTGGTAGAGAGTTCAACAATCTTGAGAGAAGGATTGAGAATCTTAATAA
GAAAATGGAAGATGGTTTCCTTGATGTGTGGACTTACAATGCTGAGTTGC
TTGTGCTTATGGAAAATGAGAGGACTCTTGATTTCCATGATTCTAATGTG
AAGAATCTTTACGATAAAGTGAGACTTCAGCTTAGGGATAATGCTAAAG
AACTTGGAAATGGTTGCTTCGAGTTCTACCATAAGTGCGATAATGAGTG
CATGGAATCTATTAGGAATGGTACTTACAATTACCCTCAGTACTCTGAA
GAAGCTAGGCTTAAGAGGGAAGAGATTTCTGGTGTTAAGTTGGAGTCT
ATTGGAACTTACCAGATTCATCATCATCATCATCATAAGGATGAGC
TTTGATGA 3′.

The protein sequence encoded for by SEQ ID NO: 88 is:

(SEQ ID NO: 89)

5′MGFVLFSQLPSFLLVSTLLLFLVISHSCRADQICIGYHANNSTEQVD
TIMEKNVTVTHAQDILEKTHNGKLCDLDGVKPLILRDCSVAGWLLGNPM
CDEFINVPEWSYIVEKANPTNDLCYPGSFNDYEELKHLLSRINHFEKIQI
IPKSSWSDHEASSGVSSACPYLGSPSFFRNVVWLIKKNSTYPTIKKSY
NNTNQEDLLVLWGIHHPNDAAEQTRLYQNPTTYISIGTSTLNQRLVPKIA
TRSKVNGQSGRMEFFWTILKPNDAINFESNGNFIAPEYAYKIVKKGDSA
IMKSELEYGNCNTKCQTPMGAINSSMPFHNIHPLTIGECPKYVKSNRLV
LATGLRNSPQRESRRKKRGLFGAIAGFIEGGWQGMVDGWYGYHHSNE
QGSGYAADKESTQKAIDGVTNKVNSIIDKMNTQFEAVGREFNNLERRIE
NLNKKMEDGFLDVWTYNAELLVLMENERTLDFHDSNVKNLYDKVRLQ
LRDNAKELGNGCFEFYHKCDNECMESIRNGTYNYPQYSEEARLKREE
ISGVKLESIGTYQIHHHHHHKDEL 3′.

The nucleotide sequence of HA from A/Vietnam/04 that was cloned into launch vectors is:

(SEQ ID NO: 90)

5′ATGGGATTCGTGCTTTTCTCTCAGCTTCCTTCTTTCCTTCTTGTGTC
TACTCTTCTTCTTTTCCTTGTGATTTCTCACTCTTGCAGGGCTGATCAA
ATCTGCATTGGATACCACGCTAACAACTCTACTGAGCAAGTGGATACA
ATTATGGAAAAGAACGTGACTGTTACTCACGCTCAGGATATTCTTGAAA
AGACTCACAACGGAAAGTTGTGCGATCTTGATGGTGTTAAGCCACTTAT
TCTTAGGGATTGCTCTGTTGCTGGATGGCTTCTTGGAAACCCAATGTGTG
ATGAGTTCATTAACGTGCCAGAGTGGTCTTATATTGTGGAGAAGGCTAAC
CCAGTGAACGATCTTTGTTACCCTGGTGATTTCAACGATTACGAAGAGCT
TAAGCACCTTCTTTCTAGGATTAACCACTTCGAGAAGATTCAGATTATTC
CAAAGTCATCTTGGTCATCTCACGAGGCTTCTCTTGGAGTTTCTTCTGCT
TGCCCATACCAGGGAAAGTCATCTTTCTTCAGGAACGTTGTTTGGCTTAT
TAAGAAGAACTCTACTTACCCAACTATTAAGAGGTCTTACAACAACACTA
ACCAGGAAGATTTGCTTGTTCTTTGGGGAATTCACCACCCAAATGATGCT
GCTGAACAGACTAAGTTGTACCAGAACCCAACTACTTACATTTCTGTGGG
AACTTCTACTCTTAACCAGAGGCTTGTGCCAAGAATTGCTACTAGGTCTA
AGGTGAACGGACAATCTGGAAGGATGGAATTCTTCTGGACTATTCTTAAG
CCAAACGATGCTATTAACTTCGAGTCTAACGGAAACTTCATTGCTCCAGA
GTACGCTTACAAGATTGTGAAGAAGGGTGATAGTACTATTATGAAGTCTG
AGCTTGAGTACGGAAACTGCAACACTAAGTGCCAAACTCCAATGGGAGCT
ATTAACTCTTCTATGCCATTCCACAACATTCACCCACTTACTATTGGAGA
GTGCCCAAAGTACGTGAAGTCTAACAGGCTTGTGCTTGCTACTGGACTTA
GGAATTCTCCACAGAGAGAAAGAAGAAGAAAGAAAAGGGGACTTTTCGG
AGCTATTGCTGGATTCATTGAGGGAGGATGGCAGGGAATGGTTGATGGAT
GGTATGGATACCATCACTCTAATGAGCAGGGATCTGGATATGCTGCTGAC
AAAGAATCTACTCAGAAAGCTATTGACGGTGTTACTAACAAGGTGAACTC
TATTATTGATAAGATGAACACTCAGTTCGAAGCTGTTGGAAGAGAGTTCA
ACAACCTTGAGAGAAGGATTGAGAACCTTAACAAGAAAATGGAAGATGGA
TTCCTTGATGTGTGGACTTACAACGCTGAGTTGCTTGTGCTTATGGAAA
ACGAGAGGACTCTTGATTTCCACGATTCTAACGTGAAGAACCTTTACGA
CAAAGTGAGGCTTCAGCTTAGGGATAACGCTAAAGAGCTTGGAAACGG
TTGCTTCGAGTTCTACCACAAGTGCGATAACGAGTGCATGGAATCTGTT
AGGAACGGAACTTACGATTACCCACAGTACTCTGAAGAAGCTAGGCTT
AAGAGGGAAGAGATTTCTGGTGTTAAGTTGGAGTCTATTGGTATCTACCA
GATTCATCACCATCACCACCACAAGGATGAGCTTTGATGA 3′.

The protein sequence encoded for by SEQ ID NO: 90 is:

(SEQ ID NO: 91)

5′MGFVLFSQLPSFLLVSTLLLFLVISHSCRADQICIGYHANNSTEQVD
TIMEKNVTVTHAQDILEKTHNGKLCDLDGVKPLILRDCSVAGWLLGNPM
CDEFINVPEWSYIVEKANPVNDLCYPGDFNDYEELKHLLSRINHFEKIQI
IPKSSWSSHEASLGVSSACPYQGKSSFFRNVVWLIKKNSTYPTIKRSYN
NTNQEDLLVLWGIHHPNDAAEQTKLYQNPTTYISVGTSTLNQRLVPRIAT
RSKVNGQSGRMEFFWTILKPNDAINFESNGNFIAPEYAYKIVKKGDSTIM
KSELEYGNCNTKCQTPMGAINSSMPFHNIHPLTIGECPKYVKSNRLVLA
TGLRNSPQRERRRKKRGLFGAIAGFIEGGWQGMVDGWYGYHHSNEQG
SGYAADKESTQKAIDGVTNKVNSIIDKMNTQFEAVGREFNNLERRIENLN
KKMEDGFLDVWTYNAELLVLMENERTLDFHDSNVKNLYDKVRLQLRD
NAKELGNGCFEFYHKCDNECMESVRNGTYDYPQYSEEARLKREEISG
VKLESIGIYQIHHHHHHKDEL 3′.

The nucleotide sequence of HA from B/Malaysia/2506/2004-like that was cloned into launch vectors is:

(SEQ ID NO: 92)

5′ATGGGATTCGTGCTTTTCTCTCAGCTTCCTTCTTTCCTTCTTGTGTC
TACTCTTCTTCTTTTCCTTGTGATTTCTCACTCTTGCAGGGCTATCTG
CACTGGAATTACTTCATCTAACTCTCCACACGTGGTTAAGACTGCTACT
CAGGGTGAAGTTAACGTGACTGGTGTTATTCCACTTACTACTACTCCAA
CTAAGTCTCACTTCGCTAACCTTAAGGGAACTGAGACTAGAGGAAAGT
TGTGCCCAAAGTGCCTTAACTGCACTGATCTTGATGTTGCTCTTGGAAG
GCCAAAGTGCACTGGAAACATTCCATCTGCTAGGGTGTCAATTCTTCA
CGAAGTGAGGCCAGTTACTTCTGGATGCTTCCCAATTATGCACGATAG
GACTAAGATTAGGCAGCTTCCAAACCTTCTTAGGGGATACGAGCACAT
TAGGCTTTCTACTCACAACGTGATTAACGCTGAGAATGCTCCAGGTGG
ACCATACAAGATTGGAACTTCAGGATCTTGCCCAAACGTGACTAACGG
AAACGGATTCTTCGCTACTATGGCTTGGGCTGTGCCAAAGAACGATAA
CAACAAGACTGCTACAAACTCTCTTACTATTGAGGTTCCTTACATCTGT
ACTGAGGGTGAAGATCAGATTACTGTGTGGGGATTCCACTCTGATAAC
GAGACTCAGATGGCTAAGTTGTACGGTGATTCTAAGCCACAGAAGTTC
ACTTCATCTGCTAACGGTGTTACTACTCACTACGTGTCTCAGATTGGAG
GATTCCCAAACCAGACTGAGGATGGTGGACTTCCACAATCTGGAAGG
ATTGTGGTGGATTACATGGTTCAGAAGTCTGGAAAGACTGGAACTATTA
CTTACCAGAGGGGTATTCTTCTTCCACAGAAAGTGTGGTGTGCTTCTGG
AAGGTCTAAAGTGATTAAGGGATCTCTTCCACTTATTGGAGAGGCTGAT
TGCCTTCATGAGAAGTACGGTGGACTTAACAAGTCTAAGCCTTACTAC
ACTGGTGAACACGCTAAGGCTATTGGAAACTGCCCAATTTGGGTTAAG
ACTCCACTTAAGTTGGCTAACGGAACTAAGTATAGGCCACCTGCTAAG
TTGCTTAAAGAGAGGGGATTCTTCGGAGCTATTGCTGGATTTCTTGAGG
GAGGATGGGAGGGAATGATTGCTGGATGGCACGGATATACTTCTCATG
GTGCTCACGGTGTTGCTGTTGCTGCTGATCTTAAGTCTACTCAAGAGGC
TATTAACAAGATTACTAAGAACCTTAACTCTCTTTCTGAGCTTGAGGTGA
AGAACCTTCAGAGACTTTCTGGTGCTATGGATGAGCTTCACAACGAGA
TTCTTGAGCTTGATGAGAAAGTGGATGATCTTAGGGCTGATACAATTTC
TTCTCAGATTGAGCTTGCTGTGCTTCTTTCTAACGAGGGAATTATTAACT
CTGAGGATGAGCACCTTCTTGCTCTTGAGAGGAAGTTGAAGAAGATGC
TTGGACCATCTGCTGTTGAGATTGGAAACGGTTGCTTCGAGACTAAGC
ACAAGTGCAACCAGACTTGCCTTGATAGAATTGCTGCTGGAACTTTCG
ATGCTGGTGAGTTCTCTCTTCCAACTTTCGATTCTCTTAACATTACTGC
TGCTTCTCTTAACGATGATGGACTTGATAACCACACTCATCACCATC
ACCACCACAAGGATGAGCTTTGA 3′.

The protein sequence encoded for by SEQ ID NO: 92 is:

(SEQ ID NO: 93)

5′MGFVLFSQLPSFLLVSTLLLFLVISHSCRAICTGITSSNSPHVVKTAT
QGEVNVTGVIPLTTTPTKSHFANLKGTETRGKLCPKCLNCTDLDVALGRP
KCTGNIPSARVSILHEVRPVTSGCFPIMHDRTKIRQLPNLLRGYEHIRLS
THNVINAENAPGGPYKIGTSGSCPNVTNGNGFFATMAWAVPKNDNNKTA
TNSLTIEVPYICTEGEDQITVWGFHSDNETQMAKLYGDSKPQKFTSSANG
VTTHYVSQIGGFPNQTEDGGLPQSGRIVVDYMVQKSGKTGTITYQRGILL
PQKVWCASGRSKVIKGSLPLIGEADCLHEKYGGLNKSKPYYTGEHAKAIG
NCPIWVKTPLKLANGTKYRPPAKLLKERGFFGAIAGFLEGGWEGMIAGW
HGYTSHGAHGVAVAADLKSTQEAINKITKNLNSLSELEVKNLQRLSGAMD
ELHNEILELDEKVDDLRADTISSQIELAVLLSNEGIINSEDEHLLALERK
LKKMLGPSAVEIGNGCFETKHKCNQTCLDRIAAGTFDAGEFSLPTFDSLN
ITAASLNDDGLDNHTHHHHHHKDEL 3′.

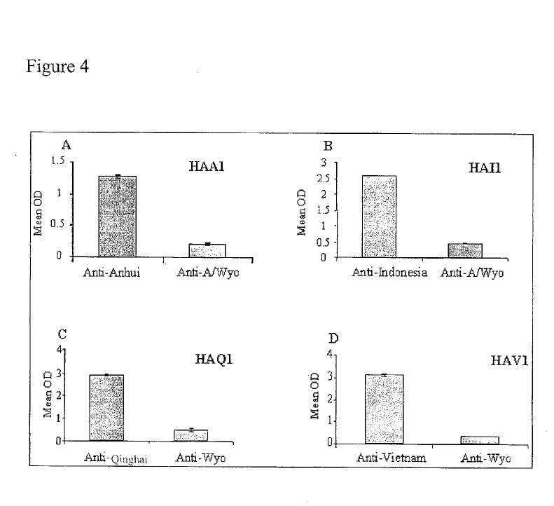

FIG. 3B presents exemplary expression data for several pandemic and seasonal influenza strains. FIG. 4 demonstrates the antigenicity of the plant-produced antigens shown in FIG. 3A using an ELISA assay. This assay was performed by coating 96 well plates with 1 μg/ml of each H5HA protein. Antigens were then detected using a 1:6000 dilution of either anti-A/Anhui/01/05 ferret sera, anti-A/Indonesia/05/2005 ferret sera, anti-A/Vietnam/1194/04 HA sheep anti-sera, or anti-A/Wyoming/03/2003 HA sheep anti-sera. All plant-produced H5HAs showed specific reactivity with anti-serum raised against homologous H5HA, but not against anti-serum generated against A/Wyoming/03/03 an H3 virus. These results suggest that the plant-produced antigens are properly folded and display authentic antigenicity.

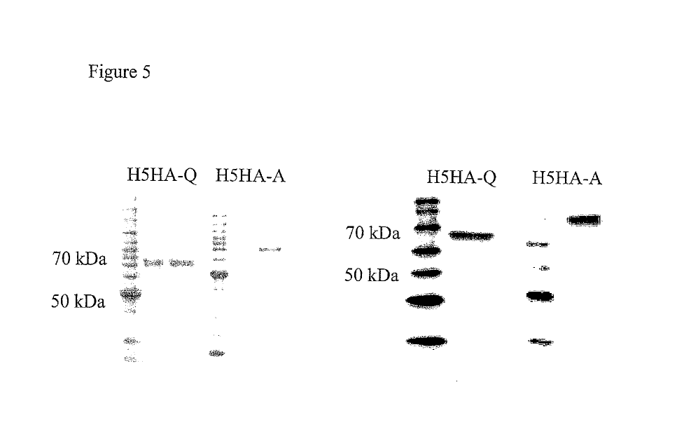

FIG. 5 presents Coomassie gels and western blots of two H5HA antigens (i.e., H5HA-A and H5HA-Q) expressed in and purified from plants. In particular, seven days post infiltration with launch vectors, H5HA-Q and H5HA-A accumulated to 478 mg/kg and 836 mg/kg of fresh leaf biomass, respectively. Proteins were extracted and characterized by Western blot assay using sheep sera raised against HA from A/Vietnam/1194/2004.

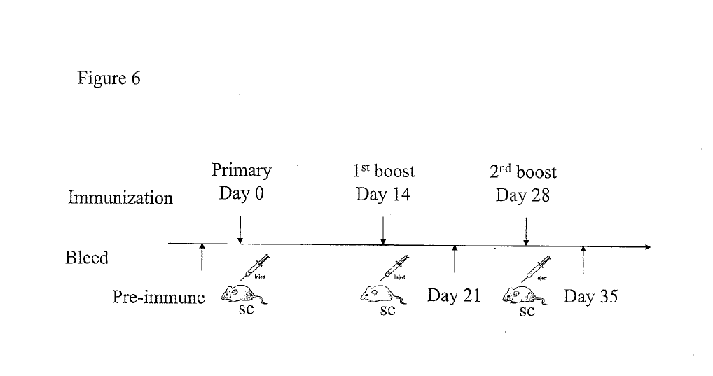

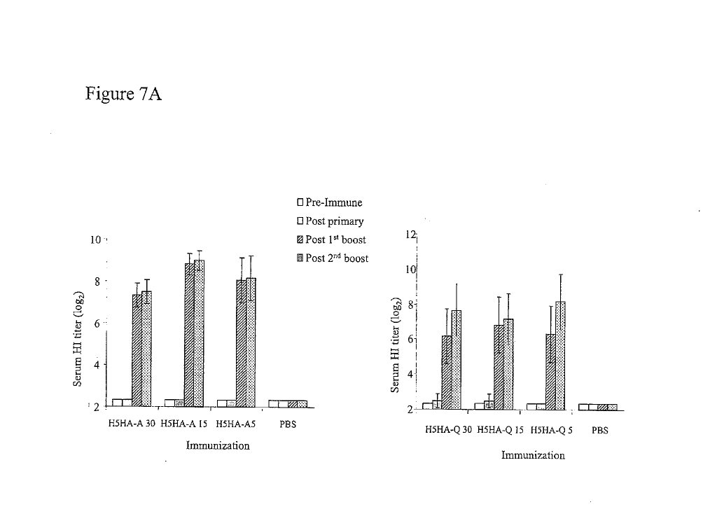

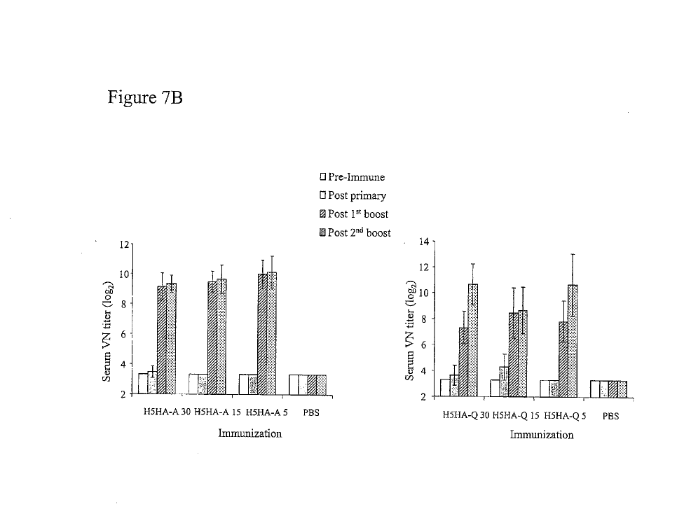

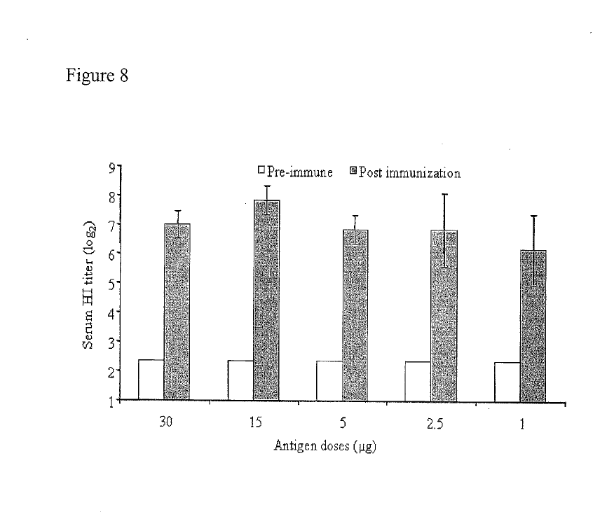

Groups of 8 week old female Balb/c mice were immunized subcutaneously with H5HA-Q or H5HA-A in the presence of 10 μg Quil A (FIG. 6). Immunizations were administered at days 0, 14, and 28. On days 21 and 35, serum was isolated from the mice and subjected to hemagglutination inhibition (HI) and virus neutralization (VN) assays (carried out essentially as described below in Example 2). As shown in FIG. 7A, serum from mice immunized with A/Anhui/01/05 or A/Bar-headedgoose/Qinghai/1A/05 HA produced in plants demonstrated significant hemagglutination inhibition activity, even when mice were immunized with doses of antigen as low as 5 μg. As shown in FIG. 7B, serum from mice immunized with A/Anhui/01/05 or A/Bar-headedgoose/Qinghai/1A/05 HA produced in plants demonstrated significant virus neutralization activity, even when mice were immunized with doses of antigen as low as 5 μg. Mice were also immunized with antigen doses as low as 2.5 μg and 1 μg of HAA. FIG. 8 demonstrates that plant-produced HA elicits high titers of HI with doses as low as 1 μg.

### Example 2

### Plant-Expressed H3HA as a Seasonal Influenza Vaccine Candidate

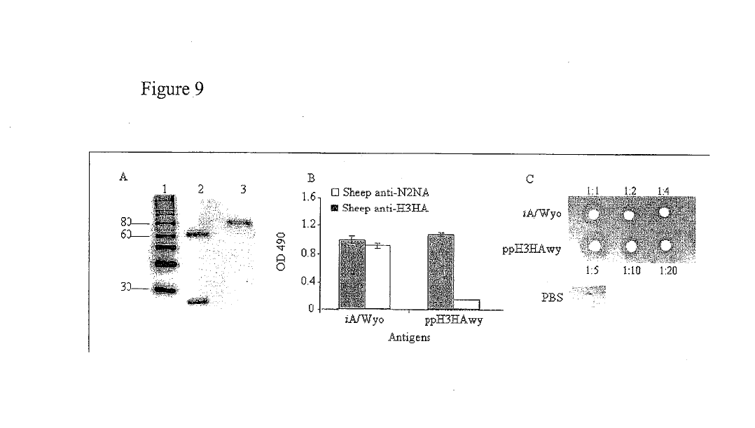

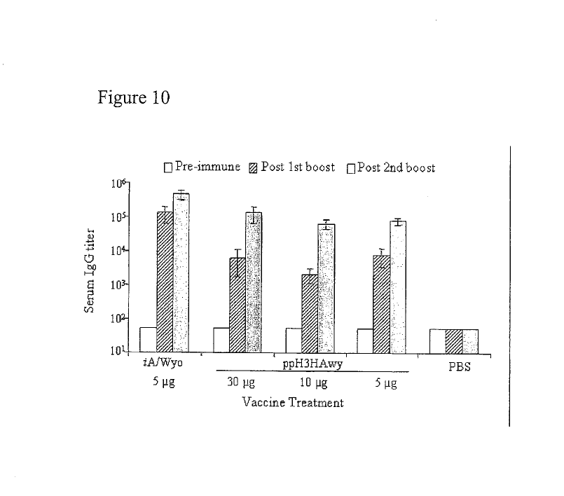

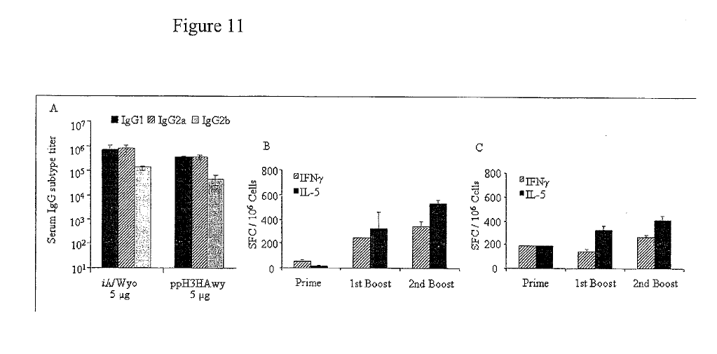

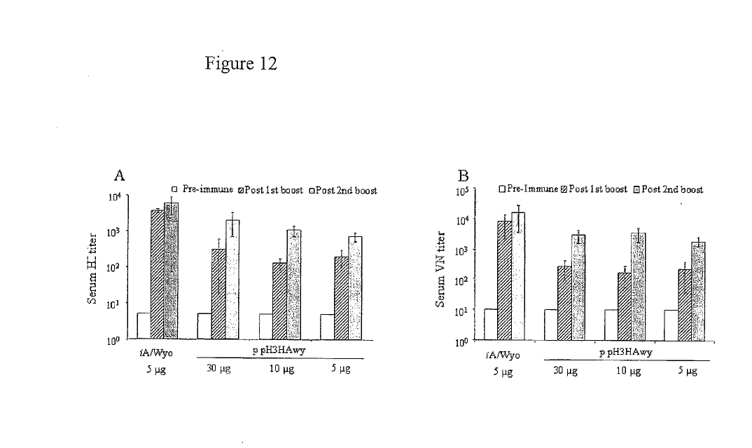

Full-length hemagglutinin (HA) protein was engineered, expressed, and purified from the A/Wyoming/03/03 (H3N2) strain of influenza in plants (FIG. 9). The antigenicity of plant-produced HA was confirmed by ELISA and single-radial immunodiffusion (SRID) assays (FIG. 9). Immunization of mice with plant-produced HA resulted in HA-specific humoral (IgG1, IgG2a, and IgG2b) and cellular (IFNγ and IL-5) immune responses (FIGS. 10 and 11). In addition, significant serum hemagglutination inhibition (HI) and virus neutralizing (VN) antibody titers were obtained with an antigen dose as low as 5 μg (FIG. 12). These results demonstrate that plant-produced HA protein is antigenic and can induce immune responses in mice that correlate with protection.

### Materials and Methods

Cloning, Expression, and Purification of Influenza HA

HA sequences encoding amino acids 17-532 of the A/Wyoming/03/03 strain of influenza virus were optimized for expression in plants and synthesized by GENEART AG (Regensburg, Germany). During synthesis sequences encoding the endoplasmic reticulum retention signal (KDEL) and the poly-histidine affinity purification tag (6×His) were included at the C-terminus. The resulting NA sequence, H3HAwy, was then cloned into launch vector pBID4 (Musiychuk et al., 2007, Influenza and Other Respiratory Viruses, 1:1; incorporated herein by reference) to obtain pBID4-H3HAwy. pBID4-H3HAwy was then introduced into Agrobacterium tumefaciens strain GV3101 by electroporation. Expression of H3HAwy in greenhouse-grown 6 week old Nicotiana benthamiana leaves was achieved by agroinfiltration with GV3101 harboring pBID4-H3HAwy. Tissue was collected 7 days after agroinfiltration and plant-produced H3HAwy (ppH3HAwy) was purified by immobilized metal ion affinity chromatography followed by anion exchange chromatography.

The nucleotide sequence of HA from A/Wyoming/03/03 (AAT08000) that was cloned into launch vectors is:

(SEQ ID NO: 94)

5′ATGGGATTCGTGCTTTTCTCTCAGCTTCCTTCTTTCCTTCTTGTGT
CTACTCTTCTTCTTTTCCTTGTGATTTCTCACTCTTGCCGTGCTCAAAA
GTTGCCAGGAAACGATAACTCTACTGCTACTCTTTGCCTTGGACAT
CACGCTGTTCCAAACGGAACTATTGTGAAAACTATTACTAACGATCAG
ATTGAGGTGACAAACGCTACTGAGCTTGTTCAGTCATCTTCTACTGGA
GGAATTTGCGATTCTCCACACCAGATTCTTGATGGAGAGAACTGCACT
CTTATTGATGCTCTTCTTGGAGATCCACAGTGCGATGGATTCCAGAAC
AAGAAGTGGGATCTTTTCGTGGAAAGGTCTAAGGCTTACTCTAACTGC
TACCCATACGATGTTCCAGATTACGCTTCTCTTAGGAGTCTTGTGGCTT
CTTCTGGAACTCTTGAGTTCAACAACGAGTCTTTCAACTGGGCTGGAG
TTACTCAGAACGGAACTTCTTCTGCTTGTAAGAGGAGGTCTAACAAGT
CTTTCTTCTCTAGGCTTAACTGGCTTACTCACCTTAAGTACAAGTACC
CAGCTCTTAACGTGACTATGCCAAACAACGAGAAGTTCGATAAGTTG
TACATTTGGGGAGTTCACCACCCAGTTACTGATTCTGATCAGATTTCT
CTTTACGCTCAGGCTTCTGGAAGGATTACTGTGTCTACTAAGAGGTCT
CAGCAGACTGTGATTCCAAACATTGGATACCGTCCAAGAGTGAGGG
ATATTTCTTCTAGGATTTCTATCTACTGGACTATTGTGAAGCCAGGAG
ATATTCTTCTTATTAACTCTACTGGAAACCTTATTGCTCCAAGGGGATA
CTTCAAGATTAGGAGTGGAAAGTCATCTATTATGAGGAGTGATGCTCC
AATTGGAAAGTGCAACTCTGAGTGCATTACTCCAAACGGATCTATTCC
AAACGATAAGCCATTCCAGAACGTGAACAGGATTACTTATGGAGCTTG
CCCAAGATACGTGAAGCAGAACACTCTTAAGTTGGCTACTGGAATGA
GGAATGTGCCAGAGAAGCAGACTAGGGGAATTTTCGGAGCTATTGCT
GGATTCATTGAGAATGGATGGGAGGGAATGGTTGATGGATGGTACGG
ATTCAGGCACCAGAATTCAGAGGGAACTGGACAAGCTGCTGATCTTA
AGTCTACTCAGGCTGCTATTAACCAGATTAACGGAAAGTTGAACAGG
CTTATTGGAAAGACTAACGAGAAGTTCCACCAGATTGAGAAGGAGTT
CTCTGAGGTTGAGGGAAGGATTCAGGATCTTGAGAAGTACGTGGAGG
ATACAAAGATTGATCTTTGGTCTTACAACGCTGAGCTTCTTGTTGCTCT
TGAGAACCAGCACACTATTGATTTGACTGATTCTGAGATGAACAAGTT
GTTCGAGAGGACTAAGAAGCAGCTTAGGGAGAACGCTGAGGATATGG
GAAATGGATGCTTCAAAATCTACCACAAGTGCGATAACGCTTGCATTG
AGTCTATTAGGAACGGAACTTACGATCACGATGTGTACCGTGATGAGG
CTCTTAACAACAGGTTCCAGATTAAGGGAGTGGAGCTTAAGTCTGGAT
ACAAGGATTGGATTCTTCATCATCACCACCACCACAAGGATGAGC
TTTGATGA.

The protein sequence encoded for by SEQ ID NO: 94 is:

(SEQ ID NO: 95)

5′MGFVLFSQLPSFLLVSTLLLFLVISHSCRAQKLPGNDNSTATLCLGHH
AVPNGTIVKTITNDQIEVTNATELVQSSSTGGICDSPHQILDGENCTLID
ALLGDPQCDGFQNKKWDLFVERSKAYSNCYPYDVPDYASLRSLVASSGT
LEFNNESFNWAGVTQNGTSSACKRRSNKSFFSRLNWLTHLKYKYPALNV
TMPNNEKFDKLYIWGVHHPVTDSDQISLYAQASGRITVSTKRSQQTVIPN
IGYRPRVRDISSRISIYWTIVKPGDILLINSTGNLIAPRGYFKIRSGKSS
IMRSDAPIGKCNSECITPNGSIPNDKPFQNVNRITYGACPRYVKQNTLK
LATGMRNVPEKQTRGIFGAIAGFIENGWEGMVDGWYGFRHQNSEGTG
QAADLKSTQAAINQINGKLNRLIGKTNEKFHQIEKEFSEVEGRIQDLEKY
VEDTKIDLWSYNAELLVALENQHTIDLTDSEMNKLFERTKKQLRENAE
DMGNGCFKIYHKCDNACIESIRNGT
YDHDVYRDEALNNRFQIKGVELKSGYKDWILHHHHHHKDEL.

Western Blot Analysis and ELISA of Purified ppH3HAwy

To characterize plant-produced HA, ppH3HAwy, purified from infiltrated N benthamiana leaves, was separated on 10% SDS-polyacrylamide gel, transferred onto polyvinylidene fluoride membrane (Millipore, Billerica, Mass.) and blocked with 0.5% I-block (Applied Biosystems, Foster City, Calif.). The membrane was then incubated with sheep anti-serum raised against HA (NIBSC, code number 03/212) followed by horseradish peroxidase (HRP)-conjugated rabbit anti-sheep antibody (Bethyl Laboratory Inc., Montgomery, Tex.). Proteins reacting with anti-HA antibody were visualized using SuperSignal West Pico Chemiluminescent Substrate (Pierce, Rockford, Ill.). The results were documented using GeneSnap software on the GeneGnome (Syngene Bioimaging, Frederick, Md.). Egg-produced formalin-inactivated A/Wyoming/03/03 virus (iA/Wyo, NIBSC, Hertforshire UK, code number 03/220) was used as positive control. For ELISA, 96-well MaxiSorp plates (NUNC, Rochester, N.Y.) were coated with 1 μg/ml of purified ppH3HAwy or with iA/Wyo. Plates were incubated with 1:1600 dilution of sheep anti-serum raised against HA (NIBSC, code number 03/212) or NA (NIBSC, code number 04/258) from A/Wyoming/03/03 virus and detected using rabbit anti-sheep IgG-HRP antibody (Bethyl Laboratory Inc.).

Single-Radial-Immunodiffusion Assay (SRID)

The concentration of ppH3HAwy was determined using the SRID assay as described by Schild at al. (1975, Bull. World HealthOrg., 52:223-31; incorporated herein by reference) with slight modification. Sheep anti-serum raised against purified A/Wyoming/03/03 HA and iA/Wyo (containing 50 μg/ml of HA) was used as reference reagents. ppH3HAwy and iA/Wyo were treated with 1% (w/v) of Zwittergent 3-14 (Calbiochem-Behring, La Jolla, USA), serially diluted, loaded into wells in a pre-made 1% agarose gel containing the reference sheep anti-HA serum, and allowed to diffuse for 48 hours. The agarose gel was incubated in PBS for 24 hours at room temperature to remove unbound antigen and serum components. The gel was then stained with Coomassie blue (Pierce, Rockford, Ill.), the diameter of precipitation rings was measured, and the antigen concentration determined.

Immunization of Mice with ppH3HAwy

Groups of eight-week old Balb/c mice, six mice per group, were immunized with ppH3HAwy subcutaneously at 2-week intervals on days 0, 14, 28. Three different antigen doses were tested: 30 μg, 10 μg, and 5 μg of ppH3HAwy/dose. Animals in control groups received either iA/Wyo (−5 μg/dose of HA) or PBS. All immunizations were performed with the addition of 10 μg of Quil A (Accurate Chemical, Westbury, N.Y.). Serum samples were collected prior to each immunization and two weeks after the third dose.

Characterization of Immune Responses

Serum Antibody Responses

HA-specific serum antibody responses were measured by ELISA using 96-well MaxiSorp plates (NUNC, Rochester, N.Y.) coated with 1.0 μg/ml of iA/Wyo. Samples of sera were tested in series of four-fold dilutions and antigen-specific antibodies were detected using HRP-conjugated goat anti-mouse IgG (Jackson Immunoresearch Laboratory Inc., West Grove, Pa.) (FIG. 10). Titers of IgG antibody subtypes were determined using goat anti-mouse IgG1, IgG2a, or IgG2b conjugated to HRP as secondary antibodies (Southern Biotechnology Associates Inc., Birmingham, Ala.) (FIG. 11). Reciprocal serum dilutions that gave mean OD values three times greater than those from pre-immune sera at a 1:50 dilution were determined as endpoint titers.

ELISPOT Analysis

The frequency of interferon-γ (IFNγ) and interleukin-5 (IL-5) secreting cells in splenocytes from ppH3HAwy immunized mice were analyzed by ELISPOT as described in Chichester et al. (2006, J. Immunol. Methods, 309:99-107; incorporated herein by reference) with slight modification (FIG. 11). In brief, spleens were collected from iA/Wyo- or ppH3HAwy-immunized mice on day 42 and homogenized into single-cell suspensions. Red blood cells were lysed using ACK buffer. Splenocytes were plated in wells of MultiScreen-IP plates (Millipore, Bedford, Mass.) coated with anti-IFNγ or anti-IL-5 monoclonal antibodies (BD Pharmingen, San Diego, Calif.) and stimulated in vitro with 5 μg/ml of insect cell-produced A/Wyoming/03/03 HA (Protein Sciences, Meriden, Conn.). Plates were incubated for 48 hours at 37° C., after which cells were discarded and biotinylated anti-IFNγ or anti-IL-5 (BD Phanningen) antibodies were added to each well. Spots were visualized using streptavidin-alkaline phosphatase followed by NBT/BCIP (Pierce). The data are expressed as the average number of spot-forming cells (SFC)/10⁶ cells.

Hemagglutination Inhibition (HI) and Virus Neutralization (VN) Assays

Serum samples from immunized mice were treated with receptor-destroying enzyme (RDE; Denka Seiken Co. Ltd., Tokyo, Japan) and an HI assay was carried out with 0.75% turkey erythrocytes, as described previously (Rowe et al., J. Clin. Microbiol., 37:937-43; incorporated herein by reference). The microneutralization assay was carried out as described previously (Rowe et al., J. Clin. Microbiol., 37:937-43) with the following modifications. MDCK cells were plated at 3×10⁴ cells/well in 96 well tissue culture plates, and incubated for 18 hours at 37° C. In parallel, RDE-treated serum samples were serially diluted and mixed with an equal volume of 2×10³ TCID₅₀/ml of A/Wyoming/03/03 influenza virus. Following 1 hour incubation at 37° C., serum-virus mixtures were added to the plated MDCK cells and further incubated for 18 hours. Plates were then washed with PBS and fixed with 80% acetone. The neutralizing endpoint titer of each sample was determined by ELISA as described previously (Rowe et al., J. Clin. Microbiol., 37:937-43; incorporated herein by reference).

Statistical Analysis

Statistical analysis of data was performed using a two-tailed t test with equal variance and significance was considered at a p-value <0.05. Samples without detectable IgG, HI, or VN titers were assigned (detection limit 50, 10, or 20, respectively) a value of 25, 5, or 10 for statistical analysis.

### Results and Discussion

Expression and In Vitro Characterization of ppH3HAwy

In order to identify the peak of target expression and determine the optimal time to harvest biomass, a time course was performed. Seven days post-infiltration, when ppH3HAwy accumulated at ˜200 mg/kg of fresh leaf tissue, was established as the time for harvest. Target protein was then purified and characterized by Western, ELISA, and SRID. FIG. 9A (lane 3) shows a protein band with a size of ˜83 kDa that is specifically recognized by sheep anti-HA serum. ELISA and SRID were performed to further characterize the antigenicity of ppH3HAwy. In an ELISA assay, ppH3HAwy showed specific reactivity with sheep anti-serum raised against homologous HA but not NA (FIG. 9B). As shown in FIG. 9C in the SRID gel, the ppH3HAwy diffusion rings were equivalent in size to those around wells loaded with iA/Wyo, indicating the antigenic activity of ppH3HAwy. Taken together these data demonstrate that the ppH3HAwy is properly folded and displays authentic antigenicity.

In Vivo Characterization of ppH3HAwy

Immunogenicity of the ppH3HAwy was evaluated in mice. Samples of sera collected after second and third dose of antigen were analyzed for target-specific antibody responses. As shown in FIG. 10, high serum IgG titers were observed following the first antigen boost for all doses tested. These titers were further enhanced following a second antigen boost reaching levels comparable to those observed for iA/Wyo. Levels of serum IgG elicited by 30, 10, or 5 μg doses of ppH3HAwy were not significantly different. Due to these findings, further characterization of the immune responses was limited to those animals immunized with the 5 μg dose of antigen. Analysis of serum IgG subtypes specific for A/Wyoming/03/03 revealed that immunization with ppH3HAwy resulted in IgG1, IgG2a, and IgG2b antibody responses (FIG. 11A), suggesting that both Th1 and Th2 responses were stimulated. This was further supported by ELISPOT data showing the production of both IFNγ and IL-5 by splenocytes from ppH3HAwy-immunized mice following in vitro re-stimulation with insect cell-produced homologous HA (FIGS. 11B and 11C). In influenza virus infections IgG1 antibody subtype plays a pivotal role in virus neutralization and protection, while, IgG2a antibody subtype has been associated with virus clearance (Huber et al., 2006, Clin. Vaccine Immunol., 13:981-90; incorporated herein by reference). Therefore, stimulation of both IgG1 and IgG2a could be important for effective influenza vaccine development. Challenge studies will further elucidate the potential contribution of IgG subtypes to protective immunity against influenza infection.

To test the functional efficacy of the antibodies generated by ppH3HAwy, HI and VN assays were performed on serum samples from vaccinated mice. All animals that received 30 μg of ppH3HAwy had serum HI titers above 40 following the first antigen boost (FIG. 12A), whereas, in groups of mice that received 10 μg or 5 μg dose of antigen, five out of six animals had serum HI antibody titers above 40. Following the second antigen boost, all animals in all groups immunized with ppH3HAwy had serum HI antibody titers above 160 (FIG. 12A), and in some animals the titers reached 2560.

To further characterize the immune responses generated by ppH3HAwy, serum VN antibody titers were measured (FIG. 12B). Serum VN titers correlated well with serum HI titers. Following a second boost, all mice immunized with ppH3HAwy had VN titers ≧640 against A/Wyoming/03/03 virus reaching levels similar to that observed in serum from mice immunized with iA/Wyo (FIG. 12B). HI and VN antibody titers remained at this high level when assessed 1 month after second boost. These data demonstrate that ppH3HAwy is immunogenic in mice, inducing HI and VN antibody responses against homologous H3N2 influenza virus at a dose as low as 5 μg. HI titers above 40 are typically regarded as the minimum titer consistent with protective immunity in humans (Hobson et al., 1972, J. Hyg. (Lond.), 70:767-77; incorporated herein by reference). Due to the quality of immune responses generated by ppH3HAwy, in particular high HI and VN titers observed even after the first boost of antigen, the present invention encompasses the recognition that plant-produced antigens may be useful for developing an effective influenza vaccine for use in humans. ppH3HAwy showed authentic antigenicity and induced anti-viral antibody responses in mice when administered with Quil A. Quil A is widely used as an adjuvant in veterinary vaccines and has been shown to enhance cellular as well as humoral immune responses (Katayama and Mine, 2006, J. Agric. Food Chem., 54:3271-6; incorporated herein by reference). In addition, saponin-based adjuvants, such as Quil A, have been proposed for use in humans and are currently being evaluated in clinical trials (Mbawuike et al., 2007, Vaccine, 25:3263-9; and Sabbatini et al., 2007, Clin. Cancer Res., 13:4170-7; both of which are incorporated herein by reference). However, the present invention encompasses the recognition that any adjuvant, such as alum or alhydrogel, could be utilized. Indeed, co-administration of alhydrogel and ppH3HAwy generated low serum IgG and HI titers in the present study.

In summary, the present invention encompasses the recognition that plant-produced HA antigens may be useful for developing influenza vaccines.

### Example 3

### Plant-Produced HA from A/Indonesia/05/05 Protects Ferrets Against Homologous Challenge Infection

This Example demonstrates immunogenicity and protective efficacy of recombinant HA from A/Indonesia/5/2005 produced in Nicotiana benthamiana plants. This plant-produced HA antigen induced serum hemagglutination inhibition (HI) and virus neutralizing (VN) antibody titers in mice. Furthermore, immunization of ferrets with this plant-produced HA provided protection against homologous virus challenge. Thus, the present invention encompasses the recognition that plant-produced HA antigens may be useful for developing influenza vaccines for use in humans.

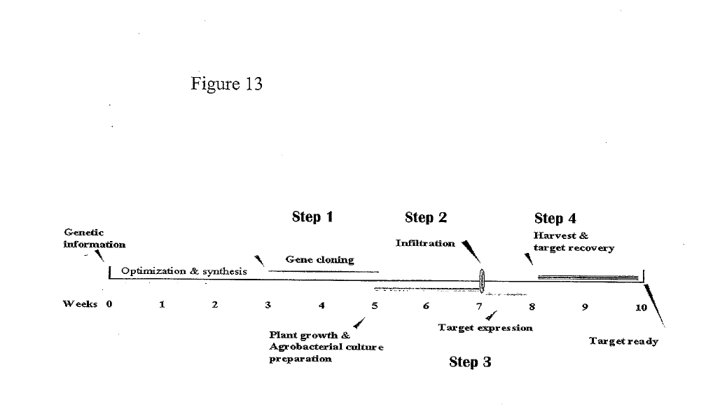

FIG. 13 outlines the general scheme for production of HA antigens in plants. H5HA-I antigen was produced in plants generally as shown in FIG. 2 and in Example 1. H5HA-I antigen was cloned into the “launch vector” system (see, e.g., Musiychuk et al., 2007, Influenza and Other Respiratory Viruses, 1:19-25; and PCT Publication WO 07/095,304; both of which are incorporated herein by reference), specifically vector pGR-D4. Launch vectors were then vacuum infiltrated into Nicotiana benthamiana and HA antigens were allowed to express and accumulate in the plant biomass. Seven days post infiltration with launch vectors, H5HA-I accumulated to ˜800 mg/kg of fresh leaf biomass.

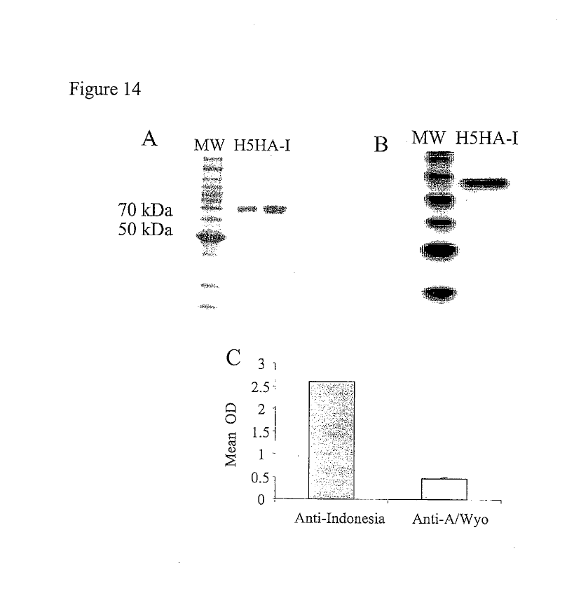

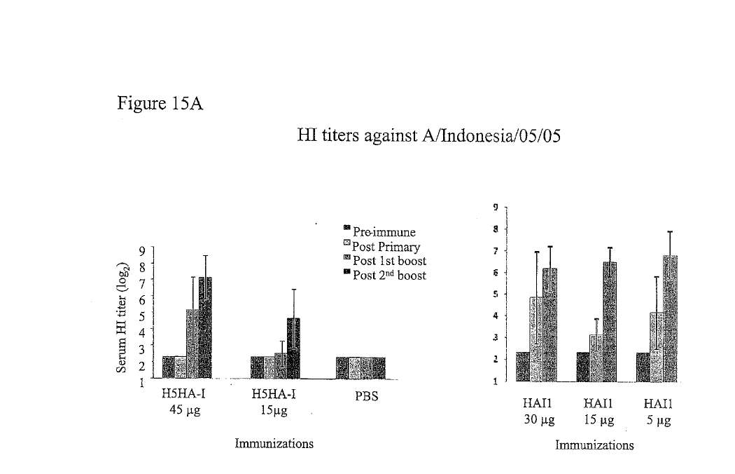

Recombinant HA antigens were purified from the plant biomass, essentially as described in Example 1. Proteins were extracted and characterized by Coomassie staining (FIG. 14A), by western blot assay using mouse anti-His antibody (FIG. 14B), and by ELISA using ferret anti-serum against A/Indonesia/05/05 or sheep anti-serum against A/Wyoming/03/03 (FIG. 14C).

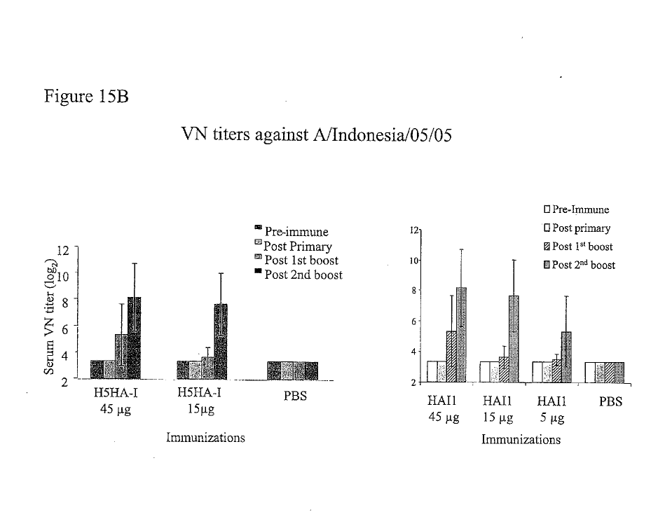

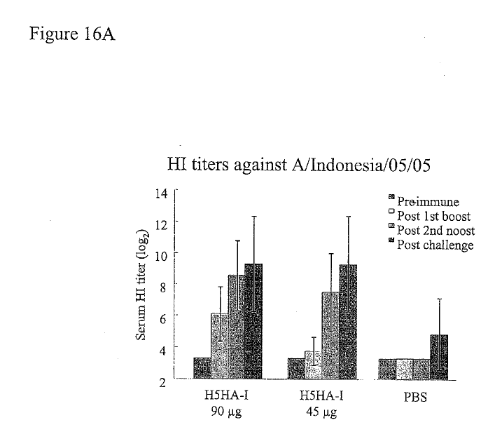

Mice were immunized with 45 μg/dose, 30 μg/dose, 15 μg/dose, or 5 μg/dose of plant-produced H5HA-I subcutaneously at 2 week intervals on day 0, 14 and 28. On days 21 and 35, serum was isolated from the mice and subjected to hemagglutination inhibition (HI) and virus neutralization (VN) assays (carried out essentially as described below in Example 2). As shown in FIG. 15A, serum from mice immunized with A/Indonesia/05/05 HA produced in plants demonstrated significant hemagglutination inhibition activity, even when mice were immunized with doses of antigen as low as 15 μg. As shown in FIG. 15B, serum from mice immunized with A/Indonesia/05/05 HA produced in plants demonstrated virus neutralization activity, even when mice were immunized with doses of antigen as low as 5

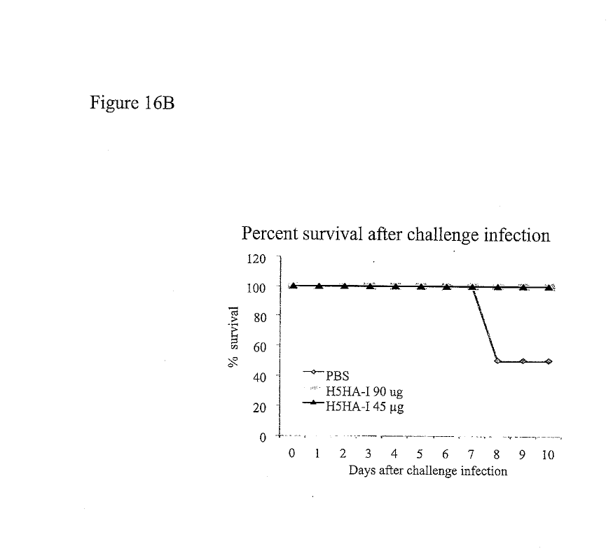

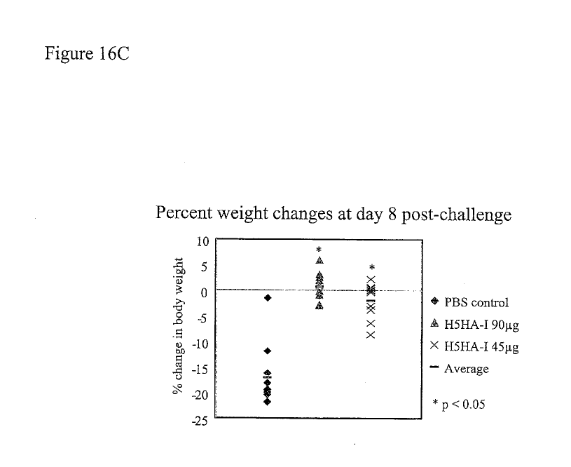

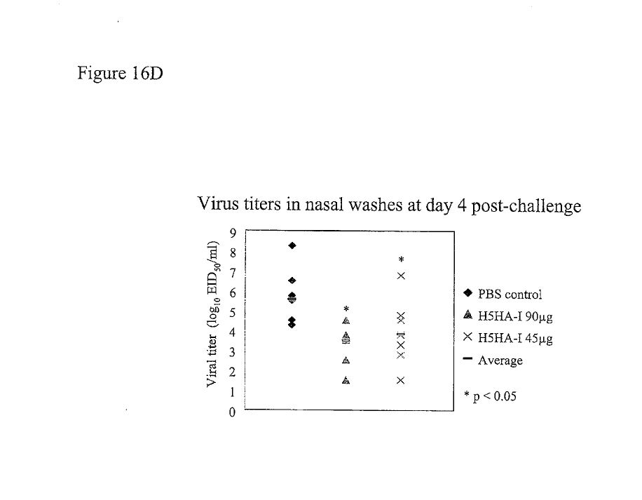

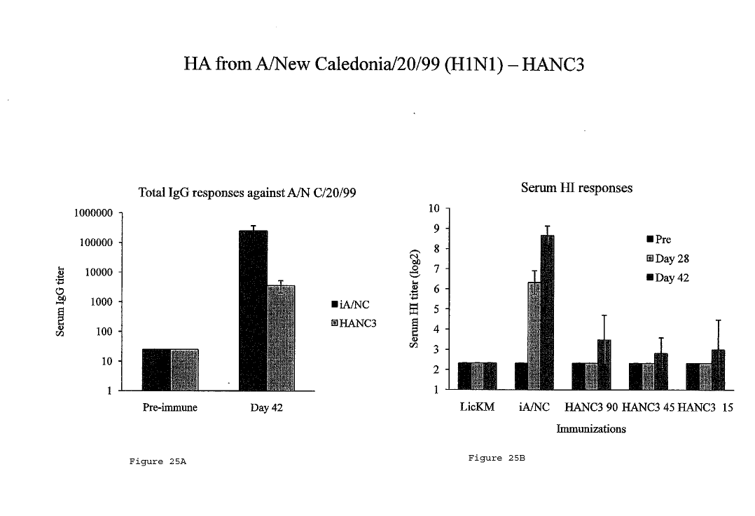

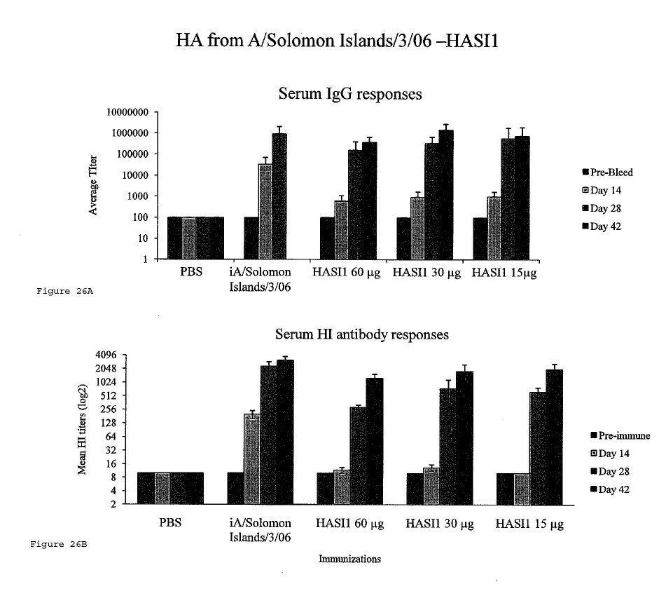

Ferrets were immunized with 90 μg/dose or 45 μg/dose of H5HA-I subcutaneously at 2 week intervals on days 0, 14, and 28. Some ferrets were immunized with a 90 μg/dose of plant-produced H5HA-I plus a 30 μg dose of NAI1 (neuraminidase antigen) subcutaneously (these results are shown in FIGS. 23-26). At ten days after the final immunization, ferrets were challenged intranasally with 10 FLD50 of A/Indonesia/05/05. As shown in FIG. 16A, serum from ferrets immunized with A/Indonesia/05/05 HA produced in plants demonstrated significant hemagglutination inhibition activity. FIG. 16B shows the percent survival of ferrets after challenge. FIG. 16C shows the percent weight change of ferrets at 8 days post-challenge. FIG. 16D shows viral titers in ferret nasal washes at 4 days post challenge.

In conclusion, recombinant HA from A/Indonesia/05/05 (H5HA-I) was successfully produced in plants. Plant-produced H5HA-I induced high titer of serum HI and VN antibodies in mice. Immunization with plant-produced H5HA-I protected ferrets from challenge infection of A/Indonesia/05/05. Based on these data, the present invention encompasses the recognition that plant-produced HA antigens may be useful for development of vaccines for use in humans.

### Example 4

### Recombinant Hemagglutinin (HA) Antigens

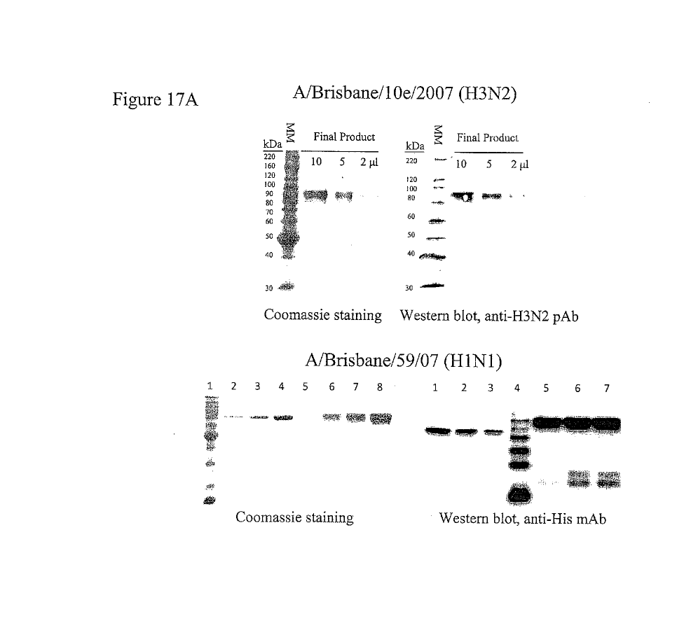

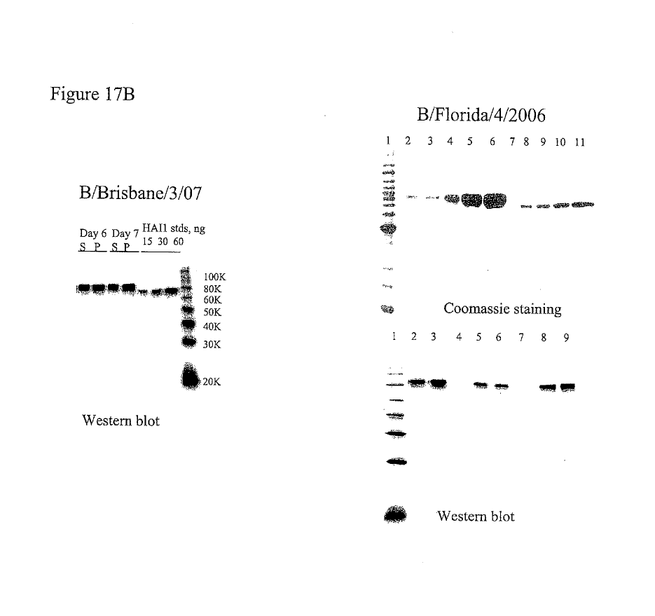

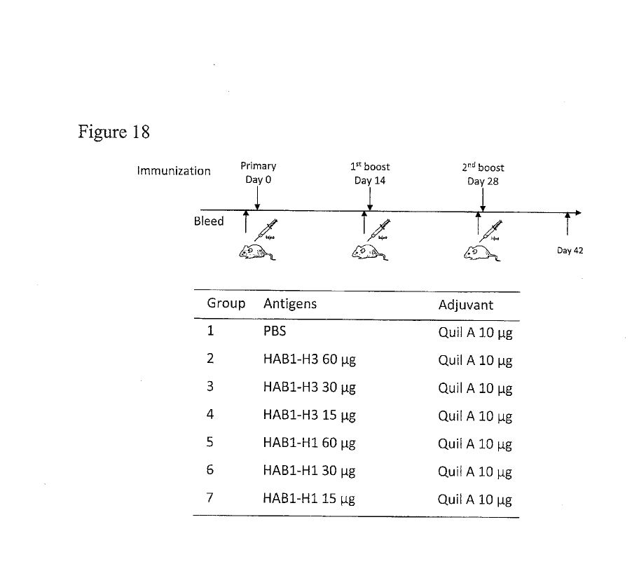

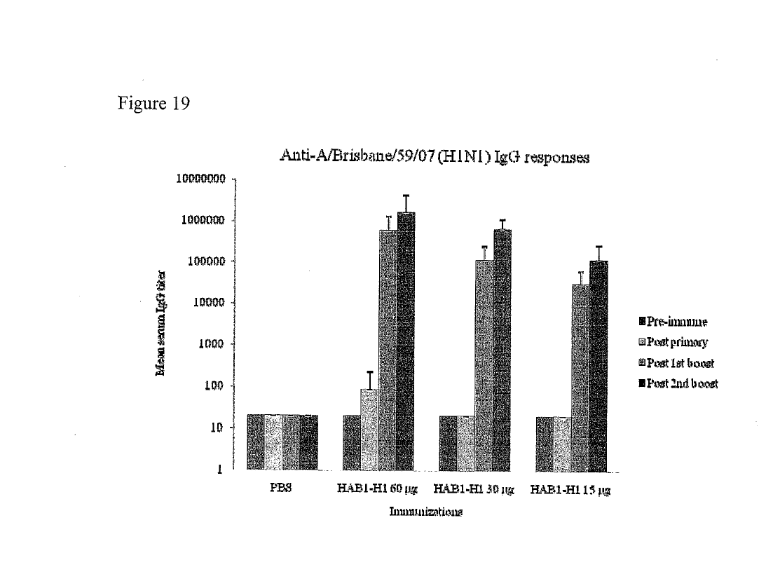

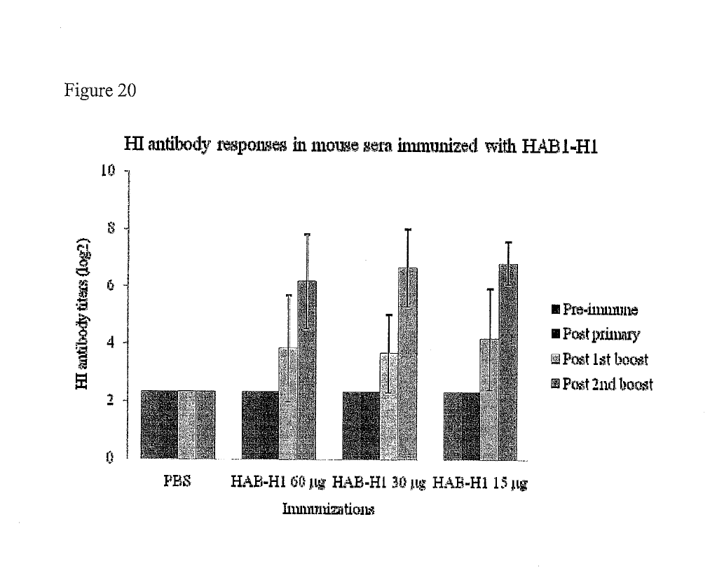

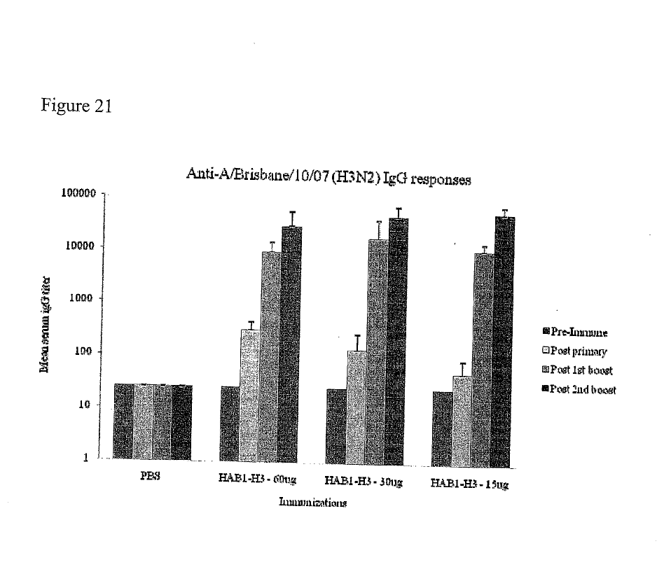

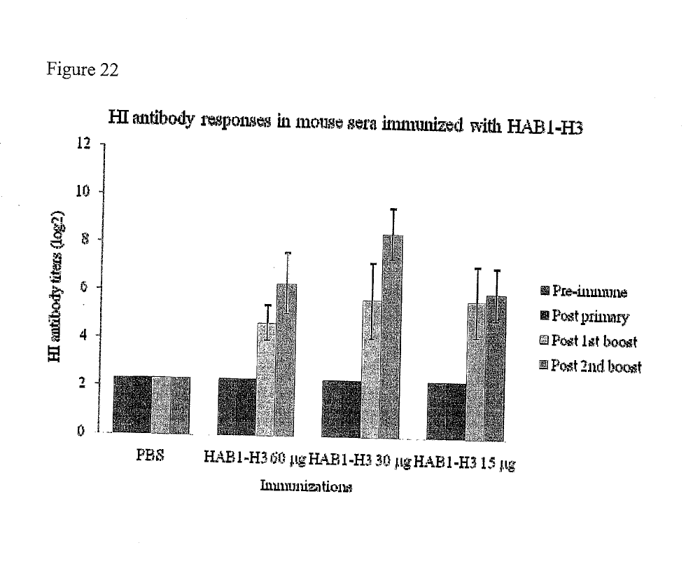

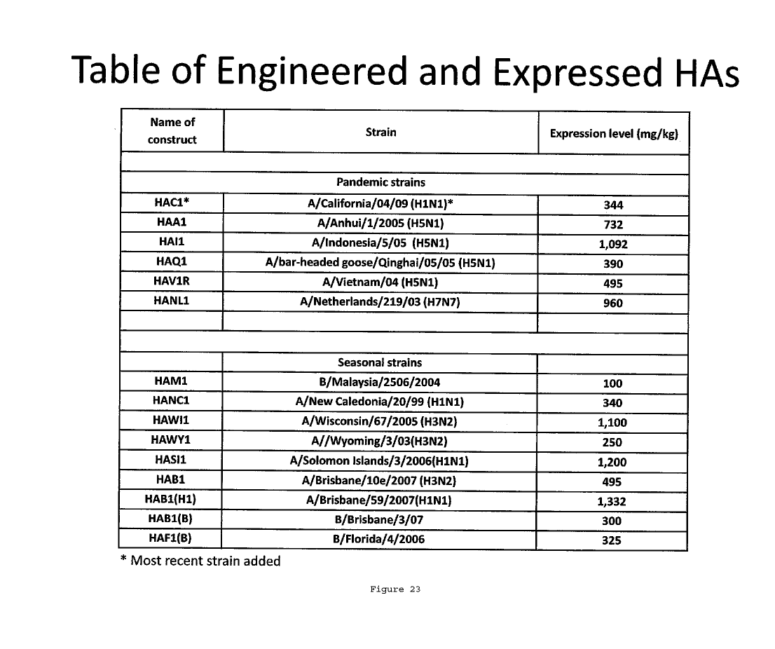

Full-length hemagglutinin (HA) protein was engineered, expressed, and purified from the A/Brisbane/10e/2007 (“HAB1-H3”), A/Brisbane/59/07 (“HAB1-H1”), B/Brisbane/3/07 (“HAB1-B”), and B/Florida/4/2006 (“HAF1-B”) strains of influenza in plants (FIG. 17). Immunization of mice (FIG. 18) with plant-produced HAB1-H3 and HAB1-H1 resulted in production of IgG antibodies (FIGS. 19 and 21), as well as significant serum hemagglutination inhibition (HI) (FIGS. 20 and 22) and virus neutralizing (VN) antibody titers. These results demonstrate that plant-produced HA protein is antigenic and can induce immune responses in mice that correlate with protection.

### Materials and Methods

Production of Proteins in Plants

The nucleotide sequence of HA from A/Brisbane/10e/2007 (“HAB1-H3”) that was cloned into launch vectors is:

(SEQ ID NO: 96)

5′ATGGGTTTCGTGCTTTTCTCTCAGCTTCCTTCTTTCCTTCTTGTGTCT
ACCCTTCTTCTTTTCCTTGTGATTTCTCACTCTTGCAGGGCTCAAAAGTT
GCCTGGAAACGATAATTCTACCGCTACCCTTTGCCTTGGTCATCATGCTG
TTCCTAACGGAACCATTGTGAAAACCATTACCAACGATCAGATTGAGGTG
ACCAATGCTACTGAGCTTGTTCAGTCATCTTCTACCGGTGAAATTTGCGA
TTCTCCTCACCAGATTCTTGATGGTGAAAACTGCACCCTTATTGATGCTT
TGCTTGGTGATCCTCAGTGTGATGGTTTCCAGAACAAGAAGTGGGATCTT
TTCGTTGAGAGGTCTAAGGCTTACTCTAACTGCTACCCTTACGATGTTCC
TGATTACGCTTCTCTTAGATCACTTGTGGCTTCATCTGGAACCCTTGAG
TTCAACAACGAGTCTTTCAATTGGACTGGTGTTACCCAGAACGGTACTT
CTTCTGCTTGCATTAGAAGGTCTAACAACTCTTTCTTCTCTAGGCTTAAC
TGGCTTACCCACCTTAAGTTCAAGTACCCTGCTCTTAATGTGACCATG
CCTAACAACGAGAAGTTCGATAAGTTGTACATTTGGGGAGTTCATCAC
CCTGGTACTGATAATGATCAGATTTTCCCTTACGCTCAGGCTTCTGGAA
GGATTACTGTGTCTACCAAGAGGTCACAGCAGACTGTGATTCCTAACATT
GGTTCTAGGCCAAGAGTGAGGAACATTCCTTCTAGGATTTCTATCTACTG
GACCATTGTGAAGCCTGGTGATATTCTTCTTATTAACTCTACCGGTAACC
TTATTGCTCCTAGGGGATACTTCAAGATTAGAAGTGGAAAGTCATCTATT
ATGAGATCAGATGCTCCTATTGGAAAGTGCAACTCTGAGTGCATTACCCC
TAACGGTTCTATTCCTAACGATAAGCCTTTCCAGAACGTGAACAGGATTA
CTTATGGTGCTTGCCCTAGATACGTGAAGCAGAACACCCTTAAGTTGGCT
ACTGGAATGAGGAATGTGCCTGAGAAGCAGACTAGGGGAATTTTCGGA
GCTATTGCTGGTTTCATTGAGAATGGATGGGAGGGAATGGTTGATGGTTG
GTACGGTTTCAGGCATCAGAACTCTGAAGGTATTGGACAGGCTGCTGATC
TTAAGTCTACCCAGGCTGCTATTGATCAGATTAACGGTAAGTTGAACAGG
CTTATTGGAAAGACCAATGAGAAGTTCCACCAGATTGAGAAAGAGTTCTC
TGAGGTTGAGGGAAGGATTCAGGATCTTGAGAAGTACGTGGAGGATACCA
AGATTGATCTTTGGTCTTACAACGCTGAGTTGCTTGTGGCTCTTGAGAAT
CAGCACACCATTGATCTTACCGATTCTGAGATGAACAAGTTGTTCGAAAA
GACCAAGAAGCAGCTTAGGGAGAACGCTGAGGATATGGGTAATGGTTG
CTTCAAAATCTACCACAAGTGCGATAACGCTTGCATTGGTTCTATTAG
GAACGGAACCTACGATCATGATGTGTACAGGGATGAGGCTCTTAATAA
CAGGTTCCAGATTAAGGGTGTTGAGCTTAAGTCTGGTTACAAGGATCAT
CACCATCACCACCACAAGGATGAGCTTTGATGA 3′.

The protein sequence encoded for by SEQ ID NO: 96 is:

(SEQ ID NO: 97)

5′MGFVLFSQLPSFLLVSTLLLFLVISHSCRAQKLPGNDNSTATLCLGHH
AVPNGTIVKTITNDQIEVTNATELVQSSSTGEICDSPHQILDGENCTLID
ALLGDPQCDGFQNKKWDLFVERSKAYSNCYPYDVPDYASLRSLVASSGTL
EFNNESFNWTGVTQNGTSSACIRRSNNSFFSRLNWLTHLKFKYPALNVTM
PNNEKFDKLYIWGVHHPGTDNDQIFPYAQASGRITVSTKRSQQTVIPNIG
SRPRVRNIPSRISIYWTIVKPGDILLINSTGNLIAPRGYFKIRSGKSSIM
RSDAPIGKCNSECITPNGSIPNDKPFQNVNRITYGACPRYVKQNTLKLAT
GMRNVPEKQTRGIFGAIAGFIENGWEGMVDGWYGFRHQNSEGIGQAA
DLKSTQAAIDQINGKLNRLIGKTNEKFHQIEKEFSEVEGRIQDLEKYVE
DTKIDLWSYNAELLVALENQHTIDLTDSEMNKLFEKTKKQLRENAEDM
GNGCFKIYHKCDNACIGSIRNGTYDHDVYRDEALNNRFQIKGVELKSGYK
DHHHHHHKDEL 3′.

The nucleotide sequence of HA from A/Brisbane/59/07 (“HAB1-H1”) that was cloned into launch vectors is:

(SEQ ID NO: 98)

5′ATGGGTTTCGTGCTTTTCTCTCAGCTTCCTTCTTTCCTTCTTGTGTCT
ACCCTTCTTCTTTTCCTTGTGATTTCTCACTCTTGCAGGGCTGATACCAT
CTGCATTGGTTACCACGCTAACAACTCTACTGATACTGTGGATACCGT
GCTTGAGAAGAATGTGACTGTGACCCACTCTGTGAACCTTTTGGAGAACT
CTCACAACGGTAAGTTGTGCCTTCTTAAGGGTATTGCTCCTCTTCAGCTT
GGAAATTGCTCTGTGGCTGGATGGATTCTTGGAAATCCTGAGTGCGAGCT
TCTTATTTCTAAAGAGTCTTGGTCTTACATTGTGGAGAAGCCTAATCCTG
AGAACGGTACTTGCTACCCTGGTCACTTTGCTGATTACGAAGAGCTTAGA
GAGCAGCTTTCTTCTGTTTCTTCTTTCGAGAGATTCGAGATTTTCCCTAA
AGAGTCATCTTGGCCTAATCATACTGTGACTGGTGTGTCTGCTTCTTGCT
CTCATAACGGTGAGTCATCTTTCTACAGGAACCTTCTTTGGCTTACCGGA
AAGAACGGTCTTTACCCTAACCTTTCTAAGTCTTACGCTAACAACAAAGA
GAAAGAGGTTTTGGTTCTTTGGGGTGTTCATCACCCTCCTAACATTGGTG
ATCAGAAGGCTCTTTACCATACCGAGAACGCTTACGTTTCTGTGGTGTCA
TCTCACTACTCTAGGAAGTTCACCCCTGAGATTGCTAAGAGGCCTAAAGT
GAGGGATCAAGAGGGAAGGATTAACTACTACTGGACCCTTCTTGAACCTG
GTGATACCATTATTTTCGAGGCTAACGGTAACCTTATTGCTCCTAGATAC
GCTTTCGCTCTTTCTAGAGGTTTCGGTTCTGGTATTATTAACTCTAACGC
TCCTATGGATAAGTGTGATGCTAAGTGCCAGACTCCTCAGGGTGCTATT
AACTCTTCTCTTCCTTTCCAGAATGTGCACCCTGTTACTATTGGTGAGTG
CCCTAAGTATGTGAGATCAGCTAAGTTGAGGATGGTGACCGGTCTTAG
GAACATTCCTTCTATTCAGTCTAGGGGACTTTTCGGAGCTATTGCTGGT
TTTATTGAGGGAGGATGGACTGGAATGGTTGATGGTTGGTACGGTTACC
ATCATCAGAATGAGCAGGGTTCTGGTTATGCTGCTGATCAGAAGTCTAC
CCAGAACGCTATTAACGGTATTACCAACAAGGTGAACTCTGTGATTGAGA
AGATGAACACCCAGTTCACTGCTGTTGGAAAAGAGTTCAACAAGTTGGA
GAGAAGGATGGAAAACCTTAACAAGAAAGTGGATGATGGTTTCATTGATA
TTTGGACCTACAACGCTGAGTTGCTTGTGCTTCTTGAGAATGAGAGGACC
CTTGATTTCCACGATTCTAACGTGAAGAACCTTTACGAGAAGGTGAAGTC
TCAGCTTAAGAACAACGCTAAAGAGATTGGAAACGGTTGCTTCGAGTTC
TACCACAAGTGCAACGATGAGTGCATGGAATCTGTGAAGAACGGAACCT
ACGATTACCCTAAGTACTCTGAAGAGTCTAAGTTGAACAGAGAAAAGAT
TGATGGTGTTAAGTTGGAGTCTATGGGAGTGTACCAGATTCATCACCATC
ACCACCACAAGGATGAGCTTTGA 3′.

The protein sequence encoded for by SEQ ID NO: 98 is:

(SEQ ID NO: 99)

5′MGFVLFSQLPSFLLVSTLLLFLVISHSCRADTICIGYHANNSTDTVDT
VLEKNVTVTHSVNLLENSHNGKLCLLKGIAPLQLGNCSVAGWILGNPECE
LLISKESWSYIVEKPNPENGTCYPGHFADYEELREQLSSVSSFERFEIFP
KESSWPNHTVTGVSASCSHNGESSFYRNLLWLTGKNGLYPNLSKSYAN
NKEKEVLVLWGVHHPPNIGDQKALYHTENAYVSVVSSHYSRKFTPEIAKR
PKVRDQEGRINYYWTLLEPGDTIIFEANGNLIAPRYAFALSRGFGSGIIN
SNAPMDKCDAKCQTPQGAINSSLPFQNVHPVTIGECPKYVRSAKLRMVTG
LRNIPSIQSRGLFGAIAGFIEGGWTGMVDGWYGYHHQNEQGSGYAADQKS
TQNAINGITNKVNSVIEKMNTQFTAVGKEFNKLERRMENLNKKVDDGFID
IWTYNAELLVLLENERTLDFHDSNVKNLYEKVKSQLKNNAKEIGNGCFEF
YHKCNDECMESVKNGTYDYPKYSEESKLNREKIDGVKLESMGVYQIHHHH
HHKDEL 3′.

The nucleotide sequence of HA from B/Brisbane/3/07 (“HAB1-B”) that was cloned into launch vectors is:

(SEQ ID NO: 100)

5′ATGGGTTTCGTGCTTTTCTCTCAGCTTCCTTCTTTCCTTCTTGTGTCT
ACCCTTCTTCTTTTCCTTGTGATTTCTCACTCTTGCAGGGCTGATAGAAT
CTGCACCGGTATTACCTCTTCTAACTCTCCTCACGTGGTTAAGACTGCTA
CTCAGGGTGAAGTTAATGTGACCGGTGTTATTCCTCTTACTACCACCCCT
ACCAAGTCTTACTTCGCTAACCTTAAGGGTACTAAGACTAGAGGAAAGTT
GTGCCCTGATTGCCTTAATTGCACCGATCTTGATGTTGCTCTTGGAAGGC
CTATGTGTGTTGGTACTACCCCTTCTGCTAAGGCTTCTATTCTTCACGAA
GTGAGACCTGTTACTTCTGGTTGCTTCCCTATTATGCACGATAGGACCAA
GATTAGGCAGCTTGCTAACCTTCTTAGGGGTTACGAGAACATTAGGCTTT
CTACCCAGAACGTGATTGATGCTGAAAAGGCTCCTGGTGGTCCTTATAGG
CTTGGAACCTCTGGTTCTTGCCCTAATGCTACCTCTAAGTCTGGTTTCTT
CGCTACTATGGCTTGGGCTGTGCCTAAGGATAACAACAAGAACGCTACCA
ATCCTCTTACTGTGGAGGTGCCATATATCTGTACCGAGGGTGAAGATCAG
ATTACTGTGTGGGGTTTCCACTCTGATGATAAGACCCAGATGAAGAACCT
TTACGGTGATTCTAACCCTCAGAAGTTCACCTCTTCTGCTAATGGTGTTA
CCACCCACTACGTGTCTCAGATTGGTGGTTTCCCTGATCAAACTGAGGA
TGGTGGACTTCCTCAGTCTGGAAGGATTGTGGTGGATTACATGATGCAAA
AGCCTGGAAAGACCGGAACTATTGTGTATCAGAGGGGAGTTCTTCTTCCT
CAGAAAGTGTGGTGTGCTTCTGGTAGGTCTAAAGTGATTAAGGGTTCTCT
TCCTCTTATTGGAGAGGCTGATTGCCTTCATGAGAAGTACGGTGGTCTTA
ACAAGTCTAAGCCTTACTACACTGGTGAACACGCTAAGGCTATTGGAAAC
TGCCCTATTTGGGTTAAGACCCCTCTTAAGTTGGCTAACGGTACTAAGTA
TAGGCCTCCTGCTAAGTTGCTTAAAGAGAGGGGATTCTTCGGAGCTATTG
CTGGTTTTCTTGAGGGAGGATGGGAGGGAATGATTGCTGGATGGCACGGT
TATACTTCTCATGGTGCTCACGGTGTTGCTGTTGCTGCTGATCTTAAGTC
TACCCAGGAAGCTATTAACAAGATTACCAAGAACCTTAACTCTCTTTCTG
AGCTTGAGGTGAAGAACCTTCAGAGACTTTCTGGTGCTATGGATGAGCTT
CACAACGAGATTCTTGAGCTTGATGAGAAAGTGGATGATCTTAGGGCTGA
TACCATTTCTTCTCAGATTGAGCTTGCTGTGCTTCTTTCTAACGAGGGTA
TCATTAACTCTGAGGATGAGCACCTTCTTGCTCTTGAGAGGAAGTTGAAG
AAGATGCTTGGTCCTTCTGCTGTGGATATTGGAAATGGTTGCTTCGAGAC
TAAGCACAAGTGCAATCAGACTTGCCTTGATAGGATTGCTGCTGGAACT
TTCAATGCTGGTGAGTTCTCTCTTCCTACCTTCGATTCTCTTAACATTAC
CGCTGCTTCTCTTAACGATGATGGTCTTGATAATCACACTCATCACC
ATCACCACCACAAGGATGAGCTTTGA 3′.

The protein sequence encoded for by SEQ ID NO: 100 is:

(SEQ ID NO: 101)

5′MGFVLFSQLPSFLLVSTLLLFLVISHSCRADRICTGITSSNSPHVVKT
ATQGEVNVTGVIPLTTTPTKSYFANLKGTKTRGKLCPDCLNCTDLDVALG
RPMCVGTTPSAKASILHEVRPVTSGCFPIMHDRTKIRQLANLLRGYENIR
LSTQNVIDAEKAPGGPYRLGTSGSCPNATSKSGFFATMAWAVPKDNNKNA
TNPLTVEVPYICTEGEDQITVWGFHSDDKTQMKNLYGDSNPQKFTSSANG
VTTHYVSQIGGFPDQTEDGGLPQSGRIVVDYMMQKPGKTGTIVYQRGVLL
PQKVWCASGRSKVIKGSLPLIGEADCLHEKYGGLNKSKPYYTGEHAKAIG
NCPIWVKTPLKLANGTKYRPPAKLLKERGFFGAIAGFLEGGWEGMIAGWH
GYTSHGAHGVAVAADLKSTQEAINKITKNLNSLSELEVKNLQRLSGAMDE
LHNEILELDEKVDDLRADTISSQIELAVLLSNEGIINSEDEHLLALERKL
KKMLGPSAVDIGNGCFETKHKCNQTCLDRIAAGTFNAGEFSLPTFDSLNI
TAASLNDDGLDNHTHHHHHHKDEL 3′.

The nucleotide sequence of HA from B/Florida/4/2006 (“HAF1-B”) (ACA33493) that was cloned into launch vectors is:

(SEQ ID NO: 102)

5′ATGGGTTTCGTGCTTTTCTCTCAGCTTCCTTCTTTCCTTCTTGTGTCT
ACCCTTCTTCTTTTCCTTGTGATTTCTCACTCTTGCAGGGCTGATAGAAT
CTGCACCGGTATTACCTCTTCTAACTCTCCTCACGTGGTTAAGACTGCT
ACTCAGGGTGAAGTTAATGTGACCGGTGTTATTCCTCTTACTACCACCC
CTACCAAGTCTTACTTCGCTAACCTTAAGGGTACTAGGACTAGAGGAA
AGTTGTGCCCTGATTGCCTTAATTGCACCGATCTTGATGTTGCTCTTGGA
AGGCCTATGTGTGTTGGTACTACCCCTTCTGCTAAGGCTTCTATTCTTCA
CGAGGTGAAGCCTGTTACTTCTGGTTGCTTCCCTATTATGCACGATAGGA
CCAAGATTAGGCAGCTTCCTAACCTTCTTAGGGGTTACGAGAACATTAGG
CTTTCTACCCAGAACGTGATTGATGCTGAAAAGGCTCCTGGTGGTCCTTA
TAGGCTTGGAACCTCTGGTTCTTGCCCTAATGCTACCTCTAAGTCTGGTT
TCTTCGCTACTATGGCTTGGGCTGTGCCTAAGGATAACAACAAGAACGCT
ACCAATCCTCTTACTGTGGAGGTGCCATATATCTGTACCGAGGGTGAAGA
TCAGATTACTGTGTGGGGTTTCCACTCTGATGATAAGACCCAGATGAAGA
ACCTTTACGGTGATTCTAACCCTCAGAAGTTCACCTCTTCTGCTAATGGT
GTTACCACCCACTACGTGTCTCAGATTGGTTCTTTCCCTGATCAAACTGA
GGATGGTGGACTTCCTCAGTCTGGAAGGATTGTGGTGGATTACATGATGC
AAAAGCCTGGAAAGACCGGAACTATTGTGTATCAGAGGGGAGTTCTTCTT
CCTCAGAAAGTGTGGTGTGCTTCTGGTAGGTCTAAAGTGATTAAGGGTTC
TCTTCCTCTTATTGGAGAGGCTGATTGCCTTCATGAGAAGTACGGTGGTC
TTAACAAGTCTAAGCCTTACTACACTGGTGAACACGCTAAGGCTATTGGA
AACTGCCCTATTTGGGTTAAGACCCCTCTTAAGTTGGCTAACGGTACTAA
GTATAGGCCTCCTGCTAAGTTGCTTAAAGAGAGGGGATTCTTCGGAGCTA
TTGCTGGTTTTCTTGAGGGAGGATGGGAGGGAATGATTGCTGGATGGCAC
GGTTATACTTCTCATGGTGCTCACGGTGTTGCTGTTGCTGCTGATCTTAA
GTCTACCCAGGAAGCTATTAACAAGATTACCAAGAACCTTAACTCTCTTT
CTGAGCTTGAGGTGAAGAACCTTCAGAGACTTTCTGGTGCTATGGATGAG
CTTCACAACGAGATTCTTGAGCTTGATGAGAAAGTGGATGATCTTAGGGC
TGATACCATTTCTTCTCAGATTGAGCTTGCTGTGCTTCTTTCTAACGAGG
GTATCATTAACTCTGAGGATGAGCACCTTCTTGCTCTTGAGAGGAAGTTG
AAGAAGATGCTTGGTCCTTCTGCTGTGGAGATTGGAAATGGTTGCTTCGA
GACTAAGCACAAGTGCAATCAGACTTGCCTTGATAGGATTGCTGCTGGA
ACTTTCAATGCTGGTGAGTTCTCTCTTCCTACCTTCGATTCTCTTAACAT
TACCGCTGCTTCTCTTAACGATGATGGTCTTGATAATCACACTCATCAC
CATCACCACCACAAGGATGAGCTTTGA 3′.

The protein sequence encoded for by SEQ ID NO: 102 is:

(SEQ ID NO: 103)

5′MGFVLFSQLPSFLLVSTLLLFLVISHSCRADRICTGITSSNSPHVVKT
ATQGEVNVTGVIPLTTTPTKSYFANLKGTRTRGKLCPDCLNCTDLDVALG
RPMCVGTTPSAKASILHEVKPVTSGCFPIMHDRTKIRQLPNLLRGYENIR
LSTQNVIDAEKAPGGPYRLGTSGSCPNATSKSGFFATMAWAVPKDNNKNA
TNPLTVEVPYICTEGEDQITVWGFHSDDKTQMKNLYGDSNPQKFTSSANG
VTTHYVSQIGSFPDQTEDGGLPQSGRIVVDYMMQKPGKTGTIVYQRGVLL
PQKVWCASGRSKVIKGSLPLIGEADCLHEKYGGLNKSKPYYTGEHAKAIG
NCPIWVKTPLKLANGTKYRPPAKLLKERGFFGAIAGFLEGGWEGMIAGWH
GYTSHGAHGVAVAADLKSTQEAINKITKNLNSLSELEVKNLQRLSGAMD
ELHNEILELDEKVDDLRADTISSQIELAVLLSNEGIINSEDEHLLALERK
LKKMLGPSAVEIGNGCFETKHKCNQTCLDRIAAGTFNAGEFSLPTFDSLN
ITAASLNDDGLDNHTHHHHHHKDEL 3′.

The nucleotide sequence of HA from A/New Calcdonia/20/99 (AAP34324) that was cloned into launch vectors is:

(SEQ ID NO: 104)

5′ATGGGATTCGTGCTTTTCTCTCAGCTTCCTTCTTTCCTTCTTGTGTCT
ACTCTTCTTCTTTTCCTTGTGATTTCTCACTCTTGCAGGGCTGATACAAT
CTGCATTGGATACCACGCTAACAACTCTACTGATACTGTGGATACTGTTC
TTGAGAAGAACGTGACTGTGACTCACTCTGTGAACCTTTTGGAGGATTCT
CACAACGGAAAGTTGTGCCTTCTTAAGGGAATTGCTCCACTTCAACTTGG
AAACTGCAGTGTGGCTGGATGGATTCTTGGAAATCCAGAGTGCGAGCTTC
TTATTTCTAAAGAGTCTTGGTCTTACATTGTGGAGACTCCAAATCCAGAG
AACGGAACTTGTTACCCAGGATACTTCGCTGATTACGAAGAGCTTAGAGA
GCAGCTTTCTTCTGTTTCTTCTTTCGAGAGATTCGAGATTTTCCCAAAAG
AGTCATCTTGGCCAAACCACACTGTTACTGGTGTTTCTGCTTCTTGCTCT
CATAACGGTAAGTCATCTTTCTACAGGAACCTTCTTTGGCTTACTGGAAA
GAACGGACTTTACCCAAACCTTTCTAAGTCTTACGTGAACAACAAAGA
GAAAGAGGTTTTGGTTCTTTGGGGAGTTCATCACCCACCAAACATTGGAA
ATCAGAGGGCTCTTTACCATACTGAGAACGCTTACGTGTCTGTGGTTTCT
TCTCACTACTCTAGAAGGTTCACTCCAGAGATTGCTAAGAGGCCAAAAG
TGAGGGATCAAGAGGGAAGGATTAACTACTACTGGACTCTTCTTGAGCCA
GGTGATACAATTATTTTCGAGGCTAACGGAAACCTTATTGCTCCATGGTA
CGCTTTTGCTTTGTCTAGGGGATTCGGATCTGGAATTATTACTTCTAACG
CTCCAATGGATGAGTGTGATGCTAAGTGCCAAACTCCACAGGGTGCTATT
AACTCTTCTCTTCCATTCCAGAACGTTCACCCAGTTACTATTGGAGAGTG
CCCAAAGTATGTGAGATCAGCTAAGTTGAGGATGGTGACTGGACTTAGGA
ACATTCCATCTATTCAGTCTAGGGGACTTTTCGGAGCTATTGCTGGATTC
ATTGAGGGAGGATGGACTGGAATGGTTGATGGATGGTACGGATACCATCA
TCAGAATGAGCAGGGATCTGGATATGCTGCTGATCAGAAGTCTACTCAGA
ACGCTATTAACGGAATTACTAACAAGGTGAACTCTGTGATTGAGAAGATG
AACACTCAGTTCACTGCTGTGGGAAAAGAGTTCAACAAGTTGGAGAGAAG
GATGGAAAACCTTAACAAGAAAGTGGATGATGGATTCCTTGATATTTGGA
CTTACAACGCTGAGTTGCTTGTGCTTCTTGAGAACGAGAGGACTCTTGAT
TTCCACGATTCTAACGTGAAGAACCTTTACGAGAAGGTGAAGTCTCAGCT
TAAGAACAACGCTAAAGAGATTGGAAACGGTTGCTTCGAGTTCTACCAC
AAGTGCAACAACGAGTGCATGGAATCTGTGAAGAACGGTACTTACGATTA
CCCAAAGTACTCTGAAGAGTCTAAGTTGAACAGAGAAAAGATTGATGGTG
TTAAGTTGGAGTCTATGGGAGTGTACCAGATTCATCACCATCACCACCA
CAAGGATGAGCTTTAA 3′.

The protein sequence encoded for by SEQ ID NO: 104 is:

(SEQ ID NO: 105)

5′MGFVLFSQLPSFLLVSTLLLFLVISHSCRADTICIGYHANNSTDTVDT
VLEKNVTVTHSVNLLEDSHNGKLCLLKGIAPLQLGNCSVAGWILGNPECE
LLISKESWSYIVETPNPENGTCYPGYFADYEELREQLSSVSSFERFEIFP
KESSWPNHTVTGVSASCSHNGKSSFYRNLLWLTGKNGLYPNLSKSYVNNK
EKEVLVLWGVHHPPNIGNQRALYHTENAYVSVVSSHYSRRFTPEIAKRPK
VRDQEGRINYYWTLLEPGDTIIFEANGNLIAPWYAFALSRGFGSGIITSN
APMDECDAKCQTPQGAINSSLPFQNVHPVTIGECPKYVRSAKLRMVTGLR
NIPSIQSRGLFGAIAGFIEGGWTGMVDGWYGYHHQNEQGSGYAADQKSTQ
NAINGITNKVNSVIEKMNTQFTAVGKEFNKLERRMENLNKKVDDGFLDIW
TYNAELLVLLENERTLDFHDSNVKNLYEKVKSQLKNNAKEIGNGCFEFYH
KCNNECMESVKNGTYDYPKYSEESKLNREKIDGVKLESMGVYQIHHHHH
HKDEL 3′.

The nucleotide sequence of HA from A/Solomon Islands/3/2006 (ABU99109) that was cloned into launch vectors is:

(SEQ ID NO: 106)

5′ATGGGTTTCGTGCTTTTCTCTCAGCTTCCTTCTTTCCTTCTTGTGTCT
ACCCTTCTTCTTTTCCTTGTGATTTCTCACTCTTGCAGGGCTGATACCAT
CTGCATTGGTTACCACGCTAACAACTCTACTGATACTGTGGATACCGTGC
TTGAGAAGAATGTGACTGTGACCCACTCTGTGAACCTTTTGGAGGATTCT
CACAACGGTAAGTTGTGCCTTCTTAAGGGTATTGCTCCTCTTCAGCTTGG
AAATTGCTCTGTGGCTGGATGGATTCTTGGAAATCCTGAGTGCGAGCTTC
TTATTTCTAGAGAGTCTTGGTCTTACATTGTGGAGAAGCCTAATCCTGAG
AACGGTACTTGCTACCCTGGTCACTTTGCTGATTACGAAGAGCTTAGAGA
GCAGCTTTCTTCTGTTTCTTCTTTCGAGAGATTCGAGATTTTCCCTAAAG
AGTCATCTTGGCCTAACCATACCACTACTGGTGTTTCTGCTTCTTGCTCA
CACAACGGTGAGTCATCTTTCTACAAGAACCTTCTTTGGCTTACCGGAAA
GAACGGTCTTTACCCTAACCTTTCTAAGTCTTACGCTAACAACAAAGAGA
AAGAGGTTTTGGTTCTTTGGGGTGTTCATCACCCTCCTAACATTGGTGAT
CAGAGGGCTCTTTACCACAAAGAGAACGCTTACGTTTCTGTGGTGTCATC
TCACTACTCTAGGAAGTTCACCCCTGAGATTGCTAAGAGGCCTAAAGTGA
GGGATCAAGAGGGAAGGATTAACTACTACTGGACCCTTCTTGAACCTGG
TGATACCATTATTTTCGAGGCTAACGGTAACCTTATTGCTCCTAGATACG
CTTTCGCTCTTTCTAGAGGTTTCGGTTCTGGTATTATTAACTCTAACGCT
CCTATGGATGAGTGTGATGCTAAGTGTCAGACTCCTCAGGGTGCTATTA
ACTCTTCTCTTCCTTTCCAGAATGTGCACCCTGTTACTATTGGTGAGTG
CCCTAAGTATGTGAGATCAGCTAAGTTGAGGATGGTGACCGGTCTTAGGA
ACATTCCTTCTATTCAGTCTAGGGGACTTTTCGGAGCTATTGCTGGTTTT
ATTGAGGGAGGATGGACTGGAATGGTTGATGGTTGGTACGGTTACCATC
ATCAGAATGAGCAGGGTTCAGGTTATGCTGCTGATCAGAAGTCTACCCAG
AACGCTATTAACGGTATTACCAACAAGGTGAACTCTGTGATTGAGAAGAT
GAACACCCAGTTCACTGCTGTTGGAAAAGAGTTCAACAAGTTGGAGAGAA
GGATGGAAAACCTTAACAAGAAAGTGGATGATGGTTTCATTGATATTTGG
ACCTACAACGCTGAGTTGCTTGTGCTTCTTGAGAATGAGAGGACCCTTGA
TTTCCACGATTCTAACGTGAAGAACCTTTACGAGAAGGTGAAGTCTCAG
CTTAAGAACAACGCTAAAGAGATTGGAAACGGTTGCTTCGAGTTCTACCA
CAAGTGCAACGATGAGTGCATGGAATCTGTGAAGAACGGAACCTACGATT
ACCCTAAGTACTCTGAAGAGTCTAAGTTGAACAGAGAAAAGATTGATGGT
GTTAAGTTGGAGTCTATGGGAGTGTACCAGATTCATCACCATCACCACCA
CAAGGATGAGCTTTGATGA 3′.

The protein sequence encoded for by SEQ ID NO: 106 is:

(SEQ ID NO: 107)

5′MGFVLFSQLPSFLLVSTLLLFLVISHSCRADTICIGYHANNSTDTVDT
VLEKNVTVTHSVNLLEDSHNGKLCLLKGIAPLQLGNCSVAGWILGNPECE
LLISRESWSYIVEKPNPENGTCYPGHFADYEELREQLSSVSSFERFEIFP
KESSWPNHTTTGVSASCSHNGESSFYKNLLWLTGKNGLYPNLSKSYANNK
EKEVLVLWGVHHPPNIGDQRALYHKENAYVSVVSSHYSRKFTPEIAKRPK
VRDQEGRINYYWTLLEPGDTIIFEANGNLIAPRYAFALSRGFGSGIINSN
APMDECDAKCQTPQGAINSSLPFQNVHPVTIGECPKYVRSAKLRMVTGLR
NIPSIQSRGLFGAIAGFIEGGWTGMVDGWYGYHHQNEQGSGYAADQKSTQ
NAINGITNKVNSVIEKMNTQFTAVGKEFNKLERRMENLNKKVDDGFIDIW
TYNAELLVLLENERTLDFHDSNVKNLYEKVKSQLKNNAKEIGNGCFEFYH
KCNDECMESVKNGTYDYPKYSEESKLNREKIDGVKLESMGVYQIHHH
HHHKDEL 3′.

The nucleotide sequence of HA from A/Wisconsin/67/2005 that was cloned into launch vectors is:

(SEQ ID NO: 108)

5′ATGGGATTCGTGCTTTTCTCTCAGCTTCCTTCTTTCCTTCTTGTGTCT
ACTCTTCTTCTTTTCCTTGTGATTTCTCACTCTTGCAGGGCTCAAAAGTT
GCCAGGAAACGATAACTCTACTGCTACTCTTTGCCTTGGACATCACGC
TGTTCCAAACGGAACTATTGTGAAAACTATTACTAACGATCAGATTGAGG
TGACAAACGCTACTGAGCTTGTTCAGTCATCTTCTACTGGTGGAATTTGC
GATTCTCCACACCAGATTCTTGATGGTGAAAACTGCACTCTTATTGATGC
TTTGCTTGGAGATCCACAGTGTGATGGATTCCAGAACAAGAAGTGGGATC
TTTTCGTTGAGAGGTCTAAGGCTTACTCTAACTGCTACCCATACGATGTT
CCAGATTACGCTTCTCTTAGATCACTTGTGGCTTCATCTGGAACTCTTGA
GTTCAACGATGAGTCTTTCAACTGGACTGGTGTTACTCAGAACGGAACTT
CATCTTCATGCAAGAGGAGGTCTAACAACTCTTTCTTCTCTAGGCTTAAC
TGGCTTACTCACCTTAAGTTCAAGTACCCAGCTCTTAACGTGACTATGC
CAAACAACGAGAAGTTCGATAAGTTGTACATTTGGGGAGTTCACCACC
CAGTTACTGATAATGATCAGATTTTCCTTTACGCTCAGGCTTCTGGAAGG
ATTACTGTGTCTACTAAGAGGTCTCAGCAGACTGTGATTCCAAACATTGG
ATCTAGGCCAAGGATTAGGAACATTCCATCTAGGATTTCTATTTACTGGA
CTATTGTGAAGCCAGGTGATATTCTTCTTATTAACTCTACTGGAAACCTT
ATTGCTCCAAGGGGATACTTCAAGATTAGAAGTGGAAAGTCATCTATTA
TGAGATCAGATGCTCCAATTGGAAAGTGCAACTCTGAGTGCATTACTCC
AAACGGTTCTATTCCAAACGATAAGCCATTCCAGAACGTGAACAGGATT
ACTTATGGTGCTTGCCCAAGATACGTGAAGCAGAACACTCTTAAGTTGG
CTACTGGAATGAGGAATGTGCCAGAGAAGCAGACTAGGGGAATTTTCGG
AGCTATTGCTGGATTCATTGAGAATGGATGGGAGGGAATGGTTGATGGA
TGGTACGGATTCAGGCATCAAAACTCTGAGGGAATTGGACAAGCTGCT
GATCTTAAGTCTACTCAGGCTGCTATTAACCAGATTAACGGAAAGTTGAA
CAGGCTTATTGGAAAGACTAATGAGAAGTTCCACCAGATTGAGAAAGAGT
TCTCTGAGGTTGAGGGAAGGATTCAGGATCTTGAGAAGTACGTGGAGGAT
ACAAAGATTGATCTTTGGTCTTACAACGCTGAGTTGCTTGTTGCTCTTGA
GAACCAGCACACTATTGATCTTACTGATTCTGAGATGAACAAGTTGTTCG
AGAGGACTAAGAAGCAGCTTAGGGAGAACGCTGAGGATATGGGAAATGG
ATGCTTCAAGATTTACCACAAGTGCGATAACGCTTGCATTGGATCTATTA
GGAACGGAACTTACGATCACGATGTGTACAGAGATGAGGCTCTTAACAA
CAGGTTCCAGATTAAGGGTGTTGAGCTTAAGTCTGGATACAAGGATCATC
ACCATCACCACCACAAGGATGAGCTTTGA 3′.

The protein sequence encoded for by SEQ ID NO: 108 is:

(SEQ ID NO: 109)

5′MGFVLFSQLPSFLLVSTLLLFLVISHSCRAQKLPGNDNSTATLCLGHH
AVPNGTIVKTITNDQIEVTNATELVQSSSTGGICDSPHQILDGENCTLID
ALLGDPQCDGFQNKKWDLFVERSKAYSNCYPYDVPDYASLRSLVASSGT
LEFNDESFNWTGVTQNGTSSSCKRRSNNSFFSRLNWLTHLKFKYPALNVT
MPNNEKFDKLYIWGVHHPVTDNDQIFLYAQASGRITVSTKRSQQTVIPNI
GSRPRIRNIPSRISIYWTIVKPGDILLINSTGNLIAPRGYFKIRSGKSSI
MRSDAPIGKCNSECITPNGSIPNDKPFQNVNRITYGACPRYVKQNTLKLA
TGMRNVPEKQTRGIFGAIAGFIENGWEGMVDGWYGFRHQNSEGIGQAADL
KSTQAAINQINGKLNRLIGKTNEKFHQIEKEFSEVEGRIQDLEKYVEDTK
IDLWSYNAELLVALENQHTIDLTDSEMNKLFERTKKQLRENAEDMGNGCF
KIYHKCDNACIGSIRNGTYDHDVYRDEALNNRFQIKGVELKSGY 3′

Antigens were produced in plants essentially as described in Example 1.

ELISA of Purified Plant-Produced H3HA or H1HA

Plant-produced HA from A/Brisbane/10/07 (H3N2) and A/Brisbane/59/07 (H1N1) were characterized by ELISA 96-well MaxiSorp plates (NUNC, Rochester, N.Y.) were coated with 1 μg/ml of purified HA from A/Brisbane/10/07, A/Brisbane/59/07, inactivated A/Brisbane/10/07 (Department of Health and Ageing Therapeutic Goods Administration (TGA) Lot#2007/79B, Australia) or inactivated A/Brisbane/59/07 (National Institute for Biological Standards and Control (NIBSC) code No. 08/100, UK). Plates were incubated with 1:1600 dilution of sheep anti-serum raised against HA from A/Brisbane/10/07 (TGA, Lot#AS393) or A/Brisbane/59/07 (NIBSC, code No. 08/112) and detected using rabbit anti-sheep IgG-HRP antibody (Bethyl Laboratory Inc.).

Immunization of Mice with Plant-Produced H3HA or H1HA

Groups of six-week old Balb/c mice, six mice per group, were immunized with plant-produced H3HA or H1HA subcutaneously at 2-week intervals on days 0, 14, 28. Three different antigen doses were tested: 60 μg/dose, 30 μg/dose, and 15 μg/dose. Animals in control groups received PBS. All immunizations were performed with the addition of 10 μg of Quil A (Accurate Chemical, Westbury, N.Y.). Serum samples were collected prior to each immunization and two weeks after the third dose.

Characterization of Immune Responses

The HA-specific serum antibody responses were measured by ELISA using 96-well MaxiSorp plates (NUNC, Rochester, N.Y.) coated with inactivated A/Brisbane/10/07 or A/Brisbane/59/07 virus. Samples of sera were tested in series of four-fold dilutions and antigen-specific antibodies were detected using HRP-conjugated goat anti-mouse IgG (Jackson Immunoresearch Laboratory Inc., West Grove, Pa.). Reciprocal serum dilutions that gave mean OD values three times greater than those from pre-immune sera at a 1:50 dilution were determined as endpoint titers.

Hemagglutination Inhibition (HI) and Virus Neutralization (VN) Assays

Serum samples from immunized mice were treated with receptor-destroying enzyme (RDE; Denka Seiken Co. Ltd., Tokyo, Japan) and an HI assay was carried out with 0.75% turkey erythrocytes, as described previously (Rowe et al., 1999, J. Clin. Microbiol., 37:937-43; incorporated herein by reference). The microneutralization assay was carried out as described previously (Rowe et al., 1999, J. Clin. Microbiol., 37:937-43) with the following modifications. MDCK cells were plated at 3×10⁴ cells/well in 96 well tissue culture plates, and incubated for 18 hours at 37° C. In parallel, RDE-treated serum samples were serially diluted and mixed with an equal volume of 2×10³ TCID₅₀/ml of A/Brisbane/10/07 or A/Brisbane/59/07 influenza virus. Following 1 hour incubation at 37° C., the serum-virus mixtures were added to the plated MDCK cells and further incubated for 18 hours. Plates were then washed with PBS and fixed with 80% acetone. The neutralizing endpoint titer of each sample was determined by ELISA as described previously (Rowe et al., 1999, J. Clin. Microbiol., 37:937-43).

Statistical Analysis

Statistical analysis of data was performed using a two-tailed t test with equal variance and significance was considered at a p-value <0.05. Samples without detectable IgG, HI or VN titers were assigned (detection limit 50, 10, or 20, respectively) a value of 25, 5, or 10 for statistical analysis.

Results and Discussion

HAB1-H3, HAB1-H1, HAB1-B, and HAF1-B antigens were all successfully produced in plants. FIG. 17A shows Coomassie brilliant blue staining and western blots of produced HAB1-H3 and HAB1-H1 proteins. Total protein expression for each construct was about 800 mg/kg plant biomass. FIG. 17B shows Coomassie brilliant blue staining and/or western blots of produced HAB1-B and HAF1-B proteins. Total protein expression for HAB1-B was about 800 mg/kg plant biomass. Total protein expression for HAF1-B was about 325 mg/kg plant biomass.

Mice were immunized with 60 μg, 30 μg, or 15 μg of plant-produced HA from A/Brisbane/59/07 (HAB1-H1) or A/Brisbane/10e/07 (HAB1-H3). See FIG. 18. Serum titers of HA-specific antibodies were determined by ELISA following prime, 1st boost, and 2nd boost of antigen. Data are represented as mean antibody titer ±standard deviation (FIGS. 19 and 21).

HI antibody titers of serum from mice immunized with HAB1-H1 were measured using homologous (FIG. 20) as well as heterologous (Table 4) H1N1 viruses. HI assays against heterologous viruses were carried out with pooled sera from HAB1-H3 30 μg immunized group (post 2nd boost). The results are shown in FIG. 20 and in Table 4:

TABLE 4
HI Antibody Titers Measured Using Heterologous Viruses

| | | A/New | A/Solomon |
|---|---|---|---|
| | A/Brisbane/59/07 | Caledonia/20/99 | Islands/3/06 |
| titer: | 181 (±89.9) | 35 (±27.9) | 175 (±57.3) |

HI antibody titers of serum from mice immunized with HAB1-H1 were measured using homologous (FIG. 22) as well as heterologous (Table 5) H1N1 viruses. HI assays against heterologous viruses were carried out with pooled sera from HAB1-H3 30 μg immunized group (post 2nd boost). The results are shown in FIG. 22 and in Table 5:

TABLE 5
HI Antibody Titers Measured Using Heterologous Viruses

| | A/Brisbane/ | A/California/ | A/New York/ | A/Sydney/ | A/Wisconsin/ | A/Wyoming/ |
|---|---|---|---|---|---|---|
| | 10/07 | 07/04 | 55/04 | 5/97 | 67/05 | 03/03 |
| titer: | 160 | 60 | 30 | 5 | 20 | 5 |

### Example 5

### Recombinant HA from A/New Calcdonia/20/99 (H1N1)-HANC3

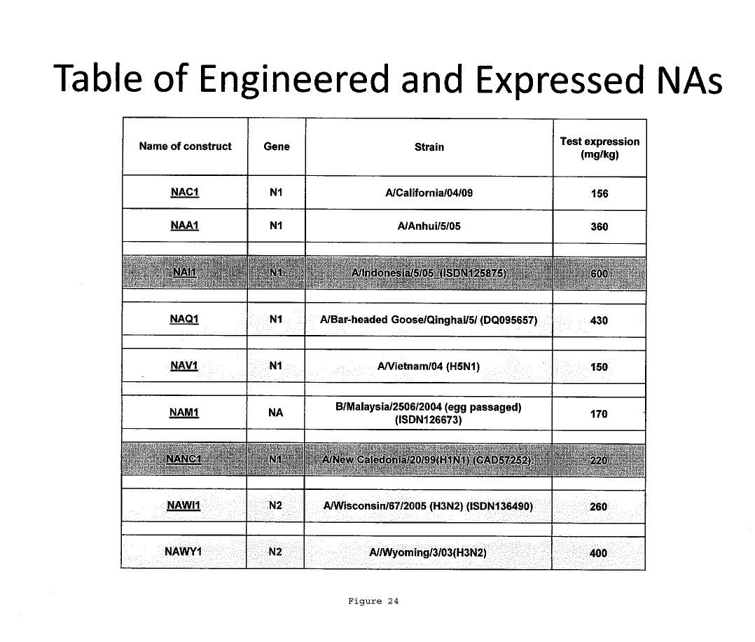

Full-length HA from A/New Calcdonia/20/99 (H1N1)-HANC3 was fused to LicKM. The fusion protein was produced in plants essentially as described in Example 1. Mice were injected three times at 14 day intervals with either 90, 45 or 15 ug HA per dose with Quil A adjuvant as described in Example 2; HA-specific serum antibody responses and hemagglutinin inhibition (HI) were measured as described in Example 2. The results of this experiment are shown in FIG. 25. FIG. 25A shows total IgG responses against A/NC/20/99 and FIG. 25B shows HI activity.

### Example 6

### Immunogenicity of Full-Length Plant-Produced has from Seasonal Influenza Strains

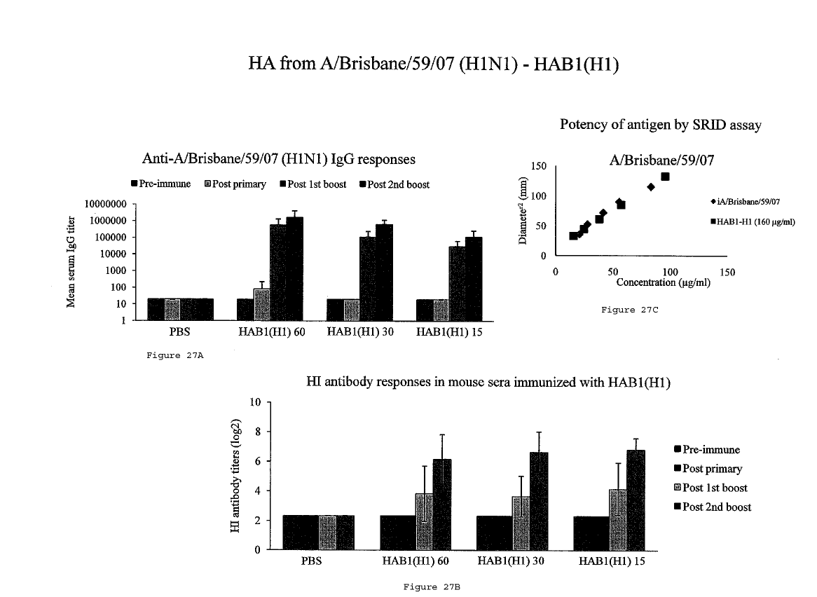

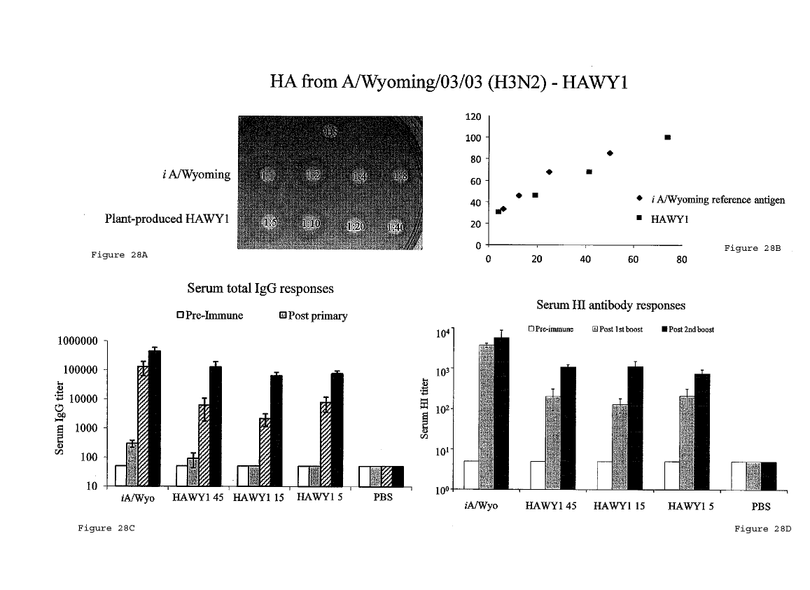

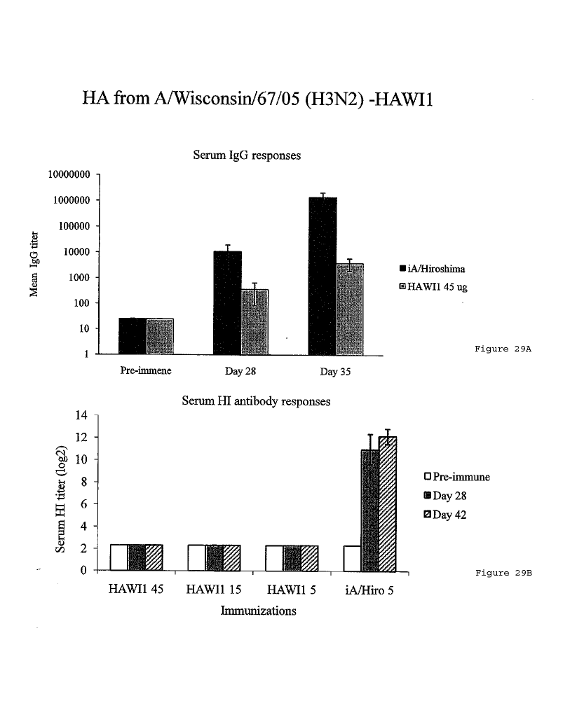

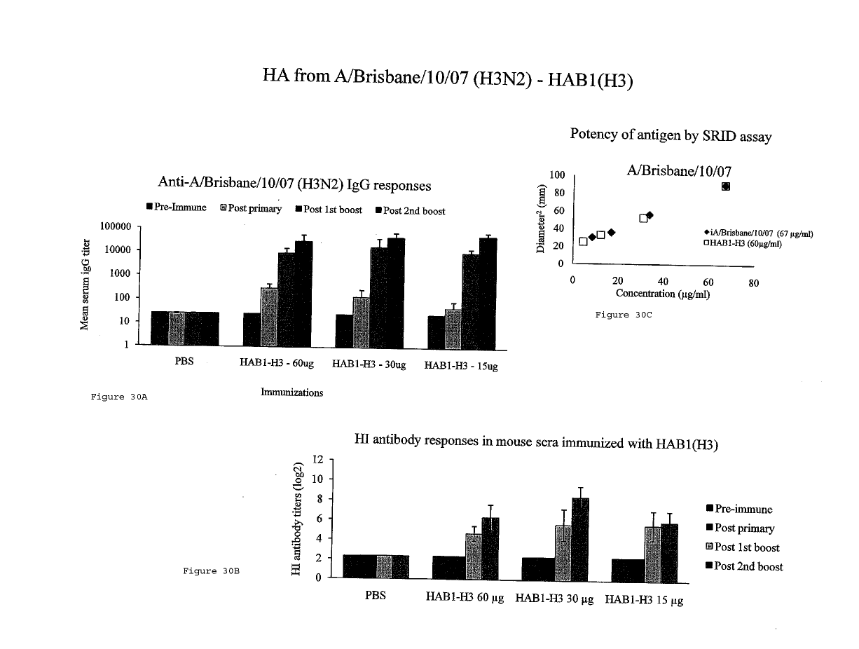

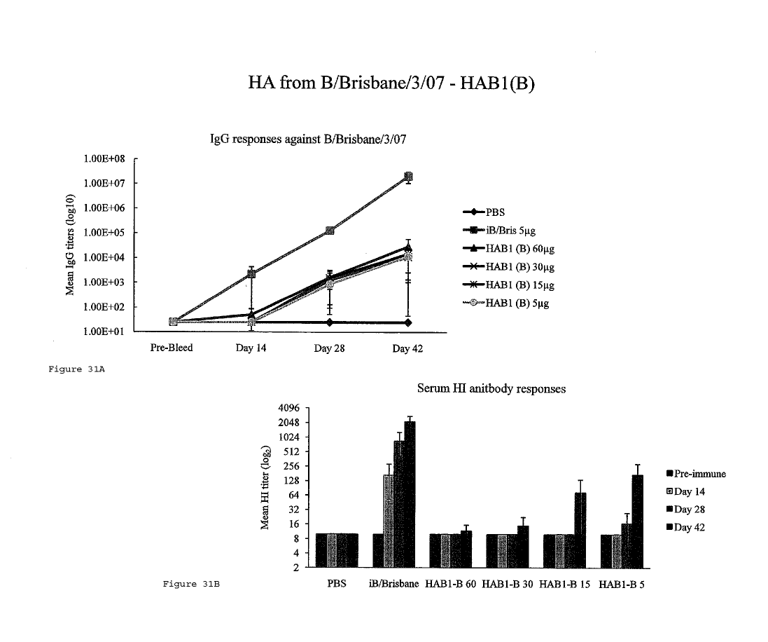

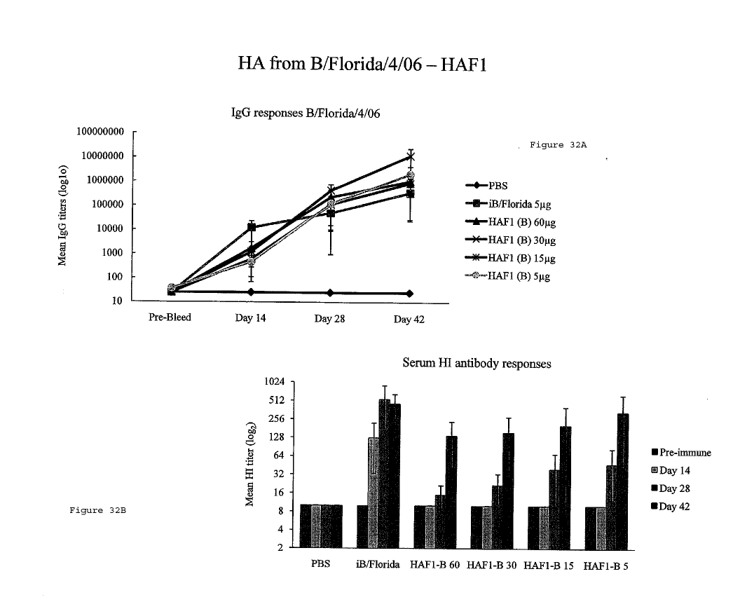

Full-length HAs from seven seasonal influenza strains, A/Solomon Islands/3/06 HASI1, A/Brisbane/59/07 (H1N1)-HAB1(H1), A/Wyoming/03/03 (H3N2)-HAWY1, A/Wisconsin/67/05 (H3N2)-HAWI1, A/Brisbane/10/07 (H3N2)-HAB1(H3), A/Brisbane/3/07 HAB1(B), B/Florida/4/06 HAF1, were assayed for the ability to stimulate an immune response in mice. Recombinant HAs were produced according to the method of Example 1 and used to immunize mice as described in Example 2. HA-specific serum antibody responses and hemagglutinin inhibition (HI) were measured as described in Example 2. The results for the strains A/Solomon Islands/3/06 HASI1, A/Brisbane/59/07 (H1N1)-HAB1(HI), A/Wyoming/03/03 (H3N2)-HAWY1, A/Wisconsin/67/05 (H3N2)-HAWI1, A/Brisbane/10/07 (H3N2)-HAB1(H3), A/Brisbane/3/07 HAB1(B), B/Florida/4/06 HAF1 are shown in FIGS. 25-32, respectively.

### Example 7

### Plant-Produced HA and NA Stimulate an Immune Response in Ferrets

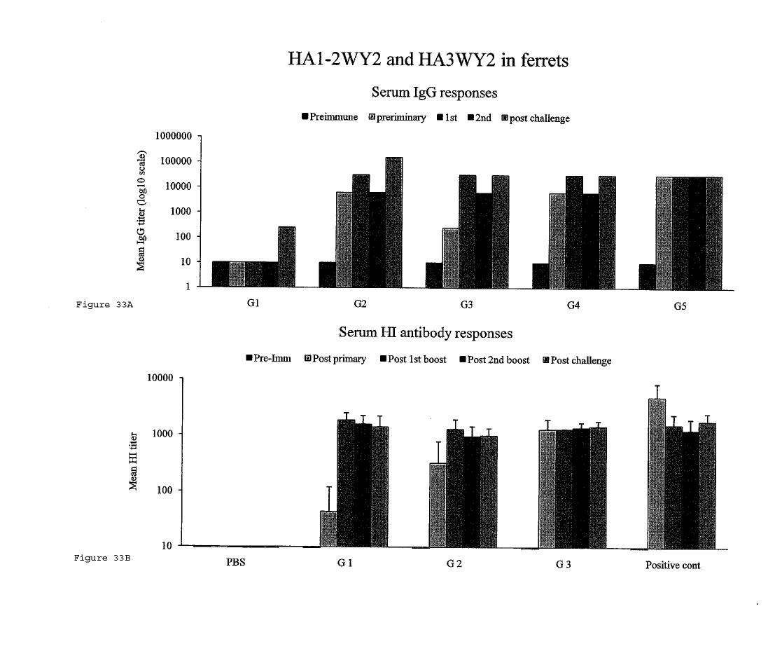

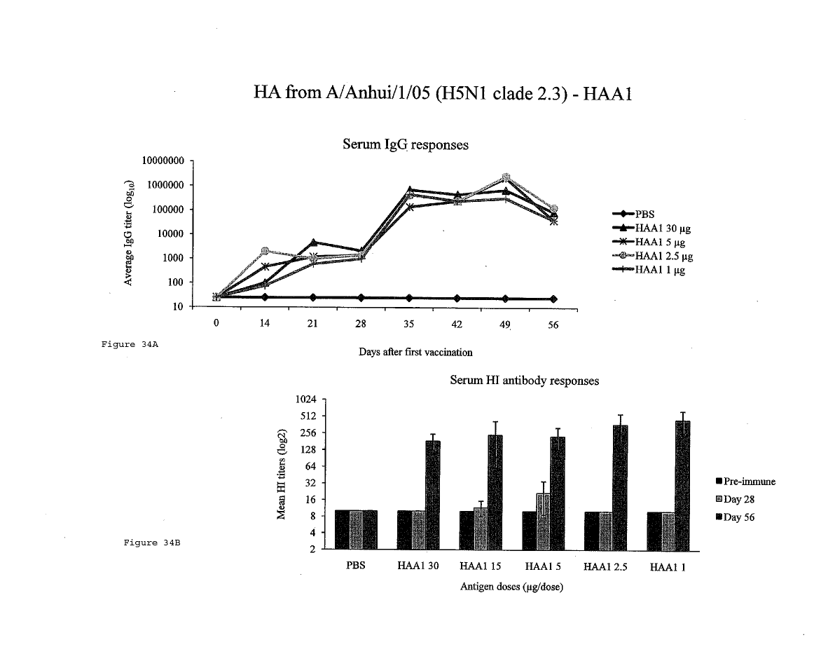

The effect of combinations of HAs and NA to induce an immune response against influenza antigens was assayed in ferrets essentially according to the method of Example 3. Recombinant HAs were produced according to the method of Example 1 and included 1) HA1-2WY2: Domain1-2 (see slide 3) of HA from A/Wyoming/03/03 fused to LicKM; 2) HA3WY2: Domain 3 of HA from A/Wyoming/03/03, fused to LicKM; and 3) full-length NA A/Wyoming/03/03. Feretts were immunized 3 times at 14-day intervals as follows: Group 1: Water with Alhydrogel adjuvant; Group 2: HA1-2WY2, HA3WY2 and NA with Alhydrogel adjuvant; Group 3: HA1-2WY2 and HA3WY2 with Alhydrogel adjuvant; Group 4: HA1-2WY2, HA3WY2 and NA without Alhydrogel adjuvant; Group 5: Live A/Wyoming/03/03 virus with Alhydrogel adjuvant. Ferret serum was collected according to the method of Example 3 and assayed for serum IgG and hemagglutination inhibition; results are shown in FIGS. 33A and 33B, respectively.

### Example 8

### Immunogenicity of Full-Length Plant-Produced HAs from Pandemic Influenza strains

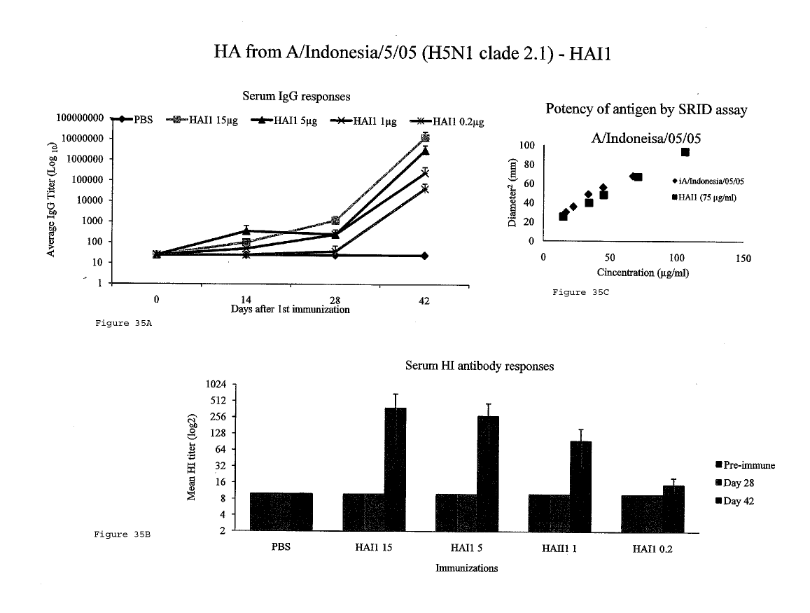

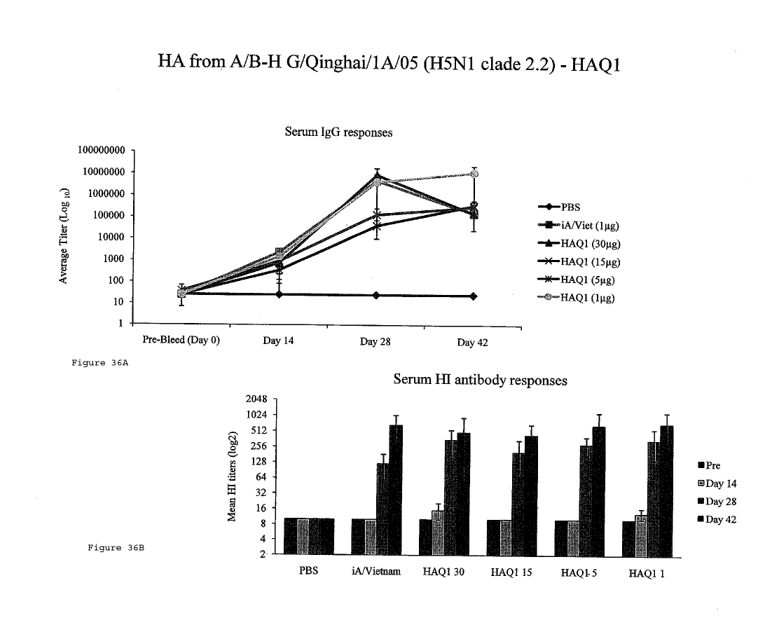

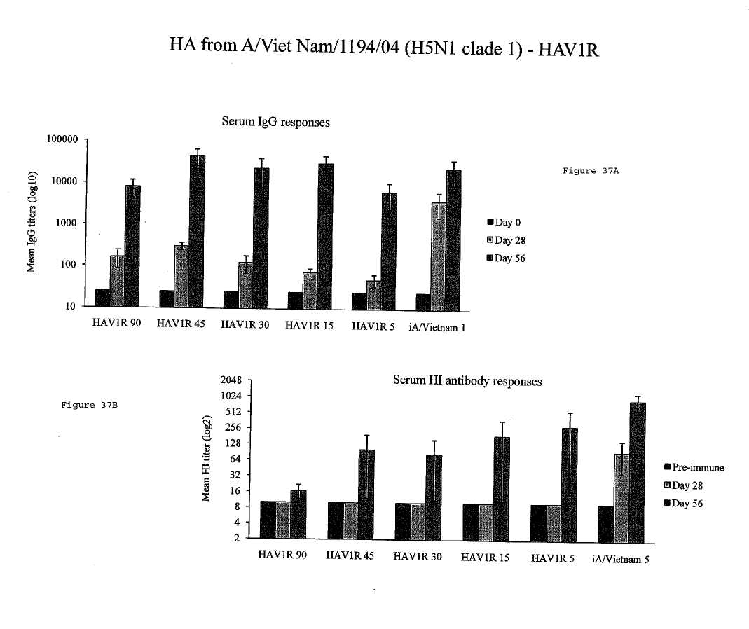

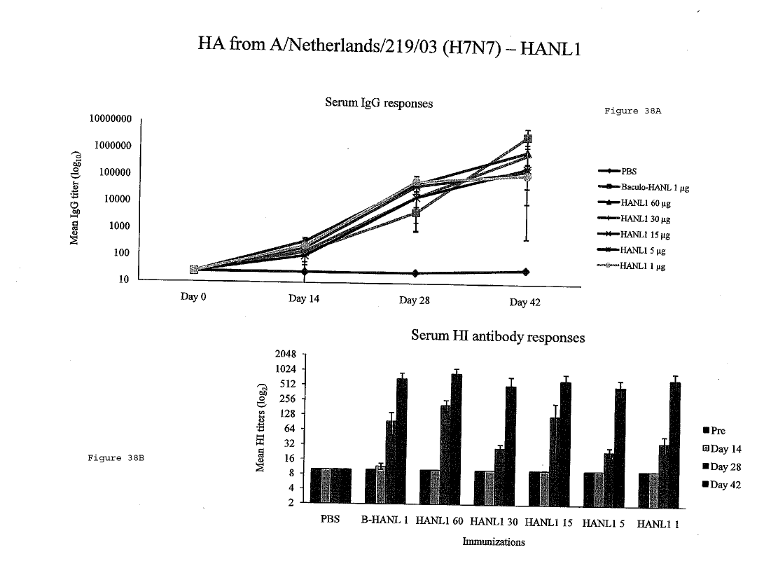

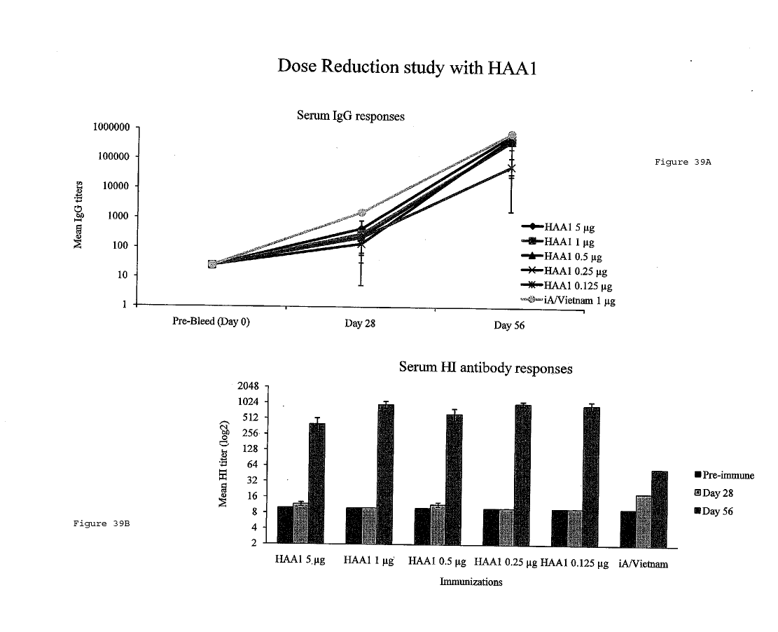

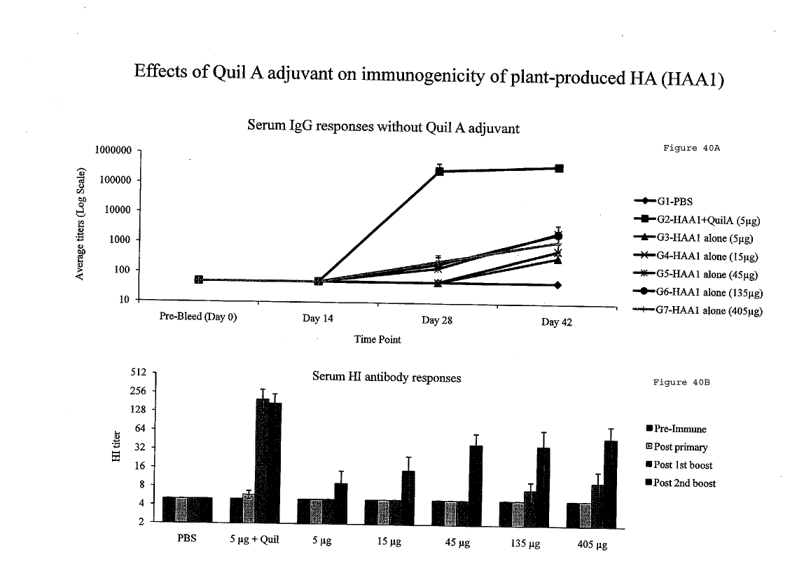

Full-length HAs from five pandemic influenza strains, A/Anhui/1/05 (H5N1 Glade 2.3), A/Indonesia/5/05 (H5N1 Glade 2.1), A/B-H G/Qinghai (H5N1 Glade 2.2), A/Viet Nam/1194/04 (H5N1 Glade 2.2), A/Netherlands/219/03 (H7N7) were assayed for the ability to stimulate an immune response in mice. Recombinant HAs were produced according to the method of Example 1 and used to immunize mice as described in Example 2. HA-specific serum antibody responses and hemagglutinin inhibition (HI) were measured as described in Example 2. The results for the strains A/Anhui/1/05 (H5N1 Glade 2.3), A/Indonesia/5/05 (H5N1 Glade 2.1), A/B-H G/Qinghai (H5N1 Glade 2.2), A/Viet Nam/1194/04 (H5N1 Glade 2.2), A/Netherlands/219/03 (H7N7) are shown in FIGS. 34-38, respectively.

### Example 9

### Dose-Response Analysis of Plant-Produced A/Anhui/1/05

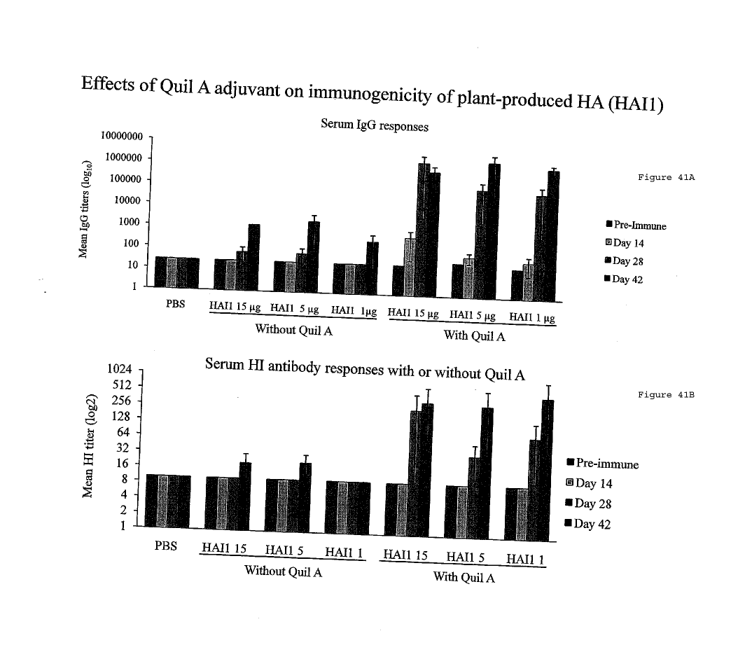

Full-length HA from A/Anhui/1/05 was produced according to the method of Example 1. Mice were injected with either 5 ug, 1 ug, 0.5 ug, 0.025 ug, or 0.125 ug of HA according to the method of Example 2. Serum samples were collected prior to each injection and HA-specific serum antibody responses and hemagglutinin inhibition (HI) were measured as described in Example 2. The results of this experiment are shown in FIG. 39.

### Example 10

### Effect of Quil a Adjuvant on Immunogenicity of Plant-Produced HA

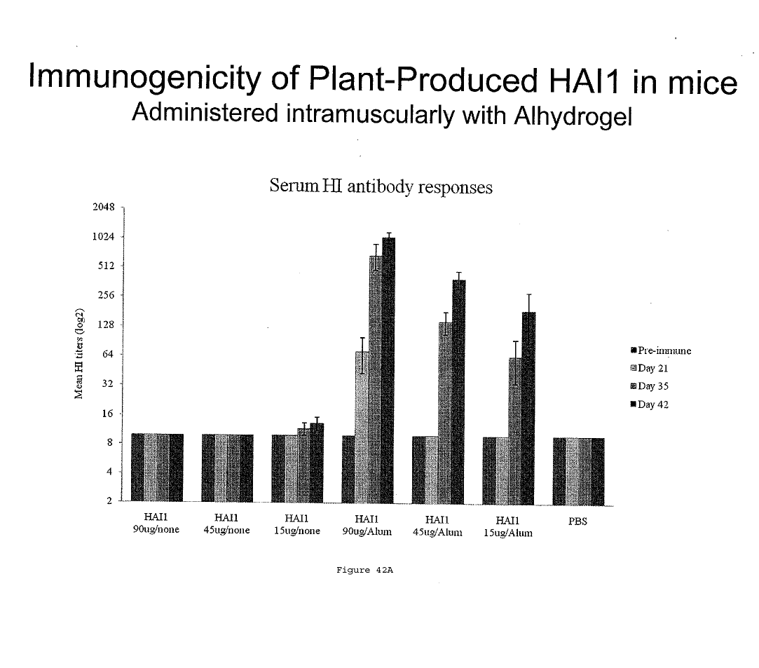

The effect of Quil A adjuvant on immunogenicity of plant-produced HA was assayed with plant-produced full-length HA from A/Anhui/1/05 and plant-produced full-length HA from A/Indonesia/5/05. HAs were produced according to the method of Example 1. For the A/Anhui/1/05 (HAA1) study, seven groups of mice were immunized essentially according to the method described in Example 1, with three injections given at 14-day intervals in the presence or absence of Quil A. The seven groups were as follows: Group 1: PBS; Group 2: 5 ug HAA1 plus Quil A; Group 3: 5 ug HAA1 without Quil A; Group 4: 15 ug HAA1 without Quil A; Group 5: 45 ug HAA1 without Quil A; Group 6: 135 ug HAA1 without Quil A; Group 7: 405 ug HAA1 without Quil A. Serum samples were collected prior to each injection and HA-specific serum antibody responses and hemagglutinin inhibition (HI) were measured as described in Example 2. The results of this experiment are shown in FIG. 40.

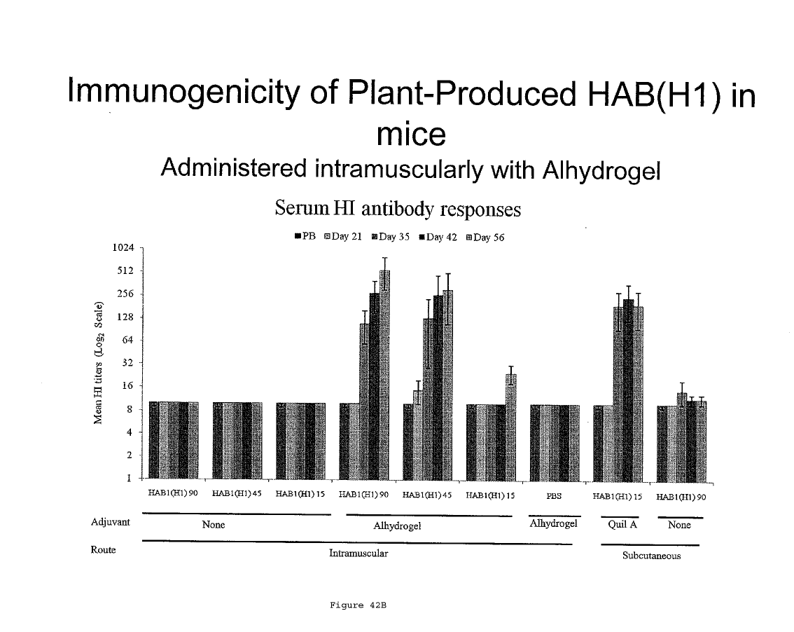

For the A/Indonesia/5/05 (HAI1) study, mice received either 5, 10, or 15 ug of plant-produced HAIL, either with or without Quil A. Serum samples were collected prior to each injection and HA-specific serum antibody responses and hemagglutinin inhibition (HI) were measured as described in Example 2. The results of this experiment are shown in FIG. 41.

### Example 11

### Effect of Alhydrogel Adjuvant on Immunogenicity of Plant-Produced HA

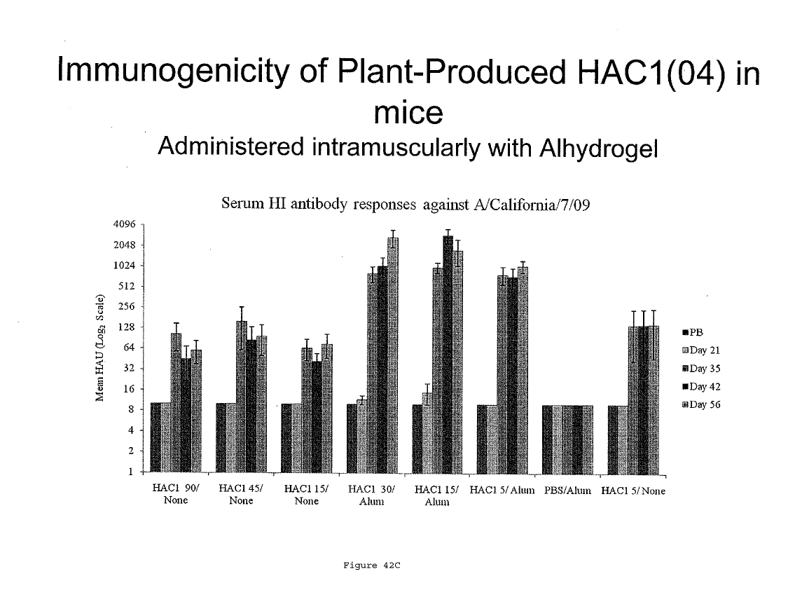

The effect of Alhydrogel on immunogenicity of three different plant-produced HAs, HAI1, HAB (B1) and HAC1(04), was assayed essentially according to the methods described in Examples 1 and 2, except that animals received three injections that were intramuscular instead of sub-cutaneous. Serum samples were collected prior to each injection and assayed for hemagglutinin inhibition activity. The results for HAI1, HAB (B1) and HAC1(04) are shown in FIGS. 42A, 42B and 42C, respectively.

### Example 12

### Production of Recombinant HAs and NAs

Recombinant HAs and NAs from various influenza strains were produced in plants according to the Method of Example 1. Expression levels (mg/kg) for twenty-one different HAs are shown in the Table in FIG. 23. Expression levels ranged from 325 mg/kg for HAF1(B) to 1332 mg/kg for HAB1(H1). Expression levels (mg/kg) for nine different NAs are shown in the Table in FIG. 24. Expression levels ranged from 150 mg/kg for NAV1 to 600 mg/kg for NAIL

### Equivalents and Scope

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any influenza subtype, Glade, strain, etc.; any influenza polypeptide antigen; any expression system; any plant production system; any method of administration; etc.) can be excluded from any one or more claims, for any reason, whether or not related to the existence of prior art.

All references cited herein are incorporated by reference. A number of embodiments of the invention have been described. Nevertheless, it will be understood that various modifications may be made without departing from the spirit and scope of the invention. Accordingly, other embodiments are within the scope of the following claims.

1. A method of making a composition that induces or enhances an immune response against an influenza polypeptide, wherein the polypeptide is a hemagglutinin polypeptide or an immunogenic portion thereof or a neuraminidase polypeptide or immunogenic portion thereof, the method comprising:
a) producing the influenza polypeptide in a plant; and
b) isolating the polypeptide; and
c) combining the polypeptide with a pharmaceutically acceptable carrier.

2. The method of claim 1, wherein the hemagglutinin polypeptide is a polypeptide having 80% or greater sequence identity to an amino acid sequence selected from the group consisting of SEQ ID NOs.: 1-35, 85, 87, 89, 91, 93, 95, 97, 99, 101, 103, 105, 107, 109, 111 and 112.

3-7. (canceled)

8. The method of claim 1, wherein the influenza polypeptide is a neuraminidase polypeptide having 80% or greater sequence identity to an amino acid sequence selected from the group consisting of SEQ ID NOs.: 36-43 and SEQ ID NO: 110.

9-13. (canceled)

14. The method of claim 1, wherein the plant transiently expresses the polypeptide.

15. The method of claim 1, wherein the transient expression is from an Agrobacterial plasmid.

16. The method of claim 1, wherein the transient expression is from an plant viral vector.

17. The method of claim 16, wherein the plant viral vector is cloned into an Agrobacterial plasmid.

18. The method of claim 1, wherein the plant is transgenic for the polypeptide.

19. The method of claim 1, further comprising combining the composition with at least one vaccine adjuvant.

20. The method of claim 19, wherein the adjuvant is selected from the group consisting of alum, Quil A, QS21, aluminum hydroxide, aluminum phosphate, mineral oil, MF59, Malp2, incomplete Freund's adjuvant, complete Freund's adjuvant, alhydrogel, 3 De-O-acylated monophosphoryl lipid A (3D-MPL), lipid A, Bortadella pertussis, Mycobacterium tuberculosis, Merck Adjuvant 65, squalene, virosomes, SBAS2, SBAS1, AS03 and unmethylated CpG sequences.

21. A method of producing an influenza polypeptide, wherein the polypeptide is a hemagglutinin polypeptide or an immunogenic portion thereof or a neuraminidase polypeptide or immunogenic portion thereof, the method comprising:
(a) providing a nucleic acid construct comprising a nucleic acid encoding an influenza polypeptide; and
(b) introducing the nucleic acid into a plant cell; and
(c) maintaining the cell under conditions permitting expression of the influenza polypeptide.

22. The method of claim 21, wherein the nucleic acid encoding the influenza polypeptide is operably linked to a regulatory region.

23. The method of claim 22, wherein the regulatory region is a viral promoter.

24. The method of claim 21, wherein the nucleic acid construct is an Agrobacterial vector.

25. The method of claim 21, wherein the nucleic acid construct comprises one or more sequences encoding a plant viral protein, wherein the plant viral protein comprises an AlMV coat protein or a TMV movement protein.

26. The method of claim 1, wherein the hemagglutinin polypeptide is a polypeptide having 80% or greater sequence identity to an amino acid sequence selected from the group consisting of SEQ ID NOs.: 1-35, 85, 87, 89, 91, 93, 95, 97, 99, 101, 103, 105, 107, 109, 111 and 112.

27-31. (canceled)

32. The method of claim 21, wherein the influenza polypeptide is a neuraminidase polypeptide having 80% or greater sequence identity to an amino acid sequence selected from the group consisting of SEQ ID NOs.: 36-43 and SEQ ID NO: 110.

33-37. (canceled)

38. The method of claim 1, wherein the method further comprises isolating the influenza polypeptide.

39. The method of claim 1, wherein the isolated influenza polypeptide is at least about 70% pure.

40-43. (canceled)

44. The method of 1, wherein the plant is from a genus selected from the group consisting of Brassica, Nicotiana, Petunia, Lycopersicon, Solanum, Capsium, Daucus, Apium, Lactuca, Sinapis or Arabidopsis.

45. The method of 1, wherein the plant is from a species selected from the group consisting of Nicotiana benthamiana, Brassica carinata, Brassica juncea, Brassica napus, Brassica nigra, Brassica oleraceae, Brassica tournifortii, Sinapis alba, and Raphanus sativus.

46. The method of 1, wherein the plant is selected from the group consisting of alfalfa, radish, mustard, mung bean, broccoli, watercress, soybean, wheat, sunflower, cabbage, clover, petunia, tomato, potato, tobacco, spinach, and lentil.

47. The method of 1, wherein the plant is a sprouted seedling.

48. The method of claim 1, wherein step (a) is performed by the method of claim 21.

49. A method of inducing or enhancing an immune response against an influenza polypeptide in a subject, the method comprising administering a therapeutically effective amount of the composition produced by the method of claim 1.

50-52. (canceled)

53. A method of inducing or enhancing an immune response against an influenza polypeptide in a subject, the method comprising feeding a plant or plant cell produced by the method of claim 21 to a subject.

54. The method of claim 49, wherein the subject is human.

55. The method of claim 49, wherein the subject is a bird, a pig or a horse.

56. The method of claim 49, wherein a single dose of the composition comprises up to about 200 μg of the influenza polypeptide.

57-64. (canceled)


SEQUENCE LISTING (115 sequences)

SEQ ID NO: 1 - protein, 552 aa, Influenza A virus
MNIQILAFIACVLTGAKGDKICLGHHAVANGTKVNTLTEKGIEVVNATETVETADVKKICTQGKRATDLGRCGLLGTLIGPPQCDQFLEFSSDLIIERREGTDVCYPGRFTNEESLRQILRRSGGIGKESMGFTYSGIRTNGAASACTRSGSSFYAEMKWLLSNSDNSAFPQMTKAYRNPRNKPALIIWGVHHSESASEQTKLYGSGNKLITVRSSKYQQSFTPSPGTRRIDFHWLLLDPNDTVTFTFNGAFIAPDRASFFRGESLGVQSDAPLDSSCRGDCFHSGGTIVSSLPFQNINSRTVGRCPRYVKQKSLLLATGMRNVPEKPKPRGLFGAIAGFIENGWEGLINGWYGFRHQNAQGEGTAADYKSTQSAIDQITGKLNRLIGKTNQQFELIDNEFNEIEQQIGNVINWTRDAMTEIWSYNAELLVAMENQHTIDLADSEMSKLYERVKKQLRENAEEDGTGCFEIFHKCDDQCMESIRNNTYDHTQYRTESLQNRIQIDPVKLSSGYKDIILWFSFGASCFILLAIAMGLVFICIKNGNMQCTICI

SEQ ID NO: 2 - protein, 552 aa, Influenza A virus
MNTQILAFIACVLTGVKGDKICLGHHAVANGTKVNTLTEKGIEVVNATETVETADVKKICTQGKRATDLGRCGLLGTLIGPPQCDQFLEFSSDLIIERREGTDVCYPGRFTNEESLRQILRRSGGIGKESMGFTYSGIRTNGATSACTRSGSSFYAEMKWLLSNSDNSAFPQMTKAYRNPRNKPALIIWGVHHSESVSEQTKLYGSGNKLITVRSSKYQQSFTPSPGARRIDFHWLLLDPNDTVTFTFNGAFIAPDRASFFRGESLGVQSDVPLDSSCRGDCFHSGGTIVSSLPFQNINSRTVGKCPRYVKQKSLLLATGMRNVPEKPKPRGLFGAIAGFIENGWEGLINGWYGFRHQNAQGEGTAADYKSTQSAIDQITGKLNRLIGKTNQQFELIDNEFNEIEQQIGNVINWTRDAMTEIWSYNAELLVAMENQHTIDLADSEMSKLYERVKKQLRENAEEDGTGCFEIFHKCDDQCMESIRNNTYDHTQYRTESLQNRIQIDPVKLSSGYKDIILWFSFGASCFLLLAIAMGLVFICIKNGNMQCTICI

SEQ ID NO: 3 - protein, 552 aa, Influenza A virus
MNIQILAFIACVLTGAKGDKICLGHHAVANGTKVNTLTEKGIEVVNATETVETANIKKICTQGKRPTDLGQCGLLGTLIGPPQCDQFLEFSSDLIIERREGTDVCYPGKFTNEESLRQILRRSGGIGKESMGFTYSGIRTNGATSACTRSGSSFYAEMKWLLSNSDNAAFPQMTKSYRNPRNKPALIIWGVHHSESVSEQTKLYGSGNKLIKVRSSKYQQSFTPNPGARRIDFHWLLLDPNDTVTFTFNGAFIAPDRASFFRGESIGVQSDAPLDSSCGGNCFHNGGTIVSSLPFQNINPRTVGKCPRYVKQKSLLLATGMRNVPEKPKKRGLFGAIAGFIENGWEGLINGWYGFRHQNAQGEGTAADYKSTQSAIDQITGKLNRLIGKTNQQFELINNEFNEVEQQIGNVINWTQDAMTEVWSYNAELLVAMENQHTIDLTDSEMSKLYERVRKQLRENAEEDGTGCFEIFHKCDDHCMESIRNNTYDHTQYRTESLQNRIQIDPVKLSGGYKDIILWFSFGASCFLLLAIAMGLVFICIKNGNMQCTICI

SEQ ID NO: 4 - protein, 560 aa, Influenza A virus
MNTQILALIAYMLIGAKGDKICLGHHAVANGTKVNTLTERGIEVVNATETVETVNIKKICTQGKRPTDLGQCGLLGTLIGPPQCDQFLEFDADLIIERREGTDVCYPGKFTNEESLRQILRGSGGIDKESMGFTYSGIRTNGVTSACRRSGSSFYAEMKWLLSNSDNAAFPQMTKSYRNPRNKPALIIWGVHHSGSATEQTKLYGSGNKLITVGSSKYQQSFTPSPGARPQVNGQSGRIDFHWLLLDPNDTVTFTFNGAFIAPDRASFFRGESLGVQSDVPLDSGCEGDCFHSRGTIVSSLPFQNINPRTVGKCPRYVKQTSLLLATGMRNVPENPKTRGLFGAIAGFIENGWEGLIDGWYGFRHQNAQGEGTAADYKSTQSAIDQITGKLNRLIDKTNQQFELIDNEFSEIEQQIGNVINWTRDSMTEVWSYNAELLVAMENQHTIDLADSEMNKLYERVRKQLRENAEEDGTGCFEIFHKCDDQCMESIRNNTYDHTQYRTESLQNRIQIDPVKLSSGYKDIILWFSFGASCFLLLAIAMGLVFICIKNGNMRCTICI

SEQ ID NO: 5 - protein, 560 aa, Influenza A virus
MNTQILALIACMLIGAKGDKICLGHHAVANGTKVNTLTERGIEVVNATETVETANIKKICTQGKRPTDLGQCGLLGTLIGPPQCDQFLEFDADLIIERREGTDVCYPGKFTNEESLRQILRGSGGIDKESMGFTYSGIRTNGATSACRRSGSSFYAEMKWLLSNSDNAAFPQMTKSYRNPRNKPALIIWGVHHSGSATEQTKLYGSGNKLITVGSSKYQQSFTPSPGARPQVNGQSGRIDFHWLLLDPNDTVTFTFNGAFIAPDRASFFRGGSLGVQSDVPLDSGCEGDCFHSGGTIVSSLPFQNINPRTVGKCPRYVKQTSLLLATGMRNVPENPKTRGLFGAIAGFIENGWEGLIDGWYGFRHQNAQGEGTAADYKSTQSAIDQITGKLNRLIDKTNQQFELIDNEFNEIEQQIGNVINWTRDSMTEVWSYNAELLVAMENQHTIDLADSEMNKLYERVRKQLRENAEEDGTGCFEIFHKCDDQCMESIRNNTYDHTQYRTESLQNRIQINPVKLSSGYKDIILWFSFGASCFLLLAIAMGLVFICIKNGNMRCTICI

SEQ ID NO: 6 - protein, 568 aa, Influenza A virus
MEKIVLLLAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLCYPGDFNDYEELKHLLSRINHFEKIQIIPKSSWSDYEASSGVSSACPYQGRSSFFRNVVWLIKKNNAYPTIKRSYNNTNQEDLLVLWGIHHPNDAAEQIRLYQNPTTYISIGTSTLNQRLVPKIATRSKVNGQSGRMEFFWTILKSNDAINFESNGNFIAPEYAYKIVKKGDSTIMKSELEYGNCNTKCQTPIGAINSSMPFHNIHPLTIGECPKYVKSNRLVLATGLRNSPQGERRRRKRGLFGAIAGFIEGGWQGMVDGWYGYHHSNEQGSGYAADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESMGIYQILSIYSTVASSLALAIMVAGLFLWMCSNGSLQCRICI

SEQ ID NO: 7 - protein, 552 aa, Influenza A virus
DQICIGYHANNSTEQVDTIMEKNVTVTHAQNILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSNKLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKIRLQLRDNAKELGNGCFEFYHRCDNECMESVRNGTYDYPQYSEEARLKREEISGVKLESIGTYQILSIYSTVASSLTLAIMVAGLSLWMCSNGSLQCRICI

SEQ ID NO: 8 - protein, 567 aa, Influenza A virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIISKSSWSDHEASSGVSSACPYQGTPSFFRNVVWLIKKNNTYPTIKRSYNNTNQEDLLILWGIHHSNNAAEQTKLYQNPTTYISVGTSTLNLRLVPKIATRSKVNGQSGRMDFFWTILKPNDAINFESNGNFIAPEYAYKIVKKGDSAIMKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 9 - protein, 563 aa, Influenza A virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKTSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRIC

SEQ ID NO: 10 - protein, 552 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILNPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 11 - protein, 566 aa, Influenza A virus
MKTIIALSYILCLVSAQKFPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEEFDKLYIWGVHHPGTDNDQIFLYAQASGRITVSTKRSQQTVIPNIRSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 12 - protein, 566 aa, Influenza A virus
MKTIIALSYILCLVFAQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFKNESFNWTGVTQNGTSSACIRRSNNSFFSRLNWLTHLKFKYPALNVTMPNKEKFDKLYIWGVHHPGTDNDQIFLYAQASGRITVSTKRSQQTVIPNIGSRLRVRDIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 13 - protein, 566 aa, Influenza A virus
MKTIIALSYILCLVFAQKLPGNDNSTATLCLGHHAVPNGTIVKTITNDQIEVTNATELVQSSSTGEICDSPHQILDGENCTLIDALLGDPQCDGFQNKKWDLFVERSKAYSKCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFLYAQASGRITVSTKRSQQTVIPNIGSRPRVRDIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 14 - protein, 566 aa, Influenza A virus
MKTIIALSYILCLVFAQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFLYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 15 - protein, 565 aa, Influenza A virus
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYHWTLLEPGDTIIFEANGNLIAPRYAFTLSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 16 - protein, 565 aa, Influenza A virus
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKTLYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 17 - protein, 565 aa, Influenza A virus
  MOD_RES (158)..(158): Any amino acid
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESAWPNHTVTGVSASCSHNGEXSFYRNLLWLTXKNGLYPNLSKSYANNKEKEVLVLWGVHHPPNIGDQKALYHTENAYVSVVSSHYSRKFTPEIAKRPKVRXQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNXKNLYEKVKSQLKNNAKEIGNGCFEFYHKCNDECMESVKNGTXDYPKYSEESKLNREKIDGVKLESMGVYQILAIYSTVASSLVLLVSLGAISFWMCSNGSLQCRICI

SEQ ID NO: 18 - protein, 565 aa, Influenza A virus
  MOD_RES (178)..(178): Any amino acid
MKVKLLVLLCTFTATYADTICIGYHANNSTDTVDTVLEKNVTVTHSVNLLENNHNGKLCLLKGIAPLQLGNCSVAGWILGNPECELLISKESWSYIVEKPNPENGTCYPGHFADYEELREQLSSVSSFERFEMFPKEGSWPNHTVTGVSASCSHNGESSFYRNLLWLTGKNGLYPNLXKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDNCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVXKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 19 - protein, 585 aa, Influenza A virus
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 20 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 21 - protein, 566 aa, Influenza A virus
MKAILVVMLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 22 - protein, 567 aa, Influenza A virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGTPSFFRNVVWLIKKNNTYPTIKRSYNNTNQEDLLILWGIHHSNDAAEQTKLYQNPTTYISVGTSTLNQRLVPKIATRSKVNGQSGRMDFFWTILKPNDAINFESNGNFIAPEYAYKIVKKGDSAIVKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 23 - protein, 568 aa, Influenza A virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 24 - protein, 560 aa, Influenza A virus
MERIVLLLAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFERIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKNCQKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNG

SEQ ID NO: 25 - protein, 568 aa, Influenza A virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 26 - protein, 584 aa, Influenza A virus
MKAIIVLLMVVTSNADRICTGITSSNSPHVVKTATQGEVNVTGVIPLTTTPTKSYFANLKGTKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 27 - protein, 565 aa, Influenza A virus
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 28 - protein, 548 aa, Influenza A virus
QKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNI

SEQ ID NO: 29 - protein, 584 aa, Influenza A virus
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 30 - protein, 586 aa, Influenza A virus
MKAIIVLLMVVTSNADRIICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 31 - protein, 565 aa, Influenza A virus
MKAKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 32 - protein, 565 aa, Influenza A virus
MKVKLLVLLCTFTATYADTICIGYHANNSTDTVDTVLEKNVTVTHSVNLLEDSHNGKLCLLKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 33 - protein, 566 aa, Influenza A virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNDESFNWTGVTQNGTSSSCKRRSNNSFFSRLNWLTHLKFKYPALNVTMPNNEKFDKLYIWGVHHPVTDNDQIFLYAQASGRITVSTKRSQQTVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 34 - protein, 566 aa, Influenza A virus
MKTIIALSYILCLVFSQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWAGVTQNGTSSACKRRSNKSFFSRLNWLTHLKYKYPALNVTMPNNEKFDKLYIWGVHHPVTDSDQISLYAQASGRITVSTKRSQQTVIPNIGYRPRVRDISSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIESIRNGTYDHDVYRDEALNNRFQIKGVELKSGYKDWILWISFAISCFLLCVALLGFIMWACQKGNIRCNICI

SEQ ID NO: 35 - protein, 569 aa, Influenza A virus
SKSRGYKMNTQILVFALVASIPTNADKICLGHHAVSNGTKVNTLTERGVEVVNATETVERTNVPRICSKGKRTVDLGQCGLLGTITGPPQCDQFLEFSADLIIERREGSDVCYPGKFVNEEALRQILRESGGIDKETMGFTYSGIRTNGTTSACRRSGSSFYAEMKWLLSNTDNAAFPQMTKSYKNTRKDPALIIWGIHHSGSTTEQTKLYGSGNKLITVGSSNYQQSFVPSPGARPQVNGQSGRIDFHWLILNPNDTVTFSFNGAFIAPDRASFLRGKSMGIQSEVQVDANCEGDCYHSGGTIISNLPFQNINSRAVGKCPRYVKQESLLLATGMKNVPEIPKRRRRGLFGAIAGFIENGWEGLIDGWYGFRHQNAQGEGTAADYKSTQSAIDQITGKLNRLIEKTNQQFELIDNEFTEVERQIGNVINWTRDSMTEVWSYNAELLVAMENQHTIDLADSEMNKLYERVKRQLRENAEEDGTGCFEIFHKCDDDCMASIRNNTYDHSKYREEAIQNRIQIDPVKLSSGYKDVILWFSFGASCFILLAIAMGLVFICVKNGNMRCTICI

SEQ ID NO: 36 - protein, 449 aa, Influenza A virus
MNPNQKIITIGSICMVTGIVSLMLQIGNMISIWVSHSIHTGNQHQSEPISNTNLLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPDAGEITCVCRDNWHGSNRPWVSFNQNLEYQIGYICSGVFGDNPRPNDGTGSCGPVSSNGAGGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 37 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDENATASFIYNGRLVDSIVSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKQETEVLWTSNSIVVFCGTSGTYGTGSWPDGADINLMPI

SEQ ID NO: 38 - protein, 449 aa, Influenza A virus
MNPNQKIITIGSICMVIGIVSLMLQIGNMISIWVSHSIQTGNQHQAEPIRNANFLTENAVASVTLAGNSSLCPVRGWAVHSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASYKIFKMEKGKVVKSVELNAPNYHYEECSCYPGAGEITCVCRDNWHGSNRPWVSFNQNLEYQIGYICSGVFGDNPRPNDGTGSCGPVSPNGAYGIKGFSFKYGNGVWIGRTKSTNSRSGFEMIWDPNGWTETDSNFSVKQDIVAITDWSGYSGSFVQHPELTGLDCIRPCFWVELIRGRPKESTIWTSGSSISFCGVNSDTVGWSWPDGAELPFTIDK

SEQ ID NO: 39 - protein, 436 aa, Influenza A virus
MNPNQKIITIGSICMVIGIVSLMLQIGNMISIWVIHSIQTGNQHQAESISNTNPLTEKAVASVTLAGNSSLCPIRGWAVHSKDNNIRIGSKGDVFVIREPFISCSHLECRTFFLTQGALLNDKHSNGTVKDRSPHRTLMSCPVGEAPSPYNSRFESVAWSASACHDGTSWLTIGISGPDNEAVAVLKYNGIITDTIKSWRNDILRTQESECACVNGSCFTVMTDGPSNGQASYKIFKMEKGKVVKSVELDAPNYHYEECSCYPDAGEITCVCRDNWHGSNRPWVSFNQNLEYQIGYICSGVFGDNPRPNDGTGSCGPMSPNGAYGVKGFSFKYGNGVWIGRTKSTNSRSGFEMIWDPNGWTGTDSSFSVKQDIVAITDWSGYSGSFVQHPELTGLDCIRPCFWVELIRGRPKESTIWTSGSSISFCGVNSDTVSWS

SEQ ID NO: 40 - protein, 449 aa, Influenza A virus
MNPNQKIITIGSICMVIGIVSLMLQIGNMISIWVSHSIQTGNQRQAEPISNTKFLTEKAVASVTLAGNSSLCPISGWAVYSKDNSIRIGSRGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASYKIFKMEKGKVVKSVELDAPNYHYEECSCYPDAGEITCVCRDNWHGSNRPWVSFNQNLEYQIGYICSGVFGDNPRPNDGTGSCGPVSPNGAYGVKGFSFKYGNGVWIGRTKSTNSRSGFEMIWDPNGWTGTDSSFSVKQDIVAITDWSGYSGSFVQHPELTGLDCIRPCFWVELIRGRPKESTIWTSGSSISFCGVNSDTVSWSWPDGAELPFTIDK

SEQ ID NO: 41 - protein, 466 aa, Influenza A virus
MLPSTIQTLTLFLTSGGVLLSLYVSASLSYLLYSDILLKFPSTEITAPTMPLDCANASNVQAVNRSATKGVTLLLPEPEWTYPRLSCPGSTFQKALLISPHRFGETKGNSAPLIIREPFIACGPKECKHFALTHYAAQPGGYYNGTRGDRNKLRHLISVKLGKIPTVENSIFHMAAWSGSACHDGKEWTYIGVDGPDNNALLKIKYGEAYTDTYHSYANNILRTQESACNCIGGNCYLMITDGSASGVSECRFLKIREGRIIKEIFPTGRIKHTEECTCGFASNKTIECACRDNSYTAKRPFVKLNVETDTAEIRLMCTETYLDTPRPDDGSITGPCESNGDKGSGGIKGGFVHQRMASKIGRWYSRTMSKTKRMGMGLYVKYDGDPWADSDALAFSGVMVSMEEPGWYSFGFEIKDKKCDVPCIGIEMVHDGGKETWHSAATAIYCLMGSGQLLWDTVTGVNMAL

SEQ ID NO: 42 - protein, 470 aa, Influenza A virus
MNPNQKIITIGSISIAIGIISLMLQIGNIISIWASHSIQTGSQNHTGVCNQRIITYENSTWVNHTYVNINNTNVVAGKDKTSVTLAGNSSLCSISGWAIYTKDNSIRIGSKGDVFVIREPFISCSHLECRTFFLTQGALLNDKHSNGTVKDRSPYRALMSCPLGEAPSPYNSKFESVAWSASACHDGMGWLTIGISGPDNGAVAVLKYNGIITETIKSWKKRILRTQESECVCVNGSCFTIMTDGPSNGAASYKIFKIEKGKVTKSIELNAPNFHYEECSCYPDTGTVMCVCRDNWHGSNRPWVSFNQNLDYQIGYICSGVFGDNPRPKDGEGSCNPVTVDGADGVKGFSYKYGNGVWIGRTKSNRLRKGFEMIWDPNGWTDTDSDFSVKQDVVAITDWSGYSGSFVQHPELTGLDCIRPCFWVELVRGLPRENTTIWTSGSSISFCGVNSDTANWSWPDGAELPFTIDK

SEQ ID NO: 43 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 44 - protein, 333 aa, Clostridium thermocellum
MKNRVISLLMASLLLVLSVIVAPFYKAEAATVVNTPFVAVFSNFDSSQWEADWANGSVFNCVWKPSQVTFSNGKMILTLDREYGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGVPQDNPTPTPTIAPSTPTNPNLPLKGDVNGDGHVNSSDYSLFKRYLLRVIDRFPVGDQSVADVNRDGRIDSTDLTMLKRYLIRAIPSL

SEQ ID NO: 45 - protein, 259 aa, Brevibacillus brevis
MVKSKYLVFISVFSLLFGVFVVGFSHQGVKAEEERPMGTAFYESFDAFDDERWSKAGVWTNGQMFNATWYPEQVTADGLMRLTIAKKTTSARNYKAGELRTNDFYHYGLFEVSMKPAKVEGTVSSFFTYTGEWDWDGDPWDEIDIEFLGKDTTRIQFNYFTNGVGGNEFYYDLGFDASESFNTYAFEWREDSITWYVNGEAVHTATENIPQTPQKIMMNLWPGVGVDGWTGVFDGDNTPVYSYYDWVRYTPLQNYQIHQ

SEQ ID NO: 46 - protein, 349 aa, Fibrobacter succinogenes
MNIKKTAVKSALAVAAAAAALTTNVSAKDFSGAELYTLEEVQYGKFEARMKMAAASGTVSSMFLYQNGSEIADGRPWVEVDIEVLGKNPGSFQSNIITGKAGAQKTSEKHHAVSPAADQAFHTYGLEWTPNYVRWTVDGQEVRKTEGGQVSNLTGTQGLRFNLWSSESAAWVGQFDESKLPLFQFINWVKVYKYTPGQGEGGSDFTLDWTDNFDTFDGSRWGKGDWTFDGNRVDLTDKNIYSRDGMLILALTRKGQESFNGQVPRDDEPAPQSSSSAPASSSSVPASSSSVPASSSSAFVPPSSSSATNAIHGMRTTPAVAKEHRNLVNAKGAKVNPNGHKRYRVNFEH

SEQ ID NO: 47 - protein, 309 aa, Streptomyces coelicolor
MVNRRDLIKWSAVALGAGAGLAGPAPAAHAADLEWEQYPVPAAPGGNRSWQLLPSHSDDFNYTGKPQTFRGRWLDQHKDGWSGPANSLYSARHSWVADGNLIVEGRRAPDGRVYCGYVTSRTPVEYPLYTEVLMRVSGLKLSSNFWLLSRDDVNEIDVIECYGNESLHGKHMNTAYHIFQRNPFTELARSQKGYFADGSYGYNGETGQVFGDGAGQPLLRNGFHRYGVHWISATEFDFYFNGRLVRRLNRSNDLRDPRSRFFDQPMHLILNTESHQWRVDRGIEPTDAELADPSINNIYYRWVRTYQAV

SEQ ID NO: 48 - protein, 682 aa, Bacillus circulans
MKPSHFTEKRFMKKVLGLFLVVVMLASVGVLPTSKVQAAGTTVTSMEYFSPADGPVISKSGVGKASYGFVMPKFNGGSATWNDVYSDVGVNVKVGNNWVDIDQAGGYIYNQNWGHWSDGGFNGYWFTLSATTEIQLYSKANGVKLEYQLVFQNINKTTITAMNPTQGPQITASFTGGAGFTYPTFNNDSAVTYEAVADDLKVYVKPVNSSSWIDIDNNAASGWIYDHNFGQFTDGGGGYWFNVTESINVKLESKTSSANLVYTITFNEPTRNSYVITPYEGTTFTADANGSIGIPLPKIDGGAPIAKELGNFVYQININGQWVDLSNSSQSKFAYSANGYNNMSDANQWGYWADYIYGLWFQPIQENMQIRIGYPLNGQAGGNIGNNFVNYTFIGNPNAPRPDVSDQEDISIGTPTDPAIAGMNLIWQDEFNGTTLDTSKWNYETGYYLNNDPATWGWGNAELQHYTNSTQNVYVQDGKLNIKAMNDSKSFPQDPNRYAQYSSGKINTKDKLSLKYGRVDFRAKLPTGDGVWPALWMLPKDSVYGTWAASGEIDVMEARGRLPGSVSGTIHFGGQWPVNQSSGGDYHFPEGQTFANDYHVYSVVWEEDNIKWYVDGKFFYKVTNQQWYSTAAPNNPNAPFDEPFYLIMNLAVGGNFDGGRTPNASDIPATMQVDYVRVYKEQ

SEQ ID NO: 49 - protein, 243 aa, Bacillus licheniformis
MSYRVKRMLMLLVTGLFLSLSTFAASASAQTGGSFYEPFNNYNTGLWQKADGYSNGNMFNCTWRANNVSMTSLGEMRLSLTSPSYNKFDCGENRSVQTYGYGLYEVNMKPAKNVGIVSSFFTYTGPTDGTPWDEIDIEFLGKDTTKVQFNYYTNGVGNHEKIVNLGFDAANSYHTYAFDWQPNSIKWYVDGQLKHTATTQIPQTPGKIMMNLWNGAGVDEWLGSYNGVTPLSRSLHWVRYTKR

SEQ ID NO: 50 - protein, 238 aa, Paenibacillus polymyxa
MMKKKSWFTLMITGVISLFFSVSAFAGNVFWEPLSYFNSSTWQKADGYSNGQMFNCTWRANNVNFTNDGKLKLSLTSPANNKFDCGEYRSTNNYGYGLYEVSMKPAKNTGIVSSFFTYTGPSHGTQWDEIDIEFLGKDTTKVQFNYYTNGVGGHEKIINLGFDASTSFHTYAFDWQPGYIKWYVDGVLKHTATTNIPSTPGKIMMNLWNGTGVDSWLGSYNGANPLYAEYDWVKYTSN

SEQ ID NO: 51 - protein, 259 aa, Brevibacillus brevis; identical to SEQ ID NO: 45

SEQ ID NO: 52 - protein, 286 aa, Rhodothermus marinus
MCTMPLMKLKKMMRRTAFLLSVLIGCSMLGSDRSDKAPHWELVWSDEFDYSGLPDPEKWDYDVGGHGWGNQELQYYTRARIENARVGGGVLIIEARHEPYEGREYTSARLVTRGKASWTYGRFEIRARLPSGRGTWPAIWMLPDRQTYGSAYWPDNGEIDIMEHVGFNPDVVHGTVHTKAYNHLLGTQRGGSIRVPTARTDFHVYAIEWTPEEIRWFVDDSLYYRFPNERLTDPEADWRHWPFDQPFHLIMNIAVGGAWGGQQGVDPEAFPAQLVVDYVRVYRWVE

SEQ ID NO: 53 - protein, 473 aa, Thermobispora bispora
MTESAMTSRAGRGRGADLVAAVVQGHAAASDAAGDLSFPDGFIWGAATAAYQIEGAWREDGRGLWDVFSHTPGKVASGHTGDIACDHYHRYADDVRLMAGLGDRVYRFSVAWPRIVPDGSGPVNPAGLDFYDRLVDELLGHGITPYPTLYHWDLPQTLEDRGGWAARDTAYRFAEYALAVHRRLGDRVRCWITLNEPWVAAFLATHRGAPGAADVPRFRAVHHLLLGHGLGLRLRSAGAGQLGLTLSLSPVIEARPGVRGGGRRVDALANRQFLDPALRGRYPEEVLKIMAGHARLGHPGRDLETIHQPVDLLGVNYYSHVRLAAEGEPANRLPGSEGIRFERPTAVTAWPGDRPDGLRTLLLRLSRDYPGVGLIITENGAAFDDRADGDRVHDPERIRYLTATLRAVHDAIMAGADLRGYFVWSVLDNFEWAYGYHKRGIVYVDYTTMRRIPRESALWYRDVVRRNGLRNGE

SEQ ID NO: 54 - protein, 387 aa, Clostridium stercorarium
MNKFLNKKWSLILTMGGIFLMATLSLIFATGKKAFNDQTSAEDIPSLAEAFRDYFPIGAAIEPGYTTGQIAELYKKHVNMLVAENAMKPASLQPTEGNFQWADADRIVQFAKENGMELRFHTLVWHNQTPTGFSLDKEGKPMVEETDPQKREENRKLLLQRLENYIRAVVLRYKDDIKSWDVVNEVIEPNDPGGMRNSPWYQITGTEYIEVAFRATREAGGSDIKLYINDYNTDDPVKRDILYELVKNLLEKGVPIDGVGHQTHIDIYNPPVERIIESIKKFAGLGLDNIITELDMSIYSWNDRSDYGDSIPDYILTLQAKRYQELFDALKENKDIVSAVVFWGISDKYSWLNGFPVKRTNAPLLFDRNFMPKPAFWAIVDPSRLRE

SEQ ID NO: 55 - protein, 755 aa, Clostridium thermocellum
MAVDIKKIIKQMTLEEKAGLCSGLDFWHTKPVERLGIPSIMMTDGPHGLRKQREDAEIADINNSVPATCFPSAAGLACSWDRELVERVGAALGEECQAENVSILLGPGANIKRSPLCGRNFEYFSEDPYLSSELAASHIKGVQSQGVGACLKHFAANNQEHRRMTVDTIVDERTLREIYFASFENAVKKARPWVVMCAYNKLNGEYCSENRYLLTEVLKNEWMHDGFVVSDWGAVNDRVSGLDAGLDLEMPTSHGITDKKIVEAVKSGKLSENILNRAVERILKVIFMALENKKENAQYDKDAHHRLARQAAAESMVLLKNEDDVLPLKKSGTIALIGAFVKKPRYQGSGSSHITPTRLDDIYEEIKKAGGDKVNLVYSEGYRLENDGIDEELINEAKKAASSSDVAVVFAGLPDEYESEGFDRTHMSIPENQNRLIEAVAEVQSNIVVVLLNGSPVEMPWIDKVKSVLEAYLGGQALGGALADVLFGEVNPSGKLAETFPVKLSHNPSYLNFPGEDDRVEYKEGLFVGYRYYDTKGIEPLFPFGHGLSYTKFEYSDISVDKKDVSDNSIINVSVKVKNVGKMAGKEIVQLYVKDVKSSVRRPEKELKGFEKVFLNPGEEKTVTFTLDKRAFAYYNTQIKDWHVESGEFLILIGRSSRDIVLKESVRVNSTVKIRKRFTVNSAVEDVMSDSSAAAVLGPVLKEITDALQIDMDNAHDMMAANIKNMPLRSLVGYSQGRLSEEMLEELVDKINNVE

SEQ ID NO: 56 - protein, 480 aa, Thermotoga maritima
MPSVKIGIIGAGSAVFSLRLVSDLCKTPGLSGSTVTLMDIDEERLDAILTIAKKYVEEVGADLKFEKTMNLDDVIIDADFVINTAMVGGHTYLEKVRQIGEKYGYYRGIDAQEFNMVSDYYTFSNYNQLKYFVDIARKIEKLSPKAWYLQAANPIFEGTTLVTRTVPIKAVGFCHGHYGVMEIVEKLGLEEEKVDWQVAGVNHGIWLNRFRYNGGNAYPLLDKWIEEKSKDWKPENPFNDQLSPAAIDMYRFYGVMPIGDTVRNSSWRYHRDLETKKKWYGEPWGGADSEIGWKWYQDTLGKVTEITKKVAKFIKENPSVRLSDLGSVLGKDLSEKQFVLEVEKILDPERKSGEQHIPFIDALLNDNKARFVVNIPNKGIIHGIDDDVVVEVPALVDKNGIHPEKIEPPLPDRVVKYYLRPRIMRMEMALEAFLTGDIRIIKELLYRDPRTKSDEQVEKVIEEILALPENEEMRKHYLKR

SEQ ID NO: 57 - protein, 225 aa, Thermomyces lanuginosus
MVGFTPVALAALAATGALAFPAGNATELEKRQTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 58 - protein, 562 aa, Acidothermus cellulolyticus
MPRALRRVPGSRVMLRVGVVVAVLALVAALANLAVPRPARAAGGGYWHTSGREILDANNVPVRIAGINWFGFETCNYVVHGLWSRDYRSMLDQIKSLGYNTIRLPYSDDILKPGTMPNSINFYQMNQDLQGLTSLQVMDKIVAYAGQIGLRIILDRHRPDCSGQSALWYTSSVSEATWISDLQALAQRYKGNPTVVGFDLHNEPHDPACWGCGDPSIDWRLAAERAGNAVLSVNPNLLIFVEGVQSYNGDSYWWGGNLQGAGQYPVVLNVPNRLVYSAHDYATSVYPQTWFSDPTFPNNMPGIWNKNWGYLFNQNIAPVWLGEFGTTLQSTTDQTWLKTLVQYLRPTAQYGADSFQWTFWSWNPDSGDTGGILKDDWQTVDTVKDGYLAPIKSSIFDPVGASASPSSQPSPSVSPSPSPSPSASRTPTPTPTPTASPTPTLTPTATPTPTASPTPSPTAASGARCTASYQVNSDWGNGFTVTVAVTNSGSVATKTWTVSWTFGGNQTITNSWNAAVTQNGQSVTARNMSYNNVIQPGQNTTFGFQASYTGSNAAPTVACAAS

SEQ ID NO: 59 - protein, 491 aa, Sulfolobus acidocaldarius
MLSFPKGFKFGWSQSGFQSEMGTPGSEDPNSDWHVWVHDRENIVSQVVSGDLPENGPGYWGNYKRFHDEAEKIGLNAVRINVEWSRIFPRPLPKPEMQTGTDKENSPVISVDLNESKLREMDNYANHEALSHYRQILEDLRNRGFHIVLNMYHWTLPIWLHDPIRVRRGDFTGPTGWLNSRTVYEFARFSAYVAWKLDDLASEYATMNEPNVVWGAGYAFPRAGFPPNYLSFRLSEIAKWNIIQAHARAYDAIKSVSKKSVGIIYANTSYYPLRPQDNEAVEIAERLNRWSFFDSIIKGEITSEGQNVREDLRNRLDWIGVNYYTRTVVTKAESGYLTLPGYGDRCERNSLSLANLPTSDFGWEFFPEGLYDVLLKYWNRYGLPLYVMENGIADDADYQRPYYLVSHIYQVHRALNEGVDVRGYLHWSLADNYEWSSGFSMRFGLLKVDYLTKRLYWRPSALVYREITRSNGIPEELEHLNRVPPIKPLRH

SEQ ID NO: 60 - protein, 510 aa, Pyrococcus woesei
MFPEKFLWGVAQSGFQFEMGDKLRRNIDTNTDWWHWVRDKTNIEKGLVSGDLPEEGINNYELYEKDHEIARKLGLNAYRIGIEWSRIFPWPTTFIDVDYSYNESYNLIEDVKITKDTLEELDEIANKREVAYYRSVINSLRSKGFKVIVNLNHFTLPYWLHDPIEARERALTNKRNGWVNPRTVIEFAKYAAYIAYKFGDIVDMWSTFNEPMVVVELGYLAPYSGFPPGVLNPEAAKLAILHMINAHALAYRQIKKFDTEKADKDSKEPAEVGIIYNNIGVAYPKDPNDSKDVKAAENDNFFHSGLFFEAIHKGKLNIEFDGETFIDAPYLKGNDWIGVNYYTREVVTYQEPMFPSIPLITFKGVQGYGYACRPGTLSKDDRPVSDIGWELYPEGMYDSIVEAHKYGVPVYVTENGIADSKDILRPYYIASHIKMTEKAFEDGYEVKGYFHWALTDNFEWALGFRMRFGLYEVNLITKERIPREKSVSIFREIVANNGVTKKIEEELLRG

SEQ ID NO: 61 - protein, 562 aa, Geobacillus thermoglucosidasius
MERVWWKEAVVYQIYPRSFYDSNGDGIGDIRGIIAKLDYLKELGVDVVWLSPVYKSPNDDNGYDISDYRDIMDEFGTMADWKTMLEEMHKRGIKLVMDLVVNHTSDEHPWFIESRKSKDNPYRDYYIWRPGKNGKEPNNWESVFSGSAWEYDEMTGEYYLHLFSKKQPDLNWENPKVRREVYEMMKFWLDKGVDGFRMDVINMISKVPELPDGEPQSGKKYASGSRYYMNGPRVHEFLQEMNREVLSKYDIMTVGETPGVTPKEGILYTDPSRRELNMVFQFEHMDLDSGPGGKWDIRPWSLADLKKTMTKWQKELEGKGWNSLYLNNHDQPRAVSRFGDDGKYRVESAKMLATFLHMMQGTPYIYQGEEIGMTNVRFPSIEDYRDIETLNMYKERVEEYGEDPQEVMEKIYYKGRDNARTPMQWDDSENAGFTAGTPWIPVNPNYKEINVKAALEDPNSVFHYYKKLIQLRKQHDIIVYGTYDLILEDDPYIYRYTRTLGNEQLIVITNFSEKTPVFRLPDHIIYKTKELLISNYDVDEAEELKEIRLRPWEARVYKIRLP

SEQ ID NO: 62 - protein, 649 aa, Pyrococcus furiosus
MGDKINFIFGIHNHQPLGNFGWVFEEAYEKCYWPFLETLEEYPNMKVAIHTSGPLIEWLQDNRPEYIDLLRSLVKRGQVEIVVAGFYEPVLASIPKEDRIEQIRLMKEWAKSIGFDARGVWLTERVWQPELVKTLKESGIDYVIVDDYHFMSAGLSKEELYWPYYTEDGGEVIAVFPIDEKLRYLIPFRPVDKVLEYLHSLIDGDESKVAVFHDDGEKFGIWPGTYEWVYEKGWLREFFDRISSDEKINLMLYTEYLEKYKPRGLVYLPIASYFEMSEWSLPAKQARLFVEFVNELKVKGIFEKYRVFVRGGIWKNFFYKYPESNYMHKRMLMVSKLVRNNPEARKYLLRAQCNDAYWHGLFGGVYLPHLRRAIWNNLIKANSYVSLGKVIRDIDYDGFEEVLIENDNFYAVFKPSYGGSLVEFSSKNRLVNYVDVLARRWEHYHGYVESQFDGVASIHELEKKIPDEIRKEVAYDKYRRFMLQDHVVPLGTTLEDFMFSRQQEIGEFPRVPYSYELLDGGIRLKREHLGIEVEKTVKLVNDGFEVEYIVNNKTGNPVLFAVELNVAVQSIMESPGVLRGKEIVVDDKYAVGKFALKFEDEMEVWKYPVKTLSQSESGWDLIQQGVSYIVPIRLEDKIRFKLKFEEASG

SEQ ID NO: 63 - protein, 824 aa, Bacillus sp.
MMLRKKTKQLISSILILVLLLSLFPAALAAEGNTREDNFKHLLGNDNVKRPSEAGALQLQEVDGQMTLVDQHGEKIQLRGMSTHGLQWFPEILNDNAYKALSNDWDSNMIRLAMYVGENGYATNPELIKQRVIDGIELAIENDMYVIVDWHVHAPGDPRDPVYAGAKDFFREIAALYPNNPHIIYELANEPSSNNNGGAGIPNNEEGWKAVKEYADPIVEMLRKSGNADDNIIIVGSPNWSQRPDLAADNPIDDHHTMYTVHFYTGSHAASTESYPSETPNSERGNVMSNTRYALENGVAVFATEWGTSQASGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSANGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEAVKEEKKEAKEEKKAVKNEAKKK

SEQ ID NO: 64 - protein, 1059 aa, Thermotoga maritima
MQVRKRRGLLDVSTAVLVGILAGFLGVVLAASGVLSFGKEASSKGDSSLETVLALSFEGTTEGVVPFGKDVVLTASQDVAADGEYSLKVENRTSPWDGVEIDLTGKVKSGADYLLSFQVYQSSDAPQLFNVVARTEDEKGERYDVILDKVVVSDHWKEILVPFSPTFEGTPAKYSLIIVASKNTNFNFYLDKVQVLAPKESGPKVIYETSFENGVGDWQPRGDVNIEASSEVAHSGKSSLFISNRQKGWQGAQINLKGILKTGKTYAFEAWVYQNSGQDQTIIMTMQRKYSSDASTQYEWIKSATVPSGQWVQLSGTYTIPAGVTVEDLTLYFESQNPTLEFYVDDVKIVDTTSAEIKIEMEPEKEIPALKEVLKDYFKVGVALPSKVFLNPKDIELITKHFNSITAENEMKPESLLAGIENGKLKFRFETADKYIQFVEENGMVIRGHTLVWHNQTPDWFFKDENGNLLSKEAMTERLKEYIHTVVGHFKGKVYAWDVVNEAVDPNQPDGLRRSTWYQIMGPDYIELAFKFAREADPDAKLFYNDYNTFEPRKRDIIYNLVKDLKEKGLIDGIGMQCHISLATDIKQIEEAIKKFSTIPGIEIHITELDMSVYRDSSSNYPEAPRTALIEQAHKMMQLFEIFKKYSNVITNVTFWGLKDDYSWRATRRNDWPLIFDKDHQAKLAYWAIVAPEVLPPLPKESRISEGEAVVVGMMDDSYLMSKPIEILDEEGNVKATIRAVWKDSTIYIYGEVQDKTKKPAEDGVAIFINPNNERTPYLQPDDTYAVLWTNWKTEVNREDVQVKKFVGPGFRRYSFEMSITIPGVEFKKDSYIGFDAAVIDDGKWYSWSDTTNSQKTNTMNYGTLKLEGIMVATAKYGTPVIDGEIDEIWNTTEEIETKAVAMGSLDKNATAKVRVLWDENYLYVLAIVKDPVLNKDNSNPWEQDSVEIFIDENNHKTGYYEDDDAQFRVNYMNEQTFGTGGSPARFKTAVKLIEGGYIVEAAIKWKTIKPTPNTVIGFNIQVNDANEKGQRVGIISWSDPTNNSWRDPSKFGNLRLIK

SEQ ID NO: 65 - protein, 512 aa, Clostridium stercorarium
MKRKVKKMAAMATSIIMAIMIILHSIPVLAGRIIYDNETGTHGGYDYELWKDYGNTIMELNDGGTFSCQWSNIGNALFRKGRKFNSDKTYQELGDIVVEYGCDYNPNGNSYLCVYGWTRNPLVEYYIVESWGSWRPPGATPKGTITQWMAGTYEIYETTRVNQPSIDGTATFQQYWSVRTSKRTSGTISVTEHFKQWERMGMRMGKMYEVALTVEGYQSSGYANVYKNEIRIGANPTPAPSQSPIRRDAFSIIEAEEYNSTNSSTLQVIGTPNNGRGIGYIENGNTVTYSNIDFGSGATGFSATVATEVNTSIQIRSDSPTGTLLGTLYVSSTGSWNTYQTVSTNISKITGVHDIVLVFSGPVNVDNFIFSRSSPVPAPGDNTRDAYSIIQAEDYDSSYGPNLQIFSLPGGGSAIGYIENGYSTTYKNIDFGDGATSVTARVATQNATTIQVRLGSPSGTLLGTIYVGSTGSFDTYRDVSATISNTAGVKDIVLVFSGPVNVDWFVFSKSGT

SEQ ID NO: 66 - protein, 457 aa, Solanum lycopersicum
MVIQRNSILLLIIIFASSISTCRSNVIDDNLFKQVYDNILEQEFAHDFQAYLSYLSKNIESNNNIDKVDKNGIKVINVLSFGAKGDGKTYDNIAFEQAWNEACSSRTPVQFVVPKNKNYLLKQITFSGPCRSSISVKIFGSLEASSKISDYKDRRLWIAFDSVQNLVVGGGGTINGNGQVWWPSSCKINKSLPCRDAPTALTFWNCKNLKVNNLKSKNAQQIHIKFESCTNVVASNLMINASAKSPNTDGVHVSNTQYIQISDTIIGTGDDCISIVSGSQNVQATNITCGPGHGISIGSLGSGNSEAYVSNVTVNEAKIIGAENGVRIKTWQGGSGQASNIKFLNVEMQDVKYPIIIDQNYCDRVEPCIQQFSAVQVKNVVYENIKGTSATKVAIKFDCSTNFPCEGIIMENINLVGESGKPSEATCKNVHFNNAEHVTPHCTSLEISEDEALLYNY

SEQ ID NO: 67 - protein, 649 aa, Clostridium thermocellum
MSRMTLKSSMKKRVLSLLIAVVFLSLTGVFPSGLIETKVSAAKITENYQFDSRIRLNSIGFIPNHSKKATIAANCSTFYVVKEDGTIVYTGTATSMFDNDTKETVYIADFSSVNEEGTYYLAVPGVGKSVNFKIAMNVYEDAFKTAMLGMYLLRCGTSVSATYNGIHYSHGPCHTNDAYLDYINGQHTKKDSTKGWHDAGDYNKYVVNAGITVGSMFLAWEHFKDQLEPVALEIPEKNNSIPDFLDELKYEIDWILTMQYPDGSGRVAHKVSTRNFGGFIMPENEHDERFFVPWSSAATADFVAMTAMAARIFRPYDPQYAEKCINAAKVSYEFLKNNPANVFANQSGFSTGEYATVSDADDRLWAAAEMWETLGDEEYLRDFENRAAQFSKKIEADFDWDNVANLGMFTYLLSERPGKNPALVQSIKDSLLSTADSIVRTSQNHGYGRTLGTTYYWGCNGTVVRQTMILQVANKISPNNDYVNAALDAISHVFGRNYYNRSYVTGLGINPPMNPHDRRSGADGIWEPWPGYLVGGGWPGPKDWVDIQDSYQTNEIAINWNAALIYALAGFVNYNSPQNEVLYGDVNDDGKVNSTDLTLLKRYVLKAVSTLPSSKAEKNADVNRDGRVNSSDVTILSRYLIRVIEKLPI

SEQ ID NO: 68 - protein, 203 aa, Sulfolobus acidocaldarius
MLRSLVLNEKLRARVLERAEEFLLNNKADEEVWFRELVLCILTSNSSFISAYKSMNYILDKILYMDEKEISILLQESGYRFYNLKAKYLYRAKNLYGKVKKTIKEIADKDQMQAREFIATHIYGIGYKEASHFLRNVGYLDLAIIDRHILRFINNLGIPIKLKSKREYLLAESLLRSIANNLNVQVGLLDLFIFFKQTNTIVK

SEQ ID NO: 69 - protein, 432 aa, Thermotoga maritima
MFKPNYHFFPITGWMNDPNGLIFWKGKYHMFYQYNPRKPEWGNICWGHAVSDDLVHWRHLPVALYPDDETHGVFSGSAVEKDGKMFLVYTYYRDPTHNKGEKETQCVAMSENGLDFVKYDGNPVISKPPEEGTHAFRDPKVNRSNGEWRMVLGSGKDEKIGRVLLYTSDDLFHWKYEGVIFEDETTKEIECPDLVRIGEKDILIYSITSTNSVLFSMGELKEGKLNVEKRGLLDHGTDFYAAQTFFGTDRVVVIGWLQSWLRTGLYPTKREGWNGVMSLPRELYVENNELKVKPVDELLALRKRKVFETAKSGTFLLDVKENSYEIVCEFSGEIELRMGNESEEVVITKSRDELIVDTTRSGVSGGEVRKSTVEDEATNRIRAFLDSCSVEFFFNDSIAFSFRIHPENVYNILSVKSNQVKLEVFELENIWL

SEQ ID NO: 70 - protein, 1021 aa, Rhodothermus marinus
MAGPHRSRAAGPPPFAVDEHVALEMVAFRGEVFAGHGLLADQRLIAHTGRPALNAQRITQQKQRDQCRGQRHRHHQGGRNLRKAHRTFHEHQSTQDQAHDAPHGQQAKTGHEGLGHEHAQAQHQQGQSNVVDRQDGEPVEAQHQKDGAQRAGNAPAGRVELEQQPVEAQHQQQEGDVRIGKRRQNAFAPPALDHVHGGPGRLQRHGLAVERHVPAVQQHQQRVQRGRQQIDHVLGHGLPGRQRLAFRDGPRRPVGVASPVLGQRPCPGHRIVQNLFRHGIDPCRVGRCRRSPSELHGMGCADVRARGHGRHMRGQRDEHPGRGRPCARRRHVDDDRDRTPQEKLYDVARGLDEPARRVHFDDEADRSVFRGLAQPAPDEPEGRRRDRLVLQRQSVNHRRGRLSRHRQQHQPQQQRPHGNQAFLGKYEKRRRKPTACLKSLRRFPDKDAPVLYFVNQLEKTKRRMTLLLVWLIFTGVAGEIRLEAEDGELLGVAVDSTLTGYSGRGYVTGFDAPEDSVRFSFEAPRGVYRVVFGVSFSSRFASYALRVDDWHQTGSLIKRGGGFFEASIGEIWLDEGAHTMAFQLMNGALDYVRLEPVSYGPPARPPAQLSDSQATASAQALFAFLLSEYGRHILAGQQQNPYRRDFDAINYVRNVTGKEPALVSFDLIDYSPTREAHGVVHYQTPEDWIAWAGRDGIVSLMWHWNAPTDLIEDPSQDCYWWYGFYTRCTTFDVAAALADTSSERYRLLLRDIDVIAAQLQKFQQADIPVLWRPLHEAAGGWFWWGAKGPEPFKQLWRLLYERLVHHHGLHNLIWVYTHEPGAAEWYPGDAYVDIVGRDVYADDPDALMRSDWNELQTLFGGRKLVALTETGTLPDVEVITDYGIWWSWFSIWTDPFLRDVDPDRLTRVYHSERVLTRDELPDWRSYVLHATTVQPAGDLALAVYPNPGAGRLHVEVGLPVAAPVVVEVFNLLGQRVFQYQAGMQPAGLWRRAFELALAPGVYLVQVRAGNLVARRRWVSVR

SEQ ID NO: 71 - protein, 549 aa, Geobacillus stearothermophilus
MLTFHRIIRKGWMFLLAFLLTALLFCPTGQPAKAAAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 72 - protein, 705 aa, Geobacillus stearothermophilus
MPTNLFFNAHHSPVGAFASFTLGFPGKSGGLDLELARPPRQNVLIGVESLHESGLYHVLPFLETAEEDESKRYDIENPDPNPQKPNILIPFAKEEIQREFHVATDTWKAGDLTFTIYSPVKAVPNPETADEEELKLALVPAVIVEMTIDNTNGTRARRAFFGFEGTDPYTSMRRIDDTCPQLRGVGQGRILSIVSKDEGVRSALHFSMEDILTAQLEENWTFGLGKVGALIVDVPAGEKKTYQFAVCFYRGGYVTAGMDASYFYTRFFQNIEEVGLYALEQAEVLKEQSFRSNKLIEKEWLSDDQTFMMAHAIRSYYGNTQLLEHEGKPIWVVNEGEYRMMNTFDLTVDQLFFELKLNPWTVKNVLDLYVERYSYEDRVRFPGEETEYPSGISFTHDMGVANTFSRPHYSSYELYGISGCFSHMTHEQLVNWVLCAAVYIEQTKDWAWRDKRLAILEQCLESMVRRDHPDPEQRNGVMGLDSTRTMGGAEITTYDSLDVSLGQARNNLYLAGKCWAAYVALEKLFRDVGKEELAALAGEQAEKCAATIVSHVTDDGYIPAIMGEGNDSKIIPAIEGLVFPYFTNCHEALDENGRFGAYIQALRNHLQYVLREGICLFPDGGWKISSTSNNSWLSKIYLCQFIARHILGWEWDEQGKRADAAHVAWLTHPTLSIWSWSDQIIAGEITGSKYYPRGVTSILWLEEGE

SEQ ID NO: 73 - protein, 330 aa, Geobacillus stearothermophilus
MCSSIPSLREVFANDFRIGAAVNPVTLEAQQSLLIRHVNSLTAENHMKFEHLQPEEGRFTFDIAIKSSTSPFSSHGVRGHTLVWHNQTPSWVFQDSQGHFVGRDVLLERMKSHISTVVQRYKGKVYCWDVINEAVADEGSEWLRSSTWRQIIGDDFIQQAFLYAHEADPEALLFYNDYNECFPEKREKIYTLVKSLRDKGIPIHGIGMQAHWSLNRPTLDEIRAAIERYASLGVILHITELDISMFEFDDHRKDLAAPTNEMVERQAERYEQIFSLFKEYRDVIQNVTFWGIADDHTWLDHFPVQGRKNWPLLFDEQHNPKPAFWRVVNI

SEQ ID NO: 74 - protein, 407 aa, Geobacillus stearothermophilus
MRNVVRKPLTIGLALTLLLPMGMTATSAKNADSYAKKPHISALNAPQLDQRYKNEFTIGAAVEPYQLQNEKDVQMLKRHFNSIVAENVMKPISIQPEEGKFNFEQADRIVKFAKANGMDIRFHTLVWHSQVPQWFFLDKEGKPMVNETDPVKREQNKQLLLKRLETHIKTIVERYKDDIKYWDVVNEVVGDDGKLRNSPWYQIAGIDYIKVAFQAARKYGGDNIKLYMNDYNTEVEPKRTALYNLVKQLKEEGVPIDGIGHQSHIQIGWPSEAEIEKTINMFAALGLDNQITELDVSMYGWPPRAYPTYDAIPKQKFLDQAARYDRLFKLYEKLSDKISNVTFWGIADNHTWLDSRADVYYDANGNVVVDPNAPYAKVEKGKGKDAPFVFGPDYKVKPAYWAIIDHK

SEQ ID NO: 75 - protein, 686 aa, Dictyoglomus thermophilum
MTKSIYFSLGIHNHQPVGNFDFVIERAYEMSYKPLINFFFKHPDFPINVHFSGFLLLWLEKNHPEYFEKLKIMAERGQIEFVSGGFYEPILPIIPDKDKVQQIKKLNKYIYDKFGQTPKGMWLAERVWEPHLVKYIAEAGIEYVVVDDAHFFSVGLKEEDLFGYYLMEEQGYKLAVFPISMKLRYLIPFADPEETITYLDKFASEDKSKIALLFDDGEKFGLWPDTYRTVYEEGWLETFVSKIKENFLLVTPVNLYTYMQRVKPKGRIYLPTASYREMMEWVLFPEAQKELEELVEKLKTENLWDKFSPYVKGGFWRNFLAKYDESNHMQKKMLYVWKKVQDSPNEEVKEKAMEEVFQGQANDAYWHGIFGGLYLPHLRTAIYEHLIKAENYLENSEIRFNIFDFDCDGNDEIIVESPFFNLYLSPNHGGSVLEWDFKTKAFNLTNVLTRRKEAYHSKLSYVTSEAQGKSIHERWTAKEEGLENILFYDNHRRVSFTEKIFESEPVLEDLWKDSSRLEVDSFYENYDYEINKDENKIRVLFSGVFRGFELCKSYILYKDKSFVDVVYEIKNVSETPISLNFGWEINLNFLAPNHPDYYFLIGDQKYPLSSFGIEKVNNWKIFSGIGIELECVLDVEASLYRYPIETVSLSEEGFERVYQGSALIHFYKVDLPVGSTWRTTIRFWVK

SEQ ID NO: 76 - protein, 1055 aa, Thermotoga neapolitana
MRKKRRGFLNASTAVLVGILAGFLGVVLAATGALGFAVRESLLLKQFLFLSFEGNTDGASPFGKDVVVTASQDVAADGEYSLKVENRTSVWDGVEIDLTGKVNTGTDYLLSFHVYQTSDSPQLFSVLARTEDEKGERYKILADKVVVPNYWKEILVPFSPTFEGTPAKFSLIITSPKKTDFVFYVDNVQVLTPKEAGPKVVYETSFEKGIGDWQPRGSDVKISISPKVAHSGKKSLFVSNRQKGWHGAQISLKGILKTGKTYAFEAWVYQESGQDQTIIMTMQRKYSSDSSTKYEWIKAATVPSGQWVQLSGTYTIPAGVTVEDLTLYFESQNPTLEFYVDDVKVVDTTSAEIKLEMNPEEEIPALKDVLKDYFRVGVALPSKVFINQKDIALISKHSNSSTAENEMKPDSLLAGIENGKLKFRFETADKYIEFAQQNGMVVRGHTLVWHNQTPEWFFKDENGNLLSKEEMTERLREYIHTVVGHFKGKVYAWDVVNEAVDPNQPDGLRRSTWYQIMGPDYIELAFKFAREADPNAKLFYNDYNTFEPKKRDIIYNLVKSLKEKGLIDGIGMQCHISLATDIRQIEEAIKKFSTIPGIEIHITELDISVYRDSTSNYSEAPRTALIEQAHKMAQLFKIFKKYSNVITNVTFWGLKDDYSWRATRRNDWPLIFDKDYQAKLAYWAIVAPEVLPPLPKESKISEGEAVVVGMMDDSYMMSKPIEIYDEEGNVKATIRAIWKDSTIYVYGEVQDATKKPAEDGVAIFINPNNERTPYLQPDDTYVVLWTNWKSEVNREDVEVKKFVGPGFRRYSFEMSITIPGVEFKKDSYIGFDVAVIDDGKWYSWSDTTNSQKTNTMNYGTLKLEGVMVATAKYGTPVIDGEIDDIWNTTEEIETKSVAMGSLEKNATAKVRVLWDEENLYVLAIVKDPVLNKDNSNPWEQDSVEIFIDENNHKTGYYEDDDAQFRVNYMNEQSFGTGASAARFKTAVKLIEGGYIVEAAIKWKTIKPSPNTVIGFNVQVNDANEKGQRVGIISWSDPTNNSWRDPSKFGNLRLIK

SEQ ID NO: 77 - protein, 431 aa, Thermus thermophilus
MDDHAEKFLWGVATSAYQIEGATQEDGRGPSIWDAFARRPGAIRDGSTGEPACDHYRRYEEDIALMQSLGVRAYRFSVAWPRILPEGRGRINPKGLAFYDRLVDRLLASGITPFLTLYHWDLPLALEERGGWRSRETAFAFAEYAEAVARALADRVPFFATLNEPWCSAFLGHWTGEHAPGLRNLEAALRAAHHLLLGHGLAVEALRAAGARRVGIVLNFAPAYGEDPEAVDVADRYHNRYFLDPILGKGYPESPFRDPPPVPILSRDLELVARPLDFLGVNYYAPVRVAPGTGTLPVRYLPPEGPATAMGWEVYPEGLHHLLKRLGREVPWPLYVTENGAAYPDLWTGEAVVEDPERVAYLEAHVEAALRAREEGVDLRGYFVWSLMDNFEWAFGYTRRFGLYYVDFPSQRRIPKRSALWYRERIARAQT

SEQ ID NO: 78 - protein, 431 aa, Thermus thermophilus
MTENAEKFLWGVATSAYQIEGATQEDGRGPSIWDAFAQRPGAIRDGSTGEPACDHYRRYEEDIALMQSLGVRAYRFSVAWPRILPEGRGRINPKGLAFYDRLVDRLLASGITPFLTLYHWDLPLALEERGGWRSRETAFAFAEYAEAVARALADRVPFFATLNEPWCSAFLGHWTGEHAPGLRNLEAALRAAHHLLLGHGLAVEALRAAGARRVGIVLNFAPAYGEDPEAVDVADRYHNRFFLDPILGKGYPESPFRDPPPVPILSRDLELVARPLDFLGVNYYAPVRVAPGTGTLPVRYLPPEGPATAMGWEVYPEGLYHLLKRLGREVPWPLYVTENGAAYPDLWTGEAVVEDPERVAYLEAHVEAALRAREEGVDLRGYFVWSLMDNFEWAFGYTRRFGLYYVDFPSQRRIPKRSALWYRERIARAQT

SEQ ID NO: 79 - protein, 280 aa, Thermus thermophilus
MAQVGRGASPLSRARVPPLPHPLDGEHLPHDPAGGGHGKASSQDAPVGQLPGHLARPAFFHYLKNSFLVCSLTTVFALAVATFAGYALARFRFPGAELFGGSVLVTQVIPGILFLIPIYIMYIYVQNWVRSALGLEVRLVGSYGGLVFTYTAFFVPLSIWILRGFFASIPKELEEAAMVDGATPFQAFHRVILPLALPGLAATAVYIFLTAWDELLFAQVLTTEATATVPVGIRNFVGNYQNRYDLVMAAATVATLPVLVLFFFVQRQLIQGLTAGAVKG

SEQ ID NO: 80 - protein, 431 aa, Thermus filiformis
MAENAEKFLWGVATSAYQIEGATQEDGRGPSIWDTFARRPGAIRDGSTGEPACDHYHRYEEDIALMQSLGVGVYRFSVAWPRILPEGRGRINPKGLAFYDRLVDRLLAAGITPFLTLYHWDLPQALEDRGGWRSRETAFAFAEYAEAVARALADRVPFFATLNEPWCSAFLGHWTGEHAPGLRNLEAALRAAHHLLLGHGLAVEALRAAGAKRVGIVLNFAPVYGEDPEAVDVADRYHNRYFLDPILGRGYPESPFQDPPPTPNLSRDLELVARPLDFLGVNYYAPVRVAPGTGPLPVRYLPPEGPVTAMGWEVYPEGLYHLLKRLGREVPWPLYITENGAAYPDLWTGEAVVEDPERVAYLEAHVEAALRAREEGVDLRGYFVWSLMDNFEWAFGYTRRFGLYYVDFPSQRRIPKRSALWYRERIARAQL

SEQ ID NO: 81 - protein, 481 aa, Thermosphaera aggregans
MKFPKDFMIGYSSSPFQFEAGIPGSEDPNSDWWVWVHDPENTAAGLVSGDFPENGPGYWNLNQNDHDLAEKLGVNTIRVGVEWSRIFPKPTFNVKVPVERDENGSIVHVDVDDKAVERLDELANKEAVNHYVEMYKDWVERGRKLILNLYHWPLPLWLHNPIMVRRMGPDRAPSGWLNEESVVEFAKYAAYIAWKMGELPVMWSTMNEPNVVYEQGYMFVKGGFPPGYLSLEAADKARRNMIQAHARAYDNIKRFSKKPVGLIYAFQWFELLEGPAEVFDKFKSSKLYYFTDIVSKGSSIINVEYRRDLANRLDWLGVNYYSRLVYKIVDDKPIILHGYGFLCTPGGISPAENPCSDFGWEVYPEGLYLLLKELYNRYGVDLIVTENGVSDSRDALRPAYLVSHVYSVWKAANEGIPVKGYLHWSLTDNYEWAQGFRQKFGLVMVDFKTKKRYLRPSALVFREIATHNGIPDELQHLTLIQ

SEQ ID NO: 82 - protein, 334 aa, Clostridium thermocellum
MKNRVISLLMASLLLVLSVIVAPFYKAEAATVVNTPFVAVFSNFDSSQWEKADWANGSVFNCVWKPSQVTFSNGKMILTLDREYGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGVPQDNPTPTPTIAPSTPTNPNLPLKGDVNGDGHVNSSDYSLFKRYLLRVIDRFPVGDQSVADVNRDGRIDSTDLTMLKRYLIRAIPSL

SEQ ID NO: 83 - protein, 263 aa, Artificial Sequence
  Synthetic polypeptide
MGFVLFSQLPSFLLVSTLLLFLVISHSCRAQNGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSKLVVNTPFVAVFSNFDSSQWEKADWANGSVFNCVWKPSQVTFSNGKMILTLDREYVDHHHHHHKDEL

SEQ ID NO: 84 - DNA, 1668 bp, Artificial Sequence
  Synthetic polynucleotide
atgggattcgtgcttttctctcagcttccttctttccttcttgtgtctactcttcttcttttccttgtgatttctcactcttgcagggctgatcagatatgcattggataccacgctaacaactctactgagcaagtggatacaattatggaaaagaacgtgactgttactcacgctcaggatattcttgaaaagactcacaacggaaagttgtgcgatcttgatggtgttaagccacttattcttagggactgcagtgttgctggatggcttcttggaaacccaatgtgcgatgagttcattaacgtgccagagtggtcttatattgtggagaaggctaacccagctaacgatctttgctacccaggaaacttcaacgattacgaagagcttaagcaccttctttctaggattaaccacttcgagaagattcagattattccaaagtcatcttggagtgatcacgaggcttcatctggtgtttcttcagcttgcccataccaaggtactccatctttcttcaggaacgttgtttggcttattaagaagaacaacacttacccaactattaagaggtcttacaacaacactaaccaggaagatttgcttattctttggggaattcaccactctaatgatgctgctgaacagactaagttgtaccagaacccaactacttacatttctgtgggaacttctactcttaaccagaggcttgtgccaaagattgctactaggtctaaggtgaacggacaatctggaaggatggatttcttctggactattcttaagccaaacgatgctattaacttcgagtctaacggaaacttcattgctccagagtacgcttacaagattgtgaagaaaggtgatagtgctattgtgaagtctgaggtggagtacggaaactgtaacactaagtgccagactccaattggagctattaactcttctatgccattccacaacattcacccacttactattggagagtgcccaaagtacgtgaagtctaacaagttggtgcttgctactggacttaggaactctccacttagagagagaagaagaaagaggggacttttcggagctattgctggattcattgagggaggatggcagggaatggttgatggatggtacggataccatcactctaatgagcagggatctggatatgctgctgataaggaatctactcagaaagctattgatggtgttactaacaaggtgaactctattattgataagatgaacactcagttcgaagctgttggaagagagttcaacaaccttgagagaaggattgagaaccttaacaagaaaatggaagatggattccttgatgtgtggacttacaacgctgagttgcttgtgcttatggaaaacgagaggactcttgatttccacgattctaacgtgaagaacctttacgataaagtgaggcttcagcttagggataacgctaaagagcttggaaacggttgcttcgagttctaccacaagtgcgataacgagtgcatggaatctgttaggaacggaacttacgattacccacagtactctgaagaagctaggcttaagagggaagagatttctggtgttaagttggagtctattggaacttaccagattcatcaccatcaccaccacaaggatgagctttga

SEQ ID NO: 85 - protein, 555 aa, Artificial Sequence
  Synthetic polypeptide
MGFVLFSQLPSFLLVSTLLLFLVISHSCRADQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGTPSFFRNVVWLIKKNNTYPTIKRSYNNTNQEDLLILWGIHHSNDAAEQTKLYQNPTTYISVGTSTLNQRLVPKIATRSKVNGQSGRMDFFWTILKPNDAINFESNGNFIAPEYAYKIVKKGDSAIVKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQIHHHHHHKDEL

SEQ ID NO: 86 - DNA, 1674 bp, Artificial Sequence
  Synthetic polynucleotide
atgggtttcgtgcttttctctcagcttccttctttccttcttgtgtctactcttcttcttttccttgtgatttctcattcttgcagggctgatcaaatctgcattggttaccatgctaacaattctactgagcaagtggatacaattatggaaaagaatgtgactgtgactcatgctcaggatattcttgaaaagactcataatggaaagttgtgcgatcttgatggtgttaagcctcttattcttagggactgcagtgttgctggttggttgcttggaaatcctatgtgcgatgagttccttaatgtgcctgagtggtcttacattgtggagaagattaatcctgctaatgatctttgctaccctggaaatttcaatgattacgaagagcttaaacatcttctttctaggattaatcatttcgagaagattcagattattcctaagtcatcttggagtgatcatgaggcttcatctggtgtttcttcagcttgcccttatcagggaaggtcatctttcttcaggaatgttgtttggcttattaagaagaataacgcttaccctactattaagaggtcttacaacaatactaatcaggaggatcttcttgttctttggggtattcatcatcctaatgatgctgctgaacagactaggctttaccagaatcctactacttacatttctgtgggaacttctactcttaatcagaggcttgtgcctaagattgctactaggtctaaagtgaatggtcagtctggaaggatggaattcttctggactattcttaagccaaatgatgctattaatttcgagtctaatggaaatttcattgctcctgagaatgcttacaagattgtgaagaagggtgatagtactattatgaagtctgagcttgagtacggtaattgcaatactaagtgccagactcctattggtgctattaattcttctatgcctttccataatattcatcctcttactattggtgagtgccctaagtacgtgaagtctaataggcttgtgcttgctactggtcttaggaattctcctcagggtgaaagaagaagaaagaagaggggacttttcggagctattgctggttttattgagggaggatggcagggaatggttgatggttggtacggttaccatcattctaatgagcagggttctggttatgctgctgataaggaatctactcagaaagctattgatggtgttactaataaggtgaactctattattgataagatgaatactcagttcgaggctgttggtagagagttcaacaatcttgagagaaggattgagaatcttaataagaaaatggaagatggtttccttgatgtgtggacttacaatgctgagttgcttgtgcttatggaaaatgagaggactcttgatttccatgattctaatgtgaagaatctttacgataaagtgaggcttcagcttagggataatgctaaagaacttggaaatggttgcttcgagttctaccatagatgcgataatgagtgcatggaatctgtgaggaatggtacttacgattaccctcagtactctgaagaagctaggcttaagagggaagagatttctggtgttaagttggagtctattggtacttaccagattcatcatcatcatcatcataaggatgagctttgatga

SEQ ID NO: 87 - protein, 556 aa, Artificial Sequence
  Synthetic polypeptide
MGFVLFSQLPSFLLVSTLLLFLVISHSCRADQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQIHHHHHHKDEL

SEQ ID NO: 88 - DNA, 1674 bp, Artificial Sequence
  Synthetic polynucleotide
atgggtttcgtgcttttctctcagcttccttctttccttcttgtgtctactcttcttcttttccttgtgatttctcattcttgcagggctgatcaaatctgcattggttaccatgctaacaattctactgagcaagtggatacaattatggaaaagaatgtgactgtgactcatgctcaggatattcttgaaaagactcataatggaaagttgtgcgatcttgatggtgttaagcctcttattcttagggactgcagtgttgctggttggttgcttggaaatcctatgtgcgatgagttcattaatgtgcctgagtggtcttacattgtggagaaggctaatcctactaatgatctttgctaccctggttctttcaatgattacgaagagcttaaacatcttctttctaggattaatcatttcgagaagattcagattattcctaagtcatcttggagtgatcatgaggcttcatctggtgtttcttcagcttgcccttaccttggatctccttctttcttcaggaatgttgtttggcttattaagaagaattctacttaccctactattaagaagtcttacaacaatactaatcaggaggatcttcttgttctttggggtattcatcatcctaatgatgctgctgaacagactaggctttaccagaatcctactacttacatttctattggtacttctactcttaatcagaggcttgtgcctaagattgctactaggtctaaagtgaatggtcagtctggaaggatggaattcttctggactattcttaagccaaatgatgctattaatttcgagtctaatggaaatttcattgctcctgagtacgcttacaagattgtgaagaaaggtgatagtgctattatgaagtctgagcttgagtacggtaattgcaatactaagtgccagactcctatgggtgctattaattcttctatgcctttccataatattcatcctcttactattggtgagtgccctaagtacgtgaagtctaataggcttgtgcttgctactggtcttaggaattctcctcagagagagtctagaagaaagaagaggggacttttcggagctattgctggttttattgagggaggatggcagggaatggttgatggttggtatggttaccatcattctaatgagcagggttctggttatgctgctgataaggaatctactcagaaagctattgatggtgttactaataaggtgaactctattattgataagatgaatactcagttcgaggctgttggtagagagttcaacaatcttgagagaaggattgagaatcttaataagaaaatggaagatggtttccttgatgtgtggacttacaatgctgagttgcttgtgcttatggaaaatgagaggactcttgatttccatgattctaatgtgaagaatctttacgataaagtgagacttcagcttagggataatgctaaagaacttggaaatggttgcttcgagttctaccataagtgcgataatgagtgcatggaatctattaggaatggtacttacaattaccctcagtactctgaagaagctaggcttaagagggaagagatttctggtgttaagttggagtctattggaacttaccagattcatcatcatcatcatcataaggatgagctttgatga

SEQ ID NO: 89 - protein, 556 aa, Artificial Sequence
  Synthetic polypeptide
MGFVLFSQLPSFLLVSTLLLFLVISHSCRADQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQIHHHHHHKDEL

SEQ ID NO: 90 - DNA, 1674 bp, Artificial Sequence
  Synthetic polynucleotide
atgggattcgtgcttttctctcagcttccttctttccttcttgtgtctactcttcttcttttccttgtgatttctcactcttgcagggctgatcaaatctgcattggataccacgctaacaactctactgagcaagtggatacaattatggaaaagaacgtgactgttactcacgctcaggatattcttgaaaagactcacaacggaaagttgtgcgatcttgatggtgttaagccacttattcttagggattgctctgttgctggatggcttcttggaaacccaatgtgtgatgagttcattaacgtgccagagtggtcttatattgtggagaaggctaacccagtgaacgatctttgttaccctggtgatttcaacgattacgaagagcttaagcaccttctttctaggattaaccacttcgagaagattcagattattccaaagtcatcttggtcatctcacgaggcttctcttggagtttcttctgcttgcccataccagggaaagtcatctttcttcaggaacgttgtttggcttattaagaagaactctacttacccaactattaagaggtcttacaacaacactaaccaggaagatttgcttgttctttggggaattcaccacccaaatgatgctgctgaacagactaagttgtaccagaacccaactacttacatttctgtgggaacttctactcttaaccagaggcttgtgccaagaattgctactaggtctaaggtgaacggacaatctggaaggatggaattcttctggactattcttaagccaaacgatgctattaacttcgagtctaacggaaacttcattgctccagagtacgcttacaagattgtgaagaagggtgatagtactattatgaagtctgagcttgagtacggaaactgcaacactaagtgccaaactccaatgggagctattaactcttctatgccattccacaacattcacccacttactattggagagtgcccaaagtacgtgaagtctaacaggcttgtgcttgctactggacttaggaattctccacagagagaaagaagaagaaagaaaaggggacttttcggagctattgctggattcattgagggaggatggcagggaatggttgatggatggtatggataccatcactctaatgagcagggatctggatatgctgctgacaaagaatctactcagaaagctattgacggtgttactaacaaggtgaactctattattgataagatgaacactcagttcgaagctgttggaagagagttcaacaaccttgagagaaggattgagaaccttaacaagaaaatggaagatggattccttgatgtgtggacttacaacgctgagttgcttgtgcttatggaaaacgagaggactcttgatttccacgattctaacgtgaagaacctttacgacaaagtgaggcttcagcttagggataacgctaaagagcttggaaacggttgcttcgagttctaccacaagtgcgataacgagtgcatggaatctgttaggaacggaacttacgattacccacagtactctgaagaagctaggcttaagagggaagagatttctggtgttaagttggagtctattggtatctaccagattcatcaccatcaccaccacaaggatgagctttgatga

SEQ ID NO: 91 - protein, 556 aa, Artificial Sequence
  Synthetic polypeptide
MGFVLFSQLPSFLLVSTLLLFLVISHSCRADQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQIHHHHHHKDEL

SEQ ID NO: 92 - DNA, 1716 bp, Artificial Sequence
  Synthetic polynucleotide
atgggattcgtgcttttctctcagcttccttctttccttcttgtgtctactcttcttcttttccttgtgatttctcactcttgcagggctatctgcactggaattacttcatctaactctccacacgtggttaagactgctactcagggtgaagttaacgtgactggtgttattccacttactactactccaactaagtctcacttcgctaaccttaagggaactgagactagaggaaagttgtgcccaaagtgccttaactgcactgatcttgatgttgctcttggaaggccaaagtgcactggaaacattccatctgctagggtgtcaattcttcacgaagtgaggccagttacttctggatgcttcccaattatgcacgataggactaagattaggcagcttccaaaccttcttaggggatacgagcacattaggctttctactcacaacgtgattaacgctgagaatgctccaggtggaccatacaagattggaacttcaggatcttgcccaaacgtgactaacggaaacggattcttcgctactatggcttgggctgtgccaaagaacgataacaacaagactgctacaaactctcttactattgaggttccttacatctgtactgagggtgaagatcagattactgtgtggggattccactctgataacgagactcagatggctaagttgtacggtgattctaagccacagaagttcacttcatctgctaacggtgttactactcactacgtgtctcagattggaggattcccaaaccagactgaggatggtggacttccacaatctggaaggattgtggtggattacatggttcagaagtctggaaagactggaactattacttaccagaggggtattcttcttccacagaaagtgtggtgtgcttctggaaggtctaaagtgattaagggatctcttccacttattggagaggctgattgccttcatgagaagtacggtggacttaacaagtctaagccttactacactggtgaacacgctaaggctattggaaactgcccaatttgggttaagactccacttaagttggctaacggaactaagtataggccacctgctaagttgcttaaagagaggggattcttcggagctattgctggatttcttgagggaggatgggagggaatgattgctggatggcacggatatacttctcatggtgctcacggtgttgctgttgctgctgatcttaagtctactcaagaggctattaacaagattactaagaaccttaactctctttctgagcttgaggtgaagaaccttcagagactttctggtgctatggatgagcttcacaacgagattcttgagcttgatgagaaagtggatgatcttagggctgatacaatttcttctcagattgagcttgctgtgcttctttctaacgagggaattattaactctgaggatgagcaccttcttgctcttgagaggaagttgaagaagatgcttggaccatctgctgttgagattggaaacggttgcttcgagactaagcacaagtgcaaccagacttgccttgatagaattgctgctggaactttcgatgctggtgagttctctcttccaactttcgattctcttaacattactgctgcttctcttaacgatgatggacttgataaccacactcatcaccatcaccaccacaaggatgagctttga

SEQ ID NO: 93 - protein, 571 aa, Artificial Sequence
  Synthetic polypeptide
MGFVLFSQLPSFLLVSTLLLFLVISHSCRAICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTHHHHHHKDEL

SEQ ID NO: 94 - DNA, 1674 bp, Artificial Sequence
  Synthetic polynucleotide
atgggattcgtgcttttctctcagcttccttctttccttcttgtgtctactcttcttcttttccttgtgatttctcactcttgccgtgctcaaaagttgccaggaaacgataactctactgctactctttgccttggacatcacgctgttccaaacggaactattgtgaaaactattactaacgatcagattgaggtgacaaacgctactgagcttgttcagtcatcttctactggaggaatttgcgattctccacaccagattcttgatggagagaactgcactcttattgatgctcttcttggagatccacagtgcgatggattccagaacaagaagtgggatcttttcgtggaaaggtctaaggcttactctaactgctacccatacgatgttccagattacgcttctcttaggagtcttgtggcttcttctggaactcttgagttcaacaacgagtctttcaactgggctggagttactcagaacggaacttcttctgcttgtaagaggaggtctaacaagtctttcttctctaggcttaactggcttactcaccttaagtacaagtacccagctcttaacgtgactatgccaaacaacgagaagttcgataagttgtacatttggggagttcaccacccagttactgattctgatcagatttctctttacgctcaggcttctggaaggattactgtgtctactaagaggtctcagcagactgtgattccaaacattggataccgtccaagagtgagggatatttcttctaggatttctatctactggactattgtgaagccaggagatattcttcttattaactctactggaaaccttattgctccaaggggatacttcaagattaggagtggaaagtcatctattatgaggagtgatgctccaattggaaagtgcaactctgagtgcattactccaaacggatctattccaaacgataagccattccagaacgtgaacaggattacttatggagcttgcccaagatacgtgaagcagaacactcttaagttggctactggaatgaggaatgtgccagagaagcagactaggggaattttcggagctattgctggattcattgagaatggatgggagggaatggttgatggatggtacggattcaggcaccagaattcagagggaactggacaagctgctgatcttaagtctactcaggctgctattaaccagattaacggaaagttgaacaggcttattggaaagactaacgagaagttccaccagattgagaaggagttctctgaggttgagggaaggattcaggatcttgagaagtacgtggaggatacaaagattgatctttggtcttacaacgctgagcttcttgttgctcttgagaaccagcacactattgatttgactgattctgagatgaacaagttgttcgagaggactaagaagcagcttagggagaacgctgaggatatgggaaatggatgcttcaaaatctaccacaagtgcgataacgcttgcattgagtctattaggaacggaacttacgatcacgatgtgtaccgtgatgaggctcttaacaacaggttccagattaagggagtggagcttaagtctggatacaaggattggattcttcatcatcaccaccaccacaaggatgagctttgatga

SEQ ID NO: 95 - protein, 556 aa, Artificial Sequence
  Synthetic polypeptide
MGFVLFSQLPSFLLVSTLLLFLVISHSCRAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWAGVTQNGTSSACKRRSNKSFFSRLNWLTHLKYKYPALNVTMPNNEKFDKLYIWGVHHPVTDSDQISLYAQASGRITVSTKRSQQTVIPNIGYRPRVRDISSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIESIRNGTYDHDVYRDEALNNRFQIKGVELKSGYKDWILHHHHHHKDEL

SEQ ID NO: 96 - DNA, 1665 bp, Artificial Sequence
  Synthetic polynucleotide
atgggtttcgtgcttttctctcagcttccttctttccttcttgtgtctacccttcttcttttccttgtgatttctcactcttgcagggctcaaaagttgcctggaaacgataattctaccgctaccctttgccttggtcatcatgctgttcctaacggaaccattgtgaaaaccattaccaacgatcagattgaggtgaccaatgctactgagcttgttcagtcatcttctaccggtgaaatttgcgattctcctcaccagattcttgatggtgaaaactgcacccttattgatgctttgcttggtgatcctcagtgtgatggtttccagaacaagaagtgggatcttttcgttgagaggtctaaggcttactctaactgctacccttacgatgttcctgattacgcttctcttagatcacttgtggcttcatctggaacccttgagttcaacaacgagtctttcaattggactggtgttacccagaacggtacttcttctgcttgcattagaaggtctaacaactctttcttctctaggcttaactggcttacccaccttaagttcaagtaccctgctcttaatgtgaccatgcctaacaacgagaagttcgataagttgtacatttggggagttcatcaccctggtactgataatgatcagattttcccttacgctcaggcttctggaaggattactgtgtctaccaagaggtcacagcagactgtgattcctaacattggttctaggccaagagtgaggaacattccttctaggatttctatctactggaccattgtgaagcctggtgatattcttcttattaactctaccggtaaccttattgctcctaggggatacttcaagattagaagtggaaagtcatctattatgagatcagatgctcctattggaaagtgcaactctgagtgcattacccctaacggttctattcctaacgataagcctttccagaacgtgaacaggattacttatggtgcttgccctagatacgtgaagcagaacacccttaagttggctactggaatgaggaatgtgcctgagaagcagactaggggaattttcggagctattgctggtttcattgagaatggatgggagggaatggttgatggttggtacggtttcaggcatcagaactctgaaggtattggacaggctgctgatcttaagtctacccaggctgctattgatcagattaacggtaagttgaacaggcttattggaaagaccaatgagaagttccaccagattgagaaagagttctctgaggttgagggaaggattcaggatcttgagaagtacgtggaggataccaagattgatctttggtcttacaacgctgagttgcttgtggctcttgagaatcagcacaccattgatcttaccgattctgagatgaacaagttgttcgaaaagaccaagaagcagcttagggagaacgctgaggatatgggtaatggttgcttcaaaatctaccacaagtgcgataacgcttgcattggttctattaggaacggaacctacgatcatgatgtgtacagggatgaggctcttaataacaggttccagattaagggtgttgagcttaagtctggttacaaggatcatcaccatcaccaccacaaggatgagctttgatga

SEQ ID NO: 97 - protein, 553 aa, Artificial Sequence
  Synthetic polypeptide
MGFVLFSQLPSFLLVSTLLLFLVISHSCRAQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDHHHHHHKDEL

SEQ ID NO: 98 - DNA, 1659 bp, Artificial Sequence
  Synthetic polynucleotide
atgggtttcgtgcttttctctcagcttccttctttccttcttgtgtctacccttcttcttttccttgtgatttctcactcttgcagggctgataccatctgcattggttaccacgctaacaactctactgatactgtggataccgtgcttgagaagaatgtgactgtgacccactctgtgaaccttttggagaactctcacaacggtaagttgtgccttcttaagggtattgctcctcttcagcttggaaattgctctgtggctggatggattcttggaaatcctgagtgcgagcttcttatttctaaagagtcttggtcttacattgtggagaagcctaatcctgagaacggtacttgctaccctggtcactttgctgattacgaagagcttagagagcagctttcttctgtttcttctttcgagagattcgagattttccctaaagagtcatcttggcctaatcatactgtgactggtgtgtctgcttcttgctctcataacggtgagtcatctttctacaggaaccttctttggcttaccggaaagaacggtctttaccctaacctttctaagtcttacgctaacaacaaagagaaagaggttttggttctttggggtgttcatcaccctcctaacattggtgatcagaaggctctttaccataccgagaacgcttacgtttctgtggtgtcatctcactactctaggaagttcacccctgagattgctaagaggcctaaagtgagggatcaagagggaaggattaactactactggacccttcttgaacctggtgataccattattttcgaggctaacggtaaccttattgctcctagatacgctttcgctctttctagaggtttcggttctggtattattaactctaacgctcctatggataagtgtgatgctaagtgccagactcctcagggtgctattaactcttctcttcctttccagaatgtgcaccctgttactattggtgagtgccctaagtatgtgagatcagctaagttgaggatggtgaccggtcttaggaacattccttctattcagtctaggggacttttcggagctattgctggttttattgagggaggatggactggaatggttgatggttggtacggttaccatcatcagaatgagcagggttctggttatgctgctgatcagaagtctacccagaacgctattaacggtattaccaacaaggtgaactctgtgattgagaagatgaacacccagttcactgctgttggaaaagagttcaacaagttggagagaaggatggaaaaccttaacaagaaagtggatgatggtttcattgatatttggacctacaacgctgagttgcttgtgcttcttgagaatgagaggacccttgatttccacgattctaacgtgaagaacctttacgagaaggtgaagtctcagcttaagaacaacgctaaagagattggaaacggttgcttcgagttctaccacaagtgcaacgatgagtgcatggaatctgtgaagaacggaacctacgattaccctaagtactctgaagagtctaagttgaacagagaaaagattgatggtgttaagttggagtctatgggagtgtaccagattcatcaccatcaccaccacaaggatgagctttga

SEQ ID NO: 99 - protein, 552 aa, Artificial Sequence
  Synthetic polypeptide
MGFVLFSQLPSFLLVSTLLLFLVISHSCRADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQIHHHHHHKDEL

SEQ ID NO: 100 - DNA, 1719 bp, Artificial Sequence
  Synthetic polynucleotide
atgggtttcgtgcttttctctcagcttccttctttccttcttgtgtctacccttcttcttttccttgtgatttctcactcttgcagggctgatagaatctgcaccggtattacctcttctaactctcctcacgtggttaagactgctactcagggtgaagttaatgtgaccggtgttattcctcttactaccacccctaccaagtcttacttcgctaaccttaagggtactaagactagaggaaagttgtgccctgattgccttaattgcaccgatcttgatgttgctcttggaaggcctatgtgtgttggtactaccccttctgctaaggcttctattcttcacgaagtgagacctgttacttctggttgcttccctattatgcacgataggaccaagattaggcagcttgctaaccttcttaggggttacgagaacattaggctttctacccagaacgtgattgatgctgaaaaggctcctggtggtccttataggcttggaacctctggttcttgccctaatgctacctctaagtctggtttcttcgctactatggcttgggctgtgcctaaggataacaacaagaacgctaccaatcctcttactgtggaggtgccatatatctgtaccgagggtgaagatcagattactgtgtggggtttccactctgatgataagacccagatgaagaacctttacggtgattctaaccctcagaagttcacctcttctgctaatggtgttaccacccactacgtgtctcagattggtggtttccctgatcaaactgaggatggtggacttcctcagtctggaaggattgtggtggattacatgatgcaaaagcctggaaagaccggaactattgtgtatcagaggggagttcttcttcctcagaaagtgtggtgtgcttctggtaggtctaaagtgattaagggttctcttcctcttattggagaggctgattgccttcatgagaagtacggtggtcttaacaagtctaagccttactacactggtgaacacgctaaggctattggaaactgccctatttgggttaagacccctcttaagttggctaacggtactaagtataggcctcctgctaagttgcttaaagagaggggattcttcggagctattgctggttttcttgagggaggatgggagggaatgattgctggatggcacggttatacttctcatggtgctcacggtgttgctgttgctgctgatcttaagtctacccaggaagctattaacaagattaccaagaaccttaactctctttctgagcttgaggtgaagaaccttcagagactttctggtgctatggatgagcttcacaacgagattcttgagcttgatgagaaagtggatgatcttagggctgataccatttcttctcagattgagcttgctgtgcttctttctaacgagggtatcattaactctgaggatgagcaccttcttgctcttgagaggaagttgaagaagatgcttggtccttctgctgtggatattggaaatggttgcttcgagactaagcacaagtgcaatcagacttgccttgataggattgctgctggaactttcaatgctggtgagttctctcttcctaccttcgattctcttaacattaccgctgcttctcttaacgatgatggtcttgataatcacactcatcaccatcaccaccacaaggatgagctttga

SEQ ID NO: 101 - protein, 572 aa, Artificial Sequence
  Synthetic polypeptide
MGFVLFSQLPSFLLVSTLLLFLVISHSCRADRICTGITSSNSPHVVKTATQGEVNVTGVIPLTTTPTKSYFANLKGTKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTHHHHHHKDEL

SEQ ID NO: 102 - DNA, 1719 bp, Artificial Sequence
  Synthetic polynucleotide
atgggtttcgtgcttttctctcagcttccttctttccttcttgtgtctacccttcttcttttccttgtgatttctcactcttgcagggctgatagaatctgcaccggtattacctcttctaactctcctcacgtggttaagactgctactcagggtgaagttaatgtgaccggtgttattcctcttactaccacccctaccaagtcttacttcgctaaccttaagggtactaggactagaggaaagttgtgccctgattgccttaattgcaccgatcttgatgttgctcttggaaggcctatgtgtgttggtactaccccttctgctaaggcttctattcttcacgaggtgaagcctgttacttctggttgcttccctattatgcacgataggaccaagattaggcagcttcctaaccttcttaggggttacgagaacattaggctttctacccagaacgtgattgatgctgaaaaggctcctggtggtccttataggcttggaacctctggttcttgccctaatgctacctctaagtctggtttcttcgctactatggcttgggctgtgcctaaggataacaacaagaacgctaccaatcctcttactgtggaggtgccatatatctgtaccgagggtgaagatcagattactgtgtggggtttccactctgatgataagacccagatgaagaacctttacggtgattctaaccctcagaagttcacctcttctgctaatggtgttaccacccactacgtgtctcagattggttctttccctgatcaaactgaggatggtggacttcctcagtctggaaggattgtggtggattacatgatgcaaaagcctggaaagaccggaactattgtgtatcagaggggagttcttcttcctcagaaagtgtggtgtgcttctggtaggtctaaagtgattaagggttctcttcctcttattggagaggctgattgccttcatgagaagtacggtggtcttaacaagtctaagccttactacactggtgaacacgctaaggctattggaaactgccctatttgggttaagacccctcttaagttggctaacggtactaagtataggcctcctgctaagttgcttaaagagaggggattcttcggagctattgctggttttcttgagggaggatgggagggaatgattgctggatggcacggttatacttctcatggtgctcacggtgttgctgttgctgctgatcttaagtctacccaggaagctattaacaagattaccaagaaccttaactctctttctgagcttgaggtgaagaaccttcagagactttctggtgctatggatgagcttcacaacgagattcttgagcttgatgagaaagtggatgatcttagggctgataccatttcttctcagattgagcttgctgtgcttctttctaacgagggtatcattaactctgaggatgagcaccttcttgctcttgagaggaagttgaagaagatgcttggtccttctgctgtggagattggaaatggttgcttcgagactaagcacaagtgcaatcagacttgccttgataggattgctgctggaactttcaatgctggtgagttctctcttcctaccttcgattctcttaacattaccgctgcttctcttaacgatgatggtcttgataatcacactcatcaccatcaccaccacaaggatgagctttga

SEQ ID NO: 103 - protein, 572 aa, Artificial Sequence
  Synthetic polypeptide
MGFVLFSQLPSFLLVSTLLLFLVISHSCRADRICTGITSSNSPHVVKTATQGEVNVTGVIPLTTTPTKSYFANLKGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEFSLPTFDSLNITAASLNDDGLDNHTHHHHHHKDEL

SEQ ID NO: 104 - DNA, 1659 bp, Artificial Sequence
  Synthetic polynucleotide
atgggattcgtgcttttctctcagcttccttctttccttcttgtgtctactcttcttcttttccttgtgatttctcactcttgcagggctgatacaatctgcattggataccacgctaacaactctactgatactgtggatactgttcttgagaagaacgtgactgtgactcactctgtgaaccttttggaggattctcacaacggaaagttgtgccttcttaagggaattgctccacttcaacttggaaactgcagtgtggctggatggattcttggaaatccagagtgcgagcttcttatttctaaagagtcttggtcttacattgtggagactccaaatccagagaacggaacttgttacccaggatacttcgctgattacgaagagcttagagagcagctttcttctgtttcttctttcgagagattcgagattttcccaaaagagtcatcttggccaaaccacactgttactggtgtttctgcttcttgctctcataacggtaagtcatctttctacaggaaccttctttggcttactggaaagaacggactttacccaaacctttctaagtcttacgtgaacaacaaagagaaagaggttttggttctttggggagttcatcacccaccaaacattggaaatcagagggctctttaccatactgagaacgcttacgtgtctgtggtttcttctcactactctagaaggttcactccagagattgctaagaggccaaaagtgagggatcaagagggaaggattaactactactggactcttcttgagccaggtgatacaattattttcgaggctaacggaaaccttattgctccatggtacgcttttgctttgtctaggggattcggatctggaattattacttctaacgctccaatggatgagtgtgatgctaagtgccaaactccacagggtgctattaactcttctcttccattccagaacgttcacccagttactattggagagtgcccaaagtatgtgagatcagctaagttgaggatggtgactggacttaggaacattccatctattcagtctaggggacttttcggagctattgctggattcattgagggaggatggactggaatggttgatggatggtacggataccatcatcagaatgagcagggatctggatatgctgctgatcagaagtctactcagaacgctattaacggaattactaacaaggtgaactctgtgattgagaagatgaacactcagttcactgctgtgggaaaagagttcaacaagttggagagaaggatggaaaaccttaacaagaaagtggatgatggattccttgatatttggacttacaacgctgagttgcttgtgcttcttgagaacgagaggactcttgatttccacgattctaacgtgaagaacctttacgagaaggtgaagtctcagcttaagaacaacgctaaagagattggaaacggttgcttcgagttctaccacaagtgcaacaacgagtgcatggaatctgtgaagaacggtacttacgattacccaaagtactctgaagagtctaagttgaacagagaaaagattgatggtgttaagttggagtctatgggagtgtaccagattcatcaccatcaccaccacaaggatgagctttaa

SEQ ID NO: 105 - protein, 552 aa, Artificial Sequence
  Synthetic polypeptide
MGFVLFSQLPSFLLVSTLLLFLVISHSCRADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQIHHHHHHKDEL

SEQ ID NO: 106 - DNA, 1662 bp, Artificial Sequence
  Synthetic polynucleotide
atgggtttcgtgcttttctctcagcttccttctttccttcttgtgtctacccttcttcttttccttgtgatttctcactcttgcagggctgataccatctgcattggttaccacgctaacaactctactgatactgtggataccgtgcttgagaagaatgtgactgtgacccactctgtgaaccttttggaggattctcacaacggtaagttgtgccttcttaagggtattgctcctcttcagcttggaaattgctctgtggctggatggattcttggaaatcctgagtgcgagcttcttatttctagagagtcttggtcttacattgtggagaagcctaatcctgagaacggtacttgctaccctggtcactttgctgattacgaagagcttagagagcagctttcttctgtttcttctttcgagagattcgagattttccctaaagagtcatcttggcctaaccataccactactggtgtttctgcttcttgctcacacaacggtgagtcatctttctacaagaaccttctttggcttaccggaaagaacggtctttaccctaacctttctaagtcttacgctaacaacaaagagaaagaggttttggttctttggggtgttcatcaccctcctaacattggtgatcagagggctctttaccacaaagagaacgcttacgtttctgtggtgtcatctcactactctaggaagttcacccctgagattgctaagaggcctaaagtgagggatcaagagggaaggattaactactactggacccttcttgaacctggtgataccattattttcgaggctaacggtaaccttattgctcctagatacgctttcgctctttctagaggtttcggttctggtattattaactctaacgctcctatggatgagtgtgatgctaagtgtcagactcctcagggtgctattaactcttctcttcctttccagaatgtgcaccctgttactattggtgagtgccctaagtatgtgagatcagctaagttgaggatggtgaccggtcttaggaacattccttctattcagtctaggggacttttcggagctattgctggttttattgagggaggatggactggaatggttgatggttggtacggttaccatcatcagaatgagcagggttcaggttatgctgctgatcagaagtctacccagaacgctattaacggtattaccaacaaggtgaactctgtgattgagaagatgaacacccagttcactgctgttggaaaagagttcaacaagttggagagaaggatggaaaaccttaacaagaaagtggatgatggtttcattgatatttggacctacaacgctgagttgcttgtgcttcttgagaatgagaggacccttgatttccacgattctaacgtgaagaacctttacgagaaggtgaagtctcagcttaagaacaacgctaaagagattggaaacggttgcttcgagttctaccacaagtgcaacgatgagtgcatggaatctgtgaagaacggaacctacgattaccctaagtactctgaagagtctaagttgaacagagaaaagattgatggtgttaagttggagtctatgggagtgtaccagattcatcaccatcaccaccacaaggatgagctttgatga

SEQ ID NO: 107 - protein, 552 aa, Artificial Sequence
  Synthetic polypeptide
MGFVLFSQLPSFLLVSTLLLFLVISHSCRADTICIGYHANNSTDTVDTVLEKNVTVTHSVNLLEDSHNGKLCLLKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQIHHHHHHKDEL

SEQ ID NO: 108 - DNA, 1662 bp, Artificial Sequence
  Synthetic polynucleotide
atgggattcgtgcttttctctcagcttccttctttccttcttgtgtctactcttcttcttttccttgtgatttctcactcttgcagggctcaaaagttgccaggaaacgataactctactgctactctttgccttggacatcacgctgttccaaacggaactattgtgaaaactattactaacgatcagattgaggtgacaaacgctactgagcttgttcagtcatcttctactggtggaatttgcgattctccacaccagattcttgatggtgaaaactgcactcttattgatgctttgcttggagatccacagtgtgatggattccagaacaagaagtgggatcttttcgttgagaggtctaaggcttactctaactgctacccatacgatgttccagattacgcttctcttagatcacttgtggcttcatctggaactcttgagttcaacgatgagtctttcaactggactggtgttactcagaacggaacttcatcttcatgcaagaggaggtctaacaactctttcttctctaggcttaactggcttactcaccttaagttcaagtacccagctcttaacgtgactatgccaaacaacgagaagttcgataagttgtacatttggggagttcaccacccagttactgataatgatcagattttcctttacgctcaggcttctggaaggattactgtgtctactaagaggtctcagcagactgtgattccaaacattggatctaggccaaggattaggaacattccatctaggatttctatttactggactattgtgaagccaggtgatattcttcttattaactctactggaaaccttattgctccaaggggatacttcaagattagaagtggaaagtcatctattatgagatcagatgctccaattggaaagtgcaactctgagtgcattactccaaacggttctattccaaacgataagccattccagaacgtgaacaggattacttatggtgcttgcccaagatacgtgaagcagaacactcttaagttggctactggaatgaggaatgtgccagagaagcagactaggggaattttcggagctattgctggattcattgagaatggatgggagggaatggttgatggatggtacggattcaggcatcaaaactctgagggaattggacaagctgctgatcttaagtctactcaggctgctattaaccagattaacggaaagttgaacaggcttattggaaagactaatgagaagttccaccagattgagaaagagttctctgaggttgagggaaggattcaggatcttgagaagtacgtggaggatacaaagattgatctttggtcttacaacgctgagttgcttgttgctcttgagaaccagcacactattgatcttactgattctgagatgaacaagttgttcgagaggactaagaagcagcttagggagaacgctgaggatatgggaaatggatgcttcaagatttaccacaagtgcgataacgcttgcattggatctattaggaacggaacttacgatcacgatgtgtacagagatgaggctcttaacaacaggttccagattaagggtgttgagcttaagtctggatacaaggatcatcaccatcaccaccacaaggatgagctttga

SEQ ID NO: 109 - protein, 541 aa, Artificial Sequence
  Synthetic polypeptide
MGFVLFSQLPSFLLVSTLLLFLVISHSCRAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNDESFNWTGVTQNGTSSSCKRRSNNSFFSRLNWLTHLKFKYPALNVTMPNNEKFDKLYIWGVHHPVTDNDQIFLYAQASGRITVSTKRSQQTVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGY

SEQ ID NO: 110 - protein, 469 aa, Influenza A virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 111 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 112 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 113 - protein, 221 aa, Alfalfa mosaic virus
MSSSQKKAGGKAGKPTKRSQNYAALRKAQLPKPPALKVPVVKPTNTILPQTGCVWQSLGTPLSLSSFNGLGARFLYSFLKDFVGPRILEEDLIYRMVFSITPSHAGTFCLTDDVTTEDGRAVAHGNPMQEFPHGAFHANEKFGFELVFTAPTHAGMQNQNFKHSYAVALCLDFDAQPEGSKNPSFRFNEVWVERKAFPRAGPLRSLITVGLLDEADDLDRH

SEQ ID NO: 114 - protein, 268 aa, Tobacco mosaic virus
MALVVKGKVNINEFIDLSKMEKILPSMFTPVKSVMCSKVDKIMVHENESLSEVNLLKGVKLIDSGYVCLAGLVVTGEWNLPDNCRGGVSVCLVDKRMERADEATLGSYYTAAAKKRFQFKVVPNYAITTQDAMKNVWQVLVNIRNVKMSAGFCPLSLEFVSVCIVYRNNIKLGLREKITNVRDGGPMELTEEVVDEFMEDVPMSIRLAKFRSRTGKKSDVRKGKNSSSDRSVPNKNYRNVKDFGGMSFKKNNLIDDDSEATVAESDSF

SEQ ID NO: 115 - protein, 6 aa, Artificial Sequence
  Synthetic 6x His tag“
HHHHHH